SEQUENCE LISTING (2056 sequences)

SEQ ID NO: 1 - protein, 1244 aa, synthetic construct
METFSGFTNLYPLSKTLRFRLIPVGETLKHFIDSGILEEDQHRAESYVKVKAIIDDYHRAYIENSLSGFELPLESTKFNSLEEYYLYHNIRNKTEEIQNLSSKVRTNLRKQVVAQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAIVNFWNTIHDTVLAEGGLKTIIASLGSYGLEGIFLKNDLQLTDISQKATGSWGKISSEIKQKIEVMNPQKKKESYETYQERIDKIFKSYKSFSLAFINECLRGEYKIEDYFLKLGAVNSSSLQKENHFSHILNTYTDVKEVIGLYSESTDTKLIQDNDSIQKIKQFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 2 - protein, 1197 aa, synthetic construct
MLSNFTNQYQLSKTLRFELKPVGDTLKHIEKSGLIAQDEIRSQEYQEVKTIIDKYHKAFIDEALQNVVLSNLEEYEALFFERNRDEKAFEKLQAVLRKEIVAHFKQHPQYKTLFKKELIKADLKNWQELSDAEKELVSHFDNFTTYFTGFHENRANMYTDEAKHSSIAYRIIHENLPIFLTNKKLFETIKQKAPHLAQETQDALLEYLSGAIVEDMFELSYFNHLLSQTHIDLYNQMIGGVKQDSLKIQGLNEKINLYRQANGLSKRELPNLKPLHKQILSDRETLSWLPESFESDEELMQGVQAYFESEVLAFECCDGKVNLLEKLPELLHQTQDYDFSKVYFKNDLALTAASQAIFKDYRIIKEALWEVNKPKKSKDLVADEEKFFNKKNSYFSIEQIDGALNSAQLSANMMHYFQSESTKVIEQIQLTYNDWKRNSSNKELLKAFLDALLSYQRLLKPLNAPNDLEKDVAFYAYFDAYFTSLCGVVKLYDKVRNFMTKKPYSLEKFKLNFENSTLLDGWDVNKESDNTAILFRKEGLYYLGIMNKKYNKVFRNISSSQDEGYQKIDYKLLPGANKMLPKVFFSDKNKEYFKPNAKLLERYKAGEHKKGDNFDLDFCHELIDFFKTSIEKHQDWKHFAYQFSPTESYEDLSGFYREVEQQGYKISYKNIAASFIDTLVAEGKLYFFQIYNKDFSPYSKGTPNMHTLYWRALFDEKNLADVIYKLNGQAEIFFRKKSIEYSQEKLQKGHHHEMLKDKFAYPIIKDRRFAFDKFQFHVPITLNFKAEGNENITPKTFEYIRSNPDNIKVIGIDRGERHLLYLSLIDAEGKIVEQFTLNQIINSYNGKDHVIDYHAKLDAKEKDRDKARKEWGTVENIKELKEGYLSHVIHKIATLIIEHGAVVAMEDLNFGFKRGRFKVEKQVYQKFEKALIDKLNYLVDKKKEPHKLGGLLNALQLTSKFQSFEKMGKQNGFLFYVPAWNTSKIDPVTGFVNLFDTRYASVEKSKAFFTKFQSICYNEAKDYFELVFDYNDFTEKAKETRSEWTLCTYGERIVSFRNAEKNHQWDSKTIHLTTEFKNLFGELHGNDVKEYILEQNSVEFFKSLIYLLKITLQMRNSITGTDIDYLVSPVADEAGNFYDSRKADTSLPKDADANGAYNIARKGLMLMHRIQNAEDLKKVNLAISNRDWLRNAQGLDK

SEQ ID NO: 3 - protein, 1252 aa, synthetic construct
MIDLKQFIGIYPVSKTLRFELRPVGKTQEWIEKNRVLEGDEQKAADYPVVKKLIDDYHKVCIHDSLNHVHFDWEPLKDAIEIFQKTKSDEAKKRLEAEQAMMRKKIAAAIKDFKHFKELTAATPSDLITSVLPEFSDDGSLKSFRGFATYFSGFQENRNNIYSQEAISTGVPYRLVHDNFPKFLSDLEVFERIKSTCPEVINQASAELQPFLEGVMIDDIFSLDFYNSLLTQNGIDFFNQVIGGVSEKDKQKYRGINEFSNLYRQQHKEIAASKKAMTMIPLFKQILSDRDTLSYIPAQIRTEDELVSSITQFYDHITHFEHDGKTINVLSEIVALLGKLDTYDPNGICITARKLTDISQKVYGKWSVIEEKMKEKAIQQYGDISVAKNKKKVDAFLSRKAYSLSDLCFDEEISFSRYYSELPQTLNAISGYWLQFNEWCKSDEKQKFLNNQTGTEVVKSLLDAMMELFHKCSVLVMPEEYEVDKSFYNEFLPLYEELDTLFLLYNKVRNYLTQKPSDVKKFKLNFESPSLASGWDQNKEMKNNAILLFKDGKSYLGVLNAKNKAKIKDAKGDVSSSSYKKMIYKLLSDPSKDLPHKIFAKGNLDFYKPSEYILEGRELGKYKKGPNFDKKFLHDFIDFYKAAISIDPDWSKFNFQYSPTESYDDIGMFFSEIKKQAYKIRFTDISEAQVNEWVDNGQLYLFQLYNKDYAEGAHGRKNLHTLYWENLFTDENLSNLVLKLNGQAELFCRPQSIKKPVSHKIGSKMLNRRDKSGMPIPESIYRSLYQYYNGKKKESELTVAEKQYIDQVIVKDVTHEIIKDRRYTRQEYFFHVPLTFNANADGNEYINEHVLNYLKDNPDVNIIGIDRGERHLIYLTLINQRGEILKQKTFNVVNSYNYQAKLEQREKERDEARKSWDSVGKIKDLKEGFLSAVIHEITNMMIENNAIVVLEDLNFGFKRGRFKVERQVYQKFEKMLIDKLNYLSFKDREAGEEGGILRGYQMAQKFISFQRLGKQSGFLFYIPAAYTSKIDPVSGFVNHFNFSDITNAEKRKDFLMKMDRIEMKNGNIEFTFDYRKFKTFQTDYQNVWTVSTFGKRIVMRIDEKGYKKMVDYEPTNDIIKAFKNKGILLSEGSDLKALIAEIEANATNAGFYSTLLYAFQKTLQMRNSNAVTEEDYILSPVAKDGHQFCSTDEANKGKDAQGNWVSKLPVDADANGAYHIALKGLYLLRNPETKKIENEKWLQFMVEKPYLE

SEQ ID NO: 4 - protein, 1287 aa, synthetic construct
MSYNREKMEEKELGKNQNFQEFIGVSPLQKTLRNELIPTETTKKNIAQLDLLTEDEVRAQNREKLKEMMDDYYRDVIDSTLRGELLIDWSYLFSCMRNHLSENSKESKRELERTQDSVRSQIHDKFAERADFKDMFGASIITKLLPTYIKQNSKYSERYDESVKIMKLYGKFTTSLTDYFETRKNIFSKEKISSAVGYRIVEENAEIFLQNQNAYDRICKIAGLDLHGLDNEITAYVDGKTLKEVCSDEGFAKVITQGGIDRYNEAIGAVNQYMNLLCQKNKALKPGQFKMKRLHKQILCKGTTSFDIPKKFENDKQVYDAVNSFTEIVTKNNDLKRLLNITQNANDYDMNKIYVVADAYSMISQFISKKWNLIEECLLDYYSDNLPGKGNAKENKVKKAVKEETYRSVSQLNEVIEKYYVEKTGQSVWKVESYISSLAEMIKLELCHEIDNDEKHNLIEDDEKISEIKELLDMYMDVFHIIKVFRVNEVLNFDETFYSEMDEIYQDMQEIVPLYNHVRNYVTQKPYKQEKYRLYFHTPTLANGWSKSKEYDNNAIILVREDKYYLGILNAKKKPSKEIMAGKEDCSEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAEQEDLHVIGIDRGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNNDGEFFDSDEYNSYIDAQKAPLPIDADANGAFCIALKGMYTANQIKENWVEGEKLPADCLKIEHASWLAFMQGERG

SEQ ID NO: 5 - protein, 1295 aa, synthetic construct
MSAVFKIKESTMKDFTHQYSLSKTLRFELKPVGETAERIEDFKNQGLKSIVEEDRQRAEDYKKMKRILDDYHKEFIEEVLNDDIFTANEMESAFEVYRKYMASKNDDKLKKEITEIFTDLRKKIAKAFENKSKEYCLYKGDFSKLINEKKTGKDKGPGKLWYWLKAKADAGVNEFGDGQTFEQAEEALAKFNNFSTYFTGFNQNRDNIYTDAEQQTAISYRVINENMTRYFDNCIRYSSIENKYPELVKQLEPLSGKFAPGNYKDYLSQTAIDIYNEAVGHKSDDINAKGINQFINEYRQRNSIKGRELPIMSVLYKQILSDINKDLIIDKFENAGELLDAVKTLHRELTDKKILLKIKQTLNEFLTEDNSEDIYIKSGTDLTAVSNAIWGEWSVIPKALEMYAENITDMNAKAREKWLKREAYHLKTVQEAIEAYLKDNEEFETRNISEYFTNFKSGENDLIQVVQSAYAKMESIFGIEDFHKDRRPVTESGEPGEGFRQVELVREYLDSLINVEHFIKPLHMFRSGKPIELEDCNSNFYDPLNEAYKELDVVFGIYNKVRNYVTQKPYSKDKFKINFQNSTLLDGWDVNKESANSSVLLLKNGKYYLGVMKQGASNILNYRPEPSDSKNKINAKKQLSEIALAGATDDYYEKMIYKLLPDPAKMLPKVFFSAKNIEFYNPSQEIIYIRENGLFKKDAGDKESLKKWIGFMKTSLLKHPEWGSYFNFEFEPAEDYQDISIFYKQVAEQGYSVTFDKIKTSYIEEKVASGELYLFEIYNKDFSPHSKGRPNLHTMYWKSLFEKENLQNLVTKLNGEAEVFFRQHSIKRNEKVVHRANRPIQNKNPLTEKKQSIFEYDLVKDRRFTKDKFFLHCPITLNFKEAGPGRFNDKVNKYIAGNPDIRIIGIDRGERHLLYYSLIDQSGRIVEQGTLNQITSTLNSGGREIPKTTDYRGLLDTKEKERDKARKSWSMIENIKELKSGYLSHIVHKLAKLMVKNNAVVVLEDLNFGFKRGRFKVEKQVYQKFEKALIEKLNYLVFKDARPAEPGHYLNAYQLTAPLESFKKLGKQSGFIYYVPAWNTSKIDPVTGFVNQFYIEKNSMQYLKNFFGKFDSIRFNPDKNYFEFGFDYKNFHNKAAKSKWTICTHGDKRSWYNRKQRKLEIHNVTENLASLLSGKGINFADGGSIKDKILSVDDASFFKSLAFNFKLTAQLRHTFEDNGEEIDCIISPVAAADGTFFCSETAKKLNMELPHDADANGAYNIARKGLMVLRQIRESGKPKPISNADWLDFAQQNED

SEQ ID NO: 6 - protein, 1245 aa, synthetic construct
MQERKKISHLTHRNSVQKTIRMQLNPVGKTMDYFQAKQILENDEKLKENYQKIKEIADRFYRNLNEDVLSKTGLDKLKDYAEIYYHCNTDAERKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVNYYKTSLMQLTDNLSDKYNEAAPLLNKSYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCRDEKYYLAIIDKSNNSILENIDFQDCDENDCYEKIIYKLLPGPNKMLPKVFFSEKCKKLLSPSDEILKIRKNGTFKKGDKFSLDDCHKLIDFYKESFKKYPNWLIYNFKFKNTNEYNDIREFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKKTSTFPYDLIKDKRYTKWQFSLHFPITMNFKAPDRAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEGGGLLHAYQLTNKLESFDKLGMQSGFIFYVRPDFTSKIDPVTGFVNLLYPRYENIDKAKDMISRFDDIGYNAGEDFFEFDIDYDKFPKTASDYRKRWTICTNGERIEAFRNPAKNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVGPVIHNDKWLKFVQENDMANN

SEQ ID NO: 7 - protein, 1281 aa, synthetic construct
MNILKENYMKEIKELTGLYSLTKTIGVELKPVGKTQELIEAKKLIEQDDQRAEDYKIVKDIIDRYHKDFIDKCLNCVKIKKDDLEKYVSLAENSNRDAEDFDKIKTKMRNQITEAFRKNSLFTNLFKKNLIKEYLPAFVSEEEKSVVNKFSKFTTYFDAFNDNRKNLYSGDAKSGTIAYRLIHENLPMFLDNIASFNAISGIGVNEYFSSIETEFTDTLEGKRLTEFFQIDFFNNTLTQKKIGNYNYIVGAVNKAVNLYKQQHKTVRVPLLKPLYKMILSDRVTPSWLPERFESDEEMLTAIKAAYESLREVLVGDNDESLRNLLLNIEHYDLEHIYIANDSGLTSISQKIFGCYDTYTLAIKDQLQRDYPATKKQREAPDLYDERIDKLYKKVGSFSIAYLNRLVDAKGHFTINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 8 - protein, 1293 aa, synthetic construct
MAKENIFNELTGKYQLSKTLRLELKPVGNTQQMLKDEDVFEKDRIIREKYRETRPHFDRLHREFIEQALKNQKLSDLGKYFQCLAKLQNNKKDKEAQEEFKRISQNLRKEVNDLFKIDPLFGEGVFALLKEKYGEKDDAFLREQDGQYVLDENKKKISIFDSWKGFTGYFTKFQETRKNFYKDDGTATAVATRIIDQNLKRFCENIQIFKSIQKKVDFKEVEDNFSVDLEDIFSLGFYSSCFLQEGIDVYNKILGGEPKTTGEKLRGLNELINRYRQDHKGEKLPFFKMLDKQILSEKEKFIESIEDDEELLKTLKEFYSSAEEKTTVLKELFNDFIKNNENYDLSEIYISREALNTISHRWVSAATLPEFEKSVYEVMKKDKPSGLSFDKDDNSYKFPDFIALSYIKGSFEKLSGEKLWKDGYFRDETRNGDKGFLIGNESLWTQFIKIFEFEFNSLFEAKNTERSVGYYHFKKDFEKIITNDFSVNPEDKVIIREFADNVLAIYQMAKYFAIEKKRKWMDQYDTGDFYNHPDFGYKTKFYDNAYEKIVKARMLLQSYLTKKPFSTDKWKLNFECGYLLNGWSSSFNTYGSLLFRTGNEYYLGVVNGSALRTEKIKRLTGNITEANSCHKMVYDFQKPDNKNVPRIFIRSKGDKFAPAVSELNLPVDSILEIYDKGLFKTENKNSPFFKPSLKKLIDYFKLGFSRHASYKHYQFKWKDSSEYKNISEFYNDTIRSCYQIKWEELNFEEVKKLTNSKDLFLFQIYNKDFSEKSTGNKNLHSIYFDGLFLDNNINAQDGVILKLSGGGEIFFRPKTDVKKLGSRTDTKGKLVIKNKRYSQDKIFLHFPIELNYSNTQESNFNKLVRNFLADNPDINIIGVDRGEKHLIYYAGIDQKGNTLKDKDDKDVLGSLNEINGVNYYKLLEERAKAREKARQDWQNIQGIKDLKMGYISLVVRKLADLIIEYNAILVLEDLNMRFKQIHGGIEKSVYQQLEKALIEKLNFLVNKGEKDPERAGHLLRAYQLTAPFSTFKDMGKQTGVLFYTQASYTSKTCPQCGFRPNIKLHFDNLENAKKMLEKINIVYKDNHFEIGYKVSDFTKTEKTSRGNILYGDRQGKDTFVISSKAAIRYKWFARNIKNNELNRGESLKEHTEKGVTIQYDITECLKILYEKNGIDHSGDITKQSIRSELPAKFYKDLLFYLYLLTNTRSSISGTEIDYINCPDCGFHSEKGFNGCIFNGDANGAYNIARKGMLILKKINQYKDQHHTMDKMGWGDLFIGIEEWDKYTQVVSRS

SEQ ID NO: 9 - protein, 1273 aa, synthetic construct
MKEIKELTGLYSLTKTIGVELKPVGKTQELIEAKKLIEQDDQRAEDYKIVKDIIDRYHKDFIDKCLNCVKIKKDDLEKYVSLAENSNRDAEDFDKIKTKMRNQITEAFRKNSLFTNLFKKNLIKEYLPAFVSEEEKSVVNKFSKFTTYFDAFNDNRKNLYSGDAKSGTIAYRLIHENLPMFLDNIASFNAISGIGVNEYFSSIETEFTDTLEGKRLTEFFQIDFFNNTLTQKKIGNYNYIVGAVNKAVNLYKQQHKTVRVPLLKPLYKMILSDRVTPSWLPERFESDEEMLTAIKAAYESLREVLVGDNDESLRNLLLNIEHYDLEHIYIANDSGLTSISQKIFGCYDTYTLAIKDQLQRDYPATKKQREAPDLYDERIDKLYKKVGSFSIAYLNRLVDAKGHFTINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 10 - protein, 1300 aa, synthetic construct
MNFQPFFQKFVHLYPISKTLRFELIPQGATQKFISEKQVLLQDEIRARKYPEMKQAIDGYHKDFIQRALSNIDSQVFEQALNTFEDLFLRSQAERATDAYKKDFETAQTKLRELIVHSFEKGEFKQEYKSLFDKNLITNLLKPWVEQQNQIGDSNYTYHEDFNKFTTYFLGFHENRKNIYSKDPHKTALAYRLIHENLPKFLENNKILLKIQNDHPSLWEQLQTLNQTMPQLFDGWDFSQLMQVSFFSNTLTQTGIDQYNTIIGGISEGENRQKIQGINELINLYNQKQDKKNRVAKLKQLYKQILSDRSTLSFLPEKFVDDTELYHAINMFYLEHLHHQSMINGHSYTLLERVQLLINELANYDLSKVYLAPNQLSTVSHQMFGDFGYIGRALNYYYMQVIQPDYEQLLASAKTTKKIEATEKLKTIFLDTPQSLVVIQAAIDEYIQLQPSTKPHTQLTDFIISLLKQYETVADDQSIKVINVFSDIEGKYSCIKGLVNTKSESKREVLQDEKLATDIKAFMDAVNNVIKLLKPFSLNEKLVASVEKDARFYSDFEEIYQSLLIFVPLYNKVRNYITQKPYSTEKFKLNFNKPTLLSGWDANKEADNLSILLRKNGNYYLAIMDTAKGANKAFEPKTLNQLKVDDTTDCYEKMVYKLLSGPSKMFPKAFKAKNNEGNYYPTPELLTSYNNNEHLKNDKNFTLASLHAYIDWCKEYINRNPSWHQFNFKFSPTQSFQDISQFYSEVSSQSYKVHFQTIPSDYIDQLVAEGKLYLFQIYNKDFSPNAKGKENLHTLYFKALFSDENLKQPVFKLSGEAEMFYRPASLQLANTTIHKAGEPMAAKNPLTPNATRTLAYDIIKDRRFTTDKYLLHVPISLNFHAQESMSIKKHNDLVRQMIKHNHQDLHVIGIDRGEKHLLYVSVIDLKGNIVYQESLNSIKSEAQNFETPYHQLLQHREEGRAQARTAWGKIENIKELKDGYLSQVVHRIQQLILKYNAIVMLEDLNFGFKRGRFKIEKQIYQKFEKALIHKLNYVVDKSTQADELGGVRKAYQLTAPFESFEKLGKQSGVLFYVPAWNTSKIDPVTGFVDLLKPKYENLDKAQAFFNAFDSIHYNAQKNYFEFKVNLKQFAGLKAQAAQAEWTICSYGDERHVYQKKNAQQGETVIVNVTEELKVLFAKNNIEVAQSVELKETICTQTQVDFFKRLMWLLQVLLALRYSSSKDKLDYILSPVANAQGEFFDSRHASVQLPQDSDANGAYHIALKGLWVIEQLKAADNLDKVKLAISNDDWLHFAQQKPYLA

SEQ ID NO: 11 - protein, 1205 aa, synthetic construct
MYYQGLTKLYPISKTIRNELIPVGKTLEHIRMNNILEADIQRKSDYERVKKLMDDYHKQLINESLQDVHLSYVEEAADLYLNASKDKDIVDKFSKCQDKLRKEIVNLLKSHENFPKIGNKEIIKLLQSLSDTEKDYNALDSFSKFYTYFTSYNEVRKNLYSDEEKSSTAAYRLINENLPKFLDNIKAYSIAKSAGVRAKELTEEEQDCLFMTETFERTLTQDGIDNYNELIGKLNFAINLYNQQNNKLKGFRKVPKMKELYKQILSEREASFVDEFVDDEALLTNVESFSAHIKEFLESDSLSRFAEVLEESGGEMVYIKNDTSKTTFSNIVFGSWNVIDERLAEEYDSANSKKKKDEKYYDKRHKELKKNKSYSVEKIVSLSTETEDVIGKYIEKLQADIIAIKETREVFEKVVLKEHDKNKSLRKNTKAIEAIKSFLDTIKDFERDIKLISGSEHEMEKNLAVYAEQENILSSIRNVDSLYNMSRNYLTQKPFSTEKFKLNFNRATLLNGWDKNKETDNLGILLVKEGKYYLGIMNTKANKSFVNPPKPKTDNVYHKVNYKLLPGPNKMLPKVFFAKSNLEYYKPSEDLLAKYQAGTHKKGENFSLEDCHSLISFFKDSLEKHPDWSEFGFKFSDTKKYDDLSGFYREVEKQGYKITYTDIDVEYIDSLVEKDELYLFQIYNKDFSPYSKGNYNLHTLYLTMLFDERNLRNVVYKLNGEAEVFYRPASIGKDELIIHKSGEEIKNKNPKRAIDKPTSTFEYDIVKDRRYTKDKFMLHIPVTMNFGVDETRRFNEVVNDAIRGDDKVRVIGIDRGERNLLYVVVVDSDGTILEQISLNSIINNEYSIETDYHKLLDEKEGDRDRARKNWTTIENIKELKEGYLSQVVNVIAKLVLKYDAIICLEDLNFGFKRGRQKVEKQVYQKFEKMLIDKLNYLVIDKSRSQENPEEVGHVLNALQLTSKFTSFKELGKQTGIIYYVPAYLTSKIDPTTGFANLFYVKYESVEKSKDFFNRFDSICFNKVAGYFEFSFDYKNFTDRACGMRSKWKVCTNGERIIKYRNEEKNSSFDDKVIVLTEEFKKLFNEYGIAFNDCMDLTDAINAIDDASFFRKLTKLFQQTLQMRNSSADGSRDYIISPVENDNGEFFNSEKCDKSKPKDADANGAFNIARKGLWVLEQLYNSSSGEKLNLAMTNAEWLEYAQQHTI

SEQ ID NO: 12 - protein, 1306 aa, synthetic construct
MAKNFEDFKRLYPLSKTLRFEAKPIGATLDNIVKSGLLEEDEHRAASYVKVKKLIDEYHKVFIDRVLDNGCLPLDDKGDNNSLAEYYESYVSKAQDEDAIKKFKEIQQNLLSIIAKKLTDDKAYANLFGNKLIESYKDKADKTKLIDSDLIQFINTAESTQLVSMSQDEAKELVKEFWGFTTYFEGFFKNRKNMYTPEEKSTGIAYRLINENLPKFIDNMEAFKKAIARPEIQANMEELYSNFSEYLNVESIQEMFLLDYYNMLLTQKQIDVYNAIIGGKTDDEHDVKIKGINEYINLYNQQHKDDKLPKLKALFKQILSDRNAISWLPEEFNSDQEVLNAIKDCYERLAENVLGDKVLKSLLGSLADYSLDGIFIRNDLQLTDISQKMFGNWGVIQNAIMQNIKHVAPARKHKESEEDYEKRIAGIFKKADSFSISYINDCLNEADPNNAYFVENYFATFGAVNTPTMQRENLFALVQNAYTEVAALLHSDYPTVKHLAQDKANVSKIKALLDAIKSLQHFVKPLLGKGDESDKDERFYGELASLWAELDTVTPLYNMIRNYMTRKPYSQKKIKLNFENPQLLGGWDANKEKDYATIILRRNGLYYLAIMDKDSRKLLGKAMPSDGECYEKMVYKFFKDVTTMIPKCSTQLKDVQAYFKVNTDDYVLNSKAFNRPLTITKEVFDLNNVLYGKYKKFQKGYLTATGDNVGYTHAVNVWIKFCMDFLDSYDSTCIYDFSSLKPESYLSLDSFYQDVNLLLYKLSFTDVSASFIDQLVEEGKMYLFQIYNKDFSEYSKGTPNMHTLYWKALFDERNLADVVYKLNGQAEMFYRKKSIENTHPTHPANHPILNKNKDNKKKESLFEYDLIKDRRYTVDKFMFHVPITMNFKSSGSENINQDVKAYLRHADDMHIIGIDRGERHLLYLVVIDLQGNIKEQFSLNEIVNDYNGNTYHTNYHDLLDVREDERLKARQSWQTIENIKELKEGYLSQVIHKITQLMVRYHAIVVLEDLSKGFMRSRQKVEKQVYQKFEKMLIDKLNYLVDKKTDVSTPGGLLNAYQLTCKSDSSQKLGKQSGFLFYIPAWNTSKIDPVTGFVNLLDTHSLNSKEKIKAFFSKFDAIRYNKDKKWFEFNLDYDKFGKKAEDTRTKWTLCTRGMRIDTFRNKEKNSQWDNQEVDLTTEMKSLLEHYYIDIHGNLKDAISTQTDKAFFTGLLHILKLTLQMRNSITGTETDYLVSPVADENGIFYDSRSCGDQLPENADANGAYNIARKGLMLVEQIKDAEDLDNVKFDISNKAWLNFAQQKPYKNG

SEQ ID NO: 13 - protein, 1306 aa, synthetic construct
MAKNFEDFKRLYSLSKTLRFEAKPIGATLDNIVKSGLLDEDEHRAASYVKVKKLIDEYHKVFIDRVLDDGCLPLENKGNNNSLAEYYESYVSRAQDEDAKKKFKEIQQNLRSVIAKKLTEDKAYANLFGNKLIESYKDKEDKKKIIDSDLIQFINTAESTQLDSMSQDEAKELVKEFWGFVTYFYGFFDNRKNMYTAEEKSTGIAYRLVNENLPKFIDNIEAFNRAITRPEIQENMGVLYSDFSEYLNVESIQEMFQLDYYNMLLTQKQIDVYNAIIGGKTDDEHDVKIKGINEYINLYNQQHKDDKLPKLKALFKQILSDRNAISWLPEEFNSDQEVLNAIKDCYERLAENVLGDKVLKSLLGSLADYSLDGIFIRNDLQLTDISQKMFGNWGVIQNAIMQNIKRVAPARKHKESEEDYEKRIAGIFKKADSFSISYINDCLNEADPNNAYFVENYFATFGAVNTPTMQRENLFALVQNAYTEVAALLHSDYPTVKHLAQDKANVSKIKALLDAIKSLQHFVKPLLGKGDESDKDERFYGELASLWAELDTVTPLYNMIRNYMTRKPYSQKKIKLNFENPQLLGGWDANKEKDYATIILRRNGLYYLAIMDKDSRKLLGKAMPSDGECYEKMVYKFFKDVTTMIPKCSTQLKDVQAYFKVNTDDYVLNSKAFNKPLTITKEVFDLNNVLYGKYKKFQKGYLTATGDNVGYTHAVNVWIKFCMDFLNSYDSTCIYDFSSLKPESYLSLDAFYQDANLLLYKLSFARASVSYINQLVEEGKMYLFQIYNKDFSEYSKGTPNMHTLYWKALFDERNLADVVYKLNGQAEMFYRKKSIENTHPTHPANHPILNKNKDNKKKESLFDYDLIKDRRYTVDKFMFHVPITMNFKSVGSENINQDVKAYLRHADDMHIIGIDRGERHLLYLVVIDLQGNIKEQYSLNEIVNEYNGNTYHTNYHDLLDVREEERLKARQSWQTIENIKELKEGYLSQVIHKITQLMVRYHAIVVLEDLSKGFMRSRQKVEKQVYQKFEKMLIDKLNYLVDKKTDVSTPGGLLNAYQLTCKSDSSQKLGKQSGFLFYIPAWNTSKIDPVTGFVNLLDTHSLNSKEKIKAFFSKFDAIRYNKDKKWFEFNLDYDKFGKKAEDTRTKWTLCTRGMRIDTFRNKEKNSQWDNQEVDLTTEMKSLLEHYYIDIHGNLKDAISAQTDKAFFTGLLHILKLTLQMRNSITGTETDYLVSPVADENGIFYDSRSCGNQLPENADANGAYNIARKGLMLIEQIKNAEDLNNVKFDISNKAWLNFAQQKPYKNG

SEQ ID NO: 14 - protein, 1274 aa, synthetic construct
MAKNFEDFKRLYSLSKTLRFEAKPIGATLDNIVKSDLLDEDEHRAASYVKVKKLIDEYHKVFIDRVLDDGCLPLENKGNNNSLAEYYESYVSRAQDEDAKKKFKEIQQNLRSVIAKKLTEDKAYANLFGNKLIESYKDKEDKKKIIDSDLIQFINTAESTQLDSMSQDEAKELVKEFWGFVTYFYGFFDNRKNMYTAEEKSTGIAYRLVNENLPKFIDNIEAFNRAITRPEIQENMGVLYSDFSEYLNVESIQEMFQLDYYNMLLTQKQIDVYNAIIGGKTDDEHDVKIKGINDYINLYNQKHKDDKLPKLKALFKQILSDRNAISWLPEEFNSDQEVLNAIKDCYERLSENVLGDKVLKSMLGSLADYSLDGIFIRNDLQLTDISQKMFGNWSVIQNAIMQNIKHVAPARKHKESEEEYENRIAGIFKKADSFSISYIDACLNETDPNNAYFVENYFATLGAVDTPTMQRENLFALVQNAYTEITALLHSDYPTEKNLAQDKANVAKIKALLDAIKSLQHFVKPLLGKGDESDKDERFYGELASLWAELDTMTPLYNMIRNYMTRKPYSQKKIKLNFENPQLLGGWDANKEKDYATIILRRNGLYYLAIMNKDSKKLLGKAMPSDGECYEKMVYKLLPGANKMLPKVFFAKSRMEDFKPSKELVEKYYNGTHKKGKNFNIQDCHNLIDYFKQSIDKHEDWSKFGFKFSDTSTYEDLSGFYREVEQQGYKLSFARVSVSYINQLVEEGKMYLFQIYNKDFSEYSKGTPNMHTLYWKALFDERNLADVVYKLNGQAEMFYRKKSIENTHPTHPANHPILNKNKDNKKKESLFGYDLIKDRRYTVDKFLFHVPITMNFKSSGSENINQDVKAYLRHADDMHIIGIDRGERHLLYLVVIDLQGNIKEQFSLNEIVNDYNGNTYHTNYHDLLDVREDERLKARQSWQTIENIKELKEGYLSQVIHKITQLMVKYHAIVVLEDLNMGFMRGRQKVEKQVYQKFEKMLIEKLNYLVDKKADASVSGGLLNAYQLTSKFDSFQKLGKQSGFLFYIPAWNTSKIDPVTGFVNLLDTRYQNVEKAKSFFSKFDAIRYNKDKEWFEFNLDYDKFGKKAEGTRTKWTLCTRGMRIDTFRNKEKNSQWDNQEVDLTAEMKSLLEHYYIDIHSNLKDAISAQTDKAFFTGLLHILKLTLQMRNSITGTETDYLVSPVVDENGIFYDSRSCGDELPENADANGAYNIARKGLMMIEQIKDAKDLDNLKFDISNKAWLNFAQQKPYKNG

SEQ ID NO: 15 - protein, 1251 aa, synthetic construct
MLFQDFTHLYPLSKTVRFELKPIGRTLEHIHAKNFLSQDETMADMYQKVKVILDDYHRDFIADMMGEVKLTKLAEFYDVYLKFRKNPKDDELQKQLKDLQAVLRKESVKPIGNGGKYKAGHDRLFGAKLFKDGKELGDLAKFVIAQEGKSSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIKHHTARHDDESVQAGKLGQYFKHGLAGVDNPIQKIHNNHSTIKGFLERERPAGERALPKIKSGKNPEMTQLRQLKELLDNALNVAHFAKLLMTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMIYKLLPGPNKMLPRVFFAKSNLDYYNPSAELLDKYAQGTHKKGDNFNLKDCHALIDFFKAGINKHPEWQNFGFKFSPTSSYRDLSDFYREVEPQGYQVKFVDINADYIDELVEQGQLYLFQIYNKDFSPKAHGKPNLHTLYFRALFSEDNLANPIYKLNGEAQIFYRKASLGMNETTIHRAGEILENKNPDNPKERVFTYDIIKDRRYTQDKFMLHVPITMNFGVQGMTIKEFNKKVNQSIRQYDDVNVIGIDRGERHLLYLTVINSKGEILEQRSLNDITTASANGTQMTTPYHKILDKREIERLNARVGWGEIETIKELKSGYLSHVVHQVSQLMLKYNAIVVLEDLNFGFKRGRFKVEKQIYQNFENALIKKLNHLELKDKADDEIGSYKNALQLTNNFTDLKNIGKQTGFLFYVPAWNTSKIDPETGFVDLLKPRYENIAQSQAFFGKFDKICYNADKDYFEFHIDYAKFTDKAKNSRQTWTICSHGDKRYVYDKTANQNKGATKGINVNDELKSLFARYHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 16 - protein, 1251 aa, synthetic construct
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNRIIGEVNSYTNKHNQICHKSERIAKLRPLHKQILSDGMGVSFLPSKFADDSEMCQAVNEFYRHYAHVFAKVQSLFDRFDDYQKDGIYVEHKNLNELSKQAFGDFALLGRVLDGYYVDVVNPEFNDKFAKAKTDNAKEKLTKEKDKFIKGVHSLASLEQAIEHYIAGHDDESVQAGKLGQYFKHGLAGVDNPIQKIHNSHSTIKGFLERERPAGERTLPKIKSDKSLEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMVYKLLPGPNKMLPKVFFAKSNLDYYNPSAELLDKYAQGTHKKGDNFNLKDCHALIDFFKASINKHPEWQHFGFEFSLTSSYQDLSDFYREVEPQGYQVKFVDIDADYIDELVEQGQLYLFQIYNKDFSPKAHGKPNLHTLYFKALFSEDNLANPIYKLNGEAEIFYRKASLDMNETTIHRAGEVLENKNPDNPKERQFVYDIIKDKRYTQDKFMLHVPITMNFGVQGMTIKEFNKKVNQSIQQYDEVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADNEIGSYKNALQLTNNFTDLKSIGKQTGFLFYVPAWNTSKIDPVTGFVDLLKPRYENIAQSQAFFDKFDKICYNADKGYFEFHIDYAKFTDKAKNSRQIWTICSHGDKRYVYDKTANQNKGATIGINVNDELKSLFARYRINDKQPNLVMDICQNNDKEFHKSLTYLLKALLALRYSNASSDEDFILSPVANDKGVFFNSALADDTQPQNADANGAYHIALKGLWLLNELKNSDDLDKVKLAIDNQTWLNFAQNR

SEQ ID NO: 17 - protein, 1251 aa, synthetic construct
MLFQDFTHLYPLSKTVRFELKPIGKTLEHIHAKNFLSQDETMADMYQKVKAILDDYHRDFITKMMSEVTLTKLPEFYEVYLALRKNPKDDTLQKQLTEIQTALREEVVKPIDSGGKYKAGYERLFGAKLFKDGKELGDLAKFVIAQEGESSPKLPQIAHFEKFSTYFTGFHDNRKNMYSSDDKHTAIAYRLIHENLPRFIDNLQILVTIKQKHSVLYDQIVNELNANGLDVSLASHLDGYHKLLTQEGITAYNRIIGEVNSYTNKHNQICHKSERIAKLRPLHKQILSDGMGVSFLPSKFADDSEMCQAVNEFYRHYAHVFAKVQSLFDRFDDYQKDGIYVEHKNLNELSKQAFGDFALLGRVLDGYYVDVVNPEFNDKFAKAKTDNAKEKLTKEKDKFIKGVHSLASLEQAIEHYIAGHDDESVQAGKLGQYFKHGLAGVDNPIQKIHNSHSTIKGFLERERPAGERTLPKIKSDKSLEMTQLRQLKELLDNALNVVHFAKLLTTKTTLDNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMVYKLLPGSNKMLPKVFFAKSNLDYYNPSAELLDKYAQGTHKKGDNFNLKDCHALIDFFKASINKHPEWQHFGFEFSLTSSYQDLSDFYREVEPQGYQVKFVDIDADYIDELVEQGQLYLFQIYNKDFSPKAHGKPNLHTLYFKALFSEDNLANPIYKLNGEAEIFYRKASLDMNETTIHRAGEVLENKNPDNPKERQFVYDIIKDKRYTQDKFMLHVPITMNFGVQGMTIKEFNKKVNQSIQQYDEVNVIGIDRGERHLLYLTVINSKGEILEQRSLNDIITTSANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADNEIGSYKNALQLTNNFTDLKSIGKQTGFLFYVPAWNTSKIDPVTGFVDLLKPRYENIAQSQAFFDKFDKICYNADKGYFEFHIDYAKFTDKAKNSRQIWTICSHGDKRYVYDKTANQNKGATIGINVNDELKSLFARYRINDKQPNLVMDICQNNDKEFHKSLTYLLKALLALRYSNASSDEDFILSPVANDKGVFFNSALADDTQPQNADANGAYHIALKGLWLLNELKNSDDLDKVKLAIDNQTWLNFAQNR

SEQ ID NO: 18 - protein, 1257 aa, synthetic construct
MKYTDFTGIYPVSKTLRFELIPQGSTVENMKREGILNNDMHRADSYKEMKKLIDEYHKVFIERCLSDFSLKYDDTGKHDSLEEYFFYYEQKRNDKTKKIFEDIQVALRKQISKRFTGDTAFKRLFKKELIKEDLPSFVKNDPVKTELIKEFSDFTTYFQEFHKNRKNMYTSDAKSTAIAYRIINENLPKFIDNINAFHIVAKVPEMQEHFKTIADELRSHLQVGDDIDKMFNLQFFNKVLTQSQLAVYNAVIGGKSEGNKKIQGINEYVNLYNQQHKKARLPMLKLLYKQILSDRVAISWLQDEFDNDQDMLDTIEAFYNKLDSNETGVLGEGKLKQILMGLDGYNLDGVFLRNDLQLSEVSQRLCGGWNIIKDAMISDLKRSVQKKKKETGADFEERVSKLFSAQNSFSIAYINQCLGQAGIRCKIQDYFACLGAKEGENEAETTPDIFDQIAEAYHGAAPILNARPSSHNLAQDIEKVKAIKALLDALKRLQRFVKPLLGRGDEGDKDSFFYGDFMPIWEVLDQLTPLYNKVRNRMTRKPYSQEKIKLNFENSTLLNGWDLNKEHDNTSVILRREGLYYLGIMNKNYNKIFDANNVETIGDCYEKMIYKLLPGPNKMLPKVFFSKSRVQEFSPSKKILEIWESKSFKKGDNFNLDDCHALIDFYKDSIAKHPDWNKFNFKFSDTQSYTNISDFYRDVNQQGYSLSFTKVSVDYVNRMVDEGKLYLFQIYNKDFSPQSKGTPNMHTLYWRMLFDERNLHNVIYKLNGEAEVFYRKASLRCDRPTHPAHQPITCKNENDSKRVCVFDYDIIKNRRYTVDKFMFHVPITINYKCTGSDNINQQVCDYLRSAGDDTHIIGIDRGERNLLYLVIIDQHGTIKEQFSLNEIVNEYKGNTYCTNYHTLLEEKEAGNKKARQDWQTIESIKELKEGYLSQVIHKISMLMQRYHAIVVLEDLNGSFMRSRQKVEKQVYQKFEHMLINKLNYLVNKQYDAAEPGGLLHALQLTSRMDSFKKLGKQSGFLFYIPAWNTSKIDPVTGFVNLFDTRYCNEAKAKEFFEKFDDISYNDERDWFEFSFDYRHFTNKPTGTRTQWTLCTQGTRVRTFRNPEKSNHWDNEEFDLTQAFKDLFNKYGIDIASGLKARIVNGQLTKETSAVKDFYESLLKLLKLTLQMRNSVTGTDIDYLVSPVADKDGIFFDSRTCGSLLPANADANGAFNIARKGLMLLRQIQQSSIDAEKIQLAPIKNEDWLEFAQEKPYL

SEQ ID NO: 19 - protein, 1245 aa, synthetic construct
METFSGFTNLYPLSKTLRFRLIPVGETLKYFIGSGILEEDQHRAESYVKVKAIIDDYHRAYIENSLSGFELPLESTGKFNSLEEYYLYHNIRNKTEEIQNLSSKVRTNLRKQVVAQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAIVNFWNTIHDTVLAEGGLKTIIASLGSYGLEGIFLKNDLQLTDISQKATGSWGKISSEIKQKIEVMNPQKKKESYETYQERIDKIFKSYKSFSLAFINECLRGEYKIEDYFLKLGAVNSSSLQKENHFSHILNTYTDVKEVIGFYSESTDTKLIRDNGSIQKIKLFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 20 - protein, 1245 aa, synthetic construct
METFSGFTNLYPLSKTLRFRLIPVGETLKHFIDSGILEEDQHRAESYVKVKAIIDDYHRAYIENSLSGFELPLESTGKFNSLEEYYLYHNIRNKTEEIQNLSSKVRTNLRKQVVVQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLPEKFENDSQVVGAMVNFWNTIHDTVLAEGGLKTIIASLGSYGLEGIFLKNDLQLTDISQKATGSWSKISSEIKQKIEVMNPQKKKESYESYQERIDKLFKSYKSFSLAFINECLRGEYKIEDYFLKLGAVNSSSLQKENHFSHILNAYTDVKEAIGFYSESTDTKLIQDNDSIQKIKQFLDAVKDLQAYVKPLLGNGDETGKDERFYGDLIEYWSLLDLITPLYNMVRNYVTQKPYSVDKIKINFQNPTLLNGWDLNKETDNTSVILRRDGKYYLAIMNNKSRKVFLKYPSGTDGNCYEKMEYKLLPGANKMLPKVFFSKSRINEFMPNERLLSNYEKGTHKKSGICFSLDDCHTLIDFFKKSLDKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKADGNGNINQKAIDYLCSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNALGKQSGFLFYIPAWNTSKMDPVTGFVNLFDVKYESVDKAKSFFSKFDSMRYNVERDMFEWKFNYGEFTKKAEGTKTDWTVCSYGNRIITFRNPDKNSQWDNKEINLTENIKLLFERFGIDLSSNLKDEIMQRTEKEFFIELISLFKLVLQMRNSWTGTDIDYLVSPVCNENGEFFDSRNVDETLPQNADANGAYNIARKGMILLDKIKKSNGEKKLALSITNREWLSFAQGCCKNG

SEQ ID NO: 21 - protein, 1245 aa, synthetic construct
METFSGFTNLYPLSKTLRFRLIPVGETLKHFIGSGILEEDQHRAESYVKVKAIIDDYHRTYIENSLSGFELPLESTGKFNSLEEYYLYHNIRNKTEEIQNLSSKVRTNLRKQVVTQLTKNEIFKRIDKKELIQSDLIDFVKNEPDANEKIALISEFRNFTVYFKGFHENRRNMYSDEEKSTSIAFRLIHENLPKFIDNMEVFAKIQNTSISENFDAIQKELCPELVTLCEMFKLGYFNKTLSQKQIDAYNTVIGGKTTSEGKKIKGLNEYINLYNQQHKQEKLPKMKLLFKQILSDRESASWLLEKFENDSQVVGAMVNFWNTIHDTVLAEGGLKTIIASLGSYGLEGIFLKNDLQLTDISQKATGSWSKISSEIKQKIEAMNPQKKKESYESYQERIDKLFKSYKSFSLAFVNECLRGEYKIEDYFLKLGAVNSSLLQKENHFSHILNTYTDVKEVIGFYSESTDTKLIQDNDSIQKIKQFLDAVKDLQAYVKPLLGNSDETGKDERFYGDLIEYWSLLDLITPLYNMVRNYVTQKPYSVDKIKINFQNPTLLNGWDLNKEMDNTSVILRRDGKYYLAIMNNKSRKVFLKYPSGTDRNCYEKMEYKLLPGANKMLPKVFFSKSRINEFMPNERLLSNYEKGTHKKSGTCFSLDDCHTLIDFFKKSLNKHEDWKNFGFKFSDTSTYEDMSGFYKEVENQGYKLSFKPIDATYVDQLVDEGKIFLFQIYNKDFSEHSKGTPNMHTLYWKMLFDETNLGDVVYKLNGEAEVFFRKASINVSHPTHPANIPIKKKNLKHKDEERILKYDLIKDKRYTVDQFQFHVPITMNFKANGNGNINQKAIDYLRSASDTHIIGIDRGERNLLYLVVIDGNGKICEQFSLNEIEVEYNGEKYSTNYHDLLNVKENERKQARQSWQSIANIKDLKEGYLSQVIHKISELMVKYNAIVVLEDLNAGFMRGRQKVEKQVYQKFEKKLIEKLNYLVFKKQSSDLPGGLMHAYQLANKFESFNTLGKQSGFLFYIPAWNTSKMDPVTGFVNLFDVKYESVDKAKSFFSKFDSIRYNVERDMFEWKFNYDEFTKKAEGTKTDWTVCSYGNRIITFRNPDKNSQWDNKEINLTENIKLLFERFGIDLSSNLKDEIMERTEKEFFIELISLFKLVLQMRNSWTGTDIDYLVSPVCNENGEFFDSRNVDETLPQNADANGAYNIARKGMILLDKIKKNNGEKKLTLSITNREWLSFAQGCCKNG

SEQ ID NO: 22 - protein, 1261 aa, synthetic construct
MLFQDFTHLYPLSKTVRFELKPIGKTLEHIHAKNFLSQDKTMADMYQKVKAILDDYHRDFIADMMGEVKLTKLAEFCDVYLKFRKNPKDDGLQKQLKDLQAVLRKEIVKPIGNGGKYKVGYDRLFGAKLFKDGKELGDLAKFVIAQESESSPKLPQIAHFEKFSTYFTGFHDNRKNMYSSDDKHTAIAYRLIHENLPRFIDNLQILATIKQKHSALYDQIASELTASGLDVSLASHLGGYHKLLTQEGITAYNRIIGEVNSYTNKHNQICHKSERIAKLRPLHKQILSDGMGVSFLPSKFADDSEMCQAVNEFYRHYADVFAKVQSLFDRFDDYQKDGIYVEHKNLNELSKRAFGDFGFLKRFLEEYYADVIDPEFNEKFAKTEPDSDEQKKLAGEKDKFVKGVHSLASLEQVIEYYTAGYDDESVQADKLGQYFKHRLAGVDNPIQKIHNSHSTIKGFLERERPAGERALPKIKSDKSPEMTQLRQLKELLDNALNVVHFAKLVSTETVLDTRSDKFYGEFRPLYVELAKITTLYNKVRDYLSQKPFSTEKYKLNFGNPTLLNGWDLNKEKDNFGVILQKDGCYYLALLDKAHKKVFDNAPNTGKSVYQKMVYKQIANARRDLACLLIINGKVVRKTKGLDDLREKYLPYDIYKIYQSESYKVLSPNFNHQDLVKYIDYNKILASGYFEYFDFRFKESSEYKSYKEFLDDVDNCGYKISFCNINADYIDELVEQGQLYLFQIYNKDFSPKAHGKPNLHTLYFKALFSEDNLANPIYKLNGEAQIFYRKASLDMNETTIHRAGEVLENKNPDNPKQRQFVYDIIKDKRYTQDKFMLHVPITMNFGVQGMTIEGFNKKVNQSIQQYDDVNVIGIDRGERHLLYLTVINSKGEILEQRSLNDIITTSANGTQMTTPYHKILNKKKEGRLQARKDWGEIETIKELKAGYLSHVVHQISQLMLKYNAIVVLEDLNFGFKRGRLKVENQVYQNFENALIKKLNHLVLKDKTDDEIGSYKNALQLTNNFTDLKSIGKQTGFLFYVPARNTSKIDPETGFVDLLKPRYENITQSQAFFGKFDKICYNTDKGYFEFHIDYAKFTDEAKNSRQTWVICSHGDKRYVYNKTANQNKGATKGINVNDELKSLFACHHINDKQPNLVMDICQNNDKEFHKSLMYLLKALLALRYSNANSDEDFILSPVANDEGVFFNSALADDTQPQNADANGAYHIALKGLWVLEQIKNSDDLDKVDLEIKDDEWRNFAQNR

SEQ ID NO: 23 - protein, 1275 aa, synthetic construct
MGKNQNFQEFIGVSPLQKTLRNELIPTETTKKNITQLDLLTEDEIRAQNREKLKEMMDDYYRDVIDSTLHAGIAVDWSYLFSCMRNHLRENSKESKRELERTQDSIRSQIYNKFAERADFKDMFGASIITKLLPTYIKQNPEYSERYDESMEILKLYGKFTTSLTDYFETRKNIFSKEKISSAVGYRIVEENAEIFLQNQNAYDRICKIAGLDLHGLDNEITAYVDGKTLKEVCSDEGFAKAITQEGIDRYNEAIGAVNQYMNLLCQKNKALKPGQFKMKRLHKQILCKGTTSFDIPKKFENDKQVYDAVNSFTEIVMKNNDLKRLLNITQNVNDYDMNKIYVAADAYSTISQFISKKWNLIEECLLDYYSDNLPGKGNAKENKVKKAVKEETYRSVSQLNELIEKYYVEKTGQSVWKVESYISRLAETITLELCHEIENDEKHNLIEDDDKISKIKELLDMYMDAFHIIKVFRVNEVLNFDETFYSEMDEIYQDMQEIVPLYNHVRNYVTQKPYKQEKYRLYFNTPTLANGWSKNKEYDNNAIILMRDDKYYLGILNAKKKPSKQTMAGKEDCLEHAYAKMNYYLLPGANKMLPKVFLSKKGIQDYHPSSYIVEGYNEKKHIKGSKNFDIRFCRDLIDYFKECIKKHPDWNKFNFEFSATETYEDISVFYREVEKQGYRVEWTYINSEDIQKLEEDGQLFLFQIYNKDFAVGSTGKPNLHTLYLKNLFSEENLRDIVLKLNGEAEIFFRKSSVQKPVIHKCGSILVNRTYEITESGTTRVQSIPESEYMELYRYFNSEKQIELSDEAKKYLDKVQCNKAKTDIVKDYRYTMDKFFIHLPITINFKVDKGNNVNAIAQQYIAEQEDLHVIGIDRGERNLIYVSVIDMYGRILEQKSFNLVEQVSSQGTKRYYDYKEKLQNREEERDKARKSWKTIGKIKELKEGYLSSVIHEIAQMVVKYNAIIAMEDLNYGFKRGRFKVERQVYQKFETMLISKLNYLADKSQAVDEPGGILRGYQMTYVPDNIKNVGRQCGIIFYVPAAYTSKIDPTTGFINAFKRDVVSTNDAKENFLMKFDSIQYDIEKGLFKFSFDYKNFATHKLTLAKTKWDVYTNGTRIQNMKVEGHWLSMEVELTTKMKELLDDSHIPYEEGQNILDDLREMKDITTIVNGILEIFWLTVQLRNSRIDNPDYDRIISPVLNNDGEFFDSDEYNSYIDAQKAPLPIDADANGAFCIALKGMYTANQIKENWVEGEKLPADCLKIEHASWLAFMQGERG

SEQ ID NO: 24 - protein, 1261 aa, synthetic construct
MNTSLFSSFTRQYPVTKTLRFELKPMGATLGHIQQKGFLHKDEELAKIYKKIKELLDEYHRAFIADTLGDAQLVGLDDFYADYQALKQDSKNSHLKDKLTKTQDNLRKQITKNFEKTPQLKERYKRLFTKELFKAGKDKGDLEKWLINHDSEPNKAEKISWIHQFENFTTYFQGFYENRKNMYSDEVKHTAIAYRLIHENLPRFVDNIQVLSKIKSDYPDLYHELNHLDSRTIDFADFKFDDMLQMDFYHHLLIQSGITAYNTLLGGKVLEGGKKLQGINELINLYGQKHKIKIAKLKPLHKQILSDGQSVSFLPKKFDNDYELCQTVNHFYREYVAIFDELVVLFQKFYDYDKDNIYINHQQLNQLSHELFADFRLLSRALDFYYCQIIDGDFNNKINNAKSQNAKEKLLKEKERYTKSNHSINELQKAINHYASHHEDTEVKVISDYFSATNIRNMIDGIHHHFSTIKGFLEKDNNQGESYLPKQKNSNDVKNLKLFLDGVLRLIHFIKPLALKSDDTLEKEEHFYGEFMPLYDKLVMFTLLYNKVRDYISQKPYNDEKIKLNFGNSTLLNGWDVNKEKDNFGVILCKEGLYYLAILDKSHKKVFDNAPKATSSHTYQKMVYKLLPGPNKMLPKVFFAKSNIGYYQPSAQLLENYEKGTHKKGSNFSLTDCHHLIDFFKSSIAKHPEWKEFGFRFSDTHTYQDLSDFYKEIEPQSYKVKFIDIDADYIDDLVEKGQLYLFQLYNKDFSKQSYGKPNLHTLYFKSLFSDDNLKNPIYKLNGEAEIFYRRASLSVSDTTIHQAGEILTPKNPNNTHNRTLSYDVIKNKRYTTDKFFLHIPITMNFGIENTGFKAFNHQVNTTLKNADKKDVHIIGIDRGERHLLYVSVIDGDGRIVEQRTLNDIVSISNNGMSMSTPYHQILDNREKERLAARTDWGDIKNIKELKAGYLSHVVHEVVQMMLKYNAMIVLEDLNFGFKHGRFKVEKQVYQNFENALIKKLNYLVLKNADNHQLGSVRKALQLTNNFTDIKSIGKQTGFIFYVPAWNTSKIDPTTGFVDLLKPRYENMAQAQSFISRFKKIAYNHQLDYFEFEFDYADFYQKTIDKKRIWTLCTYGDVRYYYDHKTKETKTVNITKELKSLLDKHDLSYQNGHNLVDELANSHDKSLLSGVMYLLKVLLALRYSHAQKNEDFILSPVMNKDGVFFDSRFADDVLPNNADANGAYHIALKGLWVLNQIQSADNMDKIDLSISNEQWLHFTQSR

SEQ ID NO: 25 - protein, 1288 aa, synthetic construct
MVGNKISNSFDSFTGINALSKTLRNELIPSDYTKRHIAESDFIAADTNKNEDQYVAKEMMDDYYRDFISKVLDNLHDIEWKNLFELMHKAKIDKSDATSKELIKIQDMLRKKIGKKFSQDPEYKVMLSAGMITKILPKYILEKYETDREDRLEAIKRFYGFTVYFKEFWASRQNVFSDKAIASSISYRIIHENAKIYMDNLDAYNRIKQIACEEIEKIEEEAYDFLQGDQLDVVYTEEAYGRFISQSGIDLYNNICGVINAHMNLYCQSKKCSRSKFKMQKLHKQILCKAETGFEIPLGFQDDAQVINAINSFNALIKEKNIISRLRTIGKSISLYDVNKIYISSKAFENVSVYIDHKWDVIASSLYKYFSEIVKGNKDNREEKIQKEIKKVKSCSLGDLQRLVNSYYKIDSTCLEHEVTEFVTKIIDEIDNFQITDFKFNDKISLIQNEQIVMDIKTYLDKYMSIYHWMKSFVIDELVDKDMEFYSELDELNEDMSEIVNLYNKVRNYVTQKPYSQEKIKLNFGSPTLADGWSKSKEFDNNAIILIRDEKIYLAIFNPRNKPAKTVISGHDVCNSETDYKKMNYYLLPGASKTLPHVFIKSRLWNESHGIPDEILRGYELGKHLKSSVNFDVEFCWKLIDYYKECISCYPNYKAYNFKFADTESYNDISEFYREVECQGYKIDWTYISSEDVEQLDRDGQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKKLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDENGRFYDSENYIFNEETELPKDADANGAYCIALKGLYNVIAIKNNWKEGEKFNRKLLSLNNYNWFDFIQNRRF

SEQ ID NO: 26 - protein, 1876 aa, synthetic construct
MVGNKISNSFDSFTGINALSKTLRNELIPSDYTKRHIAESDFIAADTNKNEDQYVAKEMMDDYYRDFISKVLDNLHDIEWKNLFELMHKAKIDKSDATSKELIKIQDMLRKKIGKKFSQDPEYKVMLSAGMITKILPKYILEKYETDREDRLEAIKRFYGFTVYFKEFWASRQNVFSDKAIASSISYRIIHENAKIYMDNLDAYNRIKQIACEEIEKIEEEAYDFLQGDQLDVVYTEEAYGRFISQSGIDLYNNICGVINAHMNLYCQSKKCSRSKFKMQKLHKQILCKAETGFEIPLGFQDDAQVINAINSFNALIKEKNIISRLRTIGKSISLYDVNKIYISSKAFENVSVYIDHKWDVIASSLYKYFSEIVKGNKDNREEKIQKEIKKVKSCSLGDLQRLVNSYYKIDSTCLEHEVTEFVTKIIDEIDNFQITDFKFNDKISLIQNEQIVMDIKTYLDKYMSIYHWMKSFVIDELVDKDMEFYSELDELNEDMSEIVNLYNKVRNYVTQKPYSQEKIKLNFGSPTLADGWSKSKEFDNNAIILIRDEKIYLAIFNPRNKPAKTVISGHDVCNSETDYKKMNYYLLPGASKTLPHVFIKSRLWNESHGIPDEILRGYELGKHLKSSVNFDVEFCWKLIDYYKECISCYPNYKAYNFKFADTESYNDISEFYREVECQGYKIDWTYISSEDVEQLDRDGQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKKLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDENGRFYDSENYIFNEETELPKDADANGAYCIALKGLYNVIAIKNNWKEGEKFNRKLLSLNNYNWFDFIQNRRFQIYLFQIYNKDFAPNSKGMDNLHTKYLKNIFSEDNLKNIVIKLNGEAELFYRKSSVKKKVEHKKGTILVNKTYKVEDNTENSKEKRVIIESVPDDCYMELVDYWRNGGIGILSDKAVQYKDKVSHYEATMDIVKDRRYTVDKFFIHLPITINFKADGRININEKVLKYIAENDELHVIGIDRGERNLLYVSVINKKGKIVEQKSFNMIESYETVTNIVRRYNYKDKLVNKESARTDARKNWKEIGKIKEIKEGYLSQVIHEISKMVLKYNAIIVMEDLNYGFKRGRFRVERQVYQKFENMLISKLAYLVDKSRKADEPGGVLRGYQLTYIPDSLEKLGSQCGIIFYVPAAYTSKIDPLTGFVNVFNFREYSNFETKLDFVRSLDSIRYDTEKRLFSISFDYDNFKTHNTTLAKTKWVIYLRGERIKKEHTSYGWKDDVWNVESRIKDLFDSSHMKYDDGHNLIEDILELESSVQKKLINELIEIIRLTVQLRNSKSERYDRTEAEYDRIVSPVMDEKGRFYDSENYIFNEETELPKDADANGAYCIALKGLYNVIAIKNNWKEGEKFNRKLLSLNNYNWFDFIQNRRF

SEQ ID NO: 27 - protein, 1245 aa, synthetic construct
MQEHKKISHLTHRNSVQKTIRMQLNPVGKTMDYFQAKQILENDEKLKEDYQKIKEIADRFYRNLNEDVLSKTGLDKLKDYAEIYYHCNTDADRKRLDECASELRKEIVKNFKNRDEYNKLFNKKMIEIVLPQHLKNEDEKEVVASFKNFTTYFTGFFTNRKNMYSDGEESTAIAYRCINENLPKHLDNVKAFEKAISKLSKNAVDDLDTTYSGLCGTNLYDVFTVDYFNFLLPQSGITEYNKIIGGYTTSDGTKVKGINEYINLYNQQVSKRYKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYKEAAPLFNESYANEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCKDEKYYLAIIDKSNNSILENIDFQDCDESDCYEKIIYKLLPGPNKMLPKVFFSEKCKKLLSPSDEILKIRKNGTFKKGDKFSLDDCHKLIDFYKESFKKYPNWLIYNFKFKKTNEYNDISEFYNDVASQGYNISKMKIPTSFIDKLVDEGKIYLFQLYNKDFSPHSKGTPNLHTLYFKMLFDERNLEDVVYKLNGEAEMFYRPASIKYDKPTHPKNTPIKNKNTLNDKRASTFPYDLIKDKRYTKWQFSLHFPITMNFKAPDRAMINDDVRNLLKSCNNNFIIGIDRGERNLLYVSIIDSNGAIIYQHSLNIIGNKFKGKTYETNYREKLETREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGMQSGFIFYVRPDFTSKIDPVTGFVNLLYPRYENIDKAKDMISRFDDIRYNAGEDFFEFDIDYDKFPKTASDYRKKWTICTNGERIEAFRNPASNNEWSYRTIILAEKFKELFDNNSINYRDSDNLKAEILSQTKGKFFEDFFKLLRLTLQMRNSNPETGEDRILSPVKDKNGNFYDSSKYDEKSNLPCDADANGAYNIARKGLWIVEQFKKSDNVSTVEPVIHNDKWLKFVQENDMANN

SEQ ID NO: 28 - protein, 1271 aa, synthetic construct
MKNLANFTNLYSLQKTLRFELKPIGKTLDWIIKKDLLKQDEILAEDYKIVKKIIDRYHKDFIDLAFESAYLQKKSSDSFTAIMEASIQSYSELYFIKEKSDRDKKAMEEISGIMRKEIVECFTGKYSEVVKKKFGNLFKKELIKEDLLNFCEPDELPIIQKFADFTTYFTGFHENRENMYSNEEKATAIANRLIRENLPRYLDNLRIIRSIQGRYKDFGWKDLESNLKRIDKNLQYSDFLTENGFVYTFSQKGIDRYNLILGGQSVESGEKIQGLNELINLYRQKNQLDRRQLPNLKELYKQILSDRTRHSFVPEKFSSDKALLRSLLDFHKEVIQNKNLFEEKQVSLLQAIRETLTDLKSFDLDRIYLTNDTSLTQISNFVFGDWSKVKTILAIYFDENIANPKDRQRQSNSYLKAKENWLKKNYYSIHELNEAISVYGKHSDEELPNTKIEDYFSGLQTKDETKKPIDVLDAIVSKYADLESLLTKEYPEDKNLKSDKGSIEKIKNYLDSIKLLQNFLKPLKPKKVQDEKDLGFYNDLELYLESLESANSLYNKVRNYLTGKEYSDEKIKLNFKNSTLLDGWDENKETSNLSVIFRDTNNYYLGILDKQNNRIFESIPEIQSGEETIQKMVYKLLPGANNMLPKVFFSEKGLLKFNPSDEITSLYSEGRFKKGDKFSINSLHTLIDFYKKSLAVHEDWSVFNFKFDETSHYEDISQFYRQVESQGYKITFKPISKKYIDTLVEDGKLYLFQIYNKDFSQNKKGGGKPNLHTIYFKSLFEKENLKDVIVKLNGQAEVFFRKKSIHYDENITRYGHHSELLKGRFSYPILKDKRFTEDKFQFHFPITLNFKSGEIKQFNARVNSYLKHNKDVKIIGIDRGERHLLYLSLIDQDGKILRQESLNLIKNDQNFKAINYQEKLHKKEIERDQARKSWGSIENIKELKEGYLSQVVHTISKLMVEHNAIVVLEDLNFGFKRGRQKVERQVYQKFEKMLIEKLNFLVFKDKEMDEPGGILKAYQLTDNFVSFEKMGKQTGFVFYVPAWNTSKIDPKTGFVNFLHLNYENVNQAKELIGKFDQIRYNQDRDWFEFQVTTDQFFTKENAPDTRTWIICSTPTKRFYSKRTVNGSVSTIEIDVNQKLKELFNDCNYQDGEDLVDRILEKDSKDFFSKLIAYLRILTSLRQNNGEQGFEERDFILSPVVGSDGKFFNSLDASSQEPKDADANGAYHIALKGLMNLHVINETDDESLGKPSWKISNKDWLNFVWQRPSLKA

SEQ ID NO: 29 - protein, 1245 aa, synthetic construct
MQEHKKISHLTHRNSVQKTIRMQLNPVGKTMDYFQAKQILENDEKLKENYQKIKEIADRFYRNLNEDVLSKTRLDKLKDYTDIYYHCNTDADRKRLDECASELRKEIVKNFKNRDEYNKLFNKKMIEIVLPKHLKNEDEKEVVTSFKNFTTYFTGFFTNRKNMYSDGEESTAIAYRCINENLPKHLDNVKAFEKAISKLSKNAIDDLDTTYSGLCGTNLYDVFTVDYFNFLLPQSGITEYNKIIGGYTTNDGTKVKGINEYINLYNQQVSKRDKIPNLKILYKQILSESEKVSFIPPKFEDDNELLSAVSEFYANDETFDGMPLKKAIDETKLLFGNLDNPSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYNEAAPLLNENYSNEKGLKNDDKSISLIKNFLDAIKEIEKFIKPLSETNITGEKNDLFYSQFTPLLDNISRIDILYDKVRNYVTQKPFSTDKIKLNFGNSQLLNGWDRNKEKDCGAVWLCKDEKYYLAIIDKSNNSILENIDFQDCDESDCYEKIIYKLLPGPNKMLPKVFFSEKCKKLLSPSDEILKIYKSGTFKTGDKFSLDDCHKLIDFYKESFKKYPNWLIYNFKFKKTNEYNDIREFYNDVALQGYNISKMKIPTSFIDKLVDEGKIYLFQLYNKDFSPHSKGTPNLHTLYFKMLFDERNLEDVVYRLNGEAEMFYRPASIKYDKPTHPKNTPIKNKNTLNDKKTSTFPYDLIKDKRYTKWQFSLHFPITMNFKAPDKAMINDDVRNLLKSCNNNFIIGIDRGERNLLYVSVIDSNGAIIYQHSLNIIGNKFKEKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGMQSGFIFYVRPDFTSKIDPVTGFVNLLYPQYENIDKAKDMISRFDEIRYNAGEDFFEFDIDYDEFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDQWLKFVQENDMANN

SEQ ID NO: 30 - protein, 1245 aa, synthetic construct
MQEHKKISHLTHRNSVQKTIRMQLNPVGKTMDYFQAKQILENDEKLKEDYQKIKEIADRFYRNLNEDVLSKTGLDKLKDYADIYYHCNTDADRKRLNECASELRKEIVKNFKNRDEYNKLFNKKMIEIVLPKHLKNEDEKEVVASFKNFTTYFTGFFTNRKNMYSDGEESTAIAYRCINENLPKHLDNVKVFEKAISKLSKNAIDDLGATYSGLCGTNLYDVFTVDYFNFLLPQSGITEYNKIIGGYTTSDGTKVKGINEYINLYNQQVSKRDKIPNLKILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIREKSIVDYYKTSLMQLTDNLSDKYKEAAPLFSENYDNEKGLKNDDKSISLIKNFLDAIKEIEKFIKPLSETNITGEKNDLFYSQFTPLLDNISRIDILYDKVRNYVTQKPFSTDKIKLNFGNSQLLNGWDKDKEREYGAVLLCKDEKYYLAIIDKSNNSILENIDFQDCNESDYYEKIVYKLLTKINGNLPRVFFSEKRKKLLSPSDEILKIYKSGTFKKGDKFSLDDCHKLIDFYKESFKKYPNWLIYNFKFKNTNEYNDISEFYNDVASQGYNISKMKIPTTFIDKLVDEGKIYLFQLYNKDFSPHSKGTPNLHTLYFKMLFDERNLEDVVYKLNGEAEMFYRPASIKYDKPTHPKNTPIKNKNTLNDKKASTFPYDLIKDKRYTKWQFSLHFPITMNFKAPDKAMINDDVRNLLKSCNNNFIIGIDRGERNLLYVSVIDSNGAIIYQHSLNIIGNKFKGKTYETNYREKLATREKDRTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYENIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDKWLKFVQENDMANN

SEQ ID NO: 31 - protein, 1244 aa, synthetic construct
MQERKKISHLTHRNSVKKTIRMQLNPVGKTMDYFQAKQILENDEKLKENYQKIKEIADRFYRNLNEDVLSKTGLDKLKDYAEIYYHCNTDADRKRLNKCASELRKEIVKNFKNRDEYNKLFDKRMIEIVLPKHLKNEDEKEVVASFKNFTTYFTGFFTNRKNMYSDGEESTAIAYRCINENLPKHLDNVKAFEKAISKLSKNAIDDLDAYSGLCGTNLYDVFTVDYFNFLLPQSGITEYNKIIGGYTTNDGTKVKGINEYINLYNQQVSKRDKIPNLQILYKQILSESEKVSFIPPKFEDDNELLSAVSEFYANDETFDGMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENDEIRKKSIVDYYKTSLMQLTDNLSDKYNEAAPLLNENYSNEKGLKNDDKSISLIKNFLDAIKEIEKFIKPLSETNITGEKNDLFYSQFTPLLDNISRIDILYDKVRNYVTQKPFSTDKIKLNFGNYQLLNGWDKDKEREYGAVLLCKDEKYYLAIIDKSNNRILENIDFQDCDESDCYEKIIYKLLPTPNKMLPKVFFAKKHKKLLSPSDEILKIYKNGTFKKGDKFSLDDCHKLIDFYKESFKKYPKWLIYNFKFKKTNGYNDIREFYNDVALQGYNISKMKIPTSFIDKLVDEGKIYLFQLYNKDFSPHSKGTPNLHTLYFKMLFDERNLEDVVYRLNGEAEMFYRPASIKYDKPTHPKNTPIKNKNTLNDKRASTFPYDLIKDKRYTKWQFSLHFPITMNFKDPDKAMINDDVRNLLKSCNNNFIIGIDRGERNLLYVSVINSNGAIIYQHSLNIIGNKFKGKTYETNYREKLATREKDRTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYEKIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKADNVSTVEPVIHNDKWLKFVQENDMANN

SEQ ID NO: 32 - protein, 1175 aa, synthetic construct
KTGLDKLKDYAEIYYHCNTDADRKRLNKCASELRKEIVKNFKNRDEYNKLFDKRMIEIVLPKHLKNEDEKEVVASFKNFTTYFTGFFTNRKNMYSDGEESTAIAYRCINENLPKHLDNVKAFEKAISKLSKNAIDDLDATYSGLCGTNLYDVFTVDYFNFLLPQSGITEYNKIIGGYTTSDGTKVKGINEYINLYNQQVSKRDKIPNLQILYKQILSESEKVSFIPPKFEDDNELLSAVSEFYANDETFDEMPLKKAIDETKLLFGNLDNSSLNGIYIQNDRSVTNLSNSMFGSWSVIEDLWNKNYDSVNSNSRIKDIQKREDKRKKAYKAEKKLSLSFLQVLISNSENNEIREKSIVDYYKTSLMQLTDNLSDKYNEVAPLLNENYSNEKGLKNDDKSISLIKNFLDAIKEIEKFIKPLSETNITGEKNDLFYSQFTPLLDNISRIDILYDKVRNYVTQKPFSTDKIKLNFGNYQLLNGWDKDKEREYGAVLLCRDEKYYLAIIDKSNNRILENIDFQDCDESDCYEKIIYKLLPTPNKMLPKVFFAKKHKKLLSPSDEILKIRKNGTFKKGDKFSLDDCHKLIDFYKESFKKYPNWLIYNFKFKKTNEYNDIREFYNDVALQGYNISKMKIPTSFIDKLVDEGKIYLFQLYNKDFSPHSKGTPNLHTLYFKMLFDERNLEDVVYKLNGEAKMFYRPASIKYDKPTHPKNTPIKNKNTLNDKKASTFPYDLIKDKRYTKWQFSLHFSITMNFKAPDKAMINDDVRNLLKSCNNNFIIGIDRGERNLLYVSVIDSNGAIIYQHSLNIIGNKFKGKTYETNYREKLATREKERTEQRRNWKAIESIKELKEGYISQAVHVICQLVVKYDAIIVMEKLTDGFKRGRTKFEKQVYQKFEKMLIDKLNYYVDKKLDPDEEGGLLHAYQLTNKLESFDKLGTQSGFIFYVRPDFTSKIDPVTGFVNLLYPRYENIDKAKDMISRFDDIRYNAGEDFFEFDIDYDKFPKTASDYRKKWTICTNGERIEAFRNPANNNEWSYRTIILAEKFKELFDNNSINYRDSDDLKAEILSQTKGKFFEDFFKLLRLTLQMRNSNPETGEDRILSPVKDKNGNFYDSSKYDEKSKLPCDADANGAYNIARKGLWIVEQFKKSDNVSTVEPVIHNDKWLKFVQENDMANN

SEQ ID NO: 33 - protein, 1278 aa, synthetic construct
MSININKFSDECRKIDFFTDLYNIQKTLRFSLIPIGATADNFEFKGRLSKEKDLLDSAKRIKEYISKYLADESDICLSQPVKLKHLDEYYELYITKDRDEQKFKSVEEKLRKELADLLKEILKRLNKKILSDYLPEYLEDDEKALEDIANLSSFSTYFNSYYDNCKNMYTDKEQSTAIPYRCINDNLPKFIDNMKAYEKALEELKPSDLEELRNNFKGVYDTTVDDMFTLDYFNCVLSQSGIDSYNAIIGNDKVKGINEYINLHNQTAEQGHKVPNLKRLYKQIGSQKKTISFLPSKFESDNELLKAVYDFYNTGDAEKNFTALKDTITEFEKIFDNLSEYNLDGVFVRNDISLTNLSQSMFNDWSVFRNLWNDQYDKVNNPEKAKDIDKYNDKRHKVYKKSESFSINQLQELIATTLEEDINSKKITDYFSCDFHRVTTEVENKYQLVKDLLSSDYPKNKNLKTSEEDVALIKDFLDSVKSLESFVKILTGTGKESGKDELFYGSFTKWFDQLRYIDKLYDKVRNYITEKPYSLDKIKLSFDNPQFLGGWQHSKETDYSAQLFMKDGLYYLGVMDKETKREFKTQYNTPENDSDTMVKIEYNQIPNPGRVIQNLMLVDGKIVKKNGRKNADGVNAVLEELKNQYLPENINRIRKTESYKTTSNNFNKDDLKAYLEYYIARTKEYYCKYNFVFKSADEYGSFNEFVDDVNNQAYQITKVKVSEKQLLSLVEQGKLYLFKIYNKDFSEYSKGKKNLHTMYFQMLFDDRNLENLVYKLQGGAEMFYRPASIKKDSEFKHDANVEIIKRTCEDKVNDKDNPTDDEKAKYYSKFDYDIVKNKRFTKDQFSLHLTLAMNCNQPDHYWLNNDVRELLKKSNKNHIIGIDRGERNLIYVTIINSDGVIVDQINFNIIENSYNGKKYKTDYQKKLNQREEDRQKARKTWKTIETIKELKDGYISQVVHQICKLIVQYDAIVVMENLNGGFKRGRTKVEKQVYQKFETMLINKLNYYVDKGTDYKECGGLLKAYQLTNKFETFERIGKQSGIIFYVDPYLTSKIDPVTGFANLLYPKYETIPKTHNFISNIDDIRYNQSEDYFEFDIDYDKFPQGSYNYRKKWTICSYGNRIKYYKDSRNKTASVVVDITEKFKETFTNAGIDFVNDNIKEKLLLVNSKELLKSFMDTLKLTVQLRNSEINSDVDYIISPIKDRNGNFYYSENYKKSNNEVPSQPQDGDANGAYNIARKGLMIINKLKKADDVTNNELLKISKKEWLEFAQKGDLGE

SEQ ID NO: 34 - protein, 1562 aa, synthetic construct
MKATSIWDNFTRKYSVSKTLRFELRPVGKTEENIVKKEIIDAEWISGKNIPKGTDADRARDYKIVKKLLNQLHILFINQALSSENVKEFEKEDKKSKTFVAWSDLLATHFDNWIQYTRDKSNSTVLKSLEKSKKDLYSKLGKLLNSKANAWKAEFISYHKIKSPDNIKIRLSASNVQILFGNTSDPIQLLKYQIELDNIKFLKDDGSEYTTKELADLLSTFEKFGTYFSGFNQNRANVYDIDGEISTSIAYRLFNQNIEFFFQNIKRWEQFTSSIGHKEAKENLKLVQWDIQSKLKELDMEIVQPRFNLKFEKLLTPQSFIYLLNQEGIDAFNTVLGGIPAEVKAEKKQGVNELINLTRQKLNEDKRKFPSLQIMYKQIMSERKTNFIDQYEDDVEMLKEIQEFSNDWNEKKKRHSASSKEIKESAIAYIQREFHETFDSLEERATVKEDFYLSEKSIQNLSIDIFGGYNTIHNLWYTEVEGMLKSGERPLTRVEKEKLKKQEYISFAQIERLISKHSQQYLDSTPKEANDRSLFKEKWKKTFKNGFKVSEYTNLKLNELISEGETFQKIDQETGKETTIKIPGLFESYENAILVESIKNQSLGTNKKESVPSIKEYLDSCLRLSKFIESFLVNSKDLKEDQSLDGCSDFQNTLTQWLNEEFDVFILYNKVRNHVTKKPGNTDKIKINFDNATLLDGWDVDKEAANFGFLLKKADNYYLGIADSSFNQDLKYFNEGERLDEIEKNRKNLEKEESKNISKIDQEKVKKYKEVIDDLKAISNLNKGRYSKAFYKQSKFTTLIPKCTTQLNEVIEHFKKFDTDYRIENKKFAKPFIITKEVFLLNNTVYDTATKKFTLKIGEDEDTKGLKKFQIGYYRATDDKKGYESALRNWITFCIEFTKSYKSCLNYNYSSLKSVSEYKSLDEFYKDLNGIGYTIDFVDISEEYINKKINEGKLYLFQIYNKDFSEKSKGKENLHTTYWKLLFDSKNLEDVVIKLNGQAEVFFRPASIHEKEKITHFKNQEIQNKNPNAVKKTSKFEYDIIKDNRFTKNKFLFHCPITLNFKADGNPYVNNEVQENIAKNPNVNIIGIDRGEKHLLYFTVINQQGQILDAGSLNSIKSEYKDKNQQSVSFETPYHKILDKKESERKEARESWQEIENIKELKAGYLSHVVHQLSNLIVKYNAIVVLEDLNKGFKRGRFKVEKQVYQKFEKSLIEKLNYLVFKDRKESNEPGHHLNAYQLTNKFLSFERLGKQSGVLFYATASYTSKVDPVTGFMQNIYDPYHKEKTREFYKNFTKIVYNGNYFEFNYDLNSVKPDSEEKRYRTNWTVCSCVIRSEYDSNSKTQKTYNVNDQLVKLFEDAKIKIENGNDLKSTILEQDDKFIRDLHFYFIAIQKMRVVDSKIEKGEDSNDYIQSPVYPFYCSKEIQPNKKGFYELPSNGDSNGAYNIARKGIVILDKIRLRVQIEKLFEDGTKIDWQKLPNLISKVKDKKLLMTVFEEWAELTHQGEVQQGDLLGKKMSKKGEQFAEFIKGLNVTKEDWEIYTQNEKVVQKQIKTWKLFSNST

SEQ ID NO: 35 - protein, 1742 aa, synthetic construct
MKAINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNGMKAINEYYKQLGAYCREEGKEKDDFFKRIDGAYCAISHLFFGEHGEIAQSDSDVELIQKLLEAYKGLQRFIKPLLGHGDEADKDNEFDAKLRKVWDELDIITPLYDKVRNWLSRKIYNPEKIKLCFENNGKLLSGWVDSRTKSDNGTQYGGYIFRKKNEIGEYDFYLGISADTKLFRRDAAISYDDGMYERLDYYQLKSKTLLGNSYVGDYGLDSMNLLSAFKNAAVKFQFEKEVVPKDKENVPKYLKRLKLDYAGFYQILMNDDKVVDAYKIMKQHILATLTSSIRVPAAIELATQKELGIDELIDEIMNLPSKSFGYFPIVTAAIEEANKRENKPLFLFKMSNKDLSYAATASKGLRKGRGTENLHSMYLKALLGMTQSVFDIGSGMVFFRHQTKGLAETTARHKANEFVANKNKLNDKKKSIFGYEIVKNKRFTVDKYLFKLSMNLNYSQPNNNKIDVNSKVREIISNGGIKNIIGIDRGERNLLYLSLIDLKGNIVMQKSLNILKDDHNAKETDYKGLLTEREGENKEARRNWKKIANIKDLKRGYLSQVVHIISKMMVEYNAIVVLEDLNPGFIRGRQKIERNVYEQFERMLIDKLNFYVDKHKGANETGGLLHALQLTSEFKNFKKSEHQNGCLFYIPAWNTSKIDPATGFVNLFNTKYTNAVEAQEFFSKFDEIRYNEEKDWFEFEFDYDKFTQKAHGTRTKWTLCTYGMRLRSFKNSAKQYNWDSEVVALTEEFKRILGEAGIDIHENLKDAICNLEGKSQKYLEPLMQFMKLLLQLRNSKAGTDEDYILSPVADENGIFYDSRSCGDQLPENADANGAYNIARKGLMLIEQIKNAEDLNNVKFDISNKAWLNFAQQKPYKNG

SEQ ID NO: 36 - protein, 1205 aa, synthetic construct
MYYQGLTKLYPISKTIRNELIPVGKTLEHIRMNNILEADIQRKSDYERVKKLMDDYHKQLINESLQDVHLSYVEEAADLYLNASKDKDIVDKFSKCQDKLRKEIVNLLKSHENFPKIGNKEIIKLLQSLSDTEKDYNALDSFSKFYTYFTSYNEVRKNLYSDEEKSSTAAYRLINENLPKFLDNIKAYSIAKSAGVRAKELTEEEQDCLFMTETFERTLTQDGIDNYNELIGKLNFAINLYNQQNNKLKGFRKVPKMKELYKQILSEREASFVDEFVDDEALLTNVESFSAHIKEFLESDSLSRFAEVLEESGGEMVYIKNDTSKTTFSNIVFGSWNVIDERLAEEYDSANSKKKKDEKYYDKRHKELKKNKSYSVEKIVSLSTETEDVIGKYIEKLQADIIAIKETREVFEKVVLKEHDKNKSLRKNTKAIEAIKSFLDTIKDFERDIKLISGSEHEMEKNLAVYAEQENILSSIRNVDSLYNMSRNYLTQKPFSTEKFKLNFNRATLLNGWDKNKETDNLGILLVKEGKYYLGIMNTKANKSFVNPPKPKTDNVYHKVNYKLLPGPNKMLPKVFFAKSNLEYYKPSEDLLAKYQAGTHKKGENFSLEDCHSLISFFKDSLEKHPDWSEFGFKFSDTKKYDDLSGFYREVEKQGYKITYTDIDVEYIDSLVEKDELYFFQIYNKDFSPYSKGNYNLHTLYLTMLFDERNLRNVVYKLNGEAEVFYRPASIGKDELIIHKSGEEIKNKNPKRAIDKPTSTFEYDIVKDRRYTKDKFMLHIPVTMNFGVDETRRFNEVVNDAIRGDDKVRVIGIDRGERNLLYVVVVDSDGTILEQISLNSIINNEYSIETDYHKLLDEKEGDRDRARKNWTTIENIKELKEGYLSQVVNVIAKLVLKYDAIICLEDLNFGFKRGRQKVEKQVYQKFEKMLIDKLNYLVIDKSRSQENPEEVGHVLNALQLTSKFTSFKELGKQTGIIYYVPAYLTSKIDPTTGFANLFYVKYESVEKSKDFFNRFDSICFNKVAGYFEFSFDYKNFTDRACGMRSKWKVCTNGERIIKYRNEEKNSSFDDKVIVLTEEFKKLFNEYGIAFNDCMDLTDAINAIDDASFFRKLTKLFQQTLQMRNSSADGSRDYIISPVENDNGEFFNSEKCDKSKPKDADANGAFNIARKGLWVLEQLYNSSSGEKLNLAMTNAEWLEYAQQHTI

SEQ ID NO: 37 - protein, 1263 aa, synthetic construct
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 38 - protein, 1334 aa, synthetic construct
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 39 - protein, 10 aa, synthetic construct
YLFQIYNKDF

SEQ ID NO: 40 - protein, 7 aa, Simian virus 40
PKKKRKV

SEQ ID NO: 41 - protein, 16 aa, unidentified
KRPAATKKAGQAKKKK

SEQ ID NO: 42 - protein, 9 aa, unidentified
PAAKRVKLD

SEQ ID NO: 43 - protein, 11 aa, unidentified
RQRRNELKRSP

SEQ ID NO: 44 - protein, 38 aa, Homo sapiens
NQSSNFGPMKGGNFGGRSSGPYGGGGQYFAKPRNQGGY

SEQ ID NO: 45 - protein, 42 aa, unidentified
RMRIZFKNKGKDTAELRRRRVEVSVELRKAKKDEQILKRRNV

SEQ ID NO: 46 - protein, 8 aa, unidentified
VSRKRPRP

SEQ ID NO: 47 - protein, 8 aa, unidentified
PPKKARED

SEQ ID NO: 48 - protein, 8 aa, Homo sapiens
PQPKKKPL

SEQ ID NO: 49 - protein, 12 aa, Mus musculus
SALIKKKKKMAP

SEQ ID NO: 50 - protein, 5 aa, unidentified
DRLRR

SEQ ID NO: 51 - protein, 7 aa, unidentified
PKQKKRK

SEQ ID NO: 52 - protein, 10 aa, Hepatitis delta virus
RKLKKKIKKL

SEQ ID NO: 53 - protein, 10 aa, Mus musculus
REKKKFLKRR

SEQ ID NO: 54 - protein, 20 aa, Homo sapiens
KRKGDEVDGVDEVAKKKSKK

SEQ ID NO: 55 - protein, 17 aa, Homo sapiens
RKCLQAGMNLEARKTKK

SEQ ID NO: 56 - protein, 9 aa, synthetic construct
PAAKKKKLD

SEQ ID NO: 57 - RNA, 19 nt, synthetic construct
  source 1..19: mol_type = other RNA
taatttctactcttgtaga

SEQ ID NO: 58 - RNA, 15 nt, synthetic construct
  source 1..15: mol_type = other RNA
atctacaacagtaga

SEQ ID NO: 59 - RNA, 15 nt, synthetic construct
  source 1..15: mol_type = other RNA
atctacaaaagtaga

SEQ ID NO: 60 - RNA, 20 nt, synthetic construct
  source 1..20: mol_type = other RNA
ggaatttctactcttgtaga

SEQ ID NO: 61 - RNA, 19 nt, synthetic construct
  source 1..19: mol_type = other RNA
taattcccactcttgtggg

SEQ ID NO: 62 - RNA, 15 nt, synthetic construct
  source 1..15: mol_type = other RNA
atctacaagagtaga

SEQ ID NO: 63 - RNA, 15 nt, synthetic construct
  source 1..15: mol_type = other RNA
atctacactagtaga

SEQ ID NO: 64 - RNA, 20 nt, synthetic construct
  source 1..20: mol_type = other RNA
taatttctactaagtgtaga

SEQ ID NO: 65 - RNA, 21 nt, synthetic construct
  source 1..21: mol_type = other RNA
taattttctacttgttgtaga

SEQ ID NO: 66 - RNA, 18 nt, synthetic construct
  source 1..18: mol_type = other RNA
aatttctactgttgtaga

SEQ ID NO: 67 - RNA, 19 nt, synthetic construct
  source 1..19: mol_type = other RNA
gaatttctactgttgtaga

SEQ ID NO: 68 - RNA, 36 nt, synthetic construct
  source 1..36: mol_type = other RNA
gtctaaaggtaccaccaaatttctactgttgtagat

SEQ ID NO: 69 - RNA, 36 nt, synthetic construct
  source 1..36: mol_type = other RNA
gcttagaacctttaaataatttctactattgtagat

SEQ ID NO: 70 - RNA, 10 nt, synthetic construct
  source 1..10: mol_type = other RNA
taatttctac

SEQ ID NO: 71 - skipped

SEQ ID NO: 72 - RNA, 11 nt, synthetic construct
  source 1..11: mol_type = other RNA
ggaatttctac

SEQ ID NO: 73 - RNA, 10 nt, synthetic construct
  source 1..10: mol_type = other RNA
taattcccac

SEQ ID NO: 74 - RNA, 12 nt, synthetic construct
  source 1..12: mol_type = other RNA
taattttctact

SEQ ID NO: 75 - skipped

SEQ ID NO: 76 - RNA, 10 nt, synthetic construct
  source 1..10: mol_type = other RNA
gaatttctac

SEQ ID NO: 77 - RNA, 10 nt, synthetic construct
  source 1..10: mol_type = other RNA
aaatttctac

SEQ ID NO: 78 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 79 - DNA, 81 bp, synthetic construct
  source 1..81: mol_type = other DNA
gtacttatataagggggtgggggcgcgttcgtcctcagtcgcgatcgaacactcgagccgagcagacgtgcctacggaccg

SEQ ID NO: 80 - DNA, 385 bp, synthetic construct
  source 1..385: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtacttatataagggggtgggggcgcgttcgtcctcagtcgcgatcgaacactcgagccgagcagacgtgcctacggaccg

SEQ ID NO: 81 - DNA, 947 bp, synthetic construct
  source 1..947: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggg

SEQ ID NO: 82 - DNA, 195 bp, synthetic construct
  source 1..195: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 83 - DNA, 1723 bp, synthetic construct
  source 1..1723: mol_type = other DNA
atctcgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 84 - DNA, 511 bp, synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 85 - DNA, 1195 bp, synthetic construct
  source 1..1195: mol_type = other DNA
gaattcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacatcatttt

SEQ ID NO: 86 - protein, 120 aa, synthetic construct
EVQLVESGGGLVQPGGSLRLSCAASGNIFSDNLMGWFRQAPGKEREFVAAINWNSRSTYYADSVKGRFTISADNSKNTAYLQMNSLKPEDTAVYYCAKDLTMVRGVPDYWGQGTLVTVSS

SEQ ID NO: 87 - protein, 118 aa, synthetic construct
EVQLVESGGGLVQPGGSLRLSCAASGFTLGDYVMGWFRQAPGKEREWVSVISSSGDFTSYADSVKGRFTISADNSKNTAYLQMNSLKPEDTAVYYCASHYYDSSGTNWGQGTLVTVSS

SEQ ID NO: 88 - protein, 120 aa, synthetic construct
EVQLVESGGGLVQPGGSLRLSCAASGFTFSSAIMGWFRQAPGKEREFVSAITWNGTRTYYADSVKGRFTISADNSKNTAYLQMNSLKPEDTAVYYCAKDLLEVGATPGNWGQGTLVTVSS

SEQ ID NO: 89 - protein, 121 aa, synthetic construct
EVQLLESGGGLVQPGGSLRLSCAASGFTFETYAMSWVRQAPGKGLEWVSGISPSGGITTYADSVKGRFTISRDNSKNTLYLQMNSLRAEDTAVYYCARREWWYDDWYLDYWGQGTLVTVSS

SEQ ID NO: 90 - protein, 121 aa, synthetic construct
EVQLLESGGGLVQPGGSLRLSCAASGFSFSTFAMSWVRQAPGKGLEWVSAISGSGGSTSYADSVKGRFTISRDNSKNTLYLQMNSLRAEDTAVYYCARRGWGSWSWYFDLWGQGTLVTVSS

SEQ ID NO: 91 - protein, 121 aa, synthetic construct
EVQLLESGGGLVQPGGSLRLSCAASGFTFGNYAMAWVRQAPGKGLEWVSAISGSGGGTSYADSVKGRFTISRDNSKNTLYLQMNSLRAEDTAVYYCARREWWYDDWYLDYWGQGTLVTVSS

SEQ ID NO: 92 - protein, 107 aa, synthetic construct
DIQMTQSPSSLSASVGDRVTITCRASQTIERRLNWYQQKPGKAPKLLIYAASDLESGVPSRFSGSGSGTDFTLTISSLQPEDFATYYCQQNNNWPTTFGQGTKVEIK

SEQ ID NO: 93 - protein, 107 aa, synthetic construct
DIQMTQSPSSLSASVGDRVTITCRASQTIGIYLNWYQQKPGKAPKLLIYDASSLHSGVPSRFSGSGSGTDFTLTISSLQPEDFATYYCQQSYSTPFTFGGGTKVEIK

SEQ ID NO: 94 - protein, 106 aa, synthetic construct
DIQMTQSPSSLSASVGDRVTITCRASQTIGDYLNWYQQKPGKAPKLLIYAVTSRASGVPSRFSGSGSGTDFTLTISSLQPEDFATYYCQQSYSTLTFGQGTKVEIK

SEQ ID NO: 95 - protein, 116 aa, synthetic construct
EVQLVESGGGLVQPGGSLRLSCAASGIAFSIDIMGWFRQAPGKEREFVAAVNWNGDSTYYADSVKGRFTISADNSKNTAYLQMNSLKPEDTAVYYCATIDGSWREWGQGTLVTVSS

SEQ ID NO: 96 - protein, 120 aa, synthetic construct
EVQLVESGGGLVQPGGSLRLSCAASGLRFDDYWMGWFRQAPGKEREFVSAINWSGVSTYYADSVKGRFTISADNSKNTAYLQMNSLKPEDTAVYYCAARQYGEYWQAAGWGQGTLVTVSS

SEQ ID NO: 97 - protein, 116 aa, synthetic construct
EVQLVESGGGLVQPGGSLRLSCAASGLTLDYYAMGWFRQAPGKEREFVAGINNGRAITYYADSVKGRFTISADNSKNTAYLQMNSLKPEDTAVYYCATIDGSWREWGQGTLVTVSS

SEQ ID NO: 98 - protein, 120 aa, synthetic construct
EVQLLESGGGLVQPGGSLRLSCAASGFTFSNFPMSWVRQAPGKGLEWVSAITGTGGSTYYADSVKGRFTISRDNSKNTLYLQMNSLRAEDTAVYYCATRTGTTGTAFDIWGQGTLVTVSS

SEQ ID NO: 99 - protein, 118 aa, synthetic construct
EVQLLESGGGLVQPGGSLRLSCAASGYTFSNYAMSWVRQAPGKGLEWVSAVSRSGGSTYYADSVKGRFTISRDNSKNTLYLQMNSLRAEDTAVYYCARDLGYYAFDFWGQGTLVTVSS

SEQ ID NO: 100 - protein, 120 aa, synthetic construct
EVQLLESGGGLVQPGGSLRLSCAASGFTFSTYAMSWVRQAPGKGLEWVSSISGSGGRTDYADSVKGRFTISRDNSKNTLYLQMNSLRAEDTAVYYCARIRSRGSSGFDPWGQGTLVTVSS

SEQ ID NO: 101 - protein, 108 aa, synthetic construct
DIQMTQSPSSLSASVGDRVTITCRASQNIGRYLNWYQQKPGKAPKLLIYDASGLQSGVPSRFSGSGSGTDFTLTISSLQPEDFATYYCQQSYSTPPWTFGGGTKVEIK

SEQ ID NO: 102 - protein, 107 aa, synthetic construct
DIQMTQSPSSLSASVGDRVTITCRASQTIYRYLNWYQQKPGKAPKLLIYHASNLQSGVPSRFSGSGSGTDFTLTISSLQPEDFATYYCQQSYTFPRSFGGGTKVEIK

SEQ ID NO: 103 - protein, 107 aa, synthetic construct
DIQMTQSPSSLSASVGDRVTITCRASQSVYSYLNWYQQKPGKAPKLLIYETSNLQSGVPSRFSGSGSGTDFTLTISSLQPEDFATYYCQQSFTSPLTFGGGTKVEIK

SEQ ID NO: 104 - protein, 120 aa, synthetic construct
EVQLLESGGGLVQPGGSLRLSCAASGFTFENYAMSWVRQAPGKGLEWVSAISGSGGHTYYADSVKGRFTISRDNSKNTLYLQMNSLRAEDTAVYYCAHSNKRTGHAFDIWGQGTLVTVSS

SEQ ID NO: 105 - protein, 121 aa, synthetic construct
EVQLLESGGGLVQPGGSLRLSCAASGFTFSRHAMSWVRQAPGKGLEWVSAITGSGASTYYADSVKGRFTISRDNSKNTLYLQMNSLRAEDTAVYYCARGGRREFHYGLDYWGQGTLVTVSS

SEQ ID NO: 106 - protein, 117 aa, synthetic construct
EVQLLESGGGLVQPGGSLRLSCAASGFTFGNYAMAWVRQAPGKGLEWVSAISGNGGSTFYADSVKGRFTISRDNSKNTLYLQMNSLRAEDTAVYYCARAGRILFDYWGQGTLVTVSS

SEQ ID NO: 107 - protein, 120 aa, synthetic construct
EVQLLESGGGLVQPGGSLRLSCAASGFTFSTYAMSWVRQAPGKGLEWVSAISRSGGNTYYADSVKGRFTISRDNSKNTLYLQMNSLRAEDTAVYYCARVRMKGYTYFDPWGQGTLVTVSS

SEQ ID NO: 108 - protein, 119 aa, synthetic construct
EVQLLESGGGLVQPGGSLRLSCAASGFTFSHYGMSWVRQAPGKGLEWVSSISGSGGSTYYVDSVKGRFTISRDNSKNTLYLQMNSLRAEDTAVYYCARSKRLIHGLDVWGQGTLVTVSS

SEQ ID NO: 109 - protein, 120 aa, synthetic construct
EVQLLESGGGLVQPGGSLRLSCAASGFTFSRYTMSWVRQAPGKGLEWVSTISGSGYSTYYADSVKGRFTISRDNSKNTLYLQMNSLRAEDTAVYYCAHSNKRTGHAFDIWGQGTLVTVSS

SEQ ID NO: 110 - protein, 107 aa, synthetic construct
DIQMTQSPSSLSASVGDRVTITCRASQSVSTFLNWYQQKPGKAPKLLIYGASILQSGVPSRFSGSGSGTDFTLTISSLQPEDFATYYCQQSYTPPLTFGGGTKVEIK

SEQ ID NO: 111 - protein, 107 aa, synthetic construct
DIQMTQSPSSLSASVGDRVTITCRASQSVSRFLNWYQQKPGKAPKLLIYAASVLQSGVPSRFSGSGSGTDFTLTISSLQPEDFATYYCQQTYSPPLTFGGGTKVEIK

SEQ ID NO: 112 - protein, 107 aa, synthetic construct
DIQMTQSPSSLSASVGDRVTITCRASQSIRRYLNWYQQKPGKAPKLLIYHTSRLQSGVPSRFSGSGSGTDFTLTISSLQPEDFATYYCAQGWGRPVTFGQGTKVEIK

SEQ ID NO: 113 - protein, 107 aa, synthetic construct
DIQMTQSPSSLSASVGDRVTITCRASQTISSSLNWYQQKPGKAPKLLIYGASSLRSGVPSRFSGSGSGTDFTLTISSLQPEDFATYYCQQTYSNPITFGGGTKVEIK

SEQ ID NO: 114 - protein, 107 aa, synthetic construct
DIQMTQSPSSLSASVGDRVTITCRTSQSISTYLNWYQQKPGKAPKLLIYGASALQTGVPSRFSGSGSGTDFTLTISSLQPEDFATYYCQQSYTAPLTFGGGTKVEIK

SEQ ID NO: 115 - protein, 107 aa, synthetic construct
DIQMTQSPSSLSASVGDRVTITCRASQTISKYLNWYQQKPGKAPKLLIYGASSLQSGVPSRFSGSGSGTDFTLTISSLQPEDFATYYCQQSYSPPITFGGGTKVEIK

SEQ ID NO: 116 - protein, 116 aa, synthetic construct
EVQLVESGGGLVQPGGSLRLSCAASGIPSIRAMGWFRQAPGKEREWVSSINSDGTSAFYADSVKGRFTISADNSKNTAYLQMNSLKPEDTAVYYCARAYGRGTYDWGQGTLVTVSS

SEQ ID NO: 117 - protein, 119 aa, synthetic construct
EVQLVESGGGLVQPGGSLRLSCAASGFTFGEYAMGWFRQAPGKEREFVASISRSGTLRAYADSVKGRFTISADNSKNTAYLQMNSLKPEDTAVYYCAKESKDYFYMDVWGQGTLVTVSS

SEQ ID NO: 118 - protein, 116 aa, synthetic construct
EVQLVESGGGLVQPGGSLRLSCAASGRTYGMGWFRQAPGKEREFVASVTSGGYTNYADSVKGRFTISADNSKNTAYLQMNSLKPEDTAVYYCARGGGTSVRAFDIWGQGTLVTVSS

SEQ ID NO: 119 - protein, 123 aa, synthetic construct
EVQLLESGGGLVQPGGSLRLSCAASGFAFAAYDMGWVRQAPGKGLEWVSSISGYGSTTYYADSVKGRFTISRDNSKNTLYLQMNSLRAEDTAVYYCARHSGYGSSYGVLFAYWGQGTLVTVSS

SEQ ID NO: 120 - protein, 123 aa, synthetic construct
EVQLLESGGGLVQPGGSLRLSCAASGFAFAAYDMGWVRQAPGKGLEWVATISGGGINTYYPDSVKGRFTISRDNSKNTLYLQMNSLRAEDTAVYYCARHSGYGSSYGVLFAYWGQGTLVTVSS

SEQ ID NO: 121 - protein, 123 aa, synthetic construct
EVQLLESGGGLVQPGGSLRLSCAASGFTFPVYNMAWVRQAPGKGLEWVSEIDALGTDTYYADSVKGRFTISRDNSKNTLYLQMNSLRAEDTAVYYCARHSGYGSSYGVLFAYWGQGTLVTVSS

SEQ ID NO: 122 - protein, 107 aa, synthetic construct
DIQMTQSPSSLSASVGDRVTITCRASQSISNNLNWYQQKPGKAPKLLIYGKNIRPSGVPSRFSGSGSGTDFTLTISSLQPEDFATYYCFQGSQFPYTFGQGTKVEIK

SEQ ID NO: 123 - protein, 109 aa, synthetic construct
DIQMTQSPSSLSASVGDRVTITCRASQDVSSGVAWYQQKPGKAPKLLIYHASQSISGVPSRFSGSGSGTDFTLTISSLQPEDFATYYCQSYDLKSLNVVFGQGTKVEIK

SEQ ID NO: 124 - protein, 107 aa, synthetic construct
DIQMTQSPSSLSASVGDRVTITCQASQSISSYLAWYQQKPGKAPKLLIYGQHNRPSGVPSRFSGSGSGTDFTLTISSLQPEDFATYYCQQSYNTPRTFGQGTKVEIK

SEQ ID NO: 125-2019 - skipped (1895 entries)

SEQ ID NO: 2020 - DNA, 1000 bp, Homo sapiens
  source 1..1000: mol_type = genomic DNA
gcctcccaaagtgctgagattatgggcatgagccaccgcacctggccctgacaagaacctttgagttaggtataatggttcaccccaatttatagataatgaacccaagtcacaggggaagtgaagtcagttgcctaaggtcagacagcagtaaatggttctctgaccctaactccactgcctccctctcataaaaacactgggtggttacagtgggcccacctggagaagtcaagctattctctccatctcaagaacattaatttaatcatcctttttaccatataagataacatcttcacaggttctgaggatgagaatgttgacatctttgggtggtcgttattcagcctatcacaggtatccagggaagaaaaaaggaatttccaaaaagagaaaatacgaacattgggaaggctaattacagatggtgactactgaagggttagtcagaagcataatggaggcagtgatgagatgacagcacagatgcatgactctagtcccagcaactcctaaaaggtaaagaaatgtatcctgccaccctcagcttctttggggtgtcctcataaaagagaggcagtaaagcagaatcagagtcagatagagaggttgtaagaagagaagcagaggtgagtaagctgtgtttcaaacccagagtcaaggctcttgcccctctgcggtgctgccgaagcccagggtgggtggggactgacatgcaactcaggtactgtgtggcagactttgtgccttggcatgaaactatgcctgcccacaggaaggggcaccattttctcattagctcaaagagacttctgctggccaattcctgtcttctcaatactgcagctctccagagacaacactgttctctattctcctgtaagtgaggcagagcctggcagtaccctctatgccacctctcactagtacaggttagcactcagggtggcccactggtgtgtgtctcagctgctggtgtgcgtgctggtgcaggtac

SEQ ID NO: 2021 - DNA, 1000 bp, Homo sapiens
  source 1..1000: mol_type = genomic DNA
tgggctgagggttgtggctggatctctttgcattgccacatccacaacagaattttgagaagtccgagaattctaaattggagcctgaccttcttcataatagtatatttgtcaaggtaggaggataaaacattttattgaacagtttgctaagctgatttaaaattttccagcatttagctatatggtatatggacctccacatgtatgatttcatttatattaaatgtccagaatagacaaatctataatgacaataaagagattagtagttgccagaggctgggaggaggggagaaacaatgagtgattgcggacgggtgtggggtttctttctggggcgataacagtgtcctggaattcaatagtgataatggatgcacactgtgaatatactaaaagccactcacactttaaaagtgtgggttttatggtaatttgaatgatatatcaagctatcaccaaaaatacacaatgggagttcagaaatgccaccccaaactatgatgatttgacatgctcattactttgaactgatgtcacttggggaaaaacagatgcaggcagagactttctctgagatctgcttatctgcctaagacagatcaagggatcctccaaaaggaactcaattgtcatgaatcccctcccctggaaccttatcaaccagggacaatgaacttagatcagagagggggagactggaggttgacatcatgcctagacagccacctcttcttctgagggctgctccaagagaacctttattacttgagaggcttctcatttgcataacaagaaacctttgttcaccatacacttcctcccctcatattctcataactggtgtcaccaccacccacgcagaagtccaaagcctctattcccttctgtacctcagggtgctatataagcttcaatcatctgacccttctttgaatctcatattttgtgggcttgcatgggtatgtacataattaaaaatggatttcctcttgtt

SEQ ID NO: 2022 - DNA, 1000 bp, Homo sapiens
  source 1..1000: mol_type = genomic DNA
atttacacacatgccacagacagaaacattttaatagacctttgcttatggaaaagtaaagcaaaaatgtaattctagaagggagaaattttagtcaattagaaaataagatggtcaggcattgtagctctcatgtgtaatcccagtgctttggaaggttgaggcgagaggattgcttgagaccaggagtttgcgaccagcctagacaacatagctggtcatataaaaaaacttcaaaaaaattagctagctgtagagctttctgcctatatttccagctactcaaggatgaggcaaaagaatcccttaagcccaggaggttgaggttgcagtgaactgtaattgcaccaccacactctagcctgggtaacagagcaaggtcccatctcctaaaaaaaaaagaaaggaaaataaaaagaaaataaactattctccataataatgtagacagcaatcctcactgtgaaccagaaggaacctcggcaaattttttagacatcaatgggatttcactatcagctgagagtgttccctttttagcatggcaagctgtttcctgaagcaatagagagaagcaagaccaaggaaaaatctagaaagagcctctctgtagaaaagcagagcaatgatctctaatcacaatgctatcaaatattccaggctaaattttcctttatagcattaaaattttcctcacatccacaagattccaatagttttcttaatgccatagcctggtgtctattctgccttgtggattcccataatgcaaaatgccattaaaaaaggaacagaccatgagaagtgggcctccgaagcacatgaagcttggtatcatcagaaagataaggggcaacagtcaggaataattgttgggacatttaataagtccctggaaattcctagaaacataatttttttttgagtctaagatgctatcattttaaggtgcaccattattttatttgctacaatgtagaaaacaataacactgccaatt

SEQ ID NO: 2023 - DNA, 1000 bp, Homo sapiens
  source 1..1000: mol_type = genomic DNA
tgattaggtaaaatatcagagacacaaatcaggttaaattgattttttattgtaattacatttaaaattttagaattcatcagtaggtatgaacaaacatatacatacatatatataatttatattataagtttattatttatactatacattataaaaataactgagagataaactttcgtttatccttaatgctaaaataattcatttaccttggagagatcagaactctgtccatttcccctacataaaaactagagagtactattgctttctctttctcgggcttactctggtctcatagaatatgcattttcattttttttcaacagaatatccgtggatagctaaaatttctgcttcctttgtcaacatttgtatttccccagtggacatttctgcaaaatttattttcatttctttgttaccagagaaactctgttggtcaagttcaatagcatcctcagcataatttcagaaggaaattacagggagcaattgaagtccatcactttcttggaggggaaatattaacaccctcacctcttgctcccaatattaggtggtaggcaggagtgagttactcattttctgaaggagcagtaactctttggacccctcgagtcacttggtaaataaactctagcactgccccgaagagtgcctcagagatttcaaggaataaatgctttaaaggtaggaaaatgctaagaaacaccatcatataagtgagttatttccaattttattttaaatacagccatatattattacatacagccacacattattaaataatgtattaatacattattattaaatacagccatatatatgtatatatgtgtgtgtgtatatatatacatatatatgtaagtatgtagctgctataccctcctgaagcaatgaatgtagctgctataccctccagaagcaatgataccctccagaggtgataacagatacaagtaacaaccacactctctggttttgacaacca

SEQ ID NO: 2024 - DNA, 1000 bp, Homo sapiens
  source 1..1000: mol_type = genomic DNA
cagagagcttccaaggcattatcccatccaaagggtaaagaggctgggatatttatcgactagctcccattcttcactggctgtaacttgtccacgtctcacagctgtaactcccttgtattccctacctatctggtgtgaggaccaagcttgtgtctgtggatagagaaagccctaaagcagaaagtctaggtgcttgcacaaaaagatcatctgcacagaatgatgatcaagagatgtgagtggggcaccacaacatttacctcaggaatctctgttcaggactcagctttggtctcaaaccttgggaagcttatacactgaggcagtgttaggatctcttttctctgccttcctgtgcttttaagtgtatttcactgtttttgatcccttgtctgccccttatatttgactatcaggctcttgaaggtctattacacttactcattgtttttaccccctgttcctatctcagtgcccaacacagagctgacagttaatatatgttggttggatgcatgtgtgggtatcttatctttttatcctttaaaagacctcacacgtagatgaaaattttaaaatcattaattcaatcatcaattcaattcaatcatctttttatcctttaaaagacctcacacattgatgaaaattttaaaatcattaattcaattgaagaggccttgtgattgacatgagtataaattggaccattattaacttcaaactaattctactatgccagaaaccatgcctgaagtattaaaacatcacgttaaaaaacaaaagacaaaaaaaaaacttatctaaaaaattacattaaataaaatagaccaaaggtaaatcttactcaagttttcaggaaaaaaaaattgttttctatactcttttctcacctattcttccttgtcacagagaagcaattattatattagactttcctttttcaatgtgtagatgacatcatatgatttaaattttttatgtatttctcttgcaa

SEQ ID NO: 2025 - DNA, 1000 bp, Homo sapiens
  source 1..1000: mol_type = genomic DNA
atcagcagcagaggctgcagaacagcggatattagtgaaaagcaaatgttgctgtctgatcgttcctgtggaagttttgtctcagaggagtacccggccgtgtgaggtgtcagtctgcccctactcgggggtgcctcccagttaggctactcaggggtcagggacccacttgaggaggcagtctgtctgttctcagatctcaagctgtgtgctgggagaaccactactctcttcaaagctgtcagacagggacatttaagtctgcagaggtttctgctgccttttgttgggctatgccctgcccccagaggtggagtctacagagacaggcaggccttgagctgcagtgggctccacccagttcgagcttcctggctgctttgtttacctacaatggtgggctcccctcccccagccttgctgctgccttgcagtttgatctcagactgctgtgctagcaatgagcgaggctccatgggcgtaggaccctccgagccaggtgggatacaatcttctagtttgctgtttgctaggaccattggaaaagcacagtattagggtgggagtgacccgattttccaggtgctgtctgtcacccctttccttggctaggaaagggaattccctgaccccttgcgcttcctgggtgaggtgatgccttgccctgcttcggctcatgctcagtgcactgcacccactgtcttgcacccactgtccgacaatccccagtgtgatgaacccggtacctcagttggaaatgcagaaatcattcatcttctgagtcactcacgctgggagctgtagactggagctgttcctattcggccatctacatgttctttcttccctcatcatcacttctttacttcttttatttcacttctggctttctgtcctcccacgctgaggaagactgatttggtggacatgtatttattctgctgagtaccagttgatgtggaagtagttgttttatagtcaacatgtttttatgactaat

SEQ ID NO: 2026 - DNA, 1000 bp, Homo sapiens
  source 1..1000: mol_type = genomic DNA
gagtgatgtctaatcacaatctgtgataggtatttgctttaaggtgcatctaataacatgacagtgattttcatctcatataaccttcattaactctggttccctgctaagataaagccttccctataagccaactgagaatactgtagtcagaatttacaggtacttcccattgtggttgttcaccttatttgtgccagtttttcttcttctttattcataccttttgccatgtgaatttgcatttcttctgggttggagtcaagtatatatttatcctttttacctttgactctgaggctggccaaaggaataaggtggatgtgacaaggtacaatttctgagcctagcccttagaggccttccatgtttccacttgttctcttgcacttgcgacgttgctgtcaaaagaacatgcaatggctagctagcagcctgtgcacctgcagtgagaaccagagccacccagttgctgcagcctgagaccaagctgctcagctaagcatagcttagatcaccattgagttctgaggtggtttgtcatacagcaatggcaatcagatatatccacacaaatataattttagtttatatttttgttactgcagttctcatcttattctgaggatacgtgacaaaataattctttcaaaaatattgatgctgtgccagattactattttgaatgaattattagacaaatacttcatatgtatcttattatgtgggtttacacattatttatcttattgatttaacttcaaaactaaactttagtttagctcttgggccctatctgggaaagggtcatcttttaatcaccattaaatcactgaagtcatcagtttattcaaagtactctgcacaaaattagcattctttagtggttgtgaaataaatagactttaaacttatcattaatattcccaatggtactatgggggaggcaaaattttctatcttcttagtggtttttttttttttggctagggctaaggat

SEQ ID NO: 2027 - DNA, 1000 bp, Homo sapiens
  source 1..1000: mol_type = genomic DNA
acgcacctgagaaatgtgttaaggattaagatgctagtgctagatgtttgattttctgaatcgaaccactattggtgagatccagaagctcaaagacatgatatacccaccttcaaataatgtttatgtaggtaatctattctcaggatttatagacactgctgttaagacctattgtcattggggtaaaaaaaaatccttattatattatacaaattattatatactattatattatagaaattatatttctattaaatagcttgtgtagaaagtaaccatatatagttagaaaaacactgatctcaagaacaggattttagatttgactctgacaatttctgttcggtcttgtataaatgtatcaatttagatttagggctttattttctaatccataaaatgtgtagcatacttctgctagctatacatttactgaagttattattttaaactatttttattttcatttttttgttttgagttataatcataattaatggattcaagtgacagagaaaagaaagtaattagtcatcttttttcagaatacagtctttgttctgaaggtatttcgtatgaatcaagtttcaaatcttcagataaattttcaccttgccaatgtgctttctgctctaaatcattcctgaattttgctatgatttttctttcttataaaatcttgacactaaattgtcaggagatatacatatatgtatatatgtaaaatatatatatcatatataaatatatataaattttgagttaaagtactattacagtattcaattctaccagtaattctaatagtatgaaaataaagtcaccagttgaagtaagacctactgacaccttctattatatttcgataattctatttgaaactaattatatagtaggacattttcattgttttcagtattaactggcactcatgtagatattgcaggccaaattttacctctaccttttggaattttctggggtagacttgagaatt

SEQ ID NO: 2028 - DNA, 1000 bp, Homo sapiens
  source 1..1000: mol_type = genomic DNA
tacatgtgtaaacagttttagcgtagatttcctcgcacttttaaattttggattcttaatttccctgtcccccctgccccccccccaaaaaaaacctgctaacgtttaaacgaacacagtttgggaaatctgcgttaagtccttcgtgggagtggggttgctcagctcacagtaggccacgaacctgaattttctcttgtctgctgcccccttttgatagatggagggaagagcaggcttccagtgcaatggacagaagagggagcctgcaagttggtaacagagtctattagggaaagagagagtcacttgaatcctcagagctgctcctgtcaactgctttgtgcagtttttgtgacttattagctgcttgtttgcactctatctacgcctgcccaggtgtgtttgggccctagagcgaagggagcacaggcgttcatttagaaacttatccctccgtccaaatattggatgcttaccatgtgcctggtgcaatgcagggtgatacaaagaggaagataagtgaggcattcttatcgaaggaccagacactcttccagcctgactatattcattacactcgtgcctgacctttctttgactctaagattcttcctttctaaatgtgaatcttaaagactgaagtctttgatctaagactgctttcttatcacatcacatccaacaaccaacttttcacagcttcccagatcccaaattctgtttagcaaggacacttggatttttttgttttttgttataaatgacctcttcaggttcatattttcactatgtccagaattcttattttattctgttttgtgctgacattggaggcagagtctgtgtcacagaatacaccactaggggttaccctggacatggaagggtattcactcggggaagaaattttaatggaatttttaatatctagagctgtcattatcctgtgatggttcacaagaaatggaacacttaaaaatttctacagaaaaaaagg

SEQ ID NO: 2029 - DNA, 1000 bp, Homo sapiens
  source 1..1000: mol_type = genomic DNA
gccacaaatttgttttctgtatctgtagatttgcatttttttccgaacatctcatatgaatagaatcacaaaatttgtgtattttgtgccaaacttctttcacttagcatactgatttcaaaattgatccaacttatagcatatatcagtactttattcctttttagggcaaagaaatcttccattacacggataccccacattttatttctctacccatcgcttgctgggcatgagttgtttgtgacaaatattcatatacatattcttgtgtggacatatgttttcgcttctcttgggtatatatctaggagtaggattgctgggtcatatggtaagtctctatttaatggtttagactcagtactttgttttctgcctttccacagctcagtttcataaagaggcaggagccttttgttcagggctccttggcagtaaggtaatttcttcttctgcattgtatccagctgacccttgctcagtgctgttctttgggggaaagatggaatgctgggaagccagcacctcttattccttctagctaacacttttacagtgacggatataatagatatcttcaactagtattgttgaattatctccctgatgctgtccaattttgcttcatatattttggggctctgttattaggtatgcatatatagtcattattgttatatctttgtggtggtgtggcctttttattattttagcacttttatatctttacctctaataacgtttttaaaaattgaacgttgattttgtctgatgttagtacaaccacttcagcttctttgtagttgctgtttgcatgacatatctttctccattcttttactttcaatctatttgtatctctgggtctaaaatgtgtagatagcacatagttgaatcttttaaaaaatacattttacaatctctgatttttattggaatgtttaatccatccacatttaatgttacgattgatggagctggacttatttctgccata

SEQ ID NO: 2030 - DNA, 1000 bp, Homo sapiens
  source 1..1000: mol_type = genomic DNA
aacacagagctaaaaccaagtaagaggcgattctccaaaagcacttcctcagcaaacagcatatctattgtgtgtgggttctttaattggctgagaactgaatttcacctttggcattaaagagaagtgtttatttttactgtcttcactgttttaatgtttaaacaaaatctaaatactgaggtgaactctatcataaaacaagtgaaacggcaacataggttgatccagaaagaagcaaattccagcatggcgggcactacatgtttcagctcatcagttatctgaatcttatggctctaaagatggatggatgagaatacataggcagaagcttcctggtgaggctggtatgattctgttgtcctatcttcaacactatccttctaccttcagggttgctgttgtaggttttatttctttggcttctgttgccagtaatggaaaaggaccacatggaagactgtatttatgtacatcatgtccaaacagaatatcctataatagtgaatcttggaagaaagcttgagagatgtggcccagcgcggtggctcacacctgtaatcccagcactttgggagactgaggtgggctgatcacgaggtcaggagttcgagaccagtgtgaccaacatggtgaaaccccatctctactaaaaagacaaaaattagccgggcctggtggtgttgcacccgtaatcccagctacccaggaggctgaggcaggagaattgcttgaacccaggaggcagaggttgcagtgagccaagatcgtaccactgcactccagccctccagcctggacaacagagcaagactctgtctcaaaagaaaaaaaaaataccagtttgagagatgtatgtgaggactgattaccgaaagcgaaagggtttagtacatctcatgagaacagagcagtcacaagtgatataaaccaaactcccttggaaatttgtaatctatcaacttctttatttaaagagaataggaggtttactgtg

SEQ ID NO: 2031 - DNA, 1000 bp, Homo sapiens
  source 1..1000: mol_type = genomic DNA
actcccactcctactaattacagcttgtgtgtccttcagtcattcacttcccttcacatgaccagcccagcagaaatgaactaccaggaacatgagctcagagcgatgggctggccacctgccaagcacctctgaatggaaagagcagaattttgcattgcctgccatgccacgtggagcaggccctgggtggctctttaggggatgggtgtggactcccacaacaaaaccaagggccatattcaaagttaaaagctctgccatagatggtatttgttgaggctgtgtgtggtagctcatgcatgtatgcccaacactttaggaggctgaggtgggaagatcacttgaggctgggagttcaagtctagcctaggcaagatagtgagatcccttctctaaaaaagataaaatattaactgggcatcatggacgtgcctgtagccccagctactggggaggctgaggcaggaggatggcttgagtccaggagtttgagactgcagtgagctgtgattgcaccattgctccctagcccgggtgacagaacaagacttttatttctttaaaaaaaaaaaaaaaaaagaaggtgtttactgcagttgctttattaaaaaaaaagtaaatgaatgttctgactgttctacttttgaaaataagtggcaaggaattagaactgtatctttcagcaacaaaatgtacactgtggttccatgtcacagccaggaatggagtcagatgtctcagaccagaatcacagctctgccacctcctgtgacatggacttgctaagctaccttgactctctggagctcactatgcccatcaataacaagaaataaataaatccgtcctgtaaggttgtcaggagaaacaaatgaggcactatatgtggaagttcctggaatagtgaccagcacagaggacgtctcaaagaaagatttgctgaaccccaaaagacaggaggactggaggaacaacaaagagacaggaaagctagcat

SEQ ID NO: 2032 - DNA, 1000 bp, Homo sapiens
  source 1..1000: mol_type = genomic DNA
aattcatagcccagccaaggaacttagaagagtagagggaagtcatttttcactcccctacaagaacattctgctgtaaagaggagctagaaataatttttgttttaaattcaaccaaacatagggataattctgaaatttggaaccaaaagaattataagtacactactggtgaatttgtgcttatctgaaatctacacatgtagctgtctttatgtatctctgtatatcgatgtttttctatatatataatcagtgaagtaagatatctagtcattcatttactcaccaagtgattgcagtggggtgacagggacagtggggggtgtggtggcgggttgccagagcatgaggagtatgcaatagaatctaagaaatcatacctacctggccaggcacagttgctcatgcctgtaatcccagcactttgggaggcagaggcaggcggatcacttgaggtcaggagttccagaccagcctggccaacatggtgaaatcccatctctactaaaaatacaaaaaatacaaaaaattagctgggtgtggtggcacatgcctgtaatcctggctactctggaggctgaggcaggagaatggcttgaacctgggaggcagaggctgcagtgagctgaaattgtactactgcactccagcctgggtgacagagtgagactccatctcaaaaaaaaaaaaaaaaaaaaaaaatcagacctgccttccatgagctcatggtatacttgaatctccataggctagttattcaggagggtatgtaatgtaactcaacaatgcacaattacttaaattcgctcaggagaattacctcattttgcccaacttgttactgtgaaaaaaaaaaaagaaagaaaatttcaggaccttccaaatttattatgccaaagggaaaagtcaagccctggaaaccaagtcatgtaacacggctgtttttcttctctggtgcatgactgttgcttcctgatctttttgttgatgttataca

SEQ ID NO: 2033 - DNA, 1000 bp, Homo sapiens
  source 1..1000: mol_type = genomic DNA
catataaattaaatatttatgttatattgaaggaatacttttagacttgtttaaacacaaatctttaaaaattacatatcactcttgcatgtacataaaaaatgaaaatataggcaattaaattaagagaggtctacagtgtctttacatcaagtctgactctactgagtccctttttgactcagagtcattaatatattgtttttttccagtaataatgtagtgatgcagcctgtcttcaaagactgctctactattgactcagattttctcccaagccattgatactagttttgaagctgatgctttttaaatcttgctgtcagacttacgggaaggttttcatacaacagggctcatattctttcctcaaattatccttacatgtaaatgttcagaatgtcgagatgatacataggccagttatgccactgtgaatatctaccaaggtcacatgtgtaatgaacaaagacagctatttctgctgctggctggcagtgatttgcaagattttgttgactgtaggacatatcctacttcaatgatgttaaaatgtgaacaaatatgcacttcagactttgtaaaatgtagcacagcacttacagagcacactaggcttctggcactcgcataaaatgaagacttggagttttagctgagtactaaaggaggaccatcctcccaccgaaggatgaagaatttaaggatatgtaagttgagctgtacttatgttcatctgtgatttttacaagtcacttattgctacatgtatcctttaaatatgcgttgtccttcctcctaaaatggtttcaccataataagtgaaatgtcagcttgtcacattaaattataaattataaattaccatcaccttagtcctctacatatccttcaacttcattatgacactgtccttcagagataaggaacagaaaggctttaatgaaaacttcagctaatgtaataattagggaaggatgagctaattaagaaacataca

SEQ ID NO: 2034 - DNA, 1000 bp, Homo sapiens
  source 1..1000: mol_type = genomic DNA
caaagtctccctagagggcaaaattgtccccattgaagaccactgggttagatagaaacttacatctcacacatggagagtccaggctggcatggtcgctctgctgtgcactgggagcccaggttcctcctcgctttgcaaattgtacaagctgccctcatcacctggatgcctacatctcacttaagagtctcagttctaggagggcacagacaatggtgtactggtaaacagactctgttaaaaaaaaaaaaaaaaaaaaccaacacaatcaggaacattttttaaaagcccagatttgtagtgtttgcagattcttatgttttaaatactcctgccatggctgatgtgaaactaccaacagtttaacaactggcttactaaatttctgaatatttaccatttgtcccttgtaagacagtattagtgggctgcagtatatcaacagagaaagggaaggaaaagatacaaccttttgttgaaggacaaaatgacatttcacttttcttcagccccactggccaaaacttagtcccatgttcaccttagctgcaggggaggctgaaatgcagtgtttattctaaacaaccatgtatccagccacaataccaggggaatttatcaccaagagaaagagagagagaatatctagtgcttgaaaattatcagtctctgccacaattttatttaaaaaataaccagaaaaatgagagtgaattttatctgagaggatcttagaaatctcagcatcgagaaggtaataaataaagagagataagtcacagacttcctgcgacagtcaagaattccccatgcagatgacaccccaggagatgccgggtgattgttcttacaatttcttcagttgaaggtaaatgtggcactagccatttattcttttagctcacgttgtttgaagtgcatcgcctatgtacttcaccctttggactcactagaaaacaaagagaattttggaattagaagaggcttaataatgtta

SEQ ID NO: 2035 - DNA, 1000 bp, Homo sapiens
  source 1..1000: mol_type = genomic DNA
aaatataaataaaacatttcttttggaaattttataattcaagctaatttaaaattatgtaaacctctatctttcatgtaatcttcttccttcttttaaaacaacatttttttggtggtcatctgttcgggagaaaatgaaattttctgtggataagcagatattcttcacggagaaagctaacattctgcattcctctattttaaaagtggaaaacatagtcctgttatttgtatttagatgtatttctcaccaaagagtgccaggctggattacagaagatctatattctgatcttgtcctttttctttgcaagcctgaggaattgtccagacacagaattccctagatccccagatttctcacctataatatgaagggttgaaagagaggtctcaatcggctttgaattttctgttctatacttctgcaccaccactgtagcactgacaattgcatgaaaatattaagctctattatgttttcagtactatccttagcttctttaaaaaattagtctagctgtgtttgtaaataaatgatgtcactggaaaaatggtttcataccattgttgtcaatagttgaatgtggcttgccctcaggaacaatgcattcttcaataatatggaggatggaaggtgtataaggactcagatagctattattctcatttgcccatgatcctttcatatccccgcctctggtttagcattctctttcttccaggggaatttctcccccattccatgcattctagtagaattttttatcacagtagattgtcctgccctgccacagaaatgggcatttgacacagtggccacaaagattggtctaagcagtaggcctgtgacccaaggtaggccaattagagttttctgtagaattttttagattcaaagtgtatgtgtgtgggggggatgactcttcttgaattttatattaggatgcatgccagaaattgttgaaaggtctttaatgtaccatgtacaggaagct

SEQ ID NO: 2036 - DNA, 1000 bp, Homo sapiens
  source 1..1000: mol_type = genomic DNA
cacctataagaggaaatatacttatgtctaggtggactccaatgtgtctgtttactgatacttatttattcattattttcaagtaaaatgtagaagtgaataacttaagagaataactatttttatgagagaaaaatacccactttcttttttattactttgttcctctagaggttcatgaataatatattgaacatgtgaggagtgaggcctgtctagctcttttcctaacatcttccactcctgtggcctcttattaggtacctttctcagtgaagatatacaataagaattttgcatgcttattgggaatttatctgtgaaaaatcactcaaatgtcattaagtcttttctgataaaccttaatcatccaacaaccagagtttttcttaaaatagctgttgctctagaagaataccatagaatgaagttgcttcctagcatggcagtcaaggatcctggttccaagtatgagctctgaagaagatagactatgttcaccgcttactatagctgagtgcccttggacaattcatttaaactgcccctaattttcttccatcatctgtaaaatgaatgtaataatagctcttaatgagtattaaattagataataagggcactggcatttattaagaacttaataaatgttagcttttgttatttcacatttttccttgatcactcctaccaggaataaaattctgggagggtataagtaggtagtgaagtgctaactggtctggttaattgttagagttctgttaaaaaaaagttatttgaaaaaagtattttggagctaggatctaatttattaatatatctggattttctttttcaattttggtgtccattattcacataagtaattgtggttttgctatattttttcctcctgaaaaattatggctatacaactaactttattgtatactgaattttggaattttttaggatttgatgttcttactggggagaggattttgaattatttaaccac

SEQ ID NO: 2037 - DNA, 1000 bp, Homo sapiens
  source 1..1000: mol_type = genomic DNA
aacaagaggaaagcatacaaatttatttaatacatgttttatgtggcacaggagccctcataaagtaataaaaaatccccaaacacagttagagctgaacatttatatactaatctggacaaaacatttatatactgcgtggacaaagagcagtaaattgtgaaaatggaacaaggcaagggggcttagactacagtagttaatcatcaagaagtgacaaaaaaaaataagggttagttaataagatttgtttaagcagatttctcccagctttagctctctgtctctggtgatcagaatgcactccttccttcagactcagtgagcacatattccacacggaagatttcttccctagcttttaggaaatccagagaaccctttttgtatctgttgtttttttttttttttaaatgtcttgtctttaactcaaaacaatttatgtgccaggatgacatatctttggataatgtgttctgaactccttcagtacatacgtatataaattaaagcaaatattttttatgataagctggcataatagtttcataatttaatcactgatttaaaaatttaattaaaattattttttaatattttgtgtaataatttttgaggagtatcttttgtgcttaatgagtggcagatgacacccatgttcttagcagcatcattcacaatagctaaaagataggaacaactgcgtattgatggatgaatggataagcaaaatgaggtatatacatataagggaatattcttcatccttaaaaaggaaggaaattctgacatatgctacaacaaggttgaacctctaaggacattatgctaaatgaaataaaccagtctcaaaaagacaaatactatgtgattccagatacataaggcacctagagacaaactgatagagacagaaagtagaatgagtgattaccaggggttgtgagaggaaaaaagagagggttgtttgatacagagtttcagttttgcaagataaaag

SEQ ID NO: 2038 - DNA, 1000 bp, Homo sapiens
  source 1..1000: mol_type = genomic DNA
aacaggagaaaagcgtacaagtttattaaatagaagttttgcagccgggcgcgctggctcacgcttgtaatcctggcactttgggaggccgaggcgggcagatcacgaggtcaggagatcgagaccacggtgaaaccccgtctctactaaaaatacaacaaattagccaggcgtggtagcgaggcaggagaatggtgtgaacccgggaggcggagcttgcctctgcactccagatcatgccactgcactccagcctgggtgacagaccaagactccgtctcaaaaaaaaaaaaaaaaaaaaaaaaaaaaaagaagttttgcatgacatgggaaccctcataaaaaaagtgaagtcccaaaaaagtggcaaaatctaaatgcttttatattatgttgacgaaagaggggcaattgtggaaaagtaactaaattatgagggttaggctaacagaagataaaaattattttaacaagttctgtttgtataaaattttctcaatttcagctacccatccttgatgattagaatgttgcattccttctggtatacaaggaacatcttccatatgggggttttatcttctgctttcagaaaaaaaaaaataactctgtgtgtggtaggatgaaggtgtcagaacattcttcttgcacctgctggttgctatctttttaaactgcatttgtttcaaaacaatccttatgacaaaggggtgtattttggggtggcatattctgtcacccgtcaatatcaaagtggtttttgagtttgtgctcatcttctttccttattctgttccttgtaaggtaagactaaatataatggaatttgccgtcacatgtctcttatatgtagtgagttttaacaggcattccaggaaatgccatatggtcttttagcttggaaattattttagaaaacgataaaatctttagtgtgaagttatttcccagatatgtatcgctaaaattatcattacaggtgctctaggtaatatgtttg

SEQ ID NO: 2039 - DNA, 1000 bp, Homo sapiens
  source 1..1000: mol_type = genomic DNA
ttagtacttccatccctttcctggctgctctaactttacaggtacttgtaagtggcaattaagcactttttcctaattccagagtcttgccccacttcagagcaacatagagtggcctagacaggctgaggtactttgccgcttcagtcatcattaatctatggtatttactgatgagaagtaaagtggtagaagaaaaaaaaattttctgttatcctgggcacttggaaatgaatgtattctcacaatctgttctcaaaacaacttactgattctggggttctggaagctctgatgtgcaggtgagccttttaaattcctcactgttggagctcctatctaggactcactggctggatgaaaacggttctttttattgctttctgaatgtctgctagacaggcgtaagcaacaccttatatctgccttctgaaaaaggtaaaagaactgggacccatccaccatgctggacagctcggcagtggcagtggcctcccccagaccctgttccgagtgctccaccaacaaactcaccagcagtcagagtctagcctctccccaaacttcaccttcatcacaattcattttaagcccttccacaacccaatcaactctagatctacttaatggataataatttgatctcatgcaaactgcactttcctcttctcagaatgatccttctaccccttaattaaacatttgagagtgaaagaagagaaaattcgggttcaaagattggtaagtctaagaaacctaaggaaaaggagttagtaaacatgttaatcaaagagtgagcacttttcggaagcgcaacattcagatacctttcttgattggattccagaagactatttctgggaagaggagatttgcatttttctaaagtcttctacccacagcctaaccaccctagggctttgaaatattttttttctgatgtgcagtcataattgaataaataaaatgattcctgatcatttcttctcttcagctttat

SEQ ID NO: 2040 - DNA, 1000 bp, Homo sapiens
  source 1..1000: mol_type = genomic DNA
tattcctgtatttctattgtacttttttgcattaagaaacattttccaatgtaacattttaatagatttttcactatttgttgagttatttttgagtggttgtacttgagcttgccatctatgtcttaacttcagatttgtactaacttaattccagggagatatagaagcattattcctacatagctctatatcaacccccttttcctgtggtattattgttatacaaggtacaccatatatgttacaaatacaattatttatagttataattattactttaaatatatcatttatgtctattaaagaagctgagagcagagaggagataaagtatatatttatagaatttgttatattaagcttcttatttgtcattctgattctctttgttctggtggacttgagtaaatatgtgatgttatttcattatgcacacacagctttgctccttgtcattttatttatgctgtctttctcaagtatattgcatttaaatacattataggaccaacaattcaaatatatttatgttgtgttatacaattgctttttaaaatcagttaagatagatgggatatgcactgatagtatggtttttaaaattatactttaagttctgggttacatatgcagaacatgctgtttggttacataggtatacacgtgccatggtggtttgctgcacccatcaacccaccacctacattaggtatttctcctaatgttatctgtcctctggcctccaaccccccgacatgccccagtgtgtgatgttcccctccctgtgtccatgtgttctcattgttcaactcccacttatgagtgagaacatgtggtgtttggttttctgatcttgtgatagtttcctgagaatgatggtttccagcttcatccatgtccctgaaaaagatatgaactcatcctagacaataattcaaacacacacacacacacacacacacacacacacacacacacgcaaatggcactagtatct

SEQ ID NO: 2041 - DNA, 1000 bp, Homo sapiens
  source 1..1000: mol_type = genomic DNA
tccagaaaacataacaattcagaacatatatttaatccctcctcaatccagatccttgttgaaacaatgaaagagtacaatatactgccatgaaaagtactgagaaaagtctacagatagtgacatggaagaaaagaaaaaatattaaatagatcaaactagttatataatttgtatctcatttctgtaaaataaatttaacatttataagtgtattagtttgttctcacattgctataataaaatacctgagactgggtaattaaaaaaaaaaacagatttaattggcacacagttctataggctgtacagagaaaacagtggcttctgcttctggggaggtttcaggaaacttccaatcatgatggaagccgaaggggaagcagacacatcttacgtggccagagcaggagcacaagtgtgaagggaagtgtctgttcatattcttcactcactttttaatggggttgtttgtttttttcttagaaatttaagttccttgtagattctggatattaggcctttgtcagatggatagattgcaaaaatgttctcccattctgcaggttgcctgttcactttgatgatagtttcttttgctgagcagaagctctttagtttaattttgcagggacatggatgaagctggaaaccattatcttcagtagactaactgttaacaggaacagaaaaccaaaaacaaacaaaagcatgaagagggaagtgtcacccacatgagaactcactattgtgatgacaacaccaaggggaatggtgttaaaccatgagaaccggcccccatgatccaatcacttcccaccaggccccacctccaatactggatattacaattcaacaagagatttgggcaggaatacagatccaaaccatatcagtaaatataataaatatatattaataaatatgtaaatatatgtatgcaagttaacaaatgaaccagttggtatgtaagtatgtatataaaggaccatagcagtta

SEQ ID NO: 2042 - DNA, 1045 bp, Homo sapiens
  source 1..1045: mol_type = genomic DNA
ctgaatactagaggagcaagtacaacaaatggaaaatgggatcaagtatgagtgagagttgctaagatgcctggtagggatgcaaaggggtagagagcctggggagagagggtgagggagggaagcactggtttctcaagcaaaagctaaaatttttctattaagatttaacctgatgctacactttggtggtgcagcaagggtctcaaatggtataaaactcaggtgatcatgctttatgtctgtctctagaaaaatgctccaaaaatgataagtagtgataatccgcagtctcgttgcataaaatcagccccaggtgaatgactaagctccatttccctaccccacccttattacaataacctcgacaccaactctagtccgtgggaagataaactaatcggagtcgcccctcaaatcttacagctgctcactcccctgcagggcaacgcccagggaccaagttagccccttaagcctaggcaaaagaatcccgcccataatcgagaagcgactcgacatggaggcgatgacgagatcacgcgaggaggaaaggagggagggcttcttccaggcccagggcggtccttacaagacgggaggcagcagagaactcccataaaggtattgcggcactcccctccccctgcccagaagggtgcggccttctctccacctcctccaccgcagctccctcaggattgcagctcgcgccggtttttggagaacaagcgcctcccacccacaaaccagccggaccgacccccgctcctcccccacccccacgagtgcctgtagcaggtcgggcttgtctcgcccttcaggcggtgggaacccggggcggagccgcggccgccgccatccagaagtctcggccggcagcccgcccccgcctccagcgcgcgcttcctgccacgttgcgcaggggcgcggggccagacactgcggcgctcggcctcggggaggaccgtaccaacgcccgcctccccgccacccccgcgccccgcgcagtggtttcgctcatgtgagactcgagccagtagca

SEQ ID NO: 2043 - DNA, 1020 bp, Homo sapiens
  source 1..1020: mol_type = genomic DNA
gccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccaggaaatgggggtgtgtcaccagataaggaatctgcctaacaggaggtgggggttagacccaatatcaggagactaggaaggaggaggcctaaggatggggcttttctgtcaccaatcctgtccctagtggccccactgtggggtggaggggacagataaaagtacccagaaccagagccacattaaccggccctgggaatataaggtggtcccagctcggggacacaggatccctggaggcagcaaacatgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaaccca

SEQ ID NO: 2044 - protein, 6 aa, synthetic construct
HHHHHH

SEQ ID NO: 2045 - protein, 7 aa, synthetic construct
GHHHHHH

SEQ ID NO: 2046 - protein, 8 aa, synthetic construct
HHHHHHHH

SEQ ID NO: 2047 - protein, 9 aa, synthetic construct
GHHHHHHHH

SEQ ID NO: 2048 - RNA, 12 nt, synthetic construct
  source 1..12: mol_type = other RNA
  modified_base 1: mod_base = OTHER; note = 2'-O-methoxy modified nucleotide with phosphorothioate linkage
  modified_base 2: mod_base = OTHER; note = Nucleotide with phosphorothioate linkage
taattcctactc

SEQ ID NO: 2049 - RNA, 29 nt, synthetic construct
  source 1..29: mol_type = other RNA
  modified_base 28: mod_base = OTHER; note = Nucleotide with phosphorothioate linkage
  modified_base 29: mod_base = OTHER; note = 2'-O-methoxy modified nucleotide
ttgtaggtttaggatggccttctccgacg

SEQ ID NO: 2050 - RNA, 29 nt, synthetic construct
  source 1..29: mol_type = other RNA
  modified_base 28: mod_base = OTHER; note = Nucleotide with phosphorothioate linkage
  modified_base 29: mod_base = OTHER; note = 2'-O-methoxy modified nucleotide
ttgtaggtgagtctctcagctggtacacg

SEQ ID NO: 2051 - DNA, 3691 bp, synthetic construct
  source 1..3691: mol_type = other DNA
tcagctgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgttgatatctcgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaattcggatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctc

SEQ ID NO: 2052 - DNA, 4884 bp, synthetic construct
  source 1..4884: mol_type = other DNA
cgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatactcttcctttttcaatattattgaagcatttatcagggttattgtctcatgagcggatacataacgcgcgtcggagaaggccatcctaagaaacctcagctgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgttgatatctcgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaattcggatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctctcgagtttcttaggatggccttctccgacgtcgactctagaggatcccgggtaccgagctcgaattcggatatcctcgagactagtgggcccgtttaaacacatgtgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggc

SEQ ID NO: 2053 - DNA, 3822 bp, synthetic construct
  source 1..3822: mol_type = other DNA
gcgtataatggactattgtgtgctgatatgtacacctgaggacacaggtacaggactcagccagctgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgttgatatctcgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaattcggatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctctcgaggaggtgagattgtgttcggcatgcctcacaggcagatctatcagcacacaatagtccattatacgc

SEQ ID NO: 2054 - DNA, 3852 bp, synthetic construct
  source 1..3852: mol_type = other DNA
gcgtataatggactattgtgtgctgatatgtacacctgaggacacaggtacaggactcagccagctgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgttgatatctcgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaattcggatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctctcgagtttcttaggatggccttctccgacgtcgacgaggtgagattgtgttcggcatgcctcacaggcagatctatcagcacacaatagtccattatacgc

SEQ ID NO: 2055 - DNA, 2885 bp, synthetic construct
  source 1..2885: mol_type = other DNA
gcgtataatggactattgtgtgctgatatgtacacctgaggacacaggtacaggactcagccctcagcaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgggcagcggcgctactaacttcagcctgctgaagcaggctggcgacgtggaggagaaccctggacctatggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctacttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccagctgagagactctaattccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcaccgtgtaccagctgagagactctaaacaggtcgacgaggtgagattgtgttcggcatgcctcacaggcagatctatcagcacacaatagtccattatacgc

SEQ ID NO: 2056 - DNA, 4540 bp, synthetic construct
  source 1..4540: mol_type = other DNA
tgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacataacgcgtcgcgaggccatatgggttaactttagagtctctcagctggtacacgcctcagcaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgggcagcggcgctactaacttcagcctgctgaagcaggctggcgacgtggaggagaaccctggacctatggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctacttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggataccagctgagagactctaattccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcaccgtgtaccagctgagagactctaaacaggtcgactctagaggatccccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttaca

(19) United States
(12) Patent Application Publication
(10) Pub. No.: US 2026/0022404 A1
(43) Pub. Date: Jan. 22, 2026

(54) COMPOSITIONS AND METHODS FOR GENOME EDITING

(71) Applicant: Celyntra Therapeutics SA, Mont-Saint-Guibert (BE)

(72) Inventors: John Schiel, Boulder, CO (US); Roland Baumgartner, Boulder, CO (US)

(73) Assignee: Celyntra Therapeutics SA, Mont-Saint-Guibert (BE)

(21) Appl. No.: 18/997,188

(22) PCT Filed: Jul. 25, 2023
(86) PCT No.: PCT/US2023/028625

§ 371 Date: Jan. 21, 2025

Related U.S. Application Data
(60) Provisional application No. 63/392,041, filed on Jul. 25, 2022.
(60) Provisional application No. 63/412,772, filed on Oct. 3, 2022.
(60) Provisional application No. 63/521,084, filed on Jun. 14, 2023.

(51) Int. Cl. C12N 15/90; C12N 9/22; C12N 15/11

(52) U.S. Cl. CPC C12N 15/907; C12N 9/226; C12N 15/11; C12N 2310/20

(57) ABSTRACT

CRISPR-Cas systems have been engineered for various purposes, such as genomic DNA cleavage, base editing, epigenome editing, and genomic imaging. Although significant developments have been made, there still remains a need for new and useful CRISPR-Cas systems as powerful precise genome targeting tools. The invention disclosed herein comprises CRISPR-Cas based compositions and methods for high integration efficiency and expression efficiency of transgenes together with high post-transfection cell viability in eukaryotic cells.

### REFERENCE TO RELATED APPLICATIONS

This application claims the benefit of and priority to U.S. Provisional Patent Application Nos. 63/392,041, filed Jul. 25, 2022, 63/412,772, filed on Oct. 3, 2022, and 63/521,084, filed on Jun. 14, 2023, the disclosure of which is hereby incorporated by reference in its entirety for all purposes.

### STATEMENT AS TO FEDERALLY FUNDED RESEARCH

None.

### BACKGROUND

CRISPR-Cas systems have been engineered for various purposes, such as genomic DNA cleavage, base editing, epigenome editing, and genomic imaging. Although significant developments have been made, there still remains a need for new and useful CRISPR-Cas systems as powerful precise genome targeting tools. The invention disclosed herein comprises CRISPR-Cas based compositions and methods for high integration and expression efficiency of transgenes together with high post-transfection cell viability in eukaryotic cells.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a schematic representation showing the structure of an exemplary single guide Type V-A CRISPR system. FIG. 1B is a schematic representation showing the structure of an exemplary dual guide Type V-A CRISPR system.

FIGS. 2A-C show a series of schematic representations showing incorporation of a protecting group (e.g., a protective nucleotide sequence or a chemical modification) (FIG. 2A), a donor template-recruiting sequence (FIG. 2B), and an editing enhancer (FIG. 2C) into a Type V-A CRISPR-Cas system. These additional elements are shown in the context of a dual guide Type V-A CRISPR system, but it is understood that they can also be present in other CRISPR systems, including a single guide Type V-A CRISPR system, a single guide Type II CRISPR system, or a dual guide Type II CRISPR system.

FIG. 3 shows a schematic of a Type V-A nucleic acid guide nuclease comprising a dual guide nucleic acid.

FIG. 4 shows a schematic of a polynucleotide comprising an upstream and a downstream PAM and target nucleotide sequence flanking a donor template in a P₁⁺T₁⁺D⁺T₂⁻P₂⁻ configuration (401). The polynucleotide can be circular, e.g., a plasmid, or linear double stranded DNA with or without covalently closed ends. The figure further shows a method for cleaving the polynucleotide with a nucleic acid guided nuclease. The nuclease can cleave at one or more target site on the polynucleotide. The figure further shows a processed donor template (402).

FIG. 5 shows a schematic of a polynucleotide comprising an upstream and a downstream PAM and target nucleotide sequence flanking a donor template in a T₁⁻P₁⁻D⁺T₂⁻P₂⁻ configuration (501). The polynucleotide can be circular, e.g., a plasmid, or linear double stranded DNA with or without covalently closed ends. The figure further shows a method for cleaving the polynucleotide with a nucleic acid guided nuclease. The nuclease can cleave at one or more target site on the polynucleotide. The figure further shows a processed donor template (502). The process donor template (502) can comprise a bound nucleic acid-guided nuclease comprising a functional domain, such as one or more nuclear localization signals (NLS). The NLS can improve nuclear import of the polynucleotide.

FIG. 6 shows a schematic of a polynucleotide comprising an upstream and a downstream PAM and target nucleotide sequence flanking a donor template in a P₁⁺T₁⁺D⁺P₂⁺T₂⁺ configuration (601). The polynucleotide can be circular, e.g., a plasmid, or linear double stranded DNA with or without covalently closed ends. The figure further shows a method for cleaving the polynucleotide with a nucleic acid guided nuclease. The nuclease can cleave at one or more target site on the polynucleotide. The figure further shows a processed donor template (602). The process donor template (602) can comprise a bound nucleic acid-guided nuclease comprising a functional domain, such as one or more nuclear localization signals (NLS). The NLS can improve nuclear import of the polynucleotide.

FIG. 7 shows a schematic of a polynucleotide comprising an upstream and a downstream PAM and target nucleotide sequence flanking a donor template in a T₁⁻P₁⁻D⁺P₂⁺T₂⁺ configuration (701). The polynucleotide can be circular, e.g., a plasmid, or linear double stranded DNA with or without covalently closed ends. The figure further shows a method for cleaving the polynucleotide with a nucleic acid guided nuclease. The nuclease can cleave at one or more target site on the polynucleotide. The figure further shows a processed donor template (702). The process donor template (702) can comprise a bound nucleic acid-guided nuclease comprising a functional domain, such as one or more nuclear localization signals (NLS). The NLS can improve nuclear import of the polynucleotide.

FIG. 8 shows an exemplary plasmid.

FIG. 9 shows cell viability (y-axis) data following knock-in of heterologous DNA using a variety of donor templates.

FIG. 10 shows gene editing data as measured by the % of edits comprising perfect HDR as a proportion of total edits (y-axis) at the TGFBR2 locus using MAD7 complexed with gR007 and a variety of donor templates.

FIG. 11 shows gene editing data as measured by the % of edits comprising perfect HDR as a proportion of total edits (y-axis) at the TGFBR2 locus using MAD7 complexed with gR008 and a variety of donor templates.

FIG. 12 shows gene editing data as measured by the % of edits comprising perfect HDR as a proportion of total edits at the FAS locus using MAD7 complexed with gR94 and a variety of donor templates.

FIG. 13 shows gene editing data as measured by GFP fluorescence and cell viability post treatment with various homology dependent repair templates.

FIG. 14 shows a schematic of a linear double stranded polynucleotide with covalently closed ends comprising an upstream and a downstream PAM and target nucleotide sequence flanking a donor template in a P₁⁺T₁⁺D⁺T₂⁻P₂⁻ configuration (1401). The figure further shows a method for cleaving the polynucleotide with a nucleic acid guided nuclease. The nuclease can cleave at one or more target site on the polynucleotide. The figure further shows a processed donor template (1402). The process donor template (1402) can comprise a bound nucleic acid-guided nuclease comprising a functional domain, such as one or more nuclear localization signals (NLS). The NLS can improve nuclear import of the polynucleotide.

FIG. 15 shows a schematic of a linear double stranded polynucleotide with covalently closed ends comprising an upstream and a downstream PAM and target nucleotide sequence flanking a donor template in a T₁⁻P₁⁻D⁺T₂⁻P₂⁻ configuration (1501). The figure further shows a method for cleaving the polynucleotide with a nucleic acid guided nuclease. The nuclease can cleave at one or more target site on the polynucleotide. The figure further shows a processed donor template (1502). The process donor template (1502) can comprise a bound nucleic acid-guided nuclease comprising a functional domain, such as one or more nuclear localization signals (NLS). The NLS can improve nuclear import of the polynucleotide.

FIG. 16 shows a schematic of a linear double stranded polynucleotide with covalently closed ends comprising an upstream and a downstream PAM and target nucleotide sequence flanking a donor template in a P₁⁺T₁⁺D⁺P₂⁺T₂⁺ configuration (1601). The figure further shows a method for cleaving the polynucleotide with a nucleic acid guided nuclease. The nuclease can cleave at one or more target site on the polynucleotide. The figure further shows a processed donor template (1602). The process donor template (1602) can comprise a bound nucleic acid-guided nuclease comprising a functional domain, such as one or more nuclear localization signals (NLS). The NLS can improve nuclear import of the polynucleotide.

FIG. 17 shows a schematic of a linear double stranded polynucleotide with covalently closed ends comprising an upstream and a downstream PAM and target nucleotide sequence flanking a donor template in a T₁⁻P₁⁻D⁺P₂⁺T₂⁺ configuration (1701). The figure further shows a method for cleaving the polynucleotide with a nucleic acid guided nuclease. The nuclease can cleave at one or more target site on the polynucleotide. The figure further shows a processed donor template (1702). The process donor template (1702) can comprise a bound nucleic acid-guided nuclease comprising a functional domain, such as one or more nuclear localization signals (NLS). The NLS can improve nuclear import of the polynucleotide.

FIG. 18 shows a schedule of a polynucleotide comprising one or more additional PAM and target sites.

FIG. 19 shows gene editing efficiency for miniplasmid and linear double-stranded DNA with covalently closed ends templates as measured by % CAR expression

(y-axis) as a function of ssODN concentration, donor template concentration, gRNA:Nuclease ratio, and nuclease concentration.

FIG. 20 shows gene editing efficiency for miniplasmid and linear double-stranded DNA with covalently closed ends templates as measured by % CAR expression (y-axis) as a function of template concentration, nuclease concentration, and ssODN concentration.

FIG. 21 shows cell viability for miniplasmid and linear double-stranded DNA with covalently closed ends templates as measured by % viable cells in a treated cell population (y-axis) as a function of template concentration, nuclease concentration, and ssODN concentration.

### DETAILED DESCRIPTION

### Outline

I. Compositions of and methods for using polynucleotides and polypeptides bound thereto
A. Polynucleotides
B. Polynucleotides and polypeptides bound thereto
C. Methods for using polynucleotides and polypeptides bound thereto
   II. Engineered non-naturally-occurring dual guide CRISPR-cas systems
A. Cas proteins
B. Guide nucleic acids
C. gNA modifications
   III. Composition and methods for targeting, editing, and/or modifying genomic DNA
A. Ribonucleoprotein (RNP) delivery and “cas RNA” delivery
B. CRISPR expression systems
C. Donor templates
D. Efficiency and specificity
E. Multiplex
F. Genomic safe harbors
   IV. Pharmaceutical compositions
   V. Therapeutic uses
A. Gene therapies
B. Immune cell engineering

### VI. Kits

### VII. Embodiments

### VIII. Examples

### IX. Equivalents

### I. Compositions of and Methods for Using Polynucleotides and Polypeptides Bound Thereto

Recent advances have been made in precise genome targeting technologies. For example, specific loci in genomic DNA can be targeted, edited, or otherwise modified by designer meganucleases, zinc finger nucleases, or transcription activator-like effectors (TALEs). Furthermore, the CRISPR-Cas systems of bacterial and archaeal adaptive immunity have been adapted for precise targeting of genomic DNA in eukaryotic cells. Compared to the earlier generations of genome editing tools, the CRISPR-Cas systems are easy to set up, scalable, and amenable to targeting multiple positions within the eukaryotic genome, thereby providing a major resource for new applications in genome engineering. In certain embodiments, provided herein are compositions, methods, and/or kits for genome engineering. In certain embodiments, provided herein are compositions, methods, and/or kits for genome engineering of eukaryotic cells. In certain embodiments, provided herein are compositions, methods, and/or kits for genome engineering of human cells. In certain embodiments, provided herein are compositions, methods, and/or kits for genome engineering of human immune or stem cells. In certain embodiments, provided herein are compositions, methods, and/or kits for efficient genome engineering. In certain embodiments, provided herein are compositions, methods, and/or kits for genome engineering resulting in improved viability cells, for example T cells, treated with compositions, methods, and/or kits as disclosed herein. In certain embodiments, provided herein are compositions, methods, and/or kits comprising polynucleotides and/or polynucleotides and polypeptides bound thereto. In certain embodiments, provided herein are compositions and method for improving editing efficiency of one or more cells treated with a composition comprising a polynucleotide as disclosed herein, for example at least 5, 10, 20, 25, 30, 40, 50, 60, 70, 80, 90, 100, 200, or 500% increased editing efficiency.

### A. Polynucleotides

In certain embodiments, provided herein are compositions comprising

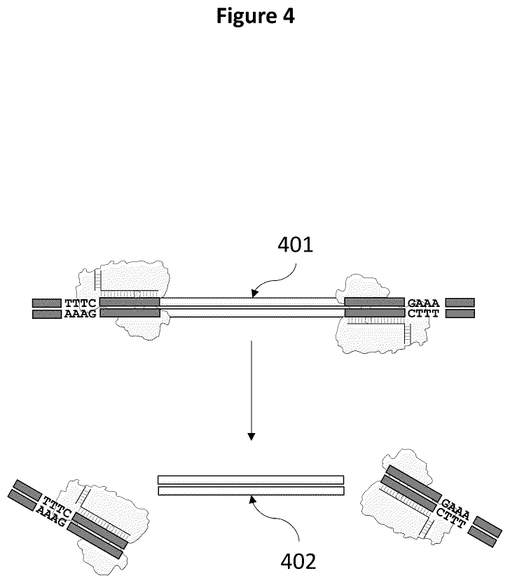

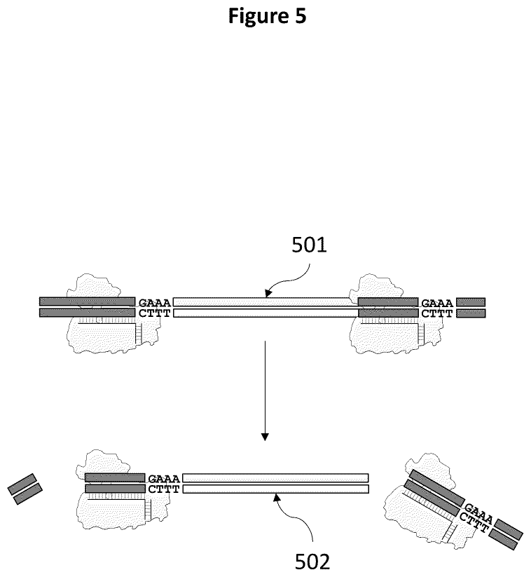

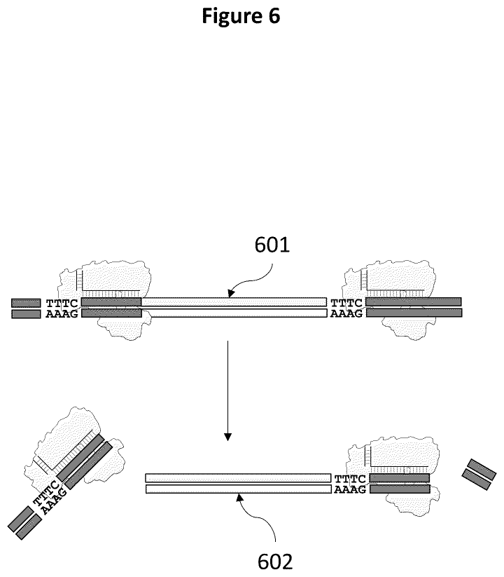

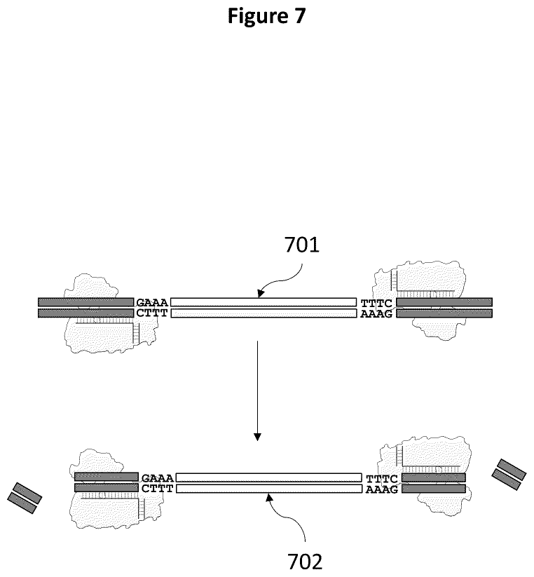

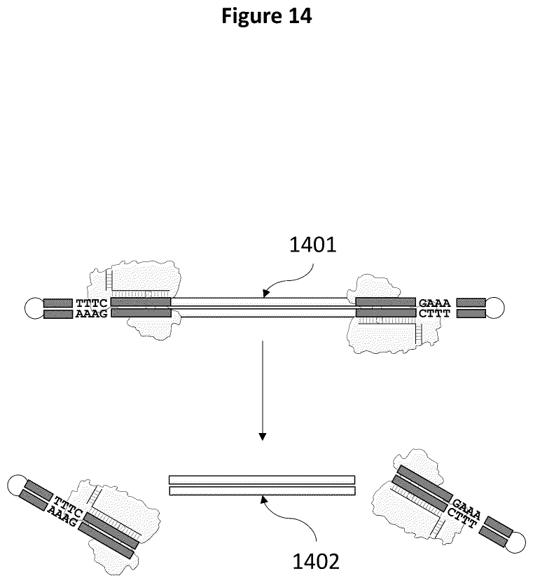

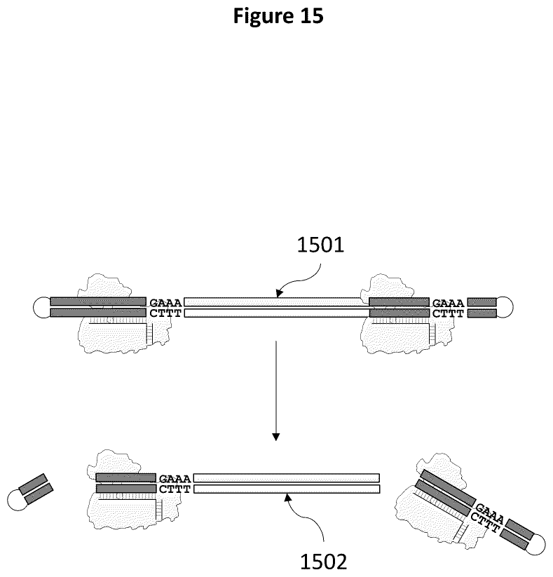

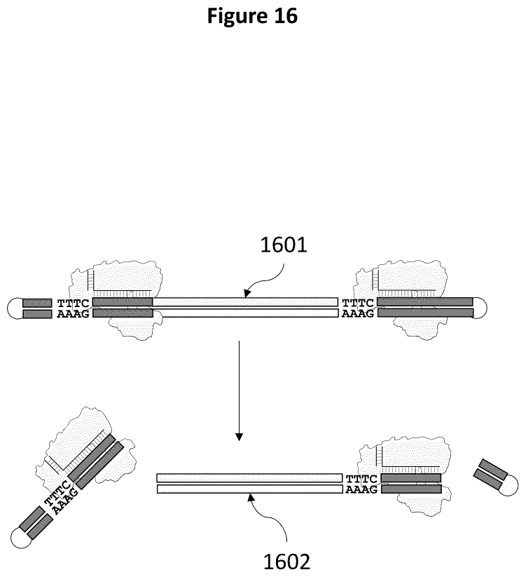

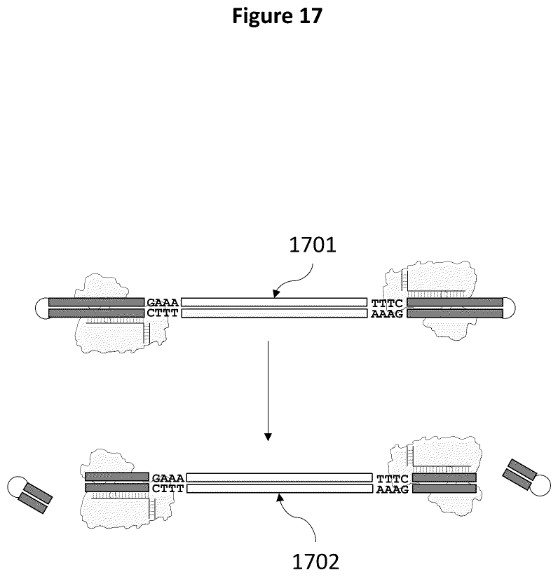

polynucleotides. The polynucleotide can be any suitable form, such as single-stranded DNA, single-stranded RNA, double-stranded DNA, or double-stranded RNA, preferably, double-stranded DNA, for example a plasmid. In certain embodiments, the linear double-stranded DNA comprises covalently closed ends (e.g., doggybone dNA, dbDNA). Exemplary linear double stranded DNA polynucleotides with covalently closed ends are shown in FIGS. 14-17. The linear double-stranded DNA with covalently closed ends can be generated with any suitable method, such as by treating a double-stranded polynucleotide with a suitable enzyme such as a protelomerase enzyme, e.g., TelN or a suitable alternative such as ligating hairpin loops to a linear double stranded DNA. In certain embodiments, the polynucleotide is single-stranded DNA or RNA, single-stranded DNA or RNA with a 5′ hairpin, single-stranded DNA or RNA with a 3′ hairpin, single-stranded DNA or RNA with a 5′ hairpin and a 3′ hairpin, a half loop, a single-stranded DNA or RNA with a bound 5′ oligo, a single-stranded DNA or RNA with a bound 5′ oligo and a 3′ hairpin, a single-stranded DNA or RNA with a bound 5′ oligo and a bound 3′ oligo, a single-stranded DNA or RNA with a bound 3′ oligo, a single-stranded DNA or RNA with a bound 3′ oligo and a 5′ hairpin, a double hairpin, a mixed loop, a mixed chain, or a combination thereof. Exemplary polynucleotide designs can be found in Shy et al. (2023) NATURE BIOTECHNOLOGY. In certain embodiments, the ligated hairpin loops comprise a nuclear localization signal bound to the hairpin loop. In certain embodiments, the polynucleotide further comprises one or more of (1) a donor template (D), (2) a first PAM (P₁) and a first target nucleotide sequence (T₁), and, optionally, (3) a second PAM (P₂) and a second target nucleotide sequence (T₂). The polynucleotide can comprise any suitable number of PAMs and target nucleotide sequences, such as at least 1, 2, 3, 4, 5, 6, 7, 8, or 9 and/or not more than 10, 9, 8, 7, 6, 5, 4, 3, or 2, for example 1-10, preferably 1-6, more preferably 1-3, even more preferably 2. As used herein, the term “target nucleotide sequence” includes a sequence in a polynucleotide to which a nucleic acid-guided nuclease can bind, e.g., a protospacer. The target nucleotide sequence comprises at least partial complementarity to the spacer sequence of the nucleic acid-guide nuclease such that the nucleic acid-guided nuclease can bind to and, optionally, cleave one or more of strand of the polynucleotide at or near the target nucleotide sequence Typically, the target nucleotide sequence lies within a target site in a target polynucleotide, and upon binding of a nucleic acid-guided nuclease complex to the target nucleotide sequence, the nuclease can, optionally, generate a strand break in one or more strands of the polynucleotide. The donor template can comprise and suitable sequence comprising any suitable number and combination of components, e.g., transgenes, as needed for the application. For example, the donor template can comprise a regulator element, such as a promoter (any suitable regulatory element can be used, for example one or more of the sequences shown in Table 5), a payload, such as a heterologous gene, e.g., a transgene or a payload, for example a chemokine receptor, a chimeric antigen receptor (CAR, for example sequence shown in Table 1) or a chimeric auto-antibody receptor (CAAR), and/or a terminator. In certain embodiments, the transgene encodes a self-cleaving peptide, such as a 2A peptide. In certain embodiments, the transgene encodes a reporter protein, such as a fluorescent protein, an antibiotic resistance marker, or the like. In certain embodiments, the heterologous gene comprises a polynucleotide encoding a polypeptide comprising a CAR or portion thereof that binds a binding partner comprising B7H3, BCMA, GPRC5D, CD8, CD8a, CD19, CD20, CD22, CD28, 4-1BB, or CD3zeta or a portion thereof. In certain embodiments, the heterologous gene comprises a polynucleotide encoding a CAR or portion thereof that comprises a polypeptide at least 60, 70, 75, 80, 85, 90, 95, 96, 97, 98, 99, 99.5, or 100% identical to any one of the amino acid sequences of SEQ ID NOs: 86-124 as shown in Table 1. In other embodiments, the donor template comprises sequence that once inserted into the target site generates one or more mutations in the gene at the target site, such as a SNP, and INDEL, or a missense mutation. In certain embodiments, the polynucleotide can further comprise one or more homology arms, for example an upstream and a downstream homology arm with respect to the donor template. In certain embodiments, the polynucleotide comprises a first PAM (P₁) and first target nucleotide sequence (T₁) either upstream or downstream of the donor template (D). In certain embodiments, the first PAM (P₁) and first target nucleotide sequence (T₁) are adjacent to but not within the donor template. The first PAM (P₁) and first target nucleotide sequence (T₁) should be oriented in such a way that the respective nucleic acid-guided nuclease can recognize the PAM and target nucleotide sequence. For example, when using a Type Va nucleic acid-guided nuclease, such as a nuclease as disclosed herein, for example cpf1 and/or MAD7, the first PAM (P₁) and first target nucleotide sequence (T₁) should be oriented such that the first PAM (P₁) is 5′ of the first target nucleotide sequence (T₁). In certain embodiments, for an upstream first PAM (P₁) and first target nucleotide sequence (T₁) for a type Va nucleic acid-guided nuclease, the first PAM (P₁) and first target nucleotide sequence (T₁) can be oriented as follows: (1) 5′ P₁⁺T₁⁺D⁺ 3′ or (2) 5′ T₁⁻P₁⁻D⁺ 3′, wherein the D⁺ signifies the sense strand of the donor template. In certain embodiments, for a downstream first PAM (P₁) and first target nucleotide sequence (T₁) for a type Va nucleic acid-guided nuclease, the first PAM (P₁) and first target nucleotide sequence (T₁) can be oriented as follows: (1) 5′ D⁺P₁⁺T₁⁺ 3′ or (2) 5′ D⁺T₁⁻P₁⁻ 3′, wherein the D⁺ signifies the sense strand of the donor template. In certain embodiments, the polynucleotide comprises a first PAM (P₁) and first target nucleotide sequence (T₁) upstream of the donor template (D) and a second PAM (P₂) and a second target nucleotide sequence (T₂) downstream of the donor template (D). In certain embodiments, the first PAM (P₁) and first target nucleotide sequence (T₁) can be oriented as follows: (1) 5′ P₁⁺T₁⁺D⁺ 3′ or (2) 5′ T₁⁻P₁⁻D⁺ 3′, and the second PAM (P₂) and a second target nucleotide sequence (T₂) can be oriented as follows: (1) 5′ D⁺P₂⁺T₂⁺ 3′ or (2) 5′ D⁺T₂⁻P₂⁻ 3′. In certain embodiments, the first and second PAM target nucleotide sequences are oriented 5′ T₁⁻P₁⁻D⁺P₂⁺T₂⁺ 3′ (as illustrated in FIG. 7, wherein the sense strand of the donor template is marked 701), 5′ T₁⁻P₁⁻D⁺T₂⁻P₂⁻ 3′ (as illustrated in FIG. 5, wherein the sense strand of the donor template is marked 501), 5′ P₁⁺T₁⁺D⁺T₂⁻P₂⁻ 3′ (as illustrated in FIG. 4, wherein the sense strand of the donor template is marked 401), or 5′ P₁⁺T₁⁺D⁺P₂⁺T₂⁺ 3′ (as illustrated in FIG. 6, wherein the sense strand of the donor template is marked 601), preferably 5′ T₁⁻P₁⁻D⁺P₂⁺T₂⁺ 3′ or 5′ P₁⁺T₁⁺D⁺T₂⁻P₂⁻ 3′.

In certain embodiments, the PAM comprises a sequence suitable for the nucleic acid-guided nuclease being used. For example, the PAM would comprise a suitable sequence to be recognized by a Type V nucleic acid-guided nuclease for an application using a Type V nucleic acid-guided nuclease as disclosed herein (for example Tables 3 and 4). In certain embodiments, the PAM comprises a sequence of CTTN or TTTN. It should be understood that any suitable PAM sequence can be used for the respective nucleic acid-guided nuclease. For example, an engineered nucleic acid-guided nuclease wherein the PAM specificity was adjusted, altered, abrogated, etc., the first PAM would comprise the respective sequence to be recognized by the engineered nucleic acid-guided nuclease.

The target nucleotide sequence can comprise any suitable sequence as necessary for the intended application.

The polynucleotide can comprise any suitable number of binding sites comprising a suitable PAM and target nucleotide sequence for a nucleic acid-guide nuclease, and, therefore, the composition can comprise a polynucleotide with any suitable number of nucleic acid-guided nuclease complexes bound. In certain embodiments, the polynucleotide comprises 1, 2, 3, 4, 5, 6, 7, 8, or 9 and/or not more than 2, 3, 4, 5, 6, 7, 8, 9, or 10 binding site, for example 1-10, preferably 1 or 2 binding sites. In an illustrative example, a composition comprising a polynucleotide comprising 2 binding sites can comprise up to 2 nucleic acid-guided nuclease complexes bound to the polynucleotide, and a composition comprising a polynucleotide comprising 3 binding sites can comprise up to 3 nucleic-acid-guided nuclease complexes bound to the polynucleotide.

In certain embodiments, provided herein are compositions comprising a plurality of polynucleotides. In certain embodiments, the composition comprises a plurality of polynucleotides as described above. In certain embodiments, the plurality of polynucleotides comprises (1) a donor template (D)ₓ, (2) a first suitable PAM (P₁)ₓ and a first target nucleotide sequence (T₁)ₓ, and (3) a second suitable PAM (P₂)ₓ and a second target nucleotide sequence (T₂)ₓ, wherein, for each integer x, the polynucleotide comprises a different donor template sequence. The donor template can comprise any suitable sequence for the intended application. In certain embodiments, the donor template comprises a sequence encoding a heterologous gene, such as a CAR or a CAAR, for example a sequence encoding a polypeptide comprising a CAR or portion thereof that binds a binding partner comprising B7H3, BCMA, GPRC5D, CD8, CD8a, CD19, CD20, CD22, CD28, 4-1BB, or CD3zeta or a portion thereof, preferably a sequence encoding a CAR or portion thereof that comprises a polypeptide at least 60, 70, 75, 80, 85, 90, 95, 96, 97, 98, 99, 99.5, or 100% identical to any one of the amino acid sequences of SEQ ID NOs: 86-124. The plurality of polynucleotides can comprise any suitable number of different polynucleotides, i.e., integers x. In certain embodiments, the number of different integers x is at least 2, 3, 4, 5, 6, 7, 8, or 9 and/or no more than 10, 9, 8, 6, 5, 4, or 3, for example 2-10, preferably 2-5. In certain embodiments, the number of different integers x is at least 10, 20, 30, 40, 50, 60, 70, 80, or 90 and/or no more than 100, 90, 80, 60, 50, 40, 30, or 20, for example 10-100, preferably 10-50.

In certain embodiments, provided herein are compositions comprising a cell comprising a polynucleotide and/or a plurality of nucleotides as described above. In certain embodiments, the cell is a human cell. In certain embodiments, the cell is a human cell or a stem cell. In certain embodiments, the human cell is an immune cell comprising a neutrophil, cosinophil, basophil, mast cell, monocyte, macrophage, dendritic cell, natural killer cell, or a lymphocyte, preferably a T cell. In certain embodiments, the human cell is a stem cell that is a human pluripotent, multipotent stem cell, embryonic stem cell, induced pluripotent stem cell (iPSC), hematopoietic stem cell, CD34+ cell, preferably an iPSC. In certain embodiments, the cell is a cell demonstrating reduced immunogenicity when placed in an allogeneic host. In certain embodiments, the cell is non-immunogenic when placed in an allogeneic host.

TABLE 1
CARs

| SEQ ID NO | Antigen | Sequence |
|---|---|---|
| 86 | BCMA | EVQLVESGGGLVQPGGSLRLSCAASGNIFSDNLMGWFRQAPGKE |
| | | REFVAAINWNSRSTYYADSVKGRFTISADNSKNTAYLQMNSLKP |
| | | EDTAVYYCAKDLTMVRGVPDYWGQGTLVTVSS |
| 87 | BCMA | EVQLVESGGGLVQPGGSLRLSCAASGFTLGDYVMGWFRQAPGKE |
| | | REWVSVISSSGDFTSYADSVKGRFTISADNSKNTAYLQMNSLKP |
| | | EDTAVYYCASHYYDSSGTNWGQGTLVTVSS |
| 88 | BCMA | EVQLVESGGGLVQPGGSLRLSCAASGFTFSSAIMGWFRQAPGKE |
| | | REFVSAITWNGTRTYYADSVKGRFTISADNSKNTAYLQMNSLKP |
| | | EDTAVYYCAKDLLEVGATPGNWGQGTLVTVSS |
| 89 | BCMA | EVQLLESGGGLVQPGGSLRLSCAASGFTFETYAMSWVRQAPGKG |
| | | LEWVSGISPSGGITTYADSVKGRFTISRDNSKNTLYLQMNSLRA |
| | | EDTAVYYCARREWWYDDWYLDYWGQGTLVTVSS |
| 90 | BCMA | EVQLLESGGGLVQPGGSLRLSCAASGFSFSTFAMSWVRQAPGKG |
| | | LEWVSAISGSGGSTSYADSVKGRFTISRDNSKNTLYLQMNSLRA |
| | | EDTAVYYCARRGWGSWSWYFDLWGQGTLVTVSS |
| 91 | BCMA | EVQLLESGGGLVQPGGSLRLSCAASGFTFGNYAMAWVRQAPGKG |
| | | LEWVSAISGSGGGTSYADSVKGRFTISRDNSKNTLYLQMNSLRA |
| | | EDTAVYYCARREWWYDDWYLDYWGQGTLVTVSS |
| 92 | BCMA | DIQMTQSPSSLSASVGDRVTITCRASQTIERRLNWYQQKPGKAP |
| | | KLLIYAASDLESGVPSRFSGSGSGTDFTLTISSLQPEDFATYYC |
| | | QQNNNWPTTFGQGTKVEIK |
| 93 | BCMA | DIQMTQSPSSLSASVGDRVTITCRASQTIGIYLNWYQQKPGKAP |
| | | KLLIYDASSLHSGVPSRFSGSGSGTDFTLTISSLQPEDFATYYC |
| | | QQSYSTPFTFGGGTKVEIK |
| 94 | BCMA | DIQMTQSPSSLSASVGDRVTITCRASQTIGDYLNWYQQKPGKAP |
| | | KLLIYAVTSRASGVPSRFSGSGSGTDFTLTISSLQPEDFATYYC |
| | | QQSYSTLTFGQGTKVEIK |
| 95 | B7H3 | EVQLVESGGGLVQPGGSLRLSCAASGIAFSIDIMGWFRQAPGKE |
| | | REFVAAVNWNGDSTYYADSVKGRFTISADNSKNTAYLQMNSLKP |
| | | EDTAVYYCATIDGSWREWGQGTLVTVSS |
| 96 | B7H3 | EVQLVESGGGLVQPGGSLRLSCAASGLRFDDYWMGWFRQAPGKE |
| | | REFVSAINWSGVSTYYADSVKGRFTISADNSKNTAYLQMNSLKP |
| | | EDTAVYYCAARQYGEYWQAAGWGQGTLVTVSS |
| 97 | B7H3 | EVQLVESGGGLVQPGGSLRLSCAASGLTLDYYAMGWFRQAPGKE |
| | | REFVAGINNGRAITYYADSVKGRFTISADNSKNTAYLQMNSLKP |
| | | EDTAVYYCATIDGSWREWGQGTLVTVSS |
| 98 | B7H3 | EVQLLESGGGLVQPGGSLRLSCAASGFTFSNFPMSWVRQAPGKG |
| | | LEWVSAITGTGGSTYYADSVKGRFTISRDNSKNTLYLQMNSLRA |
| | | EDTAVYYCATRTGTTGTAFDIWGQGTLVTVSS |
| 99 | B7H3 | EVQLLESGGGLVQPGGSLRLSCAASGYTFSNYAMSWVRQAPGKG |
| | | LEWVSAVSRSGGSTYYADSVKGRFTISRDNSKNTLYLQMNSLRA |
| | | EDTAVYYCARDLGYYAFDFWGQGTLVTVSS |
| 100 | B7H3 | EVQLLESGGGLVQPGGSLRLSCAASGFTFSTYAMSWVRQAPGKG |
| | | LEWVSSISGSGGRTDYADSVKGRFTISRDNSKNTLYLQMNSLRA |
| | | EDTAVYYCARIRSRGSSGFDPWGQGTLVTVSS |
| 101 | B7H3 | DIQMTQSPSSLSASVGDRVTITCRASQNIGRYLNWYQQKPGKAP |
| | | KLLIYDASGLQSGVPSRFSGSGSGTDFTLTISSLQPEDFATYYC |
| | | QQSYSTPPWTFGGGTKVEIK |
| 102 | B7H3 | DIQMTQSPSSLSASVGDRVTITCRASQTIYRYLNWYQQKPGKAP |
| | | KLLIYHASNLQSGVPSRFSGSGSGTDFTLTISSLQPEDFATYYC |
| | | QQSYTFPRSFGGGTKVEIK |
| 103 | B7H3 | DIQMTQSPSSLSASVGDRVTITCRASQSVYSYLNWYQQKPGKAP |
| | | KLLIYETSNLQSGVPSRFSGSGSGTDFTLTISSLQPEDFATYYC |
| | | QQSFTSPLTFGGGTKVEIK |
| 104 | CD19 | EVQLLESGGGLVQPGGSLRLSCAASGFTFENYAMSWVRQAPGKG |
| | | LEWVSAISGSGGHTYYADSVKGRFTISRDNSKNTLYLQMNSLRA |
| | | EDTAVYYCAHSNKRTGHAFDIWGQGTLVTVSS |
| 105 | CD19 | EVQLLESGGGLVQPGGSLRLSCAASGFTFSRHAMSWVRQAPGKG |
| | | LEWVSAITGSGASTYYADSVKGRFTISRDNSKNTLYLQMNSLRA |
| | | EDTAVYYCARGGRREFHYGLDYWGQGTLVTVSS |
| 106 | CD19 | EVQLLESGGGLVQPGGSLRLSCAASGFTFGNYAMAWVRQAPGKG |
| | | LEWVSAISGNGGSTFYADSVKGRFTISRDNSKNTLYLQMNSLRA |
| | | EDTAVYYCARAGRILFDYWGQGTLVTVSS |
| 107 | CD19 | EVQLLESGGGLVQPGGSLRLSCAASGFTFSTYAMSWVRQAPGKG |
| | | LEWVSAISRSGGNTYYADSVKGRFTISRDNSKNTLYLQMNSLRA |
| | | EDTAVYYCARVRMKGYTYFDPWGQGTLVTVSS |
| 108 | CD19 | EVQLLESGGGLVQPGGSLRLSCAASGFTFSHYGMSWVRQAPGKG |
| | | LEWVSSISGSGGSTYYVDSVKGRFTISRDNSKNTLYLQMNSLRA |
| | | EDTAVYYCARSKRLIHGLDVWGQGTLVTVSS |
| 109 | CD19 | EVQLLESGGGLVQPGGSLRLSCAASGFTFSRYTMSWVRQAPGKG |
| | | LEWVSTISGSGYSTYYADSVKGRFTISRDNSKNTLYLQMNSLRA |
| | | EDTAVYYCAHSNKRTGHAFDIWGQGTLVTVSS |
| 110 | CD19 | DIQMTQSPSSLSASVGDRVTITCRASQSVSTFLNWYQQKPGKAP |
| | | KLLIYGASILQSGVPSRFSGSGSGTDFTLTISSLQPEDFATYYC |
| | | QQSYTPPLTFGGGTKVEIK |
| 111 | CD19 | DIQMTQSPSSLSASVGDRVTITCRASQSVSRFLNWYQQKPGKAP |
| | | KLLIYAASVLQSGVPSRFSGSGSGTDFTLTISSLQPEDFATYYC |
| | | QQTYSPPLTFGGGTKVEIK |
| 112 | CD19 | DIQMTQSPSSLSASVGDRVTITCRASQSIRRYLNWYQQKPGKAP |
| | | KLLIYHTSRLQSGVPSRFSGSGSGTDFTLTISSLQPEDFATYYC |
| | | AQGWGRPVTFGQGTKVEIK |
| 113 | CD19 | DIQMTQSPSSLSASVGDRVTITCRASQTISSSLNWYQQKPGKAP |
| | | KLLIYGASSLRSGVPSRFSGSGSGTDFTLTISSLQPEDFATYYC |
| | | QQTYSNPITFGGGTKVEIK |
| 114 | CD19 | DIQMTQSPSSLSASVGDRVTITCRTSQSISTYLNWYQQKPGKAP |
| | | KLLIYGASALQTGVPSRFSGSGSGTDFTLTISSLQPEDFATYYC |
| | | QQSYTAPLTFGGGTKVEIK |
| 115 | CD19 | DIQMTQSPSSLSASVGDRVTITCRASQTISKYLNWYQQKPGKAP |
| | | KLLIYGASSLQSGVPSRFSGSGSGTDFTLTISSLQPEDFATYYC |
| | | QQSYSPPITFGGGTKVEIK |
| 116 | CD22 | EVQLVESGGGLVQPGGSLRLSCAASGIPSIRAMGWFRQAPGKER |
| | | EWVSSINSDGTSAFYADSVKGRFTISADNSKNTAYLQMNSLKPE |
| | | DTAVYYCARAYGRGTYDWGQGTLVTVSS |
| 117 | CD22 | EVQLVESGGGLVQPGGSLRLSCAASGFTFGEYAMGWFRQAPGKE |
| | | REFVASISRSGTLRAYADSVKGRFTISADNSKNTAYLQMNSLKP |
| | | EDTAVYYCAKESKDYFYMDVWGQGTLVTVSS |
| 118 | CD22 | EVQLVESGGGLVQPGGSLRLSCAASGRTYGMGWFRQAPGKEREF |
| | | VASVTSGGYTNYADSVKGRFTISADNSKNTAYLQMNSLKPEDTA |
| | | VYYCARGGGTSVRAFDIWGQGTLVTVSS |
| 119 | CD22 | EVQLLESGGGLVQPGGSLRLSCAASGFAFAAYDMGWVRQAPGKG |
| | | LEWVSSISGYGSTTYYADSVKGRFTISRDNSKNTLYLQMNSLRA |
| | | EDTAVYYCARHSGYGSSYGVLFAYWGQGTLVTVSS |
| 120 | CD22 | EVQLLESGGGLVQPGGSLRLSCAASGFAFAAYDMGWVRQAPGKG |
| | | LEWVATISGGGINTYYPDSVKGRFTISRDNSKNTLYLQMNSLRA |
| | | EDTAVYYCARHSGYGSSYGVLFAYWGQGTLVTVSS |
| 121 | CD22 | EVQLLESGGGLVQPGGSLRLSCAASGFTFPVYNMAWVRQAPGKG |
| | | LEWVSEIDALGTDTYYADSVKGRFTISRDNSKNTLYLQMNSLRA |
| | | EDTAVYYCARHSGYGSSYGVLFAYWGQGTLVTVSS |
| 122 | CD22 | DIQMTQSPSSLSASVGDRVTITCRASQSISNNLNWYQQKPGKAP |
| | | KLLIYGKNIRPSGVPSRFSGSGSGTDFTLTISSLQPEDFATYYC |
| | | FQGSQFPYTFGQGTKVEIK |
| 123 | CD22 | DIQMTQSPSSLSASVGDRVTITCRASQDVSSGVAWYQQKPGKAP |
| | | KLLIYHASQSISGVPSRFSGSGSGTDFTLTISSLQPEDFATYYC |
| | | QSYDLKSLNVVFGQGTKVEIK |
| 124 | CD22 | DIQMTQSPSSLSASVGDRVTITCQASQSISSYLAWYQQKPGKAP |
| | | KLLIYGQHNRPSGVPSRFSGSGSGTDFTLTISSLQPEDFATYYC |
| | | QQSYNTPRTFGQGTKVEIK |

### B. Polynucleotides and Polypeptides Bound Thereto

In certain embodiments, provided herein are compositions comprising a polynucleotide and a polypeptide bound to the polynucleotide. In certain embodiments, the polynucleotide can comprise any of the polynucleotides as disclosed herein. In preferred embodiments, the polypeptide further comprises one or more functional moieties, such as a nuclear localization sequence (NLS) or an affinity tag. In a more preferred embodiment, the composition comprises a polynucleotide and a polypeptide bound to the polynucleotide wherein the polypeptide comprises an NLS. In certain embodiments, the polypeptide comprises at least 1, 2, 3, 4, 5, 6, 7, 8, or 9 and/or not more than 10, 9, 8, 7, 6, 5, 4, 3, or 2 functional moieties on the N-terminus and/or C-terminus, such as 1-10 functional domains. In certain embodiments, the polypeptide comprises 3-7 to 4-6 functional domains on the N-terminus. Any suitable polypeptide can be used, for example a DNA-binding protein, a homing endonuclease, or a nucleic acid-guided nuclease, such as a Class I or Class II nucleic acid-guide nucleases, for example a Type V nucleic acid-guided nuclease. In preferred embodiments, the polypeptide comprises a nucleic acid-guided nuclease complexed with a compatible guide nucleic acid (gNA), i.e., a nucleic acid-guided nuclease complex, for example, a ribonucleoprotein (RNP). The polynucleotide can comprise any suitable number of binding sites comprising a suitable PAM and target nucleotide sequence for a nucleic acid-guide nuclease, and, therefore, the composition can comprise a polynucleotide with any suitable number of nucleic acid-guided nuclease complexes bound. In certain embodiments, the polynucleotide comprises 1, 2, 3, 4, 5, 6, 7, 8, or 9 and/or not more than 2, 3, 4, 5, 6, 7, 8, 9, or 10 binding sites, for example 1-10, preferably 1 or 2 binding sites. In an illustrative example, a composition comprising a polynucleotide comprising 2 binding sites can comprise up to 2 nucleic acid-guided nuclease complexes bound to the polynucleotide, and a composition comprising a polynucleotide comprising 3 binding sites can comprise up to 3 nucleic-acid-guided nuclease complexes bound to the polynucleotide. In certain embodiments, the one or more bound nucleic acid-guide nuclease complexes are the same. In other embodiments, they are different. In a preferred embodiment, the one or more nucleic acid guided nuclease complexes are the same, and, therefore the first, second, and/or additional target sequences share sufficient complementarity with the spacer sequence in the guide nucleic acid of the nucleic acid-guided nuclease complex such that the complex can bind to the target nucleotide sequence. In certain embodiments, at least one strand of the polynucleotide comprising a target nucleotide sequence is cut at or near the target nucleotide sequence upon binding of the nucleic acid-guide nuclease complex to the target nucleotide sequence. In preferred embodiments, the polynucleotide comprises double stranded DNA and both strands of the polynucleotide are cut at or near the target nucleotide sequence upon binding of the nucleic acid-guide nuclease complex to the target nucleotide sequence. In other embodiments, neither strand of the polynucleotide comprising a target nucleotide sequence is cut at or near the target nucleotide sequence upon binding of the nucleic acid-guide nuclease complex to the target nucleotide sequence. In certain cases, an engineered nucleic acid-guided nuclease complex can be used that lacks the ability to cut one or more strands of the polynucleotide. In other cases, the gNA comprises a spacer sequence that shares sufficient complementary with a target nucleotide sequence to bind to the target nucleotide sequence but not sufficient complementary to cut the polynucleotide at or near the target nucleotide sequence.

The nucleic acid-guide nuclease complex can comprise any suitable nucleic acid guide nuclease and a compatible gNA as disclosed herein.

In certain embodiments, the nucleic acid-guide nuclease comprises an engineered, non-naturally occurring nuclease. In certain embodiments, the nucleic acid-guided nuclease comprises a Class 1 or a Class 2 nucleic acid-guide nuclease, such as a Type II or a Type V, for example a Type V-A, V-B, V-C, V-D, or V-E nucleic acid-guided nuclease, preferably a Type V-A (Va) nucleic acid-guide nuclease. In certain embodiments, the nucleic acid-guide nuclease comprises a MAD, ART, or an ABW nuclease. In preferred embodiments, the nucleic acid-guided nuclease comprises an amino acid sequence at least 80, 85, 90, 95, 99, or 100% identical to an amino acid sequence of a MAD, ART, or ABW nucleic acid-guided nuclease, preferably an amino acid sequence at least 80, 85, 90, 95, 99, or 100% identical to an amino acid sequence of a MAD nuclease, more preferably an amino acid sequence at least 80, 85, 90, 95, 99, or 100% identical to an amino acid sequence of a MAD7 nuclease, even more preferably an amino acid sequence that is at least 80, 85, 90, 95, 99, or 100% identical to the amino acid sequence of SEQ ID NO: 37.

In certain embodiments, the nucleic acid-guide nuclease complex comprises at least four NLS, preferably at least 5 NLS arranged in any suitable orientation. In certain embodiments, the nucleic acid-guided nuclease comprises one N-terminal and three C-terminal NLS. In other embodiments, the nucleic acid-guided nuclease comprises five or more N-terminal NLS. The NLS can comprise any suitable sequence as disclosed herein, preferably any one of SEQ ID NOs: 40-56, more preferably SEQ ID NOs: 40, 51, and 56.

In certain embodiments, the gNA comprises an engineered, non-naturally occurring nuclease. In certain embodiments, the gNA comprises a spacer sequence heterologous to any naturally occurring spacer sequence for the respective nucleic acid-guided nuclease. In certain embodiments, the gNA comprises a single polynucleotide. In preferred embodiments, the gNA comprises a dual gNA as disclosed herein, for example a guide nucleic acid comprising a targeter nucleic acid and a modulator nucleic acid capable of binding to and activating a nucleic acid-guided nuclease, that, in a naturally occurring system, is activated by a single crRNA in the absence of a tracrRNA.

In certain embodiments, provided herein are compositions comprising polynucleotides and polypeptides bound thereto. In preferred embodiments, the polypeptide bound thereto comprises one or more NLS. The polynucleotide can comprise single-stranded DNA, single-stranded RNA, double-stranded DNA, or double-stranded RNA, preferably, double-stranded DNA, for example a plasmid. In certain embodiments, the linear double-stranded DNA comprises covalently closed ends, for example as generated by a telomerase enzyme such as TelN or a suitable alternative. In certain embodiments, the composition comprises a polynucleotide comprising one or more of (1) a donor template (D), (2) a first PAM (P₁) and a first target nucleotide sequence (T₁), and optionally a bound polypeptide comprising an NLS, and/or (3) a second PAM (P₂) and a second target nucleotide sequence (T₂) and optionally a bound polypeptide comprising an NLS. The donor template can comprise and suitable sequence comprising any suitable number and combination of components as needed for the application. For example, the donor template can comprise a sequence encoding a promoter (for example sequences shown in Table 5), a heterologous gene, such as a chimeric antigen receptor (CAR) or a chimeric auto-antibody receptor (CAAR), and/or a terminator. In certain embodiments, the heterologous gene comprises a polynucleotide encoding a polypeptide comprising a CAR or portion thereof that binds a binding partner comprising B7H3, BCMA, GPRC5D, CD8, CD8a, CD19, CD20, CD22, CD28, 4-1BB, or CD3zeta or a portion thereof. In certain embodiments, the heterologous gene comprises a polynucleotide encoding a CAR or portion thereof that comprises a polypeptide at least 60, 70, 75, 80, 85, 90, 95, 96, 97, 98, 99, 99.5, or 100% identical to any one of the amino acid sequences of SEQ ID NOs: 86-124 as shown in Table 1.

In certain embodiments, the composition comprises a polynucleotide comprising a first PAM (P₁) and first target nucleotide sequence (T₁) either upstream or downstream of the donor template (D) and a polypeptide comprising an NLS bound thereto. In certain embodiments, the first PAM (P₁) and first target nucleotide sequence (T₁) are adjacent to but not within the donor template. The first PAM (P₁) and first target nucleotide sequence (T₁) should be oriented in a manner function for the respective nucleic acid-guided nuclease. For example, when using a Type Va nucleic acid-guided nuclease, such as cpf1 or MAD7, the first PAM (P₁) and first target nucleotide sequence (T₁) should be oriented such that the first PAM (P₁) is upstream of the first target nucleotide sequence (T₁). In certain embodiments, for an upstream first PAM (P₁) and first target nucleotide sequence (T₁) for a type Va nucleic acid-guided nuclease, the first PAM (P₁) and first target nucleotide sequence (T₁) can be oriented as follows: (1) 5′ P₁⁺T₁⁺D⁺ 3′ or (2) 5′ T₁⁻P₁⁻D⁺ 3′, wherein the D⁺ signifies the sense strand of the donor template. In certain embodiments, for a downstream first PAM (P₁) and first target nucleotide sequence (T₁) for a type Va nucleic acid-guided nuclease, the first PAM (P₁) and first target nucleotide sequence (T₁) can be oriented as follows: (1) 5′ D⁺P₁⁺T₁⁺ 3′ or (2) 5′ D⁺T₁⁻P₁⁻ 3′, wherein the D⁺ signifies the sense strand of the donor template. In certain embodiments, the polynucleotide comprises a first PAM (P₁) and first target nucleotide sequence (T₁) upstream of the donor template (D) and a polypeptide comprising an NLS bound thereto and a second PAM (P₂) and a second target nucleotide sequence (T₂) downstream of the donor template (D) and a polypeptide comprising an NLS bound thereto. In certain embodiments, the first PAM (P₁) and first target nucleotide sequence (T₁) can be oriented as follows: (1) 5′ P₁⁺T₁⁺D⁺ 3′ or (2) 5′ T₁⁻P₁⁻D⁺ 3′, and the second PAM (P₂) and a second target nucleotide sequence (T₂) can be oriented as follows: (1) 5′ D⁺P₂⁺T₂⁺ 3′ or (2) 5′ D⁺T₂⁻P₂⁻ 3′. In certain embodiments, the first and second PAM target nucleotide sequences are oriented 5′ T₁⁻P₁⁻D⁺P₂⁺T₂⁺ 3′ (as illustrated in FIG. 7, wherein the sense strand of the donor template is marked 701), 5′ T₁⁻P₁⁻D⁺T₂⁻P₂⁻ 3′ (as illustrated in FIG. 5, wherein the sense strand of the donor template is marked 501), 5′ P₁⁺T₁⁺D⁺T₂⁻P₂⁻ 3′ (as illustrated in FIG. 4, wherein the sense strand of the donor template is marked 401), or 5′ P₁⁺T₁⁺D⁺P₂⁺T₂⁺ 3′ (as illustrated in FIG. 6, wherein the sense strand of the donor template is marked 601), preferably 5′ T₁⁻P₁⁻D⁺P₂⁺T₂⁺ 3′ or 5′ P₁⁺T₁⁺D⁺T₂⁻P₂⁻ 3′.

Illustrated in FIG. 4 is an exemplary composition comprising a polynucleotide comprising a donor template, a first and second PAM, and a first and second target nucleotide sequence oriented 5′ P₁⁺T₁⁺D⁺T₂⁻P₂⁻ 3′ further comprising a first nucleic acid-guided nuclease complex comprising an NLS bound to first PAM and target nucleotide sequence and a second nucleic acid-guided nuclease complex comprising an NLS bound to second PAM and target nucleotide sequence. The sense strand of the donor template is represented as 401. In certain embodiments, the nucleic acid-guide nuclease complex does not release after cleavage and the cleavage products comprise the donor template (402) and the remainder of the polynucleotide with bound nucleic acid-guide nuclease complexes. The resulting donor template (402) does not comprise a bound nucleic acid-guided nuclease complex comprising an NLS.

Illustrated in FIG. 5 is an exemplary composition comprising a polynucleotide comprising a donor template, a first and second PAM, and a first and second target nucleotide sequence oriented 5′ T₁⁻P₁⁻D⁺T₂⁻P₂⁻ 3′ further comprising a first nucleic acid-guided nuclease complex comprising an NLS bound to first PAM and target nucleotide sequence and a second nucleic acid-guided nuclease complex comprising an NLS bound to second PAM and target nucleotide sequence. The sense strand of the donor template is represented as 501. In certain embodiments, the nucleic acid-guide nuclease complex does not release after cleavage and the cleavage products comprise the donor template and a bound nucleic acid-guide nuclease complex comprising an NLS to the upstream PAM and target nucleotide sequence (502) and the remainder of the polynucleotide. The resulting donor template (502) comprises one bound nucleic acid-guided nuclease complex comprising an NLS.

Illustrated in FIG. 6 is an exemplary composition comprising a polynucleotide comprising a donor template, a first and second PAM, and a first and second target nucleotide sequence oriented 5′ P₁⁺T₁⁺D⁺P₂⁺T₂⁺ 3′ further comprising a first nucleic acid-guided nuclease complex comprising an NLS bound to first PAM and target nucleotide sequence and a second nucleic acid-guided nuclease complex comprising an NLS bound to second PAM and target nucleotide sequence. The sense strand of the donor template is represented as 601. In certain embodiments, the nucleic acid-guide nuclease complex does not release after cleavage and the cleavage products comprise the donor template and a bound nucleic acid-guide nuclease complex comprising an NLS to the downstream PAM and target nucleotide sequence (602) and the remainder of the polynucleotide. The resulting donor template (602) comprises one nucleic acid-guided nuclease complex comprising an NLS.

Illustrated in FIG. 7 is an exemplary composition comprising a polynucleotide comprising a donor template, a first and second PAM, and a first and second target nucleotide sequence oriented 5′ T₁⁻P₁⁻D⁺P₂⁺T₂⁺ 3′ further comprising a first nucleic acid-guided nuclease complex comprising an NLS bound to first PAM and target nucleotide sequence and a second nucleic acid-guided nuclease complex comprising an NLS bound to second PAM and target nucleotide sequence. The sense strand of the donor template is represented as 701. In certain embodiments, the nucleic acid-guide nuclease complex does not release after cleavage and the cleavage products comprise the donor template and two bound nucleic acid-guide nuclease complexes comprising an NLS to both the upstream and downstream PAM and target nucleotide sequence (702) and the remainder of the polynucleotide. The resulting donor template (702) comprises two nucleic acid-guided nuclease complex comprising an NLS.

Not wishing to be bound by theory, the NLS operably connected to the donor template can help facility uptake of the donor template into a nucleus of a target cell and further increase the likelihood of introduction of at least a portion of the donor template at or near a target site in the genome of the target cell upon cleavage of the genome by the nucleic acid-guide nuclease complex by homology directed repair (HDR). This efficiency of HDR of the donor template may be improved by an increase in the local concentration of the donor template (HDR template) facilitated by increased nuclear localization by the one or more bound polypeptides comprising an NLS. It is surprising an unexpected that a nucleic acid-guided nuclease complex remains bound to the polynucleotide after cleavage improving the nuclear uptake of the cleaved product.

In certain embodiments, the polynucleotide further one or more additional PAM and target sites between the donor template and the first and/or second PAM and target sites. In certain cases, the one or more additional PAM and target sites are modified in such a way that a nucleic acid-guided nuclease complex is able to bind to the one or more additional PAM and target sites and not effect one or more breaks in the polynucleotide at or near the site. Any suitable modification can be used to modify the polynucleotide to prevent the generation of one or more breaks in the polynucleotide at or near the site, such designing the guide nucleic acid to have a spacer sequence with fewer complementary nucleotides or to have one or more mismatches in the spacer sequence, for example within the seed sequence. In certain embodiments, the length of complementary nucleotides between the spacer sequence and the target site comprises at least 1, 2, 3, 4, 5, 6, 7, 8, or 9, and not more than 10, 9, 8, 7, 6, 5, 4, 3, or 2 fewer complementary nucleotides as compared to a spacer sequence with perfect complementarity, for example 1-10 fewer complementary nucleotides. In other words, the spacer sequence may comprise at least 5, 6, 7, 8, 9, 10, 11, 12, 13, 14, or, 15 and/or not more than 25 or 20 nucleotides complementary to the target sequence, for example 5-20 complementary nucleotides. In certain embodiments, the spacer sequence comprises at least 1, 2, 3, 4, 5, 6, 7, 8, or 9 and/or not more than 10, 9, 8, 7, 6, 5, 4, 3, or 2 mismatches as compared to the target sequence, for example 1-10 mismatches. Any suitable number of mismatches may be in the seed sequence, for example 1, 2, 3, 4, or 5 of the total number of mismatches may be in the seed sequence.

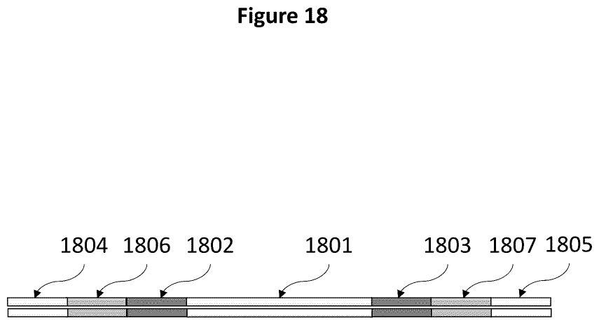

An exemplary polynucleotide comprising one or more additional PAM and target sites is shown in FIG. 18. Specifically, FIG. 18 shows a polynucleotide comprising a donor template (1801) flanked by upstream (1802) and downstream (1803) homology arms, further flanked by a first (1804) and a second (1805) PAM and target site, preferably which are designed in such a way that when the nucleic acid-guided nuclease complex binds, one or more strand breaks is effected, and further flanked by one or more additional (1806 and 1807) PAM and target sites, preferably which are designed in such a way that when the nucleic acid-guided nuclease complex binds, one or more strand breaks cannot be effected as disclosed herein. The polynucleotides comprising a donor template (1801) flanked by upstream (1802) and downstream (1803) homology arms can comprise any number of suitable additional elements, such as one or more of 1804, 1805, 1806, and/or 1807, for example 1 of 1804, 1805, 1806, and/or 1807, 2 of 1804, 1805, 1806, and/or 1807, three of 1804, 1805, 1806, and/or 1807, or all of 1804, 1805, 1806, and/or 1807.

In certain embodiments, the composition further comprises an additive that stabilizes the nucleic acid-guided nuclease complex. In certain embodiments, the one or more additives that stabilize nucleic acid-guided nuclease complexes are combined with the nuclease and the guide nucleic acid. In certain embodiments, the one or more additives that stabilize nucleic acid-guided nuclease complexes are combined with the guide nucleic acid prior to combination with the nuclease. In certain embodiments, the one or more additives that stabilize nucleic acid-guided nuclease complexes are combined with the nuclease prior to combination with the guide nucleic acid. In certain embodiments, the one or more additives that stabilize nucleic acid-guided nuclease complexes are combined with the pre-formed nucleic acid-guided nuclease complex comprising one or more nucleases and a guide nucleic acid. In certain embodiments, the one or more additives that stabilize nucleic acid-guided nuclease complexes prevent aggregation and/or support dispersion of nucleic acid-guided nuclease complexes in a population of nucleic acid-guided nuclease complexes. In certain embodiments, an RNP stabilizer may comprise any suitable protein stabilizer, such as a protein stabilizer known in the art. In certain embodiments, an RNP stabilizer comprises 1,2,3-heptanetriol, 2-Amino-2-(hydroxymethyl)-1,3-propanediol (Tris), 3-(1-pyridino)-1-propane sulfonate (NDSB 201), 3-[(3-cholamidopropyl)dimethylammonio]-1-propanesulfonate (CHAPS), 6-aminocaproic acid, adenosine diphosphate (ADP), adenosine triphosphate (ATP), alpha-cyclodextrin, amidosulfobetaine-14 (ASB-14), ammonium acetate, ammonium nitrate, ammonium sulfate, arginine, arginine ethylester, barium chloride, barium iodide, benzamidine HCl, beta-cyclodextrin, beta-mercaptoethanol (BME), biotin, calcium chloride, cesium chloride, cesium sulfate, cetyltrimethylammonium bromide (CTAB), choline chloride, citric acid, cobalt chloride, copper (II) chloride, cyclohexanol, D-sorbitol, dimethylethylammoniumpropane sulfonate (NDSB 195), dithiothreitol (DTT), erythritol, ethanol, ethylene glycol, ethylene glycol-bis(βbeta-aminoethyl ether)-N,N,N′,N′-tetraacetic acid (EGTA), ethylenediaminetetraacetic acid (EDTA), formamide, gadolinium bromide, gamma butyrolactone, glucose, glutamic acid, glutamine, glycerol, glycine, glycine betaine, glycine-glycine-glycine, guanidine HCl, guanosine triphosphate (GTP), holmium chloride, imidazole, iron (III) chloride, Jeffamine M-600, lanthanum acetate, lauryl sulfobetaine, lauryldimethylamine N-oxide (LDAO), lithium sulfate, magnesium chloride, magnesium sulfate, manganese chloride, mannitol, N-(2-hydroxyethyl) piperazine-N′-(3-propanesulfonic acid) (EPPS), N-dodecyl beta-D-maltoside (DDM), N-ethylurea, n-hexanol, N-lauryl sarcoside, N-lauryl sarcosine, N-methylformamide, N-methylurca, n-octyl-b-D-glucoside (OG: Octyl glucoside), n-penthanol, nickel chloride, non-detergent sulfo betaine (NDSB), Nonidet P40 (NP40), octyl beta-D-glucopyranoside, poly-L-glutamic acid, polyethylene glycol (for example, PEG 300, PEG 3350, PEG 4000), polyethyleneglycol lauryl ether (Brij 35), polyoxyethylene (2) oleyl ether (Brij 93), polyoxyethylene cetyl ether (Brij 56), polyvinylpyrrolidone 40 (PVP40), potassium chloride, potassium citrate, potassium nitrate, proline, putrescine, spermidine, spermine, riboflavin, samarium bromide, sarcosine, sodium acetate, sodium chloride, sodium dodecyl sulfate (SDS), sodium fluoride, sodium iodide, sodium lauroyl sarcosinate (Sarkosyl), sodium malonate, sodium molybdate, sodium selenite, sodium sulfate, sodium thiocyanate, sucrose, taurine, trehalose, tricine, triethylamine, trimethylamine N-oxide (TMAO), tris(2-carboxyethyl)phosphinc (TCEP), Triton X-100, Tween 20, Tween 60, Tween 80, urea, vitamin B12, xylitol, yttrium chloride, yttrium nitrate, zinc chloride, Zwittergent 3-08, Zwittergent 3-14, or a combination thereof. In certain embodiments, the RNP stabilizer comprises a negatively charged polymer. In certain embodiments, the RNP stabilizer comprises poly-L-glutamic acid (PGA) or a suitable alternative. In certain embodiments, provided herein are compositions, methods, and/or kits comprising poly-L-glutamic acid.

In certain embodiments, provided herein are compositions comprising a cell comprising a polynucleotide and/or a plurality of nucleotides and polypeptides bound thereto as described above. In certain embodiments, the cell is a human cell. In certain embodiments, the cell is a human cell or a stem cell. In certain embodiments, the human cell is an immune cell comprising a neutrophil, eosinophil, basophil, mast cell, monocyte, macrophage, dendritic cell, natural killer cell, or a lymphocyte, preferably a T cell. In certain embodiments, the human cell is a stem cell that is a human pluripotent, multipotent stem cell, embryonic stem cell, induced pluripotent stem cell (iPSC), hematopoietic stem cell, CD34+ cell, preferably an iPSC. In certain embodiments, the cell is a cell demonstrating reduced immunogenicity when placed in an allogeneic host. In certain embodiments, the cell is non-immunogenic when placed in an allogencic host.

### C. Methods for Using Polynucleotides and Polypeptides Bound Thereto

In certain embodiments provided herein are methods. In certain embodiments, provided herein are methods for use of any one of the compositions as disclosed herein. In certain embodiments, the provided methods are suitable for one or more genome engineering applications.

In certain embodiments, provided herein are methods for cleaving one or more polynucleotides comprising a PAM and a target nucleotide sequence adjacent to but not within a donor template. In certain embodiments, the polynucleotide comprises a circular polynucleotide and upon binding to and cutting at or near the target nucleotide sequence by a nucleic acid-guide nuclease complex, at least one strand break is generated resulting in the linearization of the circular polynucleotide. In certain embodiments, the method for preparing a linearized polynucleotide comprises contacting a polynucleotide as disclosed herein with a nucleic acid-guided nuclease complex, wherein the nucleic acid-guided nuclease complex binds to a target site on the polynucleotide and generates at least one strand break at or near the target nucleotide sequence. In certain embodiments, the polynucleotide comprises two target sites and at least one strand break is generated at or near each of the target nucleotide sequences by one or more nucleic acid-guided nuclease complex. In certain embodiments, the nucleic acid guided-nuclease complex remains bound to the polynucleotide. In certain embodiments, the composition comprising the linearized product is delivered to a cell.

In certain embodiments, provided herein are methods for engineering a genome of a cell comprising: delivering to the cell a composition comprising a polynucleotide as described in the Polynucleotides section above and a nucleic acid-guided nuclease system comprising a nucleic acid-guided nuclease and a gNA. In certain embodiments, the method for engineering a genome of a cell comprises delivering to the cell a composition comprising any one of the compositions as disclosed herein.

In certain embodiments, the method of genome engineering comprises delivering a plurality of exogenous nucleic acids into the genome of a target cell comprising contacting the target cell with a composition comprising a plurality of polynucleotides each comprising a donor template (D)ₓ and a plurality of nucleic acid-guided nuclease complexes (N)ₓ, wherein for each integer x, the donor template comprises a different sequence. In certain embodiments, polynucleotide further comprises a first suitable PAM (P₁)ₓ and a first target nucleotide sequence (T₁)ₓ adjacent to the 5′ end of the donor template but not within the donor template and a second suitable PAM (P₂)ₓ and a second target nucleotide sequence (T₂)ₓ adjacent to the 3′ end of the donor template but not within the donor template. In certain embodiments, the polynucleotide further comprises a first homology arm (HA₁)ₓ between (P₁)ₓ(T₁)ₓ and (D)ₓ and a second homology arm (HA₂)ₓ between (P₂)ₓ(T₂)ₓ and (D)ₓ, where (HA₁)ₓ and (HA₂)ₓ are capable of initiating host cell mediated recombination of at least a portion of (D)ₓ at a target site (TS)ₓ selected from a plurality of target sites of the genome of the target cell. In certain embodiments, for each target site (TS)ₓ, a plurality of nucleic acid-guided nuclease complexes (N)ₓ capable of cleaving at (TS)ₓ and at least one of (T₁)ₓ and (T₂)ₓ, wherein cleaving of (TS)ₓ and at least one of (T₁)ₓ and (T₂)ₓ results in homologous recombination of at least a portion of (D)ₓ at (TS)ₓ. In preferred embodiments, at least one of the plurality of donor templates comprises a polynucleotide encoding for a CAR or a CAAR.

In certain embodiments, the method comprises generating a composition comprising a nucleic acid-guided nuclease and gNA by combining the nucleic acid-guided nuclease and the gNA in vitro in a suitable buffer and, optionally allowing the nucleic acid-guided nuclease and gNA to form into a nucleic acid-guided nuclease complex. In certain embodiments, one or more polynucleotides are then added to the composition. In preferred embodiments, the composition comprises buffer where at least one component required for the activity of the nucleic acid-guided nuclease complex is at an activity-limiting concentration, such that upon binding of the complex to the polynucleotide, the complex is unable to generate a strand break. An exemplary component is Magnesium (Mg²⁺). Such a Mg²⁺-limiting buffer can be generated by the addition of EDTA, which chelates the Mg²⁺, rendering it unavailable to the complex. In certain embodiments, the Mg²⁺-limited composition is then delivered to a target cell, wherein the intracellular Mg²⁺ is no longer limiting. Such a composition would prevent cleavage of the one or more polynucleotide prior to delivery to the intended target cell.

In certain embodiments, the method further comprises treating a cell with an HDR enhancer (NHEJ inhibitor). In certain embodiments, the one or more additives that inhibit NHEJ are introduced to the target cell prior to delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced to the target cell after delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced to the target cell both prior to and after delivery of the nucleic acid-guided nuclease, guide nucleic acid, and/or donor template, or one or more polynucleotides encoding the nucleic acid-guided nuclease, guide nucleic acid, and/or donor template. In certain embodiments, the one or more additives that inhibit NHEJ are introduced into the cell medium, wherein the one or more NHEJ inhibitors can enter the cell.

In certain embodiments, the one or more additives that inhibit NHEJ comprise a molecule that indirectly or directly affects the interaction of p53-binding protein 1 (53BP1) with ubiquitylated histones at double stranded breaks, for example, iP53 or the like. In certain embodiments, the one or more additives that inhibit NHEJ comprise a molecule that directly or indirectly affects the interaction of Ku proteins with DNA, for example, STL127705 or the like. In certain embodiments, the one or more additives that inhibit NHEJ comprise a molecule that directly or indirectly affects the activity of DNA-dependent protein kinases, for example, M3814, KU-0060648, NU7026 or the like. In certain embodiments, the one or more additives that inhibit NHEJ comprise a molecule that directly or indirectly affects the activity of ATM-Rad3-related (ATR) proteins, for example VE-822 or the like. In certain embodiments, the one or more additives that inhibit NHEJ comprise a molecule that directly or indirectly affects the activity of ligases, e.g., ligase IV, for example SCR7 or the like. In certain embodiments, the one or more additives that inhibit NHEJ comprise a molecule that directly or indirectly affects the activity of RAD51 binding to ssDNA, for example RS-1 or the like. In certain embodiments, the one or more additives that inhibit NHEJ comprise a molecule that directly or indirectly affects the activity cell cycle stage progression, for example aphidicolin, mimosin, thymidine, hydroxy urea, nocodazole, ABT-751, XL413, or the like. In certain embodiments, the one or more additives that inhibit NHEJ comprise a molecule that directly or indirectly affects the activity beta-3-adrenergic receptors, for example L755507 or the like. In certain embodiments, the one or more additives that inhibit NHEJ comprise a molecule that directly or indirectly affects the activity of intracellular transport from endoplasmic reticulum (ER) to golgi, for example Brefeldin A or the like. In certain embodiments, the one or more additives that inhibit NHEJ comprise a molecule that directly or indirectly affects the activity histone deacetylases, for example valproic acid (VPA). In certain embodiments, the one or more additives that inhibit NHEJ comprise M3814.

### II. ENGINEERED NON-NATURALLY-OCCURRING DUAL GUIDE CRISPR-CAS SYSTEMS

A CRISPR-Cas system generally comprises a Cas protein and one or more guide nucleic acids (gNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence, also referred to herein as a target sequence, in the target strand of the target polynucleotide. Typically, both PAM recognition and target nucleotide sequence hybridization are required for stable binding of a CRISPR-Cas complex to the DNA target and, if the Cas protein has an effector function (e.g., nuclease activity), activation of the effector function. As a result, when creating a CRISPR-Cas system, a guide nucleic acid can be designed to comprise a nucleotide sequence called a spacer sequence that is at least partially complementary to and can hybridize with a target nucleotide sequence, where target nucleotide sequence is located adjacent to a PAM in an orientation operable with the Cas protein. It has been observed that not all CRISPR-Cas systems designed by these criteria are equally effective. The larger polynucleotide in which a target nucleotide sequence is located may be referred to as a target polynucleotide; e.g., a chromosome or other genomic DNA, or portion thereof, or any other suitable polynucleotide within which a target nucleotide sequence is located. The target polynucleotide in double stranded DNA comprises two strands. The strand of the DNA duplex to which the spacer sequence is complementary herein is called the “target strand,” while the strand to which the spacer sequence shares sequence identity herein is called the “non-target strand.”

Two distinct classes of CRISPR-Cas systems have been identified. Class 1 CRISPR-Cas systems utilize multi-protein effector complexes, whereas class 2 CRISPR-Cas systems utilize single-protein effectors (see, Makarova et al. (2017) CELL, 168:328). Among the types of class 2 CRISPR-Cas systems, type II and type V systems typically target DNA and type VI systems typically target RNA (id.). Naturally occurring type II effector complexes include Cas9, CRISPR RNA (crRNA), and trans-activating CRISPR RNA (tracrRNA), but the crRNA and tracrRNA can be fused as a single guide RNA in an engineered system for simplicity (see, Wang et al. (2016) ANNU. REV. BIOCHEM., 85:227). Certain naturally occurring type V systems, such as type V-A, type V-C, and type V-D systems, do not require tracrRNA and use crRNA alone as the guide for cleavage of target DNA (see, Zetsche et al. (2015) CELL, 163:759; Makarova et al. (2017) CELL, 168:328.

Naturally occurring type II CRISPR-Cas systems (e.g., CRISPR-Cas9 systems) generally comprise two guide nucleic acids, called crRNA and tracrRNA, which form a complex by nucleotide hybridization. Single guide nucleic acids capable of activating type II Cas nucleases have been developed, for example, by linking the crRNA and the tracrRNA (see, e.g., U.S. Pat. Nos. 10,266,850 and 8,906,616). Naturally occurring type II Cas proteins comprise a RuvC-like nuclease domain and an HNH endonuclease domain, and recognize a 3′ G-rich PAM located immediately downstream from the target nucleotide sequence, the orientation determined using the non-target strand (i.e., the strand not hybridized with the spacer sequence) as the coordinate. The CRISPR-Cas systems cleave a double-stranded DNA to generate a blunt end. The cleavage site is generally 3-4 nucleotides upstream from the PAM on the non-target strand.

Naturally occurring Type V-A, Type V-C, and Type V-D CRISPR-Cas systems lack a tracrRNA and rely on a single crRNA to guide the CRISPR-Cas complex to the target polynucleotide. Dual guide nucleic acids capable of activating type V-A, type V-C, or type V-D Cas nucleases have been developed, for example, by splitting the single crRNA into a targeter nucleic acid and a modulator nucleic acid (see, e.g., International (PCT) Application Publication No. WO 2021/067788). Naturally occurring type V-A Cas proteins comprise a RuvC-like nuclease domain but lack an HNH endonuclease domain, and recognize a 5′ T-rich PAM located immediately upstream from the target nucleotide sequence, the orientation determined using the non-target strand (i.e., the strand not hybridized with the spacer sequence) as the coordinate. These CRISPR-Cas systems cleave a double-stranded DNA to generate a staggered double-stranded break rather than a blunt end. The cleavage site is distant from the PAM site (e.g., separated by at least 10, 11, 12, 13, 14, or 15 nucleotides downstream from the PAM on the non-target strand and/or separated by at least 15, 16, 17, 18, or 19 nucleotides upstream from the sequence complementary to PAM on the target strand).

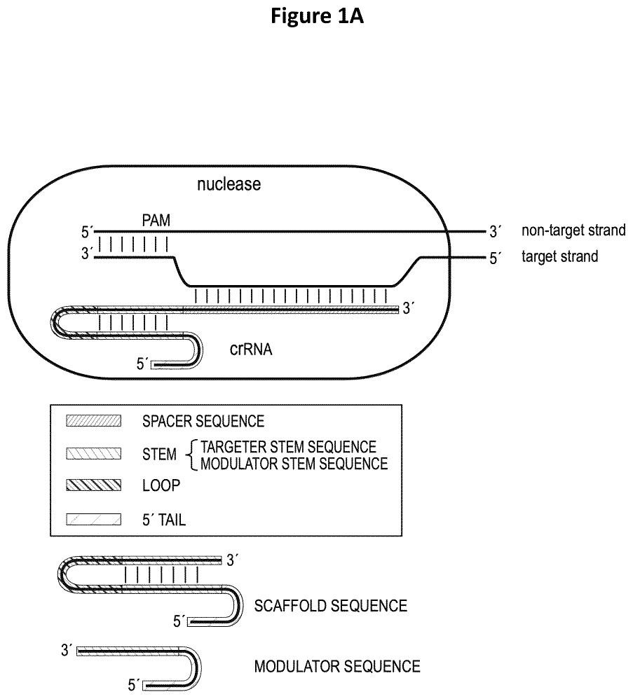

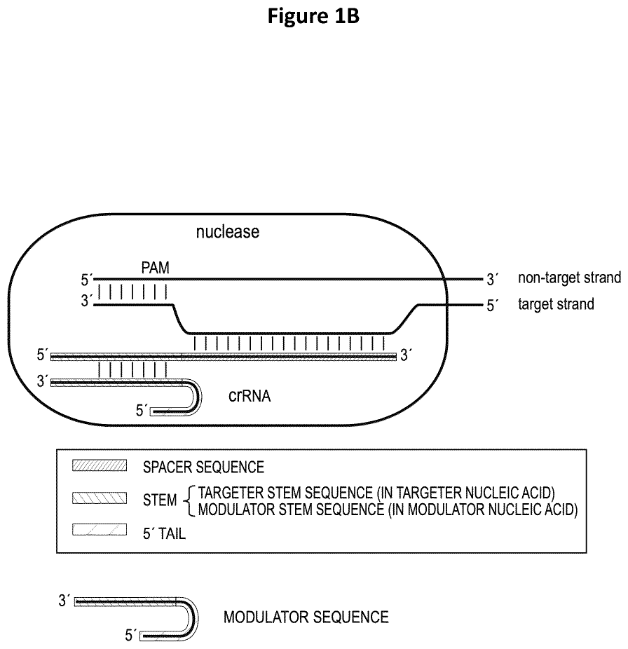

Elements in an exemplary single guide CRISPR Cas system, e.g., a type V-A CRISPR-Cas system, are shown in FIG. 1A. The single gNA can also be called a “crRNA” or “single gRNA” where it is present in the form of an RNA. It can comprise, from 5′ to 3′, an optional 5′ sequence, e.g., a tail, a modulator stem sequence, a loop, a targeter stem sequence complementary to the modulator stem sequence, and a spacer sequence that is at least partially complementary to and can hybridize with a target sequence in the target strand of the target polynucleotide. Where a 5′ tail is present, the sequence including the 5′ tail and the modulator stem sequence can also be called a “modulator sequence” herein. A fragment of the single guide nucleic acid from the optional 5′ tail to the targeter stem sequence, also called a “scaffold sequence” herein, bind the Cas protein. In addition, the PAM in the non-target strand of the target DNA binds the Cas protein.

Elements in an exemplary dual guide type CRISPR Cas system, e.g., a dual guide type V-A CRISPR-Cas system are shown in FIG. 1B. The first guide nucleic acid, which can be called a “modulator nucleic acid” herein, comprises, from 5′ to 3′, an optional 5′ tail and a modulator stem sequence. Where a 5′ tail is present, the sequence including the 5′ tail and the modulator stem sequence can also called a “modulator sequence” herein. The second guide nucleic acid, which can be called “targeter nucleic acid” herein, comprises, from 5′ to 3′, a targeter stem sequence complementary to the modulator stem sequence and a spacer sequence that is at least partially complementary to and can hybridize with the target sequence in the target strand of the target polynucleotide. The duplex between the modulator stem sequence and the targeter stem sequence, plus the optional 5′ tail, constitute a structure that binds the Cas protein. In addition, the PAM in the non-target strand of the target DNA binds the Cas protein. It is understood that, in a dual gNA, e.g., dual gRNA, the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

The terms “targeter stem sequence” and “modulator stem sequence,” as used herein, can refer to a pair of nucleotide sequences in one or more guide nucleic acids that hybridize with each other. When a targeter stem sequence and a modulator stem sequence are contained in a single guide nucleic acid, the targeter stem sequence is proximal to a spacer sequence designed to hybridize with a target nucleotide sequence, and the modulator stem sequence is proximal to the targeter stem sequence. When a targeter stem sequence and a modulator stem sequence are in separate nucleic acids, the targeter stem sequence is in the same nucleic acid as a spacer sequence designed to hybridize with a target nucleotide sequence. In a CRISPR-Cas system that naturally includes separate crRNA and tracrRNA (e.g., a type II system), the duplex formed between the targeter stem sequence and the modulator stem sequence corresponds to the duplex formed between the crRNA and the tracrRNA. In a CRISPR-Cas system that naturally includes a single crRNA but no tracrRNA (e.g., a type V-A system), the duplex formed between the targeter stem sequence and the modulator stem sequence corresponds to the stem portion of a stem-loop structure in the scaffold sequence of the crRNA. It is understood that 100% complementarity is not required between the targeter stem sequence and the modulator stem sequence. In a type V-A CRISPR-Cas system, however, the targeter stem sequence is typically 100% complementary to the modulator stem sequence.

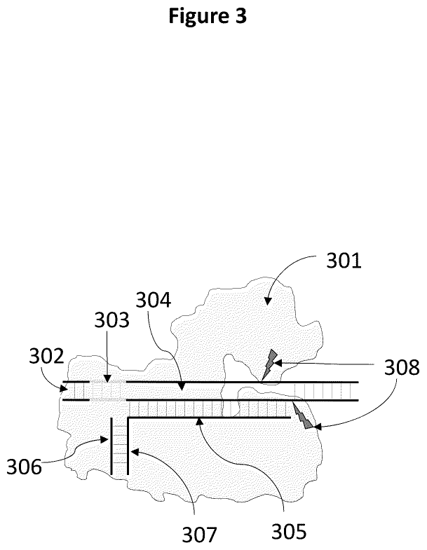

An illustrative example of a nucleic acid-guided nuclease complex is shown in FIG. 3. Specifically, FIG. 3 shows a Type V-A nucleic acid guided nuclease (301) complexed with a dual gNA comprising a modulator nucleic acid (306) and a targeter nucleic acid (307), wherein the modulator nucleic acid and targeter nucleic acid are hybridized through a stem. The targeter nucleic acid further comprises a spacer sequence (305) at least partially complementary to a target nucleotide sequence (304), i.e., a protospacer, in a target polynucleotide (302) adjacent to a suitable PAM (303). Upon binding to the target nucleotide sequence, the nucleic acid-guided nuclease complex can generate one or more strand breaks (308) in the target polynucleotide at or near the target nucleotide sequence.

### A. Cas Proteins

A guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of binding a CRISPR Associated (Cas) protein, e.g., a Cas nuclease. In certain embodiments, the guide nucleic acid, either as a single guide nucleic acid alone (targeter and modulator nucleic acids are part of a single polynucleotide) or as a dual gNA comprising separate targeter nucleic acid used in combination with a cognate modulator nucleic acid, is capable of activating a Cas nuclease. A gNA capable of activating a particular Cas nuclease is said to be “compatible” with the Cas nuclease; a Cas nuclease capable of being activated by a particular gNA is said to be “compatible” with the gNA.

The terms “CRISPR-Associated protein,” “Cas protein,” and “Cas,” as used interchangeably herein, can refer to a naturally occurring Cas protein or an engineered Cas protein. Non-limiting examples of Cas protein engineering include but are not limited to mutations and modifications of the Cas protein that alter the activity of the Cas, alter the PAM specificity, broaden the range of recognized PAMs, and/or reduce the ability to modify one or more off-target loci as compared to a corresponding unmodified Cas. In certain embodiments, the altered activity of engineered Cas comprises altered ability (e.g., specificity or kinetics) to bind a naturally occurring gNA, e.g., gRNA or engineered gNA, e.g., gRNA, altered ability (e.g., specificity or kinetics) to bind a target nucleotide sequence, altered processivity of nucleic acid scanning, and/or altered effector (e.g., nuclease) activity. A Cas protein having nuclease activity can be referred to as a “CRISPR-Associated nuclease” or “Cas nuclease,” or simply “nuclease,” as used interchangeably herein.

In certain embodiments, the Cas protein is a type V-A, type V-C, or type V-D Cas protein. In certain embodiments, the Cas protein is a type V-A Cas protein. In other embodiments, the Cas protein is a type II Cas protein, e.g., a Cas9 protein.

In certain embodiments, a type V-A Cas nucleases comprises Cpf1. Cpf1 proteins are known in the art and are described, e.g., in U.S. Pat. Nos. 9,790,490 and 10,113,179. Cpf1 orthologs can be found in various bacterial and archacal genomes. For example, in certain embodiments, the Cpf1 protein is derived from Francisella novicida U112 (Fn), Acidaminococcus sp. BV3L6 (As), Lachnospiraceae bacterium ND2006 (Lb), Lachnospiraceae bacterium MA2020 (Lb2), Candidatus Methanoplasma termitum (CMt), Moraxella bovoculi 237 (Mb), Porphyromonas crevioricanis (Pc), Prevotella disiens (Pd), Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Eubacterium eligens, Leptospira inadai, Porphyromonas macacae, Prevotella bryantii, Proteocatella sphenisci, Anaerovibrio sp. RM50, Moraxella caprae, Lachnospiraceae bacterium COE1, or Eubacterium coprostanoligenes.

In certain embodiments, a type V-A Cas nuclease comprises AsCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 3 of International (PCT) Application Publication No. WO 2021/158918. In certain embodiments, a type V-A Cas protein comprises the amino acid sequence set forth in SEQ ID NO: 3 of International (PCT) Application Publication No. WO 2021/158918.

In certain embodiments, a type V-A Cas nuclease comprises LbCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 4 of International (PCT) Application Publication No. WO 2021158918. In certain embodiments, a type V-A Cas protein comprises the amino acid sequence set forth in SEQ ID NO: 4 of International (PCT) Application Publication No. WO 2021/158918.

In certain embodiments, a type V-A Cas nuclease comprises FnCpf1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 5 of International (PCT) Application Publication No. WO 2021158918. In certain embodiments, a type V-A Cas protein comprises the amino acid sequence set forth in SEQ ID NO: 5 of International (PCT) Application Publication No. WO 2021/158918.

In certain embodiments, a type V-A Cas nuclease comprises Prevotella bryantii Cpf1 (PbCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 6 of International (PCT) Application Publication No. WO 2021/158918. In certain embodiments, a type V-A Cas protein comprises the amino acid sequence set forth in SEQ ID NO: 6 of International (PCT) Application Publication No. WO 2021/158918.

In certain embodiments, a type V-A Cas nuclease comprises Proteocatella sphenisci Cpf1 (PsCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 7 of International (PCT) Application Publication No. WO 2021158918. In certain embodiments, a type V-A Cas protein comprises the amino acid sequence set forth in SEQ ID NO: 7 of International (PCT) Application Publication No. WO 2021/158918.

In certain embodiments, a type V-A Cas nuclease comprises Anaerovibrio sp. RM50 Cpf1 (As2Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 8 of International (PCT) Application Publication No. WO 2021158918. In certain embodiments, a type V-A Cas protein comprises the amino acid sequence set forth in SEQ ID NO: 8 of International (PCT) Application Publication No. WO 2021/158918.

In certain embodiments, a type V-A Cas nuclease comprises Moraxella caprae Cpf1 (McCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 9 of International (PCT) Application Publication No. WO 2021/158918. In certain embodiments, a type V-A Cas protein comprises the amino acid sequence set forth in SEQ ID NO: 9 of International (PCT) Application Publication No. WO 2021/158918.

In certain embodiments, a type V-A Cas nuclease comprises Lachnospiraceae bacterium COE1 Cpf1 (Lb3Cpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 10 of International (PCT) Application Publication No. WO 2021158918. In certain embodiments, a type V-A Cas protein comprises the amino acid sequence set forth in SEQ ID NO: 10 of International (PCT) Application Publication No. WO 2021/158918.

In certain embodiments, a type V-A Cas nuclease comprises Eubacterium coprostanoligenes Cpf1 (EcCpf1) or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 11 of International (PCT) Application Publication No. WO 2021158918. In certain embodiments, a type V-A Cas protein comprises the amino acid sequence set forth in SEQ ID NO: 11 of International (PCT) Application Publication No. WO 2021/158918.

In certain embodiments, a type V-A Cas nuclease is not Cpf1. In certain embodiments, a type V-A Cas nuclease is not AsCpf1.

In certain embodiments, a type V-A Cas nuclease comprises MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20, or variants thereof. MAD1-MAD20 are known in the art and are described in U.S. Pat. No. 9,982,279.

In certain embodiments, a type V-A Cas nuclease comprises MAD7 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 37. In certain embodiments, a type V-A Cas protein comprises the amino acid sequence set forth in SEQ ID NO: 37.

MAD7
(SEQ ID NO: 37)
MNNGTNNFQNFIGISSLQKTLRNALIPTETTQQFIVKNGIIKEDELRGE
NRQILKDIMDDYYRGFISETLSSIDDIDWTSLFEKMEIQLKNGDNKDTL
IKEQTEYRKAIHKKFANDDRFKNMFSAKLISDILPEFVIHNNNYSASEK
EEKTQVIKLFSRFATSFKDYFKNRANCFSADDISSSSCHRIVNDNAEIF
FSNALVYRRIVKSLSNDDINKISGDMKDSLKEMSLEEIYSYEKYGEFIT
QEGISFYNDICGKVNSFMNLYCQKNKENKNLYKLQKLHKQILCIADTSY
EVPYKFESDEEVYQSVNGFLDNISSKHIVERLRKIGDNYNGYNLDKIYI
VSKFYESVSQKTYRDWETINTALEIHYNNILPGNGKSKADKVKKAVKND
LQKSITEINELVSNYKLCSDDNIKAETYIHEISHILNNFEAQELKYNPE
IHLVESELKASELKNVLDVIMNAFHWCSVFMTEELVDKDNNFYAELEEI
YDEIYPVISLYNLVRNYVTQKPYSTKKIKLNFGIPTLADGWSKSKEYSN
NAIILMRDNLYYLGIFNAKNKPDKKIIEGNTSENKGDYKKMIYNLLPGP
NKMIPKVFLSSKTGVETYKPSAYILEGYKQNKHIKSSKDFDITFCHDLI
DYFKNCIAIHPEWKNFGFDFSDTSTYEDISGFYREVELQGYKIDWTYIS
EKDIDLLQEKGQLYLFQIYNKDFSKKSTGNDNLHTMYLKNLFSEENLKD
IVLKLNGEAEIFFRKSSIKNPIIHKKGSILVNRTYEAEEKDQFGNIQIV
RKNIPENIYQELYKYFNDKSDKELSDEAAKLKNVVGHHEAATNIVKDYR
YTYDKYFLHMPITINFKANKTGFINDRILQYIAKEKDLHVIGIDRGERN
LIYVSVIDTCGNIVEQKSFNIVNGYDYQIKLKQQEGARQIARKEWKEIG
KIKEIKEGYLSLVIHEISKMVIKYNAIIAMEDLSYGFKKGRFKVERQVY
QKFETMLINKLNYLVFKDISITENGGLLKGYQLTYIPDKLKNVGHQCGC
IFYVPAAYTSKIDPTTGFVNIFKFKDLTVDAKREFIKKFDSIRYDSEKN
LFCFTFDYNNFITQNTVMSKSSWSVYTYGVRIKRRFVNGRFSNESDTID
ITKDMEKTLEMTDINWRDGHDLRQDIIDYEIVQHIFEIFRLTVQMRNSL
SELEDRDYDRLISPVLNENNIFYDSAKAGDALPKDADANGAYCIALKGL
YEIKQITENWKEDGKFSRDKLKISNKDWFDFIQNKRYL

In certain embodiments, a type V-A Cas nuclease comprises MAD2 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 38. In certain embodiments, a type V-A Cas protein comprises the amino acid sequence set forth in SEQ ID NO: 38.

MAD2
(SEQ ID NO: 38)
MSSLTKFTNKYSKQLTIKNELIPVGKTLENIKENGLIDGDEQLNENYQK
AKIIVDDFLRDFINKALNNTQIGNWRELADALNKEDEDNIEKLQDKIRG
IIVSKFETFDLFSSYSIKKDEKIIDDDNDVEEEELDLGKKTSSFKYIFK
KNLFKLVLPSYLKTTNQDKLKIISSFDNFSTYFRGFFENRKNIFTKKPI
STSIAYRIVHDNFPKFLDNIRCFNVWQTECPQLIVKADNYLKSKNVIAK
DKSLANYFTVGAYDYFLSQNGIDFYNNIIGGLPAFAGHEKIQGLNEFIN
QECQKDSELKSKLKNRHAFKMAVLFKQILSDREKSFVIDEFESDAQVID
AVKNFYAEQCKDNNVIFNLLNLIKNIAFLSDDELDGIFIEGKYLSSVSQ
KLYSDWSKLRNDIEDSANSKQGNKELAKKIKTNKGDVEKAISKYEFSLS
ELNSIVHDNTKFSDLLSCTLHKVASEKLVKVNEGDWPKHLKNNEEKQKI
KEPLDALLEIYNTLLIFNCKSFNKNGNFYVDYDRCINELSSVVYLYNKT
RNYCTKKPYNTDKFKLNFNSPQLGEGFSKSKENDCLTLLFKKDDNYYVG
IIRKGAKINFDDTQAIADNTDNCIFKMNYFLLKDAKKFIPKCSIQLKEV
KAHFKKSEDDYILSDKEKFASPLVIKKSTFLLATAHVKGKKGNIKKFQK
EYSKENPTEYRNSLNEWIAFCKEFLKTYKAATIFDITTLKKAEEYADIV
EFYKDVDNLCYKLEFCPIKTSFIENLIDNGDLYLFRINNKDFSSKSTGT
KNLHTLYLQAIFDERNLNNPTIMLNGGAELFYRKESIEQKNRITHKAGS
ILVNKVCKDGTSLDDKIRNEIYQYENKFIDTLSDEAKKVLPNVIKKEAT
HDITKDKRFTSDKFFFHCPLTINYKEGDTKQFNNEVLSFLRGNPDINII
GIDRGERNLIYVTVINQKGEILDSVSFNTVTNKSSKIEQTVDYEEKLAV
REKERIEAKRSWDSISKIATLKEGYLSAIVHEICLLMIKHNAIVVLENL
NAGFKRIRGGLSEKSVYQKFEKMLINKLNYFVSKKESDWNKPSGLLNGL
QLSDQFESFEKLGIQSGFIFYVPAAYTSKIDPTTGFANVLNLSKVRNVD
AIKSFFSNFNEISYSKKEALFKFSFDLDSLSKKGFSSFVKFSKSKWNVY
TFGERIIKPKNKQGYREDKRINLTFEMKKLLNEYKVSFDLENNLIPNLT
SANLKDTFWKELFFIFKTTLQLRNSVTNGKEDVLISPVKNAKGEFFVSG
THNKTLPQDCDANGAYHIALKGLMILERNNLVREEKDTKKIMAISNVDW
FEYVQKRRGVL

In certain embodiments, a type V-A Cas nucleases comprises Csm1. Csm1 proteins are known in the art and are described in U.S. Pat. No. 9,896,696. Csm1 orthologs can be found in various bacterial and archaeal genomes. For example, in certain embodiments, a Csm1 protein is derived from Smithella sp. SCADC (Sm), Sulfuricurvum sp. (Ss), or Microgenomates (Roizmanbacteria) bacterium (Mb).

In certain embodiments, a type V-A Cas nuclease comprises SmCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 12 of International (PCT) Application Publication No. WO 2021/158918. In certain embodiments, a type V-A Cas protein comprises the amino acid sequence set forth in SEQ ID NO: 12 of International (PCT) Application Publication No. WO 2021/158918.

In certain embodiments, a type V-A Cas nuclease comprises SsCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 13 of International (PCT) Application Publication No. WO 2021/158918. In certain embodiments, a type V-A Cas protein comprises the amino acid sequence set forth in SEQ ID NO: 13 of International (PCT) Application Publication No. WO 2021/158918.

In certain embodiments, a type V-A Cas nuclease comprises MbCsm1 or a variant thereof. In certain embodiments, a type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in SEQ ID NO: 14 of International (PCT) Application Publication No. WO 2021/158918. In certain embodiments, a type V-A Cas protein comprises the amino acid sequence set forth in SEQ ID NO: 14 of International (PCT) Application Publication No. WO 2021/158918.

In certain embodiments, the type V-A Cas nuclease comprises an ART nuclease or a variant thereof. In general, such nucleases sequences have <60% AA sequence similarity to Cas12a, <60% AA sequence similarity to a positive control nuclease, and >80% query cover. In certain embodiments, the Type V-A nuclease comprises an ART1, ART2, ART3, ART4, ART5, ART6, ART7, ART8, ART9, ART10, ART11, ART12, ART13, ART14, ART15, ART16, ART17, ART18, ART19, ART20, ART21, ART22, ART23, ART24, ART25, ART26, ART27, ART28, ART28, ART30, ART31, ART32, ART33, ART34, ART35, or ART11* (i.e., ART11_L679F, i.e., ART11 wherein leucine (L) at amino acid position 679 is replaced with phenylalanine (F)) nuclease, as shown in Table 2. In certain embodiments, the type V-A Cas protein comprises an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence designated for the individual ART nuclease as shown in Table 2. In certain embodiments, provided is a nucleic acid-guided nuclease comprising a nucleic acid-guided nuclease polypeptide having at least 85% identity to an amino acid sequence represented by SEQ ID NOs: 1-36 or a nucleic acid encoding a nucleic acid-guided nuclease polypeptide comprising at least 85% identity with the polynucleotide represented by SEQ ID NOs: 1-36. In certain embodiments, provided is a nucleic acid-guided nuclease comprising a polypeptide having at least 90% identity to the amino acid sequence represented by SEQ ID NOs: 1-36, wherein the 5 polypeptide does not contain a peptide motif of YLFQIYNKDF (SEQ ID NO: 39). In certain embodiments, provided is a nucleic acid-guided nuclease comprising a nucleic acid encoding a polypeptide having at least 90% identity to nucleic acids represented by SEQ ID NOs: 808-845 wherein an encoded polypeptide does not contain a peptide motif of YLFQIYNKDF (SEQ ID NO: 39). In certain embodiments, provided is a nucleic acid-guided nuclease wherein the 10 polypeptide comprises at least 90% identity with the amino acid sequence represented by SEQ ID NOs: 1-9. In certain embodiments, provided is a nucleic acid-guided nuclease, wherein the polypeptide comprises a polypeptide comprising at least 90% identity with the amino acid sequence represented by SEQ ID NO: 2, 11, or 36.

TABLE 2
ART nucleases

| | SEQ | |
|---|---|---|
| Name | ID NO | Amino Acid Sequence |
| ART1 | 1 | METFSGFTNLYPLSKTLRFRLIPVGETLKHFIDSGILEEDQHRAESYVK |
| | | VKAIIDDYHRAYIENSLSGFELPLESTKFNSLEEYYLYHNIRNKTEEIQ |
| | | NLSSKVRTNLRKQVVAQLTKNEIFKRIDKKELIQSDLIDFVKNEPDANE |
| | | KIALISEFRNFTVYFKGFHENRRNMYSDEEKSTSIAFRLIHENLPKFID |
| | | NMEVFAKIQNTSISENFDAIQKELCPELVTLCEMFKLGYFNKTLSQKQI |
| | | DAYNTVIGGKTTSEGKKIKGLNEYINLYNQQHKQEKLPKMKLLFKQILS |
| | | DRESASWLPEKFENDSQVVGAIVNFWNTIHDTVLAEGGLKTIIASLGSY |
| | | GLEGIFLKNDLQLTDISQKATGSWGKISSEIKQKIEVMNPQKKKESYET |
| | | YQERIDKIFKSYKSFSLAFINECLRGEYKIEDYFLKLGAVNSSSLQKEN |
| | | HFSHILNTYTDVKEVIGLYSESTDTKLIQDNDSIQKIKQFLDAVKDLQA |
| | | YVKPLLGNGDETGKDERFYGDLIEYWSLLDLITPLYNMVRNYVTQKPYS |
| | | VDKIKINFQNPTLLNGWDLNKETDNTSVILRRDGKYYLAIMNNKSRKVF |
| | | LKYPSGTDRNCYEKMEYKLLPGANKMLPKVFFSKSRINEFMPNERLLSN |
| | | YEKGTHKKSGTCFSLDDCHTLIDFFKKSLDKHEDWKNFGFKFSDTSTYE |
| | | DMSGFYKEVENQGYKLSFKPIDATYVDQLVDEGKIFLFQIYNKDFSEHS |
| | | KGTPNMHTLYWKMLFDETNLGDVVYKLNGEAEVFFRKASINVSHPTHPA |
| | | NIPIKKKNLKHKDEERILKYDLIKDKRYTVDQFQFHVPITMNFKADGNG |
| | | NINQKAIDYLRSASDTHIIGIDRGERNLLYLVVIDGNGKICEQFSLNEI |
| | | EVEYNGEKYSTNYHDLLNVKENERKQARQSWQSIANIKDLKEGYLSQVI |
| | | HKISELMVKYNAIVVLEDLNAGFMRGRQKVEKQVYQKFEKKLIEKLNYL |
| | | VFKKQSSDLPGGLMHAYQLANKFESFNTLGKQSGFLFYIPAWNTSKMDP |
| | | VTGFVNLFDVKYESVDKAKSFFSKFDSIRYNVERDMFEWKFNYGEFTKK |
| | | AEGTKTDWTVCSYGNRIITFRNPDKNSQWDNKEINLTENIKLLFERFGI |
| | | DLSSNLKDEIMQRTEKEFFIELISLFKLVLQMRNSWTGTDIDYLVSPVC |
| | | NENGEFFDSRNVDETLPQNADANGAYNIARKGMILLDKIKKSNGEKKLA |
| | | LSITNRE WLSFAQGCCKNG |
| ART2 | 2 | MLSNFTNQYQLSKTERFELKPVGDTLKHIEKSGLIAQDEIRSQEYQEVK |
| | | TIIDKYHKAFIDEALQNVVISNLEEYEALFFERNRDEKAFEKLQAVLRK |
| | | EIVAHFKQHPQYKTLFKKELIKADLKNWQELSDAEKELVSHFDNFTTYF |
| | | TGFHENRANMYTDEAKHSSIAYRIIHENLPIFLINKKLFETIKQKAPHL |
| | | AQETQDALLEYLSGAIVEDMFELSYFNHLLSQTHIDLYNQMIGGVKQDS |
| | | LKIQGINEKINLYRQANGLSKRELPNLKPLHKQILSDRETLSWLPESFE |
| | | SDEELMQGVQAYFESEVLAFECCDGKVNLLEKLPELLHQTQDYDFSKVY |
| | | FKNDLALTAASQAIFKDYRIIKEALWEVNKPKKSKDLVADEEKFFNKKN |
| | | SYFSIEQIDGALNSAQLSANMMHYFQSESTKVIEQIQLTYNDWKRNSSN |
| | | KELLKAFLDALLSYQRLLKPLNAPNDLEKDVAFYAYFDAYFTSLCGVVK |
| | | LYDKVRNFMTKKPYSLEKFKLNFENSTLLDGWDVNKESDNTAILFRKEG |
| | | LYYLGIMNKKYNKVFRNISSSQDEGYQKIDYKLLPGANKMLPKVFFSDK |
| | | NKEYFKPNAKLLERYKAGEHKKGDNFDLDFCHELIDFFKTSIEKHQDWK |
| | | HFAYQFSPTESYEDLSGFYREVEQQGYKISYKNIAASFIDTLVAEGKLY |
| | | FFQIYNKDFSPYSKGTPNMHTLYWRALFDEKNLADVIYKLNGQAEIFFR |
| | | KKSIEYSQEKLQKGHHHEMLKDKFAYPIIKDRRFAFDKFQFHVPITLNF |
| | | KAEGNENITPKTFEYIRSNPDNIKVIGIDRGERHLLYLSLIDAEGKIVE |
| | | QFTLNQIINSYNGKDHVIDYHAKLDAKEKDRDKARKEWGTVENIKELKE |
| | | GYLSHVIHKIATLIIEHGAVVAMEDLNFGFKRGRFKVEKQVYQKFEKAL |
| | | IDKLNYLVDKKKEPHKLGGLLNALQLTSKFQSFEKMGKQNGFLFYVPAW |
| | | NTSKIDPVTGFVNLFDTRYASVEKSKAFFTKFQSICYNEAKDYFELVFD |
| | | YNDFTEKAKETRSEWILCTYGERIVSFRNAEKNHQWDSKTIHLTTEFKN |
| | | LFGELHGNDVKEYILEQNSVEFFKSLIYLLKITLQMRNSITGTDIDYLV |
| | | SPVADEAGNFYDSRKADTSLPKDADANGAYNIARKGLMLMHRIQNAEDL |
| | | KKVNLAISNRDWLRNAQGLDK |
| ART3 | 3 | MIDLKQFIGIYPVSKTLRFELRPVGKTQEWIEKNRVLEGDEQKAADYPV |
| | | VKKLIDDYHKVCIHDSLNHVHFDWEPLKDAIEIFQKTKSDEAKKRLEAE |
| | | QAMMRKKIAAAIKDFKHFKELTAATPSDLITSVLPEFSDDGSLKSFRGF |
| | | ATYFSGFQENRNNIYSQEAISTGVPYRLVHDNFPKFLSDLEVFERIKST |
| | | CPEVINQASAELQPFLEGVMIDDIFSLDFYNSLLTQNGIDFFNQVIGGV |
| | | SEKDKQKYRGINEFSNLYRQQHKEIAASKKAMTMIPLFKQILSDRDTLS |
| | | YIPAQIRTEDELVSSITQFYDHITHFEHDGKTINVLSEIVALLGKLDTY |
| | | DPNGICITARKLTDISQKVYGKWSVIEEKMKEKAIQQYGDISVAKNKKK |
| | | VDAFLSRKAYSLSDLCFDEEISFSRYYSELPQTLNAISGYWLQFNEWCK |
| | | SDEKQKFLNNQTGTEVVKSLLDAMMELFHKCSVLVMPEEYEVDKSFYNE |
| | | FLPLYEELDTLFLLYNKVRNYLTQKPSDVKKFKLNFESPSLASGWDQNK |
| | | EMKNNAILLFKDGKSYLGVLNAKNKAKIKDAKGDVSSSSYKKMIYKLLS |
| | | DPSKDLPHKIFAKGNLDFYKPSEYILEGRELGKYKKGPNFDKKFLHDFI |
| | | DFYKAAISIDPDWSKFNFQYSPTESYDDIGMFFSEIKKQAYKIRFTDIS |
| | | EAQVNEWVDNGQLYLFQLYNKDYAEGAHGRKNLHTLYWENLFTDENLSN |
| | | LVLKLNGQAELFCRPQSIKKPVSHKIGSKMLNRRDKSGMPIPESIYRSL |
| | | YQYYNGKKKESELTVAEKQYIDQVIVKDVTHEIIKDRRYTRQEYFFHVP |
| | | LTFNANADGNEYINEHVLNYLKDNPDVNIIGIDRGERHLIYLTLINQRG |
| | | EILKQKTFNVVNSYNYQAKLEQREKERDEARKSWDSVGKIKDLKEGFLS |
| | | AVIHEITNMMIENNAIVVLEDLNFGFKRGRFKVERQVYQKFEKMLIDKL |
| | | NYLSFKDREAGEEGGILRGYQMAQKFISFQRLGKQSGFLFYIPAAYTSK |
| | | IDPVSGFVNHFNFSDITNAEKRKDFLMKMDRIEMKNGNIEFTFDYRKFK |
| | | TFQTDYQNVWTVSTFGKRIVMRIDEKGYKKMVDYEPINDIIKAFKNKGI |
| | | LLSEGSDLKALIAEIEANATNAGFYSTLLYAFQKTLQMRNSNAVTEEDY |
| | | ILSPVAKDGHQFCSTDEANKGKDAQGNWVSKLPVDADANGAYHIALKGL |
| | | YLLRNPETKKIENEKWLQFMVEKPYLE |
| ART4 | 4 | MSYNREKMEEKELGKNQNFQEFIGVSPLQKTLRNELIPTETTKKNIAQL |
| | | DLLTEDEVRAQNREKLKEMMDDYYRDVIDSTLRGELLIDWSYLFSCMRN |
| | | HLSENSKESKRELERTQDSVRSQIHDKFAERADFKDMFGASIITKLLPT |
| | | YIKQNSKYSERYDESVKIMKLYGKFTTSLTDYFETRKNIFSKEKISSAV |
| | | GYRIVEENAEIFLQNQNAYDRICKIAGLDLHGLDNEITAYVDGKTLKEV |
| | | CSDEGFAKVITQGGIDRYNEAIGAVNQYMNLLCQKNKALKPGQFKMKRL |
| | | HKQILCKGTTSFDIPKKFENDKQVYDAVNSFTEIVTKNNDLKRLLNITQ |
| | | NANDYDMNKIYVVADAYSMISQFISKKWNLIEECLLDYYSDNLPGKGNA |
| | | KENKVKKAVKEETYRSVSQLNEVIEKYYVEKTGQSVWKVESYISSLAEM |
| | | IKLELCHEIDNDEKHNLIEDDEKISEIKELLDMYMDVFHIIKVFRVNEV |
| | | LNFDETFYSEMDEIYQDMQEIVPLYNHVRNYVTQKPYKQEKYRLYFHTP |
| | | TLANGWSKSKEYDNNAIILVREDKYYLGILNAKKKPSKEIMAGKEDCSE |
| | | HAYAKMNYYLLPGANKMLPKVFLSKKGIQDYHPSSYIVEGYNEKKHIKG |
| | | SKNFDIRFCRDLIDYFKECIKKHPDWNKFNFEFSATETYEDISVFYREV |
| | | EKQGYRVEWTYINSEDIQKLEEDGQLFLFQIYNKDFAVGSTGKPNLHTL |
| | | YLKNLFSEENLRDIVLKLNGEAEIFFRKSSVQKPVIHKCGSILVNRTYE |
| | | ITESGTTRVQSIPESEYMELYRYFNSEKQIELSDEAKKYLDKVQCNKAK |
| | | TDIVKDYRYTMDKFFIHLPITINFKVDKGNNVNAIAQQYIAEQEDLHVI |
| | | GIDRGERNLIYVSVIDMYGRILEQKSFNLVEQVSSQGTKRYYDYKEKLQ |
| | | NREEERDKARKSWKTIGKIKELKEGYLSSVIHEIAQMVVKYNAIIAMED |
| | | LNYGFKRGRFKVERQVYQKFETMLISKLNYLADKSQAVDEPGGILRGYQ |
| | | MTYVPDNIKNVGRQCGIIFYVPAAYTSKIDPTTGFINAFKRDVVSTNDA |
| | | KENFLMKFDSIQYDIEKGLFKFSFDYKNFATHKLTLAKTKWDVYTNGTR |
| | | IQNMKVEGHWLSMEVELTTKMKELLDDSHIPYEEGQNILDDLREMKDIT |
| | | TIVNGILEIFWLTVQLRNSRIDNPDYDRIISPVLNNDGEFFDSDEYNSY |
| | | IDAQKAPLPIDADANGAFCIALKGMYTANQIKENWVEGEKLPADCLKIE |
| | | HASWLAFMQGERG |
| ART5 | 5 | MSAVFKIKESTMKDFTHQYSLSKTLRFELKPVGETAERIEDFKNQGLKS |
| | | IVEEDRQRAEDYKKMKRILDDYHKEFIEEVLNDDIFTANEMESAFEVYR |
| | | KYMASKNDDKLKKEITEIFTDLRKKIAKAFENKSKEYCLYKGDFSKLIN |
| | | EKKTGKDKGPGKLWYWLKAKADAGVNEFGDGQTFEQAEEALAKFNNFST |
| | | YFTGFNQNRDNIYTDAEQQTAISYRVINENMTRYFDNCIRYSSIENKYP |
| | | ELVKQLEPLSGKFAPGNYKDYLSQTAIDIYNEAVGHKSDDINAKGINQF |
| | | INEYRQRNSIKGRELPIMSVLYKQILSDINKDLIIDKFENAGELLDAVK |
| | | TLHRELTDKKILLKIKQTLNEFLTEDNSEDIYIKSGTDLTAVSNAIWGE |
| | | WSVIPKALEMYAENITDMNAKAREKWLKREAYHLKTVQEAIEAYLKDNE |
| | | EFETRNISEYFTNFKSGENDLIQVVQSAYAKMESIFGIEDFHKDRRPVT |
| | | ESGEPGEGFRQVELVREYLDSLINVEHFIKPLHMFRSGKPIELEDCNSN |
| | | FYDPLNEAYKELDVVFGIYNKVRNYVTQKPYSKDKFKINFQNSTLLDGW |
| | | DVNKESANSSVLLLKNGKYYLGVMKQGASNILNYRPEPSDSKNKINAKK |
| | | QLSEIALAGATDDYYEKMIYKLLPDPAKMLPKVFFSAKNIEFYNPSQEI |
| | | IYIRENGLFKKDAGDKESLKKWIGFMKTSLLKHPEWGSYFNFEFEPAED |
| | | YQDISIFYKQVAEQGYSVTFDKIKTSYIEEKVASGELYLFEIYNKDFSP |
| | | HSKGRPNLHTMYWKSLFEKENLQNLVTKLNGEAEVFFRQHSIKRNEKVV |
| | | HRANRPIQNKNPLTEKKQSIFEYDLVKDRRFTKDKFFLHCPITLNFKEA |
| | | GPGRFNDKVNKYIAGNPDIRIIGIDRGERHLLYYSLIDQSGRIVEQGTL |
| | | NQITSTLNSGGREIPKTTDYRGLLDTKEKERDKARKSWSMIENIKELKS |
| | | GYLSHIVHKLAKLMVKNNAVVVLEDLNFGFKRGRFKVEKQVYQKFEKAL |
| | | IEKLNYLVFKDARPAEPGHYLNAYQLTAPLESFKKLGKQSGFIYYVPAW |
| | | NTSKIDPVTGFVNQFYIEKNSMQYLKNFFGKFDSIRFNPDKNYFEFGFD |
| | | YKNFHNKAAKSKWTICTHGDKRSWYNRKQRKLEIHNVTENLASLLSGKG |
| | | INFADGGSIKDKILSVDDASFFKSLAFNFKLTAQLRHTFEDNGEEIDCI |
| | | ISPVAAADGTFFCSETAKKLNMELPHDADANGAYNIARKGLMVLRQIRE |
| | | SGKPKPISNADWLDFAQQNED |
| ART6 | 6 | MQERKKISHLTHRNSVQKTIRMQLNPVGKTMDYFQAKQILENDEKLKEN |
| | | YQKIKEIADRFYRNLNEDVLSKTGLDKLKDYAEIYYHCNTDAERKRLDE |
| | | CASELRKEIVKNFKNRDEYNKLFNKKMIEIVLPQHLKNEDEKEVVASFK |
| | | NFTTYFTGFFTNRKNMYSDGEESTAIAYRCINENLPKHLDNVKAFEKAI |
| | | SKLSKNAIDDLDATYSGLCGTNLYDVFTVDYFNFLLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVNYYKTSLMQLTDN |
| | | LSDKYNEAAPLLNKSYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCRDEKYYLAIIDKSNNSILENIDF |
| | | QDCDENDCYEKIIYKLLPGPNKMLPKVFFSEKCKKLLSPSDEILKIRKN |
| | | GTFKKGDKFSLDDCHKLIDFYKESFKKYPNWLIYNFKFKNTNEYNDIRE |
| | | FYNDVASQGYNISKMKIPTSFIDKLVDEGKIYLFQLYNKDFSPHSKGTP |
| | | NLHTLYFKMLFDERNLEDVVYKLNGEAEMFYRPASIKYDKPTHPKNTPI |
| | | KNKNTLNDKKTSTFPYDLIKDKRYTKWQFSLHFPITMNFKAPDRAMIND |
| | | DVRNLLKSCNNNFIIGIDRGERNLLYVSVIDSNGAIIYQHSLNIIGNKF |
| | | KGKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEGGGLLHAYQLTNKLESFDKLGMQSGFIFYVRPDFTSKIDPVTGF |
| | | VNLLYPRYENIDKAKDMISRFDDIGYNAGEDFFEFDIDYDKFPKTASDY |
| | | RKRWTICTNGERIEAFRNPAKNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKSDNVSTVG |
| | | PVIHNDKWLKFVQENDMANN |
| ART7 | 7 | MNILKENYMKEIKELTGLYSLTKTIGVELKPVGKTQELIEAKKLIEQDD |
| | | QRAEDYKIVKDIIDRYHKDFIDKCLNCVKIKKDDLEKYVSLAENSNRDA |
| | | EDFDKIKTKMRNQITEAFRKNSLFTNLFKKNLIKEYLPAFVSEEEKSVV |
| | | NKFSKFTTYFDAFNDNRKNLYSGDAKSGTIAYRLIHENLPMFLDNIASF |
| | | NAISGIGVNEYFSSIETEFTDTLEGKRLTEFFQIDFFNNTLTQKKIGNY |
| | | NYIVGAVNKAVNLYKQQHKTVRVPLLKPLYKMILSDRVTPSWLPERFES |
| | | DEEMLTAIKAAYESLREVLVGDNDESLRNLLLNIEHYDLEHIYIANDSG |
| | | LTSISQKIFGCYDTYTLAIKDQLQRDYPATKKQREAPDLYDERIDKLYK |
| | | KVGSFSIAYLNRLVDAKGHFTINEYYKQLGAYCREEGKEKDDFFKRIDG |
| | | AYCAISHLFFGEHGEIAQSDSDVELIQKLLEAYKGLQRFIKPLLGHGDE |
| | | ADKDNEFDAKLRKVWDELDIITPLYDKVRNWLSRKIYNPEKIKLCFENN |
| | | GKLLSGWVDSRTKSDNGTQYGGYIFRKKNEIGEYDFYLGISADTKLFRR |
| | | DAAISYDDGMYERLDYYQLKSKTLLGNSYVGDYGLDSMNLLSAFKNAAV |
| | | KFQFEKEVVPKDKENVPKYLKRLKLDYAGFYQILMNDDKVVDAYKIMKQ |
| | | HILATLTSSIRVPAAIELATQKELGIDELIDEIMNLPSKSFGYFPIVTA |
| | | AIEEANKRENKPLFLFKMSNKDLSYAATASKGLRKGRGTENLHSMYLKA |
| | | LLGMTQSVFDIGSGMVFFRHQTKGLAETTARHKANEFVANKNKLNDKKK |
| | | SIFGYEIVKNKRFTVDKYLFKLSMNLNYSQPNNNKIDVNSKVREIISNG |
| | | GIKNIIGIDRGERNLLYLSLIDLKGNIVMQKSLNILKDDHNAKETDYKG |
| | | LLTEREGENKEARRNWKKIANIKDLKRGYLSQVVHIISKMMVEYNAIVV |
| | | LEDLNPGFIRGRQKIERNVYEQFERMLIDKLNFYVDKHKGANETGGLLH |
| | | ALQLTSEFKNFKKSEHQNGCLFYIPAWNTSKIDPATGFVNLFNTKYTNA |
| | | VEAQEFFSKFDEIRYNEEKDWFEFEFDYDKFTQKAHGTRTKWTLCTYGM |
| | | RLRSFKNSAKQYNWDSEVVALTEEFKRILGEAGIDIHENLKDAICNLEG |
| | | KSQKYLEPLMQFMKLLLQLRNSKAGTDEDYILSPVADENGIFYDSRSCG |
| | | DQLPENADANGAYNIARKGLMLIEQIKNAEDLNNVKFDISNKAWINFAQ |
| | | QKPYKNG |
| ART8 | 8 | MAKENIFNELTGKYQLSKTLRLELKPVGNTQQMLKDEDVFEKDRIIREK |
| | | YRETRPHFDRLHREFIEQALKNQKLSDLGKYFQCLAKLQNNKKDKEAQE |
| | | EFKRISQNLRKEVNDLFKIDPLFGEGVFALLKEKYGEKDDAFLREQDGQ |
| | | YVLDENKKKISIFDSWKGFTGYFTKFQETRKNFYKDDGTATAVATRIID |
| | | QNLKRFCENIQIFKSIQKKVDFKEVEDNFSVDLEDIFSLGFYSSCFLQE |
| | | GIDVYNKILGGEPKTTGEKLRGLNELINRYRQDHKGEKLPFFKMLDKQI |
| | | LSEKEKFIESIEDDEELLKTLKEFYSSAEEKTTVLKELFNDFIKNNENY |
| | | DLSEIYISREALNTISHRWVSAATLPEFEKSVYEVMKKDKPSGLSFDKD |
| | | DNSYKFPDFIALSYIKGSFEKLSGEKLWKDGYFRDETRNGDKGFLIGNE |
| | | SLWTQFIKIFEFEFNSLFEAKNTERSVGYYHFKKDFEKIITNDFSVNPE |
| | | DKVIIREFADNVLAIYQMAKYFAIEKKRKWMDQYDTGDFYNHPDFGYKT |
| | | KFYDNAYEKIVKARMLLQSYLTKKPFSTDKWKLNFECGYLLNGWSSSFN |
| | | TYGSLLFRTGNEYYLGVVNGSALRTEKIKRLTGNITEANSCHKMVYDFQ |
| | | KPDNKNVPRIFIRSKGDKFAPAVSELNLPVDSILEIYDKGLFKTENKNS |
| | | PFFKPSLKKLIDYFKLGFSRHASYKHYQFKWKDSSEYKNISEFYNDTIR |
| | | SCYQIKWEELNFEEVKKLTNSKDLFLFQIYNKDFSEKSTGNKNLHSIYF |
| | | DGLFLDNNINAQDGVILKLSGGGEIFFRPKTDVKKLGSRTDTKGKLVIK |
| | | NKRYSQDKIFLHFPIELNYSNTQESNFNKLVRNFLADNPDINIIGVDRG |
| | | EKHLIYYAGIDQKGNTLKDKDDKDVLGSLNEINGVNYYKLLEERAKARE |
| | | KARQDWQNIQGIKDLKMGYISLVVRKLADLIIEYNAILVLEDLNMRFKQ |
| | | IHGGIEKSVYQQLEKALIEKLNFLVNKGEKDPERAGHLLRAYQLTAPFS |
| | | TFKDMGKQTGVLFYTQASYTSKTCPQCGFRPNIKLHFDNLENAKKMLEK |
| | | INIVYKDNHFEIGYKVSDFTKTEKTSRGNILYGDRQGKDTFVISSKAAI |
| | | RYKWFARNIKNNELNRGESLKEHTEKGVTIQYDITECLKILYEKNGIDH |
| | | SGDITKQSIRSELPAKFYKDLLFYLYLLTNTRSSISGTEIDYINCPDCG |
| | | FHSEKGFNGCIFNGDANGAYNIARKGMLILKKINQYKDQHHTMDKMGWG |
| | | DLFIGIEEWDKYTQVVSRS |
| ART9 | 9 | MKEIKELTGLYSLTKTIGVELKPVGKTQELIEAKKLIEQDDQRAEDYKI |
| | | VKDIIDRYHKDFIDKCLNCVKIKKDDLEKYVSLAENSNRDAEDFDKIKT |
| | | KMRNQITEAFRKNSLFTNLFKKNLIKEYLPAFVSEEEKSVVNKFSKFTT |
| | | YFDAFNDNRKNLYSGDAKSGTIAYRLIHENLPMFLDNIASFNAISGIGV |
| | | NEYFSSIETEFTDTLEGKRLTEFFQIDFFNNTLTQKKIGNYNYIVGAVN |
| | | KAVNLYKQQHKTVRVPLLKPLYKMILSDRVTPSWLPERFESDEEMLTAI |
| | | KAAYESLREVLVGDNDESLRNLLLNIEHYDLEHIYIANDSGLTSISQKI |
| | | FGCYDTYTLAIKDQLQRDYPATKKQREAPDLYDERIDKLYKKVGSFSIA |
| | | YLNRLVDAKGHFTINEYYKQLGAYCREEGKEKDDFFKRIDGAYCAISHL |
| | | FFGEHGEIAQSDSDVELIQKLLEAYKGLQRFIKPLLGHGDEADKDNEFD |
| | | AKLRKVWDELDIITPLYDKVRNWLSRKIYNPEKIKLCFENNGKLLSGWV |
| | | DSRTKSDNGTQYGGYIFRKKNEIGEYDFYLGISADTKLFRRDAAISYDD |
| | | GMYERLDYYQLKSKTLLGNSYVGDYGLDSMNLLSAFKNAAVKFQFEKEV |
| | | VPKDKENVPKYLKRLKLDYAGFYQILMNDDKVVDAYKIMKQHILATLTS |
| | | SIRVPAAIELATQKELGIDELIDEIMNLPSKSFGYFPIVTAAIEEANKR |
| | | ENKPLFLFKMSNKDLSYAATASKGLRKGRGTENLHSMYLKALLGMTQSV |
| | | FDIGSGMVFFRHQTKGLAETTARHKANEFVANKNKLNDKKKSIFGYEIV |
| | | KNKRFTVDKYLFKLSMNLNYSQPNNNKIDVNSKVREIISNGGIKNIIGI |
| | | DRGERNLLYLSLIDLKGNIVMQKSLNILKDDHNAKETDYKGLLTEREGE |
| | | NKEARRNWKKIANIKDLKRGYLSQVVHIISKMMVEYNAIVVLEDLNPGF |
| | | IRGRQKIERNVYEQFERMLIDKLNFYVDKHKGANETGGLLHALQLTSEF |
| | | KNFKKSEHQNGCLFYIPAWNTSKIDPATGFVNLFNTKYTNAVEAQEFFS |
| | | KFDEIRYNEEKDWFEFEFDYDKFTQKAHGTRTKWTLCTYGMRLRSFKNS |
| | | AKQYNWDSEVVALTEEFKRILGEAGIDIHENLKDAICNLEGKSQKYLEP |
| | | LMQFMKLLLQLRNSKAGTDEDYILSPVADENGIFYDSRSCGDQLPENAD |
| | | ANGAYNIARKGLMLIEQIKNAEDLNNVKFDISNKAWLNFAQQKPYKNG |
| ART10 | 10 | MNFQPFFQKFVHLYPISKTLRFELIPQGATQKFISEKQVLLQDEIRARK |
| | | YPEMKQAIDGYHKDFIQRALSNIDSQVFEQALNTFEDLFLRSQAERATD |
| | | AYKKDFETAQTKLRELIVHSFEKGEFKQEYKSLFDKNLITNLLKPWVEQ |
| | | QNQIGDSNYTYHEDFNKFTTYFLGFHENRKNIYSKDPHKTALAYRLIHE |
| | | NLPKFLENNKILLKIQNDHPSLWEQLQTLNQTMPQLFDGWDFSQLMQVS |
| | | FFSNTLTQTGIDQYNTIIGGISEGENRQKIQGINELINLYNQKQDKKNR |
| | | VAKLKQLYKQILSDRSTLSFLPEKFVDDTELYHAINMFYLEHLHHQSMI |
| | | NGHSYTLLERVQLLINELANYDLSKVYLAPNQLSTVSHQMFGDFGYIGR |
| | | ALNYYYMQVIQPDYEQLLASAKTTKKIEATEKLKTIFLDTPQSLVVIQA |
| | | AIDEYIQLQPSTKPHTQLTDFIISLLKQYETVADDQSIKVINVFSDIEG |
| | | KYSCIKGLVNTKSESKREVLQDEKLATDIKAFMDAVNNVIKLLKPFSLN |
| | | EKLVASVEKDARFYSDFEEIYQSLLIFVPLYNKVRNYITQKPYSTEKFK |
| | | LNFNKPTLLSGWDANKEADNLSILLRKNGNYYLAIMDTAKGANKAFEPK |
| | | TLNQLKVDDTTDCYEKMVYKLLSGPSKMFPKAFKAKNNEGNYYPTPELL |
| | | TSYNNNEHLKNDKNFTLASLHAYIDWCKEYINRNPSWHQFNFKFSPTQS |
| | | FQDISQFYSEVSSQSYKVHFQTIPSDYIDQLVAEGKLYLFQIYNKDFSP |
| | | NAKGKENLHTLYFKALFSDENLKQPVFKLSGEAEMFYRPASLQLANTTI |
| | | HKAGEPMAAKNPLTPNATRTLAYDIIKDRRFTTDKYLLHVPISLNFHAQ |
| | | ESMSIKKHNDLVRQMIKHNHQDLHVIGIDRGEKHLLYVSVIDLKGNIVY |
| | | QESLNSIKSEAQNFETPYHQLLQHREEGRAQARTAWGKIENIKELKDGY |
| | | LSQVVHRIQQLILKYNAIVMLEDLNFGFKRGRFKIEKQIYQKFEKALIH |
| | | KLNYVVDKSTQADELGGVRKAYQLTAPFESFEKLGKQSGVLFYVPAWNT |
| | | SKIDPVTGFVDLLKPKYENLDKAQAFFNAFDSIHYNAQKNYFEFKVNLK |
| | | QFAGLKAQAAQAEWTICSYGDERHVYQKKNAQQGETVIVNVTEELKVLF |
| | | AKNNIEVAQSVELKETICTQTQVDFFKRLMWLLQVLLALRYSSSKDKLD |
| | | YILSPVANAQGEFFDSRHASVQLPQDSDANGAYHIALKGLWVIEQLKAA |
| | | DNLDKVKLAISNDDWLHFAQQKPYLA |
| ART11 | 11 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKFSKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKFLDNIKAYSIAKSAGV |
| | | RAKELTEEEQDCLFMTETFERTLTQDGIDNYNELIGKLNFAINLYNQQN |
| | | NKLKGFRKVPKMKELYKQILSEREASFVDEFVDDEALLTNVESFSAHIK |
| | | EFLESDSLSRFAEVLEESGGEMVYIKNDTSKTTFSNIVFGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSFLDT |
| | | IKDFERDIKLISGSEHEMEKNLAVYAEQENILSSIRNVDSLYNMSRNYL |
| | | TQKPFSTEKFKLNFNRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKFSD |
| | | TKKYDDLSGFYREVEKQGYKITYTDIDVEYIDSLVEKDELYLFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRFNEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFFNRFDSICFNKVAGYFEF |
| | | SFDYKNFTDRACGMRSKWKVCTNGERIIKYRNEEKNSSFDDKVIVLTEE |
| | | FKKLFNEYGIAFNDCMDLTDAINAIDDASFFRKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |
| ART12 | 12 | MAKNFEDFKRLYPLSKTLRFEAKPIGATLDNIVKSGLLEEDEHRAASYV |
| | | KVKKLIDEYHKVFIDRVLDNGCLPLDDKGDNNSLAEYYESYVSKAQDED |
| | | AIKKFKEIQQNLLSIIAKKLTDDKAYANLFGNKLIESYKDKADKTKLID |
| | | SDLIQFINTAESTQLVSMSQDEAKELVKEFWGFTTYFEGFFKNRKNMYT |
| | | PEEKSTGIAYRLINENLPKFIDNMEAFKKAIARPEIQANMEELYSNFSE |
| | | YLNVESIQEMFLLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMFGN |
| | | WGVIQNAIMQNIKHVAPARKHKESEEDYEKRIAGIFKKADSFSISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNRPLTITKEVFDL |
| | | NNVLYGKYKKFQKGYLTATGDNVGYTHAVNVWIKFCMDFLDSYDSTCIY |
| | | DFSSLKPESYLSLDSFYQDVNLLLYKLSFTDVSASFIDQLVEEGKMYLF |
| | | QIYNKDFSEYSKGTPNMHTLYWKALFDERNLADVVYKLNGQAEMFYRKK |
| | | SIENTHPTHPANHPILNKNKDNKKKESLFEYDLIKDRRYTVDKFMFHVP |
| | | ITMNFKSSGSENINQDVKAYLRHADDMHIIGIDRGERHLLYLVVIDLQG |
| | | NIKEQFSLNEIVNDYNGNTYHTNYHDLLDVREDERLKARQSWQTIENIK |
| | | ELKEGYLSQVIHKITQLMVRYHAIVVLEDLSKGFMRSRQKVEKQVYQKF |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKFDAIRYNKDKKWF |
| | | EFNLDYDKFGKKAEDTRTKWTLCTRGMRIDTFRNKEKNSQWDNQEVDLT |
| | | TEMKSLLEHYYIDIHGNLKDAISTQTDKAFFTGLLHILKLTLQMRNSIT |
| | | GTETDYLVSPVADENGIFYDSRSCGDQLPENADANGAYNIARKGLMLVE |
| | | QIKDAEDLDNVKFDISNKAWLNFAQQKPYKNG |
| ART13 | 13 | MAKNFEDFKRLYSLSKTLRFEAKPIGATLDNIVKSGLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDFSE |
| | | YLNVESIQEMFQLDYYNMLLTQKQIDVYNAIIGGKTDDEHDVKIKGINE |
| | | YINLYNQQHKDDKLPKLKALFKQILSDRNAISWLPEEFNSDQEVLNAIK |
| | | DCYERLAENVLGDKVLKSLLGSLADYSLDGIFIRNDLQLTDISQKMFGN |
| | | WGVIQNAIMQNIKRVAPARKHKESEEDYEKRIAGIFKKADSFSISYIND |
| | | CLNEADPNNAYFVENYFATFGAVNTPTMQRENLFALVQNAYTEVAALLH |
| | | SDYPTVKHLAQDKANVSKIKALLDAIKSLQHFVKPLLGKGDESDKDERF |
| | | YGELASLWAELDTVTPLYNMIRNYMTRKPYSQKKIKLNFENPQLLGGWD |
| | | ANKEKDYATIILRRNGLYYLAIMDKDSRKLLGKAMPSDGECYEKMVYKF |
| | | FKDVTTMIPKCSTQLKDVQAYFKVNTDDYVLNSKAFNKPLTITKEVFDL |
| | | NNVLYGKYKKFQKGYLTATGDNVGYTHAVNVWIKFCMDFLNSYDSTCIY |
| | | DFSSLKPESYLSLDAFYQDANLLLYKLSFARASVSYINQLVEEGKMYLF |
| | | QIYNKDFSEYSKGTPNMHTLYWKALFDERNLADVVYKLNGQAEMFYRKK |
| | | SIENTHPTHPANHPILNKNKDNKKKESLFDYDLIKDRRYTVDKFMFHVP |
| | | ITMNFKSVGSENINQDVKAYLRHADDMHIIGIDRGERHLLYLVVIDLQG |
| | | NIKEQYSLNEIVNEYNGNTYHTNYHDLLDVREEERLKARQSWQTIENIK |
| | | ELKEGYLSQVIHKITQLMVRYHAIVVLEDLSKGFMRSRQKVEKQVYQKF |
| | | EKMLIDKLNYLVDKKTDVSTPGGLLNAYQLTCKSDSSQKLGKQSGFLFY |
| | | IPAWNTSKIDPVTGFVNLLDTHSLNSKEKIKAFFSKFDAIRYNKDKKWF |
| | | EFNLDYDKFGKKAEDTRTKWTLCTRGMRIDTFRNKEKNSQWDNQEVDLT |
| | | TEMKSLLEHYYIDIHGNLKDAISAQTDKAFFTGLLHILKLTLQMRNSIT |
| | | GTETDYLVSPVADENGIFYDSRSCGNQLPENADANGAYNIARKGLMLIE |
| | | QIKNAEDLNNVKFDISNKAWLNFAQQKPYKNG |
| ART14 | 14 | MAKNFEDFKRLYSLSKTLRFEAKPIGATLDNIVKSDLLDEDEHRAASYV |
| | | KVKKLIDEYHKVFIDRVLDDGCLPLENKGNNNSLAEYYESYVSRAQDED |
| | | AKKKFKEIQQNLRSVIAKKLTEDKAYANLFGNKLIESYKDKEDKKKIID |
| | | SDLIQFINTAESTQLDSMSQDEAKELVKEFWGFVTYFYGFFDNRKNMYT |
| | | AEEKSTGIAYRLVNENLPKFIDNIEAFNRAITRPEIQENMGVLYSDFSE |
| | | YLNVESIQEMFQLDYYNMLLTQKQIDVYNAIIGGKTDDEHDVKIKGIND |
| | | YINLYNQKHKDDKLPKLKALFKQILSDRNAISWLPEEFNSDQEVLNAIK |
| | | DCYERLSENVLGDKVLKSMLGSLADYSLDGIFIRNDLQLTDISQKMFGN |
| | | WSVIQNAIMQNIKHVAPARKHKESEEEYENRIAGIFKKADSFSISYIDA |
| | | CLNETDPNNAYFVENYFATLGAVDTPTMQRENLFALVQNAYTEITALLH |
| | | SDYPTEKNLAQDKANVAKIKALLDAIKSLQHFVKPLLGKGDESDKDERF |
| | | YGELASLWAELDTMTPLYNMIRNYMTRKPYSQKKIKLNFENPQLLGGWD |
| | | ANKEKDYATIILRRNGLYYLAIMNKDSKKLLGKAMPSDGECYEKMVYKL |
| | | LPGANKMLPKVFFAKSRMEDFKPSKELVEKYYNGTHKKGKNFNIQDCHN |
| | | LIDYFKQSIDKHEDWSKFGFKFSDTSTYEDLSGFYREVEQQGYKLSFAR |
| | | VSVSYINQLVEEGKMYLFQIYNKDFSEYSKGTPNMHTLYWKALFDERNL |
| | | ADVVYKLNGQAEMFYRKKSIENTHPTHPANHPILNKNKDNKKKESLFGY |
| | | DLIKDRRYTVDKFLFHVPITMNFKSSGSENINQDVKAYLRHADDMHIIG |
| | | IDRGERHLLYLVVIDLQGNIKEQFSLNEIVNDYNGNTYHTNYHDLLDVR |
| | | EDERLKARQSWQTIENIKELKEGYLSQVIHKITQLMVKYHAIVVLEDLN |
| | | MGFMRGRQKVEKQVYQKFEKMLIEKLNYLVDKKADASVSGGLLNAYQLT |
| | | SKFDSFQKLGKQSGFLFYIPAWNTSKIDPVTGFVNLLDTRYQNVEKAKS |
| | | FFSKFDAIRYNKDKEWFEFNLDYDKFGKKAEGTRTKWTLCTRGMRIDTF |
| | | RNKEKNSQWDNQEVDLTAEMKSLLEHYYIDIHSNLKDAISAQTDKAFFT |
| | | GLLHILKLTLQMRNSITGTETDYLVSPVVDENGIFYDSRSCGDELPENA |
| | | DANGAYNIARKGLMMIEQIKDAKDLDNLKFDISNKAWLNFAQQKPYKNG |
| ART15 | 15 | MLFQDFTHLYPLSKTVRFELKPIGRTLEHIHAKNFLSQDETMADMYQKV |
| | | KVILDDYHRDFIADMMGEVKLTKLAEFYDVYLKFRKNPKDDELQKQLKD |
| | | LQAVLRKESVKPIGNGGKYKAGHDRLFGAKLFKDGKELGDLAKFVIAQE |
| | | GKSSPKLAHLAHFEKFSTYFTGFHDNRKNMYSDEDKHTAIAYRLIHENL |
| | | PRFIDNLQILTTIKQKHSALYDQIINELTASGLDVSLASHLDGYHKLLT |
| | | QEGITAYNRIIGEVNGYTNKHNQICHKSERIAKLRPLHKQILSDGMGVS |
| | | FLPSKFADDSEMCQAVNEFYRHYADVFAKVQSLFDGFDDHQKDGIYVEH |
| | | KNLNELSKQAFGDFALLGRVLDGYYVDVVNPEFNERFAKAKTDNAKAKL |
| | | TKEKDKFIKGVHSLASLEQAIKHHTARHDDESVQAGKLGQYFKHGLAGV |
| | | DNPIQKIHNNHSTIKGFLERERPAGERALPKIKSGKNPEMTQLRQLKEL |
| | | LDNALNVAHFAKLLMTKTTLDNQDGNFYGEFGVLYDELAKIPTLYNKVR |
| | | DYLSQKPFSTEKYKLNFGNPTLLNGWDLNKEKDNFGVILQKDGCYYLAL |
| | | LDKAHKKVFDNAPNTGKNVYQKMIYKLLPGPNKMLPRVFFAKSNLDYYN |
| | | PSAELLDKYAQGTHKKGDNFNLKDCHALIDFFKAGINKHPEWQNFGFKF |
| | | SPTSSYRDLSDFYREVEPQGYQVKFVDINADYIDELVEQGQLYLFQIYN |
| | | KDFSPKAHGKPNLHTLYFRALFSEDNLANPIYKLNGEAQIFYRKASLGM |
| | | NETTIHRAGEILENKNPDNPKERVFTYDIIKDRRYTQDKFMLHVPITMN |
| | | FGVQGMTIKEFNKKVNQSIRQYDDVNVIGIDRGERHLLYLTVINSKGEI |
| | | LEQRSLNDITTASANGTQMTTPYHKILDKREIERLNARVGWGEIETIKE |
| | | LKSGYLSHVVHQVSQLMLKYNAIVVLEDLNFGFKRGRFKVEKQIYQNFE |
| | | NALIKKLNHLELKDKADDEIGSYKNALQLTNNFTDLKNIGKQTGFLFYV |
| | | PAWNTSKIDPETGFVDLLKPRYENIAQSQAFFGKFDKICYNADKDYFEF |
| | | HIDYAKFTDKAKNSRQTWTICSHGDKRYVYDKTANQNKGATKGINVNDE |
| | | LKSLFARYHINEKQPNLVMDICQNNDKEFHKSLMYLLKTLLALRYSNAS |
| | | SDEDFILSPVANDEGVFFNSALADDTQPQNADANGAYHIALKGLWLLNE |
| | | LKNSDDLNKVKLAIDNQTWLNFAQNR |
| ART16 | 16 | MLFQDFTHLYPLSKTVRFELKPIGKTLEHIHAKNFLSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKFVIAQE |
| | | GESSPKLPQIAHFEKFSTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLFDRFDDYQKDGIYVEH |
| | | KNLNELSKQAFGDFALLGRVLDGYYVDVVNPEFNDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVFDNAPNTGKSVYQKMVYKLLPGPNKMLPKVFFAKSNLDYYN |
| | | PSAELLDKYAQGTHKKGDNFNLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKFMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGRFKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFFDKFDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART17 | 17 | MLFQDFTHLYPLSKTVRFELKPIGKTLEHIHAKNFLSQDETMADMYQKV |
| | | KAILDDYHRDFITKMMSEVTLTKLPEFYEVYLALRKNPKDDTLQKQLTE |
| | | IQTALREEVVKPIDSGGKYKAGYERLFGAKLFKDGKELGDLAKFVIAQE |
| | | GESSPKLPQIAHFEKFSTYFTGFHDNRKNMYSSDDKHTAIAYRLIHENL |
| | | PRFIDNLQILVTIKQKHSVLYDQIVNELNANGLDVSLASHLDGYHKLLT |
| | | QEGITAYNRIIGEVNSYTNKHNQICHKSERIAKLRPLHKQILSDGMGVS |
| | | FLPSKFADDSEMCQAVNEFYRHYAHVFAKVQSLFDRFDDYQKDGIYVEH |
| | | KNLNELSKQAFGDFALLGRVLDGYYVDVVNPEFNDKFAKAKTDNAKEKL |
| | | TKEKDKFIKGVHSLASLEQAIEHYIAGHDDESVQAGKLGQYFKHGLAGV |
| | | DNPIQKIHNSHSTIKGFLERERPAGERTLPKIKSDKSLEMTQLRQLKEL |
| | | LDNALNVVHFAKLLTTKTTLDNQDGNFYGEFGALYDELAKIATLYNKVR |
| | | DYLSQKPFSTEKYKLNFGNPTLLNGWDLNKEKDNFGVILQKDGCYYLAL |
| | | LDKAHKKVFDNAPNTGKSVYQKMVYKLLPGSNKMLPKVFFAKSNLDYYN |
| | | PSAELLDKYAQGTHKKGDNFNLKDCHALIDFFKASINKHPEWQHFGFEF |
| | | SLTSSYQDLSDFYREVEPQGYQVKFVDIDADYIDELVEQGQLYLFQIYN |
| | | KDFSPKAHGKPNLHTLYFKALFSEDNLANPIYKLNGEAEIFYRKASLDM |
| | | NETTIHRAGEVLENKNPDNPKERQFVYDIIKDKRYTQDKFMLHVPITMN |
| | | FGVQGMTIKEFNKKVNQSIQQYDEVNVIGIDRGERHLLYLTVINSKGEI |
| | | LEQRSLNDIITTSANGTQMTTPYHKILDKREIERLNARVGWGEIETIKE |
| | | LKSGYLSHVVHQISQLMLKYNAIVVLEDLNFGFKRGREKVEKQIYQNFE |
| | | NALIKKLNHLVLKDKADNEIGSYKNALQLTNNFTDLKSIGKQTGFLFYV |
| | | PAWNTSKIDPVTGFVDLLKPRYENIAQSQAFFDKFDKICYNADKGYFEF |
| | | HIDYAKFTDKAKNSRQIWTICSHGDKRYVYDKTANQNKGATIGINVNDE |
| | | LKSLFARYRINDKQPNLVMDICQNNDKEFHKSLTYLLKALLALRYSNAS |
| | | SDEDFILSPVANDKGVFFNSALADDTQPQNADANGAYHIALKGLWLLNE |
| | | LKNSDDLDKVKLAIDNQTWLNFAQNR |
| ART18 | 18 | MKYTDFTGIYPVSKTLRFELIPQGSTVENMKREGILNNDMHRADSYKEM |
| | | KKLIDEYHKVFIERCLSDFSLKYDDTGKHDSLEEYFFYYEQKRNDKTKK |
| | | IFEDIQVALRKQISKRFTGDTAFKRLFKKELIKEDLPSFVKNDPVKTEL |
| | | IKEFSDFTTYFQEFHKNRKNMYTSDAKSTAIAYRIINENLPKFIDNINA |
| | | FHIVAKVPEMQEHFKTIADELRSHLQVGDDIDKMFNLQFFNKVLTQSQL |
| | | AVYNAVIGGKSEGNKKIQGINEYVNLYNQQHKKARLPMLKLLYKQILSD |
| | | RVAISWLQDEFDNDQDMLDTIEAFYNKLDSNETGVLGEGKLKQILMGLD |
| | | GYNLDGVFLRNDLQLSEVSQRLCGGWNIIKDAMISDLKRSVQKKKKETG |
| | | ADFEERVSKLFSAQNSFSIAYINQCLGQAGIRCKIQDYFACLGAKEGEN |
| | | EAETTPDIFDQIAEAYHGAAPILNARPSSHNLAQDIEKVKAIKALLDAL |
| | | KRLQRFVKPLLGRGDEGDKDSFFYGDFMPIWEVLDQLTPLYNKVRNRMT |
| | | RKPYSQEKIKLNFENSTLLNGWDLNKEHDNTSVILRREGLYYLGIMNKN |
| | | YNKIFDANNVETIGDCYEKMIYKLLPGPNKMLPKVFFSKSRVQEFSPSK |
| | | KILEIWESKSFKKGDNFNLDDCHALIDFYKDSIAKHPDWNKFNFKFSDT |
| | | QSYTNISDFYRDVNQQGYSLSFTKVSVDYVNRMVDEGKLYLFQIYNKDF |
| | | SPQSKGTPNMHTLYWRMLFDERNLHNVIYKLNGEAEVFYRKASLRCDRP |
| | | THPAHQPITCKNENDSKRVCVFDYDIIKNRRYTVDKFMFHVPITINYKC |
| | | TGSDNINQQVCDYLRSAGDDTHIIGIDRGERNLLYLVIIDQHGTIKEQF |
| | | SLNEIVNEYKGNTYCTNYHTLLEEKEAGNKKARQDWQTIESIKELKEGY |
| | | LSQVIHKISMLMQRYHAIVVLEDLNGSFMRSRQKVEKQVYQKFEHMLIN |
| | | KLNYLVNKQYDAAEPGGLLHALQLTSRMDSFKKLGKQSGFLFYIPAWNT |
| | | SKIDPVTGFVNLFDTRYCNEAKAKEFFEKFDDISYNDERDWFEFSFDYR |
| | | HFTNKPTGTRTQWTLCTQGTRVRTFRNPEKSNHWDNEEFDLTQAFKDLF |
| | | NKYGIDIASGLKARIVNGQLTKETSAVKDFYESLLKLLKLTLQMRNSVT |
| | | GTDIDYLVSPVADKDGIFFDSRTCGSLLPANADANGAFNIARKGLMLLR |
| | | QIQQSSIDAEKIQLAPIKNEDWLEFAQEKPYL |
| ART19 | 19 | METFSGFTNLYPLSKTLRFRLIPVGETLKYFIGSGILEEDQHRAESYVK |
| | | VKAIIDDYHRAYIENSLSGFELPLESTGKFNSLEEYYLYHNIRNKTEEI |
| | | QNLSSKVRTNLRKQVVAQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMEKLGYFNKTLSQKQ |
| | | IDAYNTVIGGKTTSEGKKIKGLNEYINLYNQQHKQEKLPKMKLLFKQIL |
| | | SDRESASWLPEKFENDSQVVGAIVNFWNTIHDTVLAEGGLKTIIASLGS |
| | | YGLEGIFLKNDLQLTDISQKATGSWGKISSEIKQKIEVMNPQKKKESYE |
| | | TYQERIDKIFKSYKSFSLAFINECLRGEYKIEDYFLKLGAVNSSSLQKE |
| | | NHFSHILNTYTDVKEVIGFYSESTDTKLIRDNGSIQKIKLFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLDKHEDWKNFGFKFSDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKADGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGFMRGRQKVEKQVYQKFEKKLIEKLNY |
| | | LVFKKQSSDLPGGLMHAYQLANKFESFNTLGKQSGFLFYIPAWNTSKMD |
| | | PVTGFVNLFDVKYESVDKAKSFFSKFDSIRYNVERDMFEWKFNYGEFTK |
| | | KAEGTKTDWTVCSYGNRIITFRNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART20 | 20 | METFSGFTNLYPLSKTLRFRLIPVGETLKHFIDSGILEEDQHRAESYVK |
| | | VKAIIDDYHRAYIENSLSGFELPLESTGKFNSLEEYYLYHNIRNKTEEI |
| | | QNLSSKVRTNLRKQVVVQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMFKLGYFNKTLSQKQ |
| | | IDAYNTVIGGKTTSEGKKIKGLNEYINLYNQQHKQEKLPKMKLLFKQIL |
| | | SDRESASWLPEKFENDSQVVGAMVNFWNTIHDTVLAEGGLKTIIASLGS |
| | | YGLEGIFLKNDLQLTDISQKATGSWSKISSEIKQKIEVMNPQKKKESYE |
| | | SYQERIDKLFKSYKSFSLAFINECLRGEYKIEDYFLKLGAVNSSSLQKE |
| | | NHFSHILNAYTDVKEAIGFYSESTDTKLIQDNDSIQKIKQFLDAVKDLQ |
| | | AYVKPLLGNGDETGKDERFYGDLIEYWSLLDLITPLYNMVRNYVTQKPY |
| | | SVDKIKINFQNPTLLNGWDLNKETDNTSVILRRDGKYYLAIMNNKSRKV |
| | | FLKYPSGTDGNCYEKMEYKLLPGANKMLPKVFFSKSRINEFMPNERLLS |
| | | NYEKGTHKKSGICFSLDDCHTLIDFFKKSLDKHEDWKNFGFKFSDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKADGN |
| | | GNINQKAIDYLCSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGFMRGRQKVEKQVYQKFEKKLIEKLNY |
| | | LVFKKQSSDLPGGLMHAYQLANKFESFNALGKQSGFLFYIPAWNTSKMD |
| | | PVTGFVNLFDVKYESVDKAKSFFSKFDSMRYNVERDMFEWKFNYGEFTK |
| | | KAEGTKTDWTVCSYGNRIITFRNPDKNSQWDNKEINLTENIKLLFERFG |
| | | IDLSSNLKDEIMQRTEKEFFIELISLFKLVLQMRNSWTGTDIDYLVSPV |
| | | CNENGEFFDSRNVDETLPQNADANGAYNIARKGMILLDKIKKSNGEKKL |
| | | ALSITNREWLSFAQGCCKNG |
| ART21 | 21 | METFSGFTNLYPLSKTLRFRLIPVGETLKHFIGSGILEEDQHRAESYVK |
| | | VKAIIDDYHRTYIENSLSGFELPLESTGKFNSLEEYYLYHNIRNKTEEI |
| | | QNLSSKVRTNLRKQVVTQLTKNEIFKRIDKKELIQSDLIDFVKNEPDAN |
| | | EKIALISEFRNFTVYFKGFHENRRNMYSDEEKSTSIAFRLIHENLPKFI |
| | | DNMEVFAKIQNTSISENFDAIQKELCPELVTLCEMFKLGYFNKTLSQKQ |
| | | IDAYNTVIGGKTTSEGKKIKGLNEYINLYNQQHKQEKLPKMKLLFKQIL |
| | | SDRESASWLLEKFENDSQVVGAMVNFWNTIHDTVLAEGGLKTIIASLGS |
| | | YGLEGIFLKNDLQLTDISQKATGSWSKISSEIKQKIEAMNPQKKKESYE |
| | | SYQERIDKLFKSYKSFSLAFVNECLRGEYKIEDYFLKLGAVNSSLLQKE |
| | | NHFSHILNTYTDVKEVIGFYSESTDTKLIQDNDSIQKIKQFLDAVKDLQ |
| | | AYVKPLLGNSDETGKDERFYGDLIEYWSLLDLITPLYNMVRNYVTQKPY |
| | | SVDKIKINFQNPTLLNGWDLNKEMDNTSVILRRDGKYYLAIMNNKSRKV |
| | | FLKYPSGTDRNCYEKMEYKLLPGANKMLPKVFFSKSRINEFMPNERLLS |
| | | NYEKGTHKKSGTCFSLDDCHTLIDFFKKSLNKHEDWKNFGFKFSDTSTY |
| | | EDMSGFYKEVENQGYKLSFKPIDATYVDQLVDEGKIFLFQIYNKDFSEH |
| | | SKGTPNMHTLYWKMLFDETNLGDVVYKLNGEAEVFFRKASINVSHPTHP |
| | | ANIPIKKKNLKHKDEERILKYDLIKDKRYTVDQFQFHVPITMNFKANGN |
| | | GNINQKAIDYLRSASDTHIIGIDRGERNLLYLVVIDGNGKICEQFSLNE |
| | | IEVEYNGEKYSTNYHDLLNVKENERKQARQSWQSIANIKDLKEGYLSQV |
| | | IHKISELMVKYNAIVVLEDLNAGFMRGRQKVEKQVYQKFEKKLIEKLNY |
| | | LVFKKQSSDLPGGLMHAYQLANKFESFNTLGKQSGFLFYIPAWNTSKMD |
| | | PVTGFVNLFDVKYESVDKAKSFFSKFDSIRYNVERDMFEWKFNYDEFTK |
| | | KAEGTKTDWTVCSYGNRIITFRNPDKNSQWDNKEINLTENIKLLFERFG |
| | | IDLSSNLKDEIMERTEKEFFIELISLFKLVLQMRNSWTGTDIDYLVSPV |
| | | CNENGEFFDSRNVDETLPQNADANGAYNIARKGMILLDKIKKNNGEKKL |
| | | TLSITNREWLSFAQGCCKNG |
| ART22 | 22 | MLFQDFTHLYPLSKTVRFELKPIGKTLEHIHAKNFLSQDKTMADMYQKV |
| | | KAILDDYHRDFIADMMGEVKLTKLAEFCDVYLKFRKNPKDDGLQKQLKD |
| | | LQAVLRKEIVKPIGNGGKYKVGYDRLFGAKLFKDGKELGDLAKFVIAQE |
| | | SESSPKLPQIAHFEKFSTYFTGFHDNRKNMYSSDDKHTAIAYRLIHENL |
| | | PRFIDNLQILATIKQKHSALYDQIASELTASGLDVSLASHLGGYHKLLT |
| | | QEGITAYNRIIGEVNSYTNKHNQICHKSERIAKLRPLHKQILSDGMGVS |
| | | FLPSKFADDSEMCQAVNEFYRHYADVFAKVQSLFDRFDDYQKDGIYVEH |
| | | KNLNELSKRAFGDFGFLKRFLEEYYADVIDPEFNEKFAKTEPDSDEQKK |
| | | LAGEKDKFVKGVHSLASLEQVIEYYTAGYDDESVQADKLGQYFKHRLAG |
| | | VDNPIQKIHNSHSTIKGFLERERPAGERALPKIKSDKSPEMTQLRQLKE |
| | | LLDNALNVVHFAKLVSTETVLDTRSDKFYGEFRPLYVELAKITTLYNKV |
| | | RDYLSQKPFSTEKYKLNFGNPTLLNGWDLNKEKDNFGVILQKDGCYYLA |
| | | LLDKAHKKVFDNAPNTGKSVYQKMVYKQIANARRDLACLLIINGKVVRK |
| | | TKGLDDLREKYLPYDIYKIYQSESYKVLSPNFNHQDLVKYIDYNKILAS |
| | | GYFEYFDFRFKESSEYKSYKEFLDDVDNCGYKISFCNINADYIDELVEQ |
| | | GQLYLFQIYNKDFSPKAHGKPNLHTLYFKALFSEDNLANPIYKLNGEAQ |
| | | IFYRKASLDMNETTIHRAGEVLENKNPDNPKQRQFVYDIIKDKRYTQDK |
| | | FMLHVPITMNFGVQGMTIEGFNKKVNQSIQQYDDVNVIGIDRGERHLLY |
| | | LTVINSKGEILEQRSLNDIITTSANGTQMTTPYHKILNKKKEGRLQARK |
| | | DWGEIETIKELKAGYLSHVVHQISQLMLKYNAIVVLEDLNFGFKRGRLK |
| | | VENQVYQNFENALIKKLNHLVLKDKTDDEIGSYKNALQLTNNFTDLKSI |
| | | GKQTGFLFYVPARNTSKIDPETGFVDLLKPRYENITQSQAFFGKFDKIC |
| | | YNTDKGYFEFHIDYAKFTDEAKNSRQTWVICSHGDKRYVYNKTANQNKG |
| | | ATKGINVNDELKSLFACHHINDKQPNLVMDICQNNDKEFHKSLMYLLKA |
| | | LLALRYSNANSDEDFILSPVANDEGVFFNSALADDTQPQNADANGAYHI |
| | | ALKGLWVLEQIKNSDDLDKVDLEIKDDEWRNFAQNR |
| ART23 | 23 | MGKNQNFQEFIGVSPLQKTLRNELIPTETTKKNITQLDLLTEDEIRAQN |
| | | REKLKEMMDDYYRDVIDSTLHAGIAVDWSYLFSCMRNHLRENSKESKRE |
| | | LERTQDSIRSQIYNKFAERADFKDMFGASIITKLLPTYIKQNPEYSERY |
| | | DESMEILKLYGKFTTSLTDYFETRKNIFSKEKISSAVGYRIVEENAEIF |
| | | LQNQNAYDRICKIAGLDLHGLDNEITAYVDGKTLKEVCSDEGFAKAITQ |
| | | EGIDRYNEAIGAVNQYMNLLCQKNKALKPGQFKMKRLHKQILCKGTTSF |
| | | DIPKKFENDKQVYDAVNSFTEIVMKNNDLKRLLNITQNVNDYDMNKIYV |
| | | AADAYSTISQFISKKWNLIEECLLDYYSDNLPGKGNAKENKVKKAVKEE |
| | | TYRSVSQLNELIEKYYVEKTGQSVWKVESYISRLAETITLELCHEIEND |
| | | EKHNLIEDDDKISKIKELLDMYMDAFHIIKVFRVNEVLNFDETFYSEMD |
| | | EIYQDMQEIVPLYNHVRNYVTQKPYKQEKYRLYFNTPTLANGWSKNKEY |
| | | DNNAIILMRDDKYYLGILNAKKKPSKQTMAGKEDCLEHAYAKMNYYLLP |
| | | GANKMLPKVFLSKKGIQDYHPSSYIVEGYNEKKHIKGSKNFDIRFCRDL |
| | | IDYFKECIKKHPDWNKFNFEFSATETYEDISVFYREVEKQGYRVEWTYI |
| | | NSEDIQKLEEDGQLFLFQIYNKDFAVGSTGKPNLHTLYLKNLFSEENLR |
| | | DIVLKLNGEAEIFFRKSSVQKPVIHKCGSILVNRTYEITESGTTRVQSI |
| | | PESEYMELYRYFNSEKQIELSDEAKKYLDKVQCNKAKTDIVKDYRYTMD |
| | | KFFIHLPITINFKVDKGNNVNAIAQQYIAEQEDLHVIGIDRGERNLIYV |
| | | SVIDMYGRILEQKSFNLVEQVSSQGTKRYYDYKEKLQNREEERDKARKS |
| | | WKTIGKIKELKEGYLSSVIHEIAQMVVKYNAIIAMEDLNYGFKRGRFKV |
| | | ERQVYQKFETMLISKLNYLADKSQAVDEPGGILRGYQMTYVPDNIKNVG |
| | | RQCGIIFYVPAAYTSKIDPTTGFINAFKRDVVSTNDAKENFLMKFDSIQ |
| | | YDIEKGLFKFSFDYKNFATHKLTLAKTKWDVYINGTRIQNMKVEGHWLS |
| | | MEVELTTKMKELLDDSHIPYEEGQNILDDLREMKDITTIVNGILEIFWL |
| | | TVQLRNSRIDNPDYDRIISPVLNNDGEFFDSDEYNSYIDAQKAPLPIDA |
| | | DANGAFCIALKGMYTANQIKENWVEGEKLPADCLKIEHASWLAFMQGER |
| | | G |
| ART24 | 24 | MNTSLFSSFTRQYPVTKTLRFELKPMGATLGHIQQKGFLHKDEELAKIY |
| | | KKIKELLDEYHRAFIADTLGDAQLVGLDDFYADYQALKQDSKNSHLKDK |
| | | LTKTQDNLRKQITKNFEKTPQLKERYKRLFTKELFKAGKDKGDLEKWLI |
| | | NHDSEPNKAEKISWIHQFENFTTYFQGFYENRKNMYSDEVKHTAIAYRL |
| | | IHENLPRFVDNIQVLSKIKSDYPDLYHELNHLDSRTIDFADFKFDDMLQ |
| | | MDFYHHLLIQSGITAYNTLLGGKVLEGGKKLQGINELINLYGQKHKIKI |
| | | AKLKPLHKQILSDGQSVSFLPKKFDNDYELCQTVNHFYREYVAIFDELV |
| | | VLFQKFYDYDKDNIYINHQQLNQLSHELFADFRLLSRALDFYYCQIIDG |
| | | DFNNKINNAKSQNAKEKLLKEKERYTKSNHSINELQKAINHYASHHEDT |
| | | EVKVISDYFSATNIRNMIDGIHHHFSTIKGFLEKDNNQGESYLPKQKNS |
| | | NDVKNLKLFLDGVLRLIHFIKPLALKSDDTLEKEEHFYGEFMPLYDKLV |
| | | MFTLLYNKVRDYISQKPYNDEKIKLNFGNSTLLNGWDVNKEKDNFGVIL |
| | | CKEGLYYLAILDKSHKKVFDNAPKATSSHTYQKMVYKLLPGPNKMLPKV |
| | | FFAKSNIGYYQPSAQLLENYEKGTHKKGSNFSLTDCHHLIDFFKSSIAK |
| | | HPEWKEFGFRFSDTHTYQDLSDFYKEIEPQSYKVKFIDIDADYIDDLVE |
| | | KGQLYLFQLYNKDFSKQSYGKPNLHTLYFKSLFSDDNLKNPIYKLNGEA |
| | | EIFYRRASLSVSDTTIHQAGEILTPKNPNNTHNRTLSYDVIKNKRYTTD |
| | | KFFLHIPITMNFGIENTGFKAFNHQVNTTLKNADKKDVHIIGIDRGERH |
| | | LLYVSVIDGDGRIVEQRTLNDIVSISNNGMSMSTPYHQILDNREKERLA |
| | | ARTDWGDIKNIKELKAGYLSHVVHEVVQMMLKYNAMIVLEDLNFGFKHG |
| | | RFKVEKQVYQNFENALIKKLNYLVLKNADNHQLGSVRKALQLINNFTDI |
| | | KSIGKQTGFIFYVPAWNTSKIDPTTGFVDLLKPRYENMAQAQSFISRFK |
| | | KIAYNHQLDYFEFEFDYADFYQKTIDKKRIWTLCTYGDVRYYYDHKTKE |
| | | TKTVNITKELKSLLDKHDLSYQNGHNLVDELANSHDKSLLSGVMYLLKV |
| | | LLALRYSHAQKNEDFILSPVMNKDGVFFDSRFADDVLPNNADANGAYHI |
| | | ALKGLWVLNQIQSADNMDKIDLSISNEQWLHFTQSR |
| ART25 | 25 | MVGNKISNSFDSFTGINALSKTLRNELIPSDYTKRHIAESDFIAADTNK |
| | | NEDQYVAKEMMDDYYRDFISKVLDNLHDIEWKNLFELMHKAKIDKSDAT |
| | | SKELIKIQDMLRKKIGKKFSQDPEYKVMLSAGMITKILPKYILEKYETD |
| | | REDRLEAIKRFYGFTVYFKEFWASRQNVFSDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSFNALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDFKFN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNFDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSFNMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVFNFREYSNFETKLD |
| | | FVRSLDSIRYDTEKKLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKFNRKLLSL |
| | | NNYNWFDFIQNRRF |
| ART26 | 26 | MVGNKISNSFDSFTGINALSKTLRNELIPSDYTKRHIAESDFIAADTNK |
| | | NEDQYVAKEMMDDYYRDFISKVLDNLHDIEWKNLFELMHKAKIDKSDAT |
| | | SKELIKIQDMLRKKIGKKFSQDPEYKVMLSAGMITKILPKYILEKYETD |
| | | REDRLEAIKRFYGFTVYFKEFWASRQNVFSDKAIASSISYRIIHENAKI |
| | | YMDNLDAYNRIKQIACEEIEKIEEEAYDFLQGDQLDVVYTEEAYGRFIS |
| | | QSGIDLYNNICGVINAHMNLYCQSKKCSRSKFKMQKLHKQILCKAETGF |
| | | EIPLGFQDDAQVINAINSFNALIKEKNIISRLRTIGKSISLYDVNKIYI |
| | | SSKAFENVSVYIDHKWDVIASSLYKYFSEIVKGNKDNREEKIQKEIKKV |
| | | KSCSLGDLQRLVNSYYKIDSTCLEHEVTEFVTKIIDEIDNFQITDFKFN |
| | | DKISLIQNEQIVMDIKTYLDKYMSIYHWMKSFVIDELVDKDMEFYSELD |
| | | ELNEDMSEIVNLYNKVRNYVTQKPYSQEKIKLNFGSPTLADGWSKSKEF |
| | | DNNAIILIRDEKIYLAIFNPRNKPAKTVISGHDVCNSETDYKKMNYYLL |
| | | PGASKTLPHVFIKSRLWNESHGIPDEILRGYELGKHLKSSVNFDVEFCW |
| | | KLIDYYKECISCYPNYKAYNFKFADTESYNDISEFYREVECQGYKIDWT |
| | | YISSEDVEQLDRDGQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSFNMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVFNFREYSNFETKLD |
| | | FVRSLDSIRYDTEKKLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDENGRFYDSEN |
| | | YIFNEETELPKDADANGAYCIALKGLYNVIAIKNNWKEGEKFNRKLLSL |
| | | NNYNWFDFIQNRRFQIYLFQIYNKDFAPNSKGMDNLHTKYLKNIFSEDN |
| | | LKNIVIKLNGEAELFYRKSSVKKKVEHKKGTILVNKTYKVEDNTENSKE |
| | | KRVIIESVPDDCYMELVDYWRNGGIGILSDKAVQYKDKVSHYEATMDIV |
| | | KDRRYTVDKFFIHLPITINFKADGRININEKVLKYIAENDELHVIGIDR |
| | | GERNLLYVSVINKKGKIVEQKSFNMIESYETVTNIVRRYNYKDKLVNKE |
| | | SARTDARKNWKEIGKIKEIKEGYLSQVIHEISKMVLKYNAIIVMEDLNY |
| | | GFKRGRFRVERQVYQKFENMLISKLAYLVDKSRKADEPGGVLRGYQLTY |
| | | IPDSLEKLGSQCGIIFYVPAAYTSKIDPLTGFVNVFNFREYSNFETKLD |
| | | FVRSLDSIRYDTEKRLFSISFDYDNFKTHNTTLAKTKWVIYLRGERIKK |
| | | EHTSYGWKDDVWNVESRIKDLFDSSHMKYDDGHNLIEDILELESSVQKK |
| | | LINELIEIIRLTVQLRNSKSERYDRTEAEYDRIVSPVMDEKGRFYDSEN |
| | | YIFNEETELPKDADANGAYCIALKGLYNVIAIKNNWKEGEKFNRKLLSL |
| | | NNYNWFDFIQNRRF |
| ART27 | 27 | MQEHKKISHLTHRNSVQKTIRMQLNPVGKTMDYFQAKQILENDEKLKED |
| | | YQKIKEIADRFYRNLNEDVLSKTGLDKLKDYAEIYYHCNTDADRKRLDE |
| | | CASELRKEIVKNFKNRDEYNKLFNKKMIEIVLPQHLKNEDEKEVVASFK |
| | | NFTTYFTGFFTNRKNMYSDGEESTAIAYRCINENLPKHLDNVKAFEKAI |
| | | SKLSKNAVDDLDTTYSGLCGTNLYDVFTVDYFNFLLPQSGITEYNKIIG |
| | | GYTTSDGTKVKGINEYINLYNQQVSKRYKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLFNESYANEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDF |
| | | QDCDESDCYEKIIYKLLPGPNKMLPKVFFSEKCKKLLSPSDEILKIRKN |
| | | GTFKKGDKFSLDDCHKLIDFYKESFKKYPNWLIYNFKFKKTNEYNDISE |
| | | FYNDVASQGYNISKMKIPTSFIDKLVDEGKIYLFQLYNKDFSPHSKGTP |
| | | NLHTLYFKMLFDERNLEDVVYKLNGEAEMFYRPASIKYDKPTHPKNTPI |
| | | KNKNTLNDKRASTFPYDLIKDKRYTKWQFSLHFPITMNFKAPDRAMIND |
| | | DVRNLLKSCNNNFIIGIDRGERNLLYVSIIDSNGAIIYQHSLNIIGNKF |
| | | KGKTYETNYREKLETREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPRYENIDKAKDMISRFDDIRYNAGEDFFEFDIDYDKFPKTASDY |
| | | RKKWTICTNGERIEAFRNPASNNEWSYRTIILAEKFKELFDNNSINYRD |
| | | SDNLKAEILSQTKGKFFEDFFKLLRLTLQMRNSNPETGEDRILSPVKDK |
| | | NGNFYDSSKYDEKSNLPCDADANGAYNIARKGLWIVEQFKKSDNVSTVE |
| | | PVIHNDKWLKFVQENDMANN |
| ART28 | 28 | MKNLANFTNLYSLQKTLRFELKPIGKILDWIIKKDLLKQDEILAEDYKI |
| | | VKKIIDRYHKDFIDLAFESAYLQKKSSDSFTAIMEASIQSYSELYFIKE |
| | | KSDRDKKAMEEISGIMRKEIVECFTGKYSEVVKKKFGNLFKKELIKEDL |
| | | LNFCEPDELPIIQKFADFTTYFTGFHENRENMYSNEEKATAIANRLIRE |
| | | NLPRYLDNLRIIRSIQGRYKDFGWKDLESNLKRIDKNLQYSDFLTENGF |
| | | VYTFSQKGIDRYNLILGGQSVESGEKIQGLNELINLYRQKNQLDRRQLP |
| | | NLKELYKQILSDRTRHSFVPEKFSSDKALLRSLLDFHKEVIQNKNLFEE |
| | | KQVSLLQAIRETLTDLKSFDLDRIYLTNDTSLTQISNFVFGDWSKVKTI |
| | | LAIYFDENIANPKDRQRQSNSYLKAKENWLKKNYYSIHELNEAISVYGK |
| | | HSDEELPNTKIEDYFSGLQTKDETKKPIDVLDAIVSKYADLESLLTKEY |
| | | PEDKNLKSDKGSIEKIKNYLDSIKLLQNFLKPLKPKKVQDEKDLGFYND |
| | | LELYLESLESANSLYNKVRNYLTGKEYSDEKIKLNFKNSTLLDGWDENK |
| | | ETSNLSVIFRDTNNYYLGILDKQNNRIFESIPEIQSGEETIQKMVYKLL |
| | | PGANNMLPKVFFSEKGLLKFNPSDEITSLYSEGRFKKGDKFSINSLHTL |
| | | IDFYKKSLAVHEDWSVFNFKFDETSHYEDISQFYRQVESQGYKITFKPI |
| | | SKKYIDTLVEDGKLYLFQIYNKDFSQNKKGGGKPNLHTIYFKSLFEKEN |
| | | LKDVIVKLNGQAEVFFRKKSIHYDENITRYGHHSELLKGRFSYPILKDK |
| | | RFTEDKFQFHFPITLNFKSGEIKQFNARVNSYLKHNKDVKIIGIDRGER |
| | | HLLYLSLIDQDGKILRQESLNLIKNDQNFKAINYQEKLHKKEIERDQAR |
| | | KSWGSIENIKELKEGYLSQVVHTISKLMVEHNAIVVLEDLNFGFKRGRQ |
| | | KVERQVYQKFEKMLIEKLNFLVFKDKEMDEPGGILKAYQLTDNFVSFEK |
| | | MGKQTGFVFYVPAWNTSKIDPKTGFVNFLHLNYENVNQAKELIGKFDQI |
| | | RYNQDRDWFEFQVTTDQFFTKENAPDTRTWIICSTPTKRFYSKRTVNGS |
| | | VSTIEIDVNQKLKELFNDCNYQDGEDLVDRILEKDSKDFFSKLIAYLRI |
| | | LTSLRQNNGEQGFEERDFILSPVVGSDGKFFNSLDASSQEPKDADANGA |
| | | YHIALKGLMNLHVINETDDESLGKPSWKISNKDWLNFVWQRPSLKA |
| ART29 | 29 | MQEHKKISHLTHRNSVQKTIRMQLNPVGKTMDYFQAKQILENDEKLKEN |
| | | YQKIKEIADRFYRNLNEDVLSKTRLDKLKDYTDIYYHCNTDADRKRLDE |
| | | CASELRKEIVKNFKNRDEYNKLFNKKMIEIVLPKHLKNEDEKEVVTSFK |
| | | NFTTYFTGFFTNRKNMYSDGEESTAIAYRCINENLPKHLDNVKAFEKAI |
| | | SKLSKNAIDDLDTTYSGLCGTNLYDVFTVDYFNFLLPQSGITEYNKIIG |
| | | GYTTNDGTKVKGINEYINLYNQQVSKRDKIPNLKILYKQILSESEKVSF |
| | | IPPKFEDDNELLSAVSEFYANDETFDGMPLKKAIDETKLLFGNLDNPSL |
| | | NGIYIQNDRSVTNLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYNEAAPLLNENYSNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDRNKEKDCGAVWLCKDEKYYLAIIDKSNNSILENIDF |
| | | QDCDESDCYEKIIYKLLPGPNKMLPKVFFSEKCKKLLSPSDEILKIYKS |
| | | GTFKTGDKFSLDDCHKLIDFYKESFKKYPNWLIYNFKFKKTNEYNDIRE |
| | | FYNDVALQGYNISKMKIPTSFIDKLVDEGKIYLFQLYNKDFSPHSKGTP |
| | | NLHTLYFKMLFDERNLEDVVYRLNGEAEMFYRPASIKYDKPTHPKNTPI |
| | | KNKNTLNDKKTSTFPYDLIKDKRYTKWQFSLHFPITMNFKAPDKAMIND |
| | | DVRNLLKSCNNNFIIGIDRGERNLLYVSVIDSNGAIIYQHSLNIIGNKF |
| | | KEKTYETNYREKLATREKERTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGMQSGFIFYVRPDFTSKIDPVTGF |
| | | VNLLYPQYENIDKAKDMISRFDEIRYNAGEDFFEFDIDYDEFPKTASDY |
| | | RKKWTICTNGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDQWLKFVQENDMANN |
| ART30 | 30 | MQEHKKISHLTHRNSVQKTIRMQLNPVGKTMDYFQAKQILENDEKLKED |
| | | YQKIKEIADRFYRNLNEDVLSKTGLDKLKDYADIYYHCNTDADRKRLNE |
| | | CASELRKEIVKNFKNRDEYNKLFNKKMIEIVLPKHLKNEDEKEVVASFK |
| | | NFTTYFTGFFTNRKNMYSDGEESTAIAYRCINENLPKHLDNVKVFEKAI |
| | | SKLSKNAIDDLGATYSGLCGTNLYDVFTVDYFNFLLPQSGITEYNKIIG |
| | | GYTTSDGTKVKGINEYINLYNQQVSKRDKIPNLKILYKQILSESEKVSF |
| | | IPPKFEDDNELLSAVSEFYANDETFDGMPLKKAIDETKLLFGNLDNSSL |
| | | NGIYIQNDRSVINLSNSMFGSWSVIEDLWNKNYDSVNSNSRIKDIQKRE |
| | | DKRKKAYKAEKKLSLSFLQVLISNSENDEIREKSIVDYYKTSLMQLTDN |
| | | LSDKYKEAAPLFSENYDNEKGLKNDDKSISLIKNFLDAIKEIEKFIKPL |
| | | SETNITGEKNDLFYSQFTPLLDNISRIDILYDKVRNYVTQKPFSTDKIK |
| | | LNFGNSQLLNGWDKDKEREYGAVLLCKDEKYYLAIIDKSNNSILENIDF |
| | | QDCNESDYYEKIVYKLLTKINGNLPRVFFSEKRKKLLSPSDEILKIYKS |
| | | GTFKKGDKFSLDDCHKLIDFYKESFKKYPNWLIYNFKFKNTNEYNDISE |
| | | FYNDVASQGYNISKMKIPTTFIDKLVDEGKIYLFQLYNKDFSPHSKGTP |
| | | NLHTLYFKMLFDERNLEDVVYKLNGEAEMFYRPASIKYDKPTHPKNTPI |
| | | KNKNTLNDKKASTFPYDLIKDKRYTKWQFSLHFPITMNFKAPDKAMIND |
| | | DVRNLLKSCNNNFIIGIDRGERNLLYVSVIDSNGAIIYQHSLNIIGNKF |
| | | KGKTYETNYREKLATREKDRTEQRRNWKAIESIKELKEGYISQAVHVIC |
| | | QLVVKYDAIIVMEKLTDGFKRGRTKFEKQVYQKFEKMLIDKLNYYVDKK |
| | | LDPDEEGGLLHAYQLTNKLESFDKLGTQSGFIFYVRPDFTSKIDPVTGF |
| | | VNLLYPRYENIDKAKDMISRFDDIRYNAGEDFFEFDIDYDKFPKTASDY |
| | | RKKWTICINGERIEAFRNPANNNEWSYRTIILAEKFKELFDNNSINYRD |
| | | SDDLKAEILSQTKGKFFEDFFKLLRLTLQMRNSNPETGEDRILSPVKDK |
| | | NGNFYDSSKYDEKSKLPCDADANGAYNIARKGLWIVEQFKKADNVSTVE |
| | | PVIHNDKWLKFVQENDMANN |
| ART31 | 31 | MQERKKISHLTHRNSVKKTIRMQLNPVGKTMDYFQAKQILENDEKLKEN |
| | | YQKIKEIADRFYRNLNEDVLSKTGLDKLKDYAEIYYHCNTDADRKRLNK |
| | | CASELRKEIVKNFKNRDEYNKLFDKRMIEIVLPKHLKNEDEKEVVASFK |
| | | NFTTYFTGFFTNRKNMYSDGEESTAIAYRCINENLPKHLDNVKAFEKAI |
| | | SKLSKNAIDDLDAYSGLCGTNLYDVFTVDYFNFLLPQSGITEYNKIIGG |
| | | YTTNDGTKVKGINEYINLYNQQVSKRDKIPNLQILYKQILSESEKVSFI |
| | | PPKFEDDNELLSAVSEFYANDETFDGMPLKKAIDETKLLFGNLDNSSLN |
| | | GIYIQNDRSVTNLSNSMFGSWSVIEDLWNKNYDSVNSNSRIKDIQKRED |
| | | KRKKAYKAEKKLSLSFLQVLISNSENDEIRKKSIVDYYKTSLMQLTDNL |
| | | SDKYNEAAPLLNENYSNEKGLKNDDKSISLIKNFLDAIKEIEKFIKPLS |
| | | ETNITGEKNDLFYSQFTPLLDNISRIDILYDKVRNYVTQKPFSTDKIKL |
| | | NFGNYQLLNGWDKDKEREYGAVLLCKDEKYYLAIIDKSNNRILENIDFQ |
| | | DCDESDCYEKIIYKLLPTPNKMLPKVFFAKKHKKLLSPSDEILKIYKNG |
| | | TFKKGDKFSLDDCHKLIDFYKESFKKYPKWLIYNFKFKKTNGYNDIREF |
| | | YNDVALQGYNISKMKIPTSFIDKLVDEGKIYLFQLYNKDFSPHSKGTPN |
| | | LHTLYFKMLFDERNLEDVVYRLNGEAEMFYRPASIKYDKPTHPKNTPIK |
| | | NKNTLNDKRASTFPYDLIKDKRYTKWQFSLHFPITMNFKDPDKAMINDD |
| | | VRNLLKSCNNNFIIGIDRGERNLLYVSVINSNGAIIYQHSLNIIGNKFK |
| | | GKTYETNYREKLATREKDRTEQRRNWKAIESIKELKEGYISQAVHVICQ |
| | | LVVKYDAIIVMEKLTDGFKRGRTKFEKQVYQKFEKMLIDKLNYYVDKKL |
| | | DPDEEGGLLHAYQLTNKLESFDKLGTQSGFIFYVRPDFTSKIDPVTGFV |
| | | NLLYPRYEKIDKAKDMISRFDDIRYNAGEDFFEFDIDYDKFPKTASDYR |
| | | KKWTICTNGERIEAFRNPANNNEWSYRTIILAEKFKELFDNNSINYRDS |
| | | DDLKAEILSQTKGKFFEDFFKLLRLTLQMRNSNPETGEDRILSPVKDKN |
| | | GNFYDSSKYDEKSKLPCDADANGAYNIARKGLWIVEQFKKADNVSTVEP |
| | | VIHNDKWLKFVQENDMANN |
| ART32 | 32 | KTGLDKLKDYAEIYYHCNTDADRKRLNKCASELRKEIVKNFKNRDEYNK |
| | | LFDKRMIEIVLPKHLKNEDEKEVVASFKNFTTYFTGFFTNRKNMYSDGE |
| | | ESTAIAYRCINENLPKHLDNVKAFEKAISKLSKNAIDDLDATYSGLCGT |
| | | NLYDVFTVDYFNFLLPQSGITEYNKIIGGYTTSDGTKVKGINEYINLYN |
| | | QQVSKRDKIPNLQILYKQILSESEKVSFIPPKFEDDNELLSAVSEFYAN |
| | | DETFDEMPLKKAIDETKLLFGNLDNSSLNGIYIQNDRSVTNLSNSMFGS |
| | | WSVIEDLWNKNYDSVNSNSRIKDIQKREDKRKKAYKAEKKLSLSFLQVL |
| | | ISNSENNEIREKSIVDYYKTSLMQLTDNLSDKYNEVAPLLNENYSNEKG |
| | | LKNDDKSISLIKNFLDAIKEIEKFIKPLSETNITGEKNDLFYSQFTPLL |
| | | DNISRIDILYDKVRNYVTQKPFSTDKIKLNFGNYQLLNGWDKDKEREYG |
| | | AVLLCRDEKYYLAIIDKSNNRILENIDFQDCDESDCYEKIIYKLLPTPN |
| | | KMLPKVFFAKKHKKLLSPSDEILKIRKNGTFKKGDKFSLDDCHKLIDFY |
| | | KESFKKYPNWLIYNFKFKKTNEYNDIREFYNDVALQGYNISKMKIPTSF |
| | | IDKLVDEGKIYLFQLYNKDFSPHSKGTPNLHTLYFKMLFDERNLEDVVY |
| | | KLNGEAKMFYRPASIKYDKPTHPKNTPIKNKNTLNDKKASTFPYDLIKD |
| | | KRYTKWQFSLHFSITMNFKAPDKAMINDDVRNLLKSCNNNFIIGIDRGE |
| | | RNLLYVSVIDSNGAIIYQHSLNIIGNKFKGKTYETNYREKLATREKERT |
| | | EQRRNWKAIESIKELKEGYISQAVHVICQLVVKYDAIIVMEKLTDGFKR |
| | | GRTKFEKQVYQKFEKMLIDKLNYYVDKKLDPDEEGGLLHAYQLTNKLES |
| | | FDKLGTQSGFIFYVRPDFTSKIDPVTGFVNLLYPRYENIDKAKDMISRF |
| | | DDIRYNAGEDFFEFDIDYDKFPKTASDYRKKWTICTNGERIEAFRNPAN |
| | | NNEWSYRTIILAEKFKELFDNNSINYRDSDDLKAEILSQTKGKFFEDFF |
| | | KLLRLTLQMRNSNPETGEDRILSPVKDKNGNFYDSSKYDEKSKLPCDAD |
| | | ANGAYNIARKGLWIVEQFKKSDNVSTVEPVIHNDKWLKFVQENDMANN |
| ART33 | 33 | MSININKFSDECRKIDFFTDLYNIQKTLRFSLIPIGATADNFEFKGRLS |
| | | KEKDLLDSAKRIKEYISKYLADESDICLSQPVKLKHLDEYYELYITKDR |
| | | DEQKFKSVEEKLRKELADLLKEILKRLNKKILSDYLPEYLEDDEKALED |
| | | IANLSSFSTYFNSYYDNCKNMYTDKEQSTAIPYRCINDNLPKFIDNMKA |
| | | YEKALEELKPSDLEELRNNFKGVYDTTVDDMFTLDYFNCVLSQSGIDSY |
| | | NAIIGNDKVKGINEYINLHNQTAEQGHKVPNLKRLYKQIGSQKKTISFL |
| | | PSKFESDNELLKAVYDFYNTGDAEKNFTALKDTITEFEKIFDNLSEYNL |
| | | DGVFVRNDISLTNLSQSMFNDWSVFRNLWNDQYDKVNNPEKAKDIDKYN |
| | | DKRHKVYKKSESFSINQLQELIATTLEEDINSKKITDYFSCDFHRVTTE |
| | | VENKYQLVKDLLSSDYPKNKNLKTSEEDVALIKDFLDSVKSLESFVKIL |
| | | TGTGKESGKDELFYGSFTKWFDQLRYIDKLYDKVRNYITEKPYSLDKIK |
| | | LSFDNPQFLGGWQHSKETDYSAQLFMKDGLYYLGVMDKETKREFKTQYN |
| | | TPENDSDTMVKIEYNQIPNPGRVIQNLMLVDGKIVKKNGRKNADGVNAV |
| | | LEELKNQYLPENINRIRKTESYKTTSNNFNKDDLKAYLEYYIARTKEYY |
| | | CKYNFVFKSADEYGSFNEFVDDVNNQAYQITKVKVSEKQLLSLVEQGKL |
| | | YLFKIYNKDFSEYSKGKKNLHTMYFQMLFDDRNLENLVYKLQGGAEMFY |
| | | RPASIKKDSEFKHDANVEIIKRTCEDKVNDKDNPTDDEKAKYYSKFDYD |
| | | IVKNKRFTKDQFSLHLTLAMNCNQPDHYWLNNDVRELLKKSNKNHIIGI |
| | | DRGERNLIYVTIINSDGVIVDQINFNIIENSYNGKKYKTDYQKKLNQRE |
| | | EDRQKARKTWKTIETIKELKDGYISQVVHQICKLIVQYDAIVVMENLNG |
| | | GFKRGRTKVEKQVYQKFETMLINKLNYYVDKGTDYKECGGLLKAYQLTN |
| | | KFETFERIGKQSGIIFYVDPYLTSKIDPVTGFANLLYPKYETIPKTHNF |
| | | ISNIDDIRYNQSEDYFEFDIDYDKFPQGSYNYRKKWTICSYGNRIKYYK |
| | | DSRNKTASVVVDITEKFKETFTNAGIDFVNDNIKEKLLLVNSKELLKSF |
| | | MDTLKLTVQLRNSEINSDVDYIISPIKDRNGNFYYSENYKKSNNEVPSQ |
| | | PQDGDANGAYNIARKGLMIINKLKKADDVTNNELLKISKKEWLEFAQKG |
| | | DLGE |
| ART34 | 34 | MKATSIWDNFTRKYSVSKTLRFELRPVGKTEENIVKKEIIDAEWISGKN |
| | | IPKGTDADRARDYKIVKKLLNQLHILFINQALSSENVKEFEKEDKKSKT |
| | | FVAWSDLLATHFDNWIQYTRDKSNSTVLKSLEKSKKDLYSKLGKLLNSK |
| | | ANAWKAEFISYHKIKSPDNIKIRLSASNVQILFGNTSDPIQLLKYQIEL |
| | | DNIKFLKDDGSEYTTKELADLLSTFEKFGTYFSGFNQNRANVYDIDGEI |
| | | STSIAYRLFNQNIEFFFQNIKRWEQFTSSIGHKEAKENLKLVQWDIQSK |
| | | LKELDMEIVQPRFNLKFEKLLTPQSFIYLLNQEGIDAFNTVLGGIPAEV |
| | | KAEKKQGVNELINLTRQKLNEDKRKFPSLQIMYKQIMSERKINFIDQYE |
| | | DDVEMLKEIQEFSNDWNEKKKRHSASSKEIKESAIAYIQREFHETFDSL |
| | | EERATVKEDFYLSEKSIQNLSIDIFGGYNTIHNLWYTEVEGMLKSGERP |
| | | LTRVEKEKLKKQEYISFAQIERLISKHSQQYLDSTPKEANDRSLFKEKW |
| | | KKTFKNGFKVSEYTNLKLNELISEGETFQKIDQETGKETTIKIPGLFES |
| | | YENAILVESIKNQSLGTNKKESVPSIKEYLDSCLRLSKFIESFLVNSKD |
| | | LKEDQSLDGCSDFQNTLTQWLNEEFDVFILYNKVRNHVTKKPGNTDKIK |
| | | INFDNATLLDGWDVDKEAANFGFLLKKADNYYLGIADSSFNQDLKYFNE |
| | | GERLDEIEKNRKNLEKEESKNISKIDQEKVKKYKEVIDDLKAISNLNKG |
| | | RYSKAFYKQSKFTTLIPKCTTQLNEVIEHFKKFDTDYRIENKKFAKPFI |
| | | ITKEVFLLNNTVYDTATKKFTLKIGEDEDTKGLKKFQIGYYRATDDKKG |
| | | YESALRNWITFCIEFTKSYKSCLNYNYSSLKSVSEYKSLDEFYKDLNGI |
| | | GYTIDFVDISEEYINKKINEGKLYLFQIYNKDFSEKSKGKENLHTTYWK |
| | | LLFDSKNLEDVVIKLNGQAEVFFRPASIHEKEKITHFKNQEIQNKNPNA |
| | | VKKTSKFEYDIIKDNRFTKNKFLFHCPITLNFKADGNPYVNNEVQENIA |
| | | KNPNVNIIGIDRGEKHLLYFTVINQQGQILDAGSLNSIKSEYKDKNQQS |
| | | VSFETPYHKILDKKESERKEARESWQEIENIKELKAGYLSHVVHQLSNL |
| | | IVKYNAIVVLEDLNKGFKRGRFKVEKQVYQKFEKSLIEKLNYLVFKDRK |
| | | ESNEPGHHLNAYQLTNKFLSFERLGKQSGVLFYATASYTSKVDPVTGFM |
| | | QNIYDPYHKEKTREFYKNFTKIVYNGNYFEFNYDLNSVKPDSEEKRYRT |
| | | NWTVCSCVIRSEYDSNSKTQKTYNVNDQLVKLFEDAKIKIENGNDLKST |
| | | ILEQDDKFIRDLHFYFIAIQKMRVVDSKIEKGEDSNDYIQSPVYPFYCS |
| | | KEIQPNKKGFYELPSNGDSNGAYNIARKGIVILDKIRLRVQIEKLFEDG |
| | | TKIDWQKLPNLISKVKDKKLLMTVFEEWAELTHQGEVQQGDLLGKKMSK |
| | | KGEQFAEFIKGLNVTKEDWEIYTQNEKVVQKQIKTWKLFSNST |
| ART35 | 35 | MKAINEYYKQLGAYCREEGKEKDDFFKRIDGAYCAISHLFFGEHGEIAQ |
| | | SDSDVELIQKLLEAYKGLQRFIKPLLGHGDEADKDNEFDAKLRKVWDEL |
| | | DIITPLYDKVRNWLSRKIYNPEKIKLCFENNGKLLSGWVDSRTKSDNGT |
| | | QYGGYIFRKKNEIGEYDFYLGISADTKLFRRDAAISYDDGMYERLDYYQ |
| | | LKSKTLLGNSYVGDYGLDSMNLLSAFKNAAVKFQFEKEVVPKDKENVPK |
| | | YLKRLKLDYAGFYQILMNDDKVVDAYKIMKQHILATLTSSIRVPAAIEL |
| | | ATQKELGIDELIDEIMNLPSKSFGYFPIVTAAIEEANKRENKPLFLFKM |
| | | SNKDLSYAATASKGLRKGRGTENLHSMYLKALLGMTQSVFDIGSGMVFF |
| | | RHQTKGLAETTARHKANEFVANKNKLNDKKKSIFGYEIVKNKRFTVDKY |
| | | LFKLSMNLNYSQPNNNKIDVNSKVREIISNGGIKNIIGIDRGERNLLYL |
| | | SLIDLKGNIVMQKSLNILKDDHNAKETDYKGLLTEREGENKEARRNWKK |
| | | IANIKDLKRGYLSQVVHIISKMMVEYNAIVVLEDLNPGFIRGRQKIERN |
| | | VYEQFERMLIDKLNFYVDKHKGANETGGLLHALQLTSEFKNFKKSEHQN |
| | | GCLFYIPAWNTSKIDPATGFVNLFNTKYTNAVEAQEFFSKEDEIRYNEE |
| | | KDWFEFEFDYDKFTQKAHGTRTKWTLCTYGMRLRSFKNSAKQYNWDSEV |
| | | VALTEEFKRILGEAGIDIHENLKDAICNLEGKSQKYLEPLMQFMKLLLQ |
| | | LRNSKAGTDEDYILSPVADENGIFYDSRSCGDQLPENADANGAYNIARK |
| | | GLMLIEQIKNAEDLNNVKFDISNKAWLNFAQQKPYKNGMKAINEYYKQL |
| | | GAYCREEGKEKDDFFKRIDGAYCAISHLFFGEHGEIAQSDSDVELIQKL |
| | | LEAYKGLQRFIKPLLGHGDEADKDNEFDAKLRKVWDELDIITPLYDKVR |
| | | NWLSRKIYNPEKIKLCFENNGKLLSGWVDSRTKSDNGTQYGGYIFRKKN |
| | | EIGEYDFYLGISADTKLFRRDAAISYDDGMYERLDYYQLKSKTLLGNSY |
| | | VGDYGLDSMNLLSAFKNAAVKFQFEKEVVPKDKENVPKYLKRLKLDYAG |
| | | FYQILMNDDKVVDAYKIMKQHILATLTSSIRVPAAIELATQKELGIDEL |
| | | IDEIMNLPSKSFGYFPIVTAAIEEANKRENKPLFLFKMSNKDLSYAATA |
| | | SKGLRKGRGTENLHSMYLKALLGMTQSVFDIGSGMVFFRHQTKGLAETT |
| | | ARHKANEFVANKNKLNDKKKSIFGYEIVKNKRFTVDKYLFKLSMNLNYS |
| | | QPNNNKIDVNSKVREIISNGGIKNIIGIDRGERNLLYLSLIDLKGNIVM |
| | | QKSLNILKDDHNAKETDYKGLLTEREGENKEARRNWKKIANIKDLKRGY |
| | | LSQVVHIISKMMVEYNAIVVLEDLNPGFIRGRQKIERNVYEQFERMLID |
| | | KLNFYVDKHKGANETGGLLHALQLTSEFKNFKKSEHQNGCLFYIPAWNT |
| | | SKIDPATGFVNLFNTKYTNAVEAQEFFSKFDEIRYNEEKDWFEFEFDYD |
| | | KFTQKAHGTRTKWTLCTYGMRLRSFKNSAKQYNWDSEVVALTEEFKRIL |
| | | GEAGIDIHENLKDAICNLEGKSQKYLEPLMQFMKLLLQLRNSKAGTDED |
| | | YILSPVADENGIFYDSRSCGDQLPENADANGAYNIARKGLMLIEQIKNA |
| | | EDLNNVKFDISNKAWLNFAQQKPYKNG |
| ART11* | 36 | MYYQGLTKLYPISKTIRNELIPVGKTLEHIRMNNILEADIQRKSDYERV |
| | | KKLMDDYHKQLINESLQDVHLSYVEEAADLYLNASKDKDIVDKFSKCQD |
| | | KLRKEIVNLLKSHENFPKIGNKEIIKLLQSLSDTEKDYNALDSFSKFYT |
| | | YFTSYNEVRKNLYSDEEKSSTAAYRLINENLPKFLDNIKAYSIAKSAGV |
| | | RAKELTEEEQDCLFMTETFERTLTQDGIDNYNELIGKLNFAINLYNQQN |
| | | NKLKGFRKVPKMKELYKQILSEREASFVDEFVDDEALLTNVESFSAHIK |
| | | EFLESDSLSRFAEVLEESGGEMVYIKNDTSKTTFSNIVFGSWNVIDERL |
| | | AEEYDSANSKKKKDEKYYDKRHKELKKNKSYSVEKIVSLSTETEDVIGK |
| | | YIEKLQADIIAIKETREVFEKVVLKEHDKNKSLRKNTKAIEAIKSFLDT |
| | | IKDFERDIKLISGSEHEMEKNLAVYAEQENILSSIRNVDSLYNMSRNYL |
| | | TQKPFSTEKFKLNFNRATLLNGWDKNKETDNLGILLVKEGKYYLGIMNT |
| | | KANKSFVNPPKPKTDNVYHKVNYKLLPGPNKMLPKVFFAKSNLEYYKPS |
| | | EDLLAKYQAGTHKKGENFSLEDCHSLISFFKDSLEKHPDWSEFGFKFSD |
| | | TKKYDDLSGFYREVEKQGYKITYTDIDVEYIDSLVEKDELYFFQIYNKD |
| | | FSPYSKGNYNLHTLYLTMLFDERNLRNVVYKLNGEAEVFYRPASIGKDE |
| | | LIIHKSGEEIKNKNPKRAIDKPTSTFEYDIVKDRRYTKDKFMLHIPVTM |
| | | NFGVDETRRFNEVVNDAIRGDDKVRVIGIDRGERNLLYVVVVDSDGTIL |
| | | EQISLNSIINNEYSIETDYHKLLDEKEGDRDRARKNWTTIENIKELKEG |
| | | YLSQVVNVIAKLVLKYDAIICLEDLNFGFKRGRQKVEKQVYQKFEKMLI |
| | | DKLNYLVIDKSRSQENPEEVGHVLNALQLTSKFTSFKELGKQTGIIYYV |
| | | PAYLTSKIDPTTGFANLFYVKYESVEKSKDFFNRFDSICFNKVAGYFEF |
| | | SFDYKNFTDRACGMRSKWKVCINGERIIKYRNEEKNSSFDDKVIVLTEE |
| | | FKKLFNEYGIAFNDCMDLTDAINAIDDASFFRKLTKLFQQTLQMRNSSA |
| | | DGSRDYIISPVENDNGEFFNSEKCDKSKPKDADANGAFNIARKGLWVLE |
| | | QLYNSSSGEKLNLAMTNAEWLEYAQQHTI |

In certain embodiments, a Cas nuclease comprises ABW1 (SEQ ID NO: 3), ABW2 (SEQ ID NO: 16), ABW3 (SEQ ID NO: 29), ABW4 (SEQ ID NO: 42), ABW5 (SEQ ID NO: 55), ABW6 (SEQ ID NO: 68), ABW7 (SEQ ID NO: 81), ABW8 (SEQ ID NO: 94), or ABW9 (SEQ ID NO: 107) (all SEQ ID NOs for ABW1-9 and variants thereof from International (PCT) Application Publication No. WO 2021/108324), or variants thereof, such as any one of variants 1-10 of ABW1 (SEQ ID NOs: 4-13, respectively), any one of variants 1-10 of ABW2 (SEQ ID NOs: 17-26, respectively), any one of variants 1-10 of ABW3 (SEQ ID NOs: 30-39, respectively), any one of variants 1-10 of ABW4 (SEQ ID NOs: 43-52, respectively), any one of variants 1-10 of ABW5 (SEQ ID NOs: 56-65, respectively), any one of variants 1-10 of ABW6 (SEQ ID NOs: 69-78, respectively), any one of variants 1-10 of ABW7 (SEQ ID NOs: 82-91, respectively), any one of variants 1-10 of ABW8 (SEQ ID NOs: 95-104, respectively), any one of variants 1-10 of ABW9 (SEQ ID NOs: 108-117, respectively). ABW1-ABW9, and variants thereof are known in the art and are described in International (PCT) Application Publication No. WO 2021/108324.

More type V-A Cas nucleases and their corresponding naturally occurring CRISPR-Cas systems can be identified by computational and experimental methods known in the art, e.g., as described in U.S. Pat. No. 9,790,490 and Shmakov et al. (2015) MOL. CELL, 60:385. Exemplary computational methods include analysis of putative Cas proteins by homology modeling, structural BLAST, PSI-BLAST, or HHPred, and analysis of putative CRISPR loci by identification of CRISPR arrays. Exemplary experimental methods include in vitro cleavage assays and in-cell nuclease assays (e.g., the Surveyor assay) as described in Zetsche et al. (2015) CELL, 163:759.

In certain embodiments, the Cas protein is a Cas nuclease that directs cleavage of one or both strands at the target locus, such as the target strand (i.e., the strand having the target nucleotide sequence that is at least partially complementary to and can hybridize with a single guide nucleic acid or dual guide nucleic acids) and/or the non-target strand. In certain embodiments, the Cas nuclease directs cleavage of one or both strands within about 1, 2, 3, 4, 5, 6, 7, 8, 9, 10, 15, 20, 25, 50, 100, 200, 500, or more nucleotides from the first or last nucleotide of the target nucleotide sequence or its complementary sequence. In certain embodiments, the cleavage is staggered, i.e. generating sticky ends. In certain embodiments, the cleavage generates a staggered cut with a 5′ overhang. In certain embodiments, the cleavage generates a staggered cut with a 5′ overhang of 1 to 5 nucleotides, e.g., of 4 or 5 nucleotides. In certain embodiments, the cleavage site is distant from the PAM, e.g., the cleavage occurs after the 18th nucleotide on the non-target strand and after the 23rd nucleotide on the target strand.

In certain embodiments, a composition provided herein comprises a Cas nuclease that a compatible guide nucleic acid (gNA), e.g., a gRNA, is capable of activating. In certain embodiments, a composition provided herein further comprises a Cas protein that is related to the Cas nuclease that a compatible guide nucleic acid (gNA), e.g., a gRNA, is capable of activating. For example, in certain embodiments, a Cas protein comprises an amino acid sequence at least 80% (e.g., at least 85%, at least 90%, at least 91%, at least 92%, at least 93%, at least 94%, at least 95%, at least 96%, at least 97%, at least 98%, or at least 99%) identical to the Cas nuclease amino acid sequence. In certain embodiments, a Cas protein comprises a nuclease-inactive mutant of the Cas nuclease. In certain embodiments, a Cas protein further comprises an effector domain.

In certain embodiments, a Cas protein lacks substantially all DNA cleavage activity. Such a Cas protein can be generated, e.g., by introducing one or more mutations to an active Cas nuclease (e.g., a naturally occurring Cas nuclease). A mutated Cas protein is considered to lack substantially all DNA cleavage activity when the DNA cleavage activity of the protein has no more than about 25%, 10%, 5%, 1%, 0.1%, 0.01%, or less of the DNA cleavage activity of the corresponding non-mutated form, for example, nil or negligible as compared with the non-mutated form. Thus, a Cas protein may comprise one or more mutations (e.g., a mutation in the RuvC domain of a type V-A Cas protein) and be used as a generic DNA binding protein with or without fusion to an effector domain. Exemplary mutations include D908A, E993A, and D1263A with reference to the amino acid positions in AsCpf1; D832A, E925A, and D1180A with reference to the amino acid positions in LbCpf1; and D917A, E1006A, and D1255A with reference to the amino acid position numbering of the FnCpf1. More mutations can be designed and generated according to the crystal structure described in Yamano et al. (2016) CELL, 165:949.

It is understood that a Cas protein, rather than losing nuclease activity to cleave all DNA, may lose the ability to cleave only the target strand or only the non-target strand of a double-stranded DNA, thereby being functional as a nickase (see, Gao et al. (2016) CELL RES., 26:901). Accordingly, in certain embodiments, a Cas nuclease is a Cas nickase. In certain embodiments, a Cas nuclease has the activity to cleave the non-target strand but lacks substantially the activity to cleave the target strand, e.g., by a mutation in the Nuc domain. In certain embodiments, a Cas nuclease has the cleavage activity to cleave the target strand but lacks substantially the activity to cleave the non-target strand.

In certain embodiments, a Cas nuclease has the activity to cleave a double-stranded DNA and result in a double-strand break.

Cas proteins that lack substantially all DNA cleavage activity or have the ability to cleave only one strand may also be identified from naturally occurring systems. For example, certain naturally occurring CRISPR-Cas systems may retain the ability to bind the target nucleotide sequence but lose entire or partial DNA cleavage activity in eukaryotic (e.g., mammalian or human) cells. Such type V-A proteins are disclosed, for example, in Kim et al. (2017) ACS SYNTH. BIOL. 6 (7): 1273-82 and Zhang et al. (2017) CELL DISCOV. 3:17018.

The activity of a Cas protein (e.g., Cas nuclease) can be altered, e.g., by creating an engineered Cas protein. In certain embodiments, altered activity of an engineered Cas protein comprises increased targeting efficiency and/or decreased off-target binding. While not wishing to be bound by theory, it is hypothesized that off-target binding can be recognized by the Cas protein, for example, by the presence of one or more mismatches between the spacer sequence and the target nucleotide sequence, which may affect the stability and/or conformation of the CRISPR-Cas complex. In certain embodiments, altered activity comprises modified binding, e.g., increased binding to the target locus (e.g., the target strand or the non-target strand) and/or decreased binding to off-target loci. In certain embodiments, altered activity comprises altered charge in a region of the protein that associates with a single guide nucleic acid or dual guide nucleic acids. In certain embodiments, altered activity of an engineered Cas protein comprises altered charge in a region of the protein that associates with the target strand and/or the non-target strand. In certain embodiments, altered activity of an engineered Cas protein comprises altered charge in a region of the protein that associates with an off-target locus. The altered charge can include decreased positive charge, decreased negative charge, increased positive charge, or increased negative charge. For example, decreased negative charge and increased positive charge may generally strengthen binding to the nucleic acid(s) whereas decreased positive charge and increased negative charge may weaken binding to the nucleic acid(s). In certain embodiments, altered activity comprises increased or decreased steric hindrance between the protein and a single guide nucleic acid or dual guide nucleic acids. In certain embodiments, altered activity comprises increased or decreased steric hindrance between the protein and the target strand and/or the non-target strand. In certain embodiments, altered activity comprises increased or decreased steric hindrance between the protein and an off-target locus. In certain embodiments, a modification or mutation comprises one or more substitutions of Lys, His, Arg, Glu, Asp, Ser, Gly, and/or Thr. In certain embodiments, a modification or mutation comprises one or more substitutions with Gly, Ala, Ile, Glu, and/or Asp. In certain embodiments, modification or mutation comprises one or more amino acid substitutions in the groove between the WED and RuvC domain of the Cas protein (e.g., a type V-A Cas protein).

In certain embodiments, altered activity of an engineered Cas protein comprises increased nuclease activity to cleave the target locus. In certain embodiments, altered activity of an engineered Cas protein comprises decreased nuclease activity to cleave an off-target locus. In certain embodiments, altered activity of an engineered Cas protein comprises altered helicase kinetics. In certain embodiments, an engineered Cas protein comprises a modification that alters formation of the CRISPR complex.

In certain embodiments, a protospacer adjacent motif (PAM) or PAM-like motif directs binding of a Cas protein complex to a target locus. Many Cas proteins have PAM specificity. The precise sequence and length requirements for the PAM differ depending on the Cas protein used. PAM sequences are typically 2-5 base pairs in length and are adjacent to (but located on a different strand of target DNA from) the target nucleotide sequence. PAM sequences can be identified using any suitable method, such as testing cleavage, targeting, or modification of oligonucleotides having the target nucleotide sequence and different PAM sequences.

Exemplary PAM sequences are provided in Tables 2 and 3. In certain embodiments, a Cas protein comprises MAD7 and the PAM is TTTN, wherein N is A, C, G, or T. In certain embodiments, a Cas protein comprises MAD7 and the PAM is CTTN, wherein N is A, C, G, or T. In certain embodiments, a Cas protein comprises AsCpf1 and the PAM is TTTN, wherein N is A, C, G, or T. In certain embodiments, a Cas protein comprises FnCpf1 and the PAM is 5′ TTN, wherein N is A, C, G, or T. PAM sequences for certain other type V-A Cas proteins are disclosed in Zetsche et al. (2015) CELL, 163:759 and U.S. Pat. No. 9,982,279. Further, engineering of the PAM Interacting (PI) domain of a Cas protein may allow programing of PAM specificity, improve target site recognition fidelity, and/or increase the versatility of an engineered, non-naturally occurring system. Exemplary approaches to alter the PAM specificity of Cpf1 are described in Gao et al. (2017) NAT. BIOTECHNOL., 35:789.

In certain embodiments, an engineered Cas protein comprises a modification that alters the Cas protein specificity in concert with modification to targeting range. Cas mutants can be designed to have increased target specificity as well as accommodating modifications in PAM recognition, for example by choosing mutations that alter PAM specificity (e.g., in the PI domain) and combining those mutations with groove mutations that increase (or if desired, decrease) specificity for the on-target locus versus off-target loci. The Cas modifications described herein can be used to counter loss of specificity resulting from alteration of PAM recognition, enhance gain of specificity resulting from alteration of PAM recognition, counter gain of specificity resulting from alteration of PAM recognition, or enhance loss of specificity resulting from alteration of PAM recognition.

In certain embodiments, an engineered Cas protein comprises one or more nuclear localization signal (NLS) motifs. In certain embodiments, an engineered Cas protein comprises at least 2 (e.g., at least 3, at least 4, at least 5, at least 6, at least 7, at least 8, at least 9, or at least 10) NLS motifs. Non-limiting examples of NLS motifs include: the NLS of SV40 large T-antigen, having the amino acid sequence of PKKKRKV (SEQ ID NO: 40); the NLS from nucleoplasmin, e.g., the nucleoplasmin bipartite NLS having the amino acid sequence of KRPAATKKAGQAKKKK (SEQ ID NO: 41); the c-myc NLS, having the amino acid sequence of PAAKRVKLD (SEQ ID NO: 42) or RQRRNELKRSP (SEQ ID NO: 43); the hRNPA1 M9 NLS, having the amino acid sequence of NOSSNFGPMKGGNFGGRSSGPYGGGGQYFAKPRNQGGY (SEQ ID NO: 44); the importin-α IBB domain NLS, having the amino acid sequence of RMRIZFKNKGKDTAELRRRRVEVSVELRKAKKDEQILKRRNV (SEQ ID NO: 45); the myoma T protein NLS, having the amino acid sequence of VSRKRPRP (SEQ ID NO: 46) or PPKKARED (SEQ ID NO: 47); the human p53 NLS, having the amino acid sequence of PQPKKKPL (SEQ ID NO: 48); the mouse c-abl IV NLS, having the amino acid sequence of SALIKKKKKMAP (SEQ ID NO: 49); the influenza virus NS1 NLS, having the amino acid sequence of DRLRR (SEQ ID NO: 50) or PKQKKRK (SEQ ID NO: 51); the hepatitis virus δ antigen NLS, having the amino acid sequence of RKLKKKIKKL (SEQ ID NO: 52); the mouse M×1 protein NLS, having the amino acid sequence of REKKKFLKRR (SEQ ID NO: 53); the human poly(ADP-ribose) polymerase NLS, having the amino acid sequence of KRKGDEVDGVDEVAKKKSKK (SEQ ID NO: 54); the human glucocorticoid receptor NLS, having the amino acid sequence of RKCLQAGMNLEARKTKK (SEQ ID NO: 55), and synthetic NLS motifs such as PAAKKKKLD (SEQ ID NO: 56).

In general, the one or more NLS motifs are of sufficient strength to drive accumulation of the Cas protein in a detectable amount in the nucleus of a eukaryotic cell. The strength of nuclear localization activity may derive from the number of NLS motif(s) in the Cas protein, the particular NLS motif(s) used, the position(s) of the NLS motif(s), or a combination of these and/or other factors. In certain embodiments, an engineered Cas protein comprises at least 1 (e.g., at least 2, at least 3, at least 4, at least 5, at least 6, at least 7, at least 8, at least 9, or at least 10) NLS motif(s) at or near the N-terminus (e.g., within about 1, 2, 3, 4, 5, 10, 15, 20, 25, 30, 40, 50, or more amino acids along the polypeptide chain from the N-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus (e.g., within about 1, 2, 3, 4, 5, 10, 15, 20, 25, 30, 40, 50, or more amino acids along the polypeptide chain from the C-terminus). In certain embodiments, an engineered Cas protein comprises at least 1 (e.g., at least 2, at least 3, at least 4, at least 5, at least 6, at least 7, at least 8, at least 9, or at least 10) NLS motif(s) at or near the C-terminus and at least 1 (e.g., at least 2, at least 3, at least 4, at least 5, at least 6, at least 7, at least 8, at least 9, or at least 10) NLS motif(s) at or near the N-terminus. In certain embodiments, the engineered Cas protein comprises one, two, or three NLS motifs at or near the C-terminus. In certain embodiments, the engineered Cas protein comprises one NLS motif at or near the N-terminus and one, two, or three NLS motifs at or near the C-terminus. In certain embodiments, the engineered Cas protein comprises a nucleoplasmin NLS at or near the C-terminus.

Detection of accumulation in the nucleus may be performed by any suitable technique. For example, a detectable marker may be fused to a nucleic acid-targeting protein, such that location within a cell may be visualized. Cell nuclei may also be isolated from cells, the contents of which may then be analyzed by any suitable process for detecting the protein, such as immunohistochemistry, Western blot, or enzyme activity assay. Accumulation in the nucleus may also be determined indirectly, such as by an assay that detects the effect of the nuclear import of a Cas protein complex (e.g., assay for DNA cleavage or mutation at the target locus, or assay for altered gene expression activity) as compared to a control not exposed to the Cas protein or exposed to a Cas protein lacking one or more of the NLS motifs.

A Cas protein may comprise a chimeric Cas protein, e.g., a Cas protein having enhanced function by being a chimera. Chimeric Cas proteins may be new Cas proteins containing fragments from more than one naturally occurring Cas protein or variants thereof. For example, fragments of multiple type V-A Cas homologs (e.g., orthologs) may be fused to form a chimeric Cas protein. In certain embodiments, a chimeric Cas protein comprises fragments of Cpf1 orthologs from multiple species and/or strains.

In certain embodiments, a Cas protein comprises one or more effector domains. The one or more effector domains may be located at or near the N-terminus of the Cas protein and/or at or near the C-terminus of the Cas protein. In certain embodiments, an effector domain comprised in the Cas protein is a transcriptional activation domain (e.g., VP64), a transcriptional repression domain (e.g., a KRAB domain or an SID domain), an exogenous nuclease domain (e.g., FokI), a deaminase domain (e.g., cytidine deaminase or adenine deaminase), or a reverse transcriptase domain (e.g., a high fidelity reverse transcriptase domain). Other activities of effector domains include but are not limited to methylase activity, demethylase activity, transcription release factor activity, translational initiation activity, translational activation activity, translational repression activity, histone modification (e.g., acetylation or demethylation) activity, single-stranded RNA cleavage activity, double-strand RNA cleavage activity, single-strand DNA cleavage activity, double-strand DNA cleavage activity, and nucleic acid binding activity.

In certain embodiments, a Cas protein comprises one or more protein domains that enhance homology-directed repair (HDR) and/or inhibit non-homologous end joining (NHEJ). Exemplary protein domains having such functions are described in Jayavaradhan et al. (2019) NAT. COMMUN. 10 (1): 2866 and Janssen et al. (2019) MOL. THER. NUCLEIC ACIDS 16:141-54. In certain embodiments, a Cas protein comprises a dominant negative version of p53-binding protein 1 (53BP1), for example, a fragment of 53BP1 comprising a minimum focus forming region (e.g., amino acids 1231-1644 of human 53BP1). In certain embodiments, a Cas protein comprises a motif that is targeted by APC-Cdh1, such as amino acids 1-110 of human Geminin, thereby resulting in degradation of the fusion protein during the HDR non-permissive G1 phase of the cell cycle.

In certain embodiments, a Cas protein comprises an inducible or controllable domain. Non-limiting examples of inducers or controllers include light, hormones, and small molecule drugs. In certain embodiments, a Cas protein comprises a light inducible or controllable domain. In certain embodiments, a Cas protein comprises a chemically inducible or controllable domain.

In certain embodiments, a Cas protein comprises a tag protein or peptide for case of tracking and/or purification. Non-limiting examples of tag proteins and peptides include fluorescent proteins (e.g., green fluorescent protein (GFP), YFP, RFP, CFP, mCherry, tdTomato), HIS tags (e.g., 6×His tag, or gly-6×His; 8×His, or gly-8×His), hemagglutinin (HA) tag, FLAG tag, 3×FLAG tag, and Myc tag.

In certain embodiments, a Cas protein is conjugated to a non-protein moiety, such as a fluorophore useful for genomic imaging. In certain embodiments, a Cas protein is covalently conjugated to the non-protein moiety. The terms “CRISPR-Associated protein,” “Cas protein,” “Cas,” “CRISPR-Associated nuclease,” and “Cas nuclease” are used herein to include such conjugates despite the presence of one or more non-protein moieties.

### B. Guide Nucleic Acids

A guide nucleic acid can be a single gNA (sgNA, e.g., sgRNA), in which the gNA is a single polynucleotide, or a dual gNA (e.g., dual gRNA), in which the gNA comprises two separate polynucleotides (these can in some cases be covalently linked, but not via a conventional internucleotide linkage). In certain embodiments, a single guide nucleic acid is capable of activating a Cas nuclease alone (e.g., in the absence of a tracrRNA).

In general, a gNA comprises a modulator nucleic acid and a targeter nucleic acid. In a sgNA the modulator and targeter nucleic acids are part of a single polynucleotide. In a dual gNA the modulator and targeter nucleic acids are separate, e.g., not joined by a conventional nucleotide linkage, such as not joined at all. The targeter nucleic acid comprises a spacer sequence and a targeter stem sequence. The modulator nucleic acid comprises a modulator stem sequence and, generally, further nucleotides, such as nucleotides comprising a 5′ tail. The modulator stem sequence and targeter stem sequence can each comprise any suitable number of nucleotides and are of sufficient complementarity that they can hybridize. In a single gNA there may be additional NTs between the targeter stem sequence and the modulator stem sequence; these can, in certain cases, form secondary structure, such as a loop.

In certain embodiments, the guide nucleic acid comprises a targeter nucleic acid that, in combination with a modulator nucleic acid, is capable of binding a Cas protein. In certain embodiments, the guide nucleic acid comprises a targeter nucleic acid that, in combination with a modulator nucleic acid, is capable of activating a Cas nuclease. In certain embodiments, the system further comprises the Cas protein that the targeter nucleic acid and the modulator nucleic acid are capable of binding or the Cas nuclease that the targeter nucleic acid and the modulator nucleic acid are capable of activating.

It is contemplated that the single or dual guide nucleic acids need to be the compatible with a Cas protein (e.g., Cas nuclease) to provide an operative CRISPR system. For example, the targeter stem sequence and the modulator stem sequence can be derived from a naturally occurring crRNA capable of activating a Cas nuclease in the absence of a tracrRNA. Alternatively, the targeter stem sequence and the modulator stem sequence can be derived from a naturally occurring set of crRNA and tracrRNA, respectively, that are capable of activating a Cas nuclease. In certain embodiments, the nucleotide sequences of the targeter stem sequence and the modulator stem sequence are identical to the corresponding stem sequences of a stem-loop structure in such naturally occurring crRNA.

Guide nucleic acid sequences that are operative with a type II or type V Cas protein are known in the art and are disclosed, for example, in U.S. Pat. Nos. 9,790,490, 9,896,696, 10,113,179, and 10,266,850, and U.S. Patent Application Publication No. 2014/0242664. It is understood that these sequences are merely illustrative, and other guide nucleic acid sequences may also be used with these Cas proteins.

TABLE 3
Type V-A Cas Protein and Corresponding Single Guide
Nucleic Acid Sequences

| Cas Protein | Scaffold Sequence¹ | PAM² |
|---|---|---|
| MAD7 (SEQ ID | UAAUUUCUACUCUUGUAGA (SEQ ID NO: 57), | 5' TTTN |
| NO: 37) | AUCUACAACAGUAGA (SEQ ID NO: 58), | or 5' |
| | AUCUACAAAAGUAGA (SEQ ID NO: 59), | CTTN |
| | GGAAUUUCUACUCUUGUAGA (SEQ ID NO: 60), | |
| | UAAUUCCCACUCUUGUGGG (SEQ ID NO: 61) | |
| MAD2 (SEQ ID | AUCUACAAGAGUAGA (SEQ ID NO: 62), | 5' TTTN |
| NO: 38) | AUCUACAACAGUAGA (SEQ ID NO: 58), | |
| | AUCUACAAAAGUAGA (SEQ ID NO: 59), | |
| | AUCUACACUAGUAGA (SEQ ID NO: 63) | |
| AsCpf1 (SEQ | UAAUUUCUACUCUUGUAGA (SEQ ID NO: 57) | 5' TTTN |
| ID NO: 3 of | | |
| WO | | |
| 2021/158918) | | |
| LbCpf1 (SEQ | UAAUUUCUACUAAGUGUAGA (SEQ ID NO: 64) | 5' TTTN |
| ID NO: 4 of | | |
| WO | | |
| 2021/158918) | | |
| FnCpf1 (SEQ | UAAUUUUCUACUUGUUGUAGA (SEQ ID NO: 65) | 5' TTN |
| ID NO: 5 of | | |
| WO | | |
| 2021/158918) | | |
| PbCpf1 (SEQ | AAUUUCUACUGUUGUAGA (SEQ ID NO: 66) | 5' TTTC |
| ID NO: 6 of | | |
| WO | | |
| 2021/158918) | | |
| PsCpf1 (SEQ | AAUUUCUACUGUUGUAGA (SEQ ID NO: 66) | 5' TTTC |
| ID NO: 7 of | | |
| WO | | |
| 2021/158918) | | |
| As2Cpf1 (SEQ | AAUUUCUACUGUUGUAGA (SEQ ID NO: 66) | 5' TTTC |
| ID NO: 8 of | | |
| WO | | |
| 2021/158918) | | |
| McCpf1 (SEQ | GAAUUUCUACUGUUGUAGA (SEQ ID NO: 67) | 5' TTTC |
| ID NO: 9 of | | |
| WO | | |
| 2021/158918) | | |
| Lb3Cpf1 (SEQ | GAAUUUCUACUGUUGUAGA (SEQ ID NO: 67) | 5' TTTC |
| ID NO: 10 of | | |
| WO | | |
| 2021/158918) | | |
| EcCpf1 (SEQ | GAAUUUCUACUGUUGUAGA (SEQ ID NO: 67) | 5' TTTC |
| ID NO: 11 of | | |
| WO | | |
| 2021/158918) | | |
| SmCsm1 (SEQ | GAAUUUCUACUGUUGUAGA (SEQ ID NO: 67) | 5' TTTC |
| ID NO: 12 of | | |
| WO | | |
| 2021/158918) | | |
| SsCsm1 (SEQ | GAAUUUCUACUGUUGUAGA (SEQ ID NO: 67) | 5' TTTC |
| ID NO: 13 of | | |
| WO | | |
| 2021/158918) | | |
| MbCsm1 (SEQ | GAAUUUCUACUGUUGUAGA (SEQ ID NO: 67) | 5' TTTC |
| ID NO: 14 of | | |
| WO | | |
| 2021/158918) | | |
| ART2 (SEQ ID | GUCUAAAGGUACCACCAAAUUUCUACUGUUGUAGAU | 5' TTTN |
| NO: 2 | (SEQ ID NO: 68) | or 5' |
| | | NTTN |
| ART11 (SEQ ID | GCUUAGAACCUUUAAAUAAUUUCUACUAUUGUAGAU | 5' TTTN |
| NO: 11 | (SEQ ID NO: 69) | or 5' |
| | | NTTN |
| ART11* (SEQ | GCUUAGAACCUUUAAAUAAUUUCUACUAUUGUAGAU | 5' TTTN |
| ID NO: 36 | (SEQ ID NO: 69) | or 5' |
| | | NTTN |

¹The modulator sequence in the scaffold sequence is underlined; the targeter stem sequence in the scaffold sequence is bold-underlined. It is understood that a “scaffold sequence“ listed herein constitutes a portion of a single guide nucleic acid. Additional nucleotide sequences, other than the spacer sequence, can be comprised in the single guide nucleic acid.
²In the consensus PAM sequences, N represents A, C, G, or T. Where the PAM sequence is preceded by “5',” it means that the PAM is located immediately upstream of the target nucleotide sequence when using the non-target strand (i.e., the strand not hybridized with the spacer sequence) as the coordinate.

TABLE 4
Type V-A Cas Protein and Corresponding Dual Guide Nucleic
Acid Sequences

| | | Targeter | |
|---|---|---|---|
| | | Stem | |
| Cas Protein | Modulator Sequence¹ | Sequence | PAM² |
| MAD7 (SEQ ID NO: | UAAUUUCUAC (SEQ ID NO: | GUAGA | 5' TTTN |
| 37) | 70) | | or 5' |
| | AUCUAC (SEQ ID NO: 71) | GUAGA | CTTN |
| | GGAAUUUCUAC (SEQ ID NO: | GUAGA | |
| | 72) | | |
| | UAAUUCCCAC (SEQ ID NO: | GUGGG | |
| | 73) | | |
| MAD2 (SEQ ID NO: | AUCUAC (SEQ ID NO: 71) | GUAGA | 5' TTTN |
| 38) | | | |
| AsCpf1 (SEQ ID NO: | UAAUUUCUAC (SEQ ID NO: | GUAGA | 5' TTTN |
| 3 of WO | 70) | | |
| 2021/158918) | | | |
| LbCpf1 (SEQ ID NO: | UAAUUUCUAC (SEQ ID NO: | GUAGA | 5' TTTN |
| 4 of WO | 70) | | |
| 2021/158918) | | | |
| FnCpf1 (SEQ ID NO: | UAAUUUUCUACU (SEQ ID NO: | GUAGA | 5' TTN |
| 5 of WO | 74) | | |
| 2021/158918) | | | |
| PbCpf1 (SEQ ID NO: | AAUUUCUAC (SEQ ID NO: 75) | GUAGA | 5' TTTC |
| 6 of WO | | | |
| 2021/158918) | | | |
| PsCpf1 (SEQ ID NO: | AAUUUCUAC (SEQ ID NO: 75) | GUAGA | 5' TTTC |
| 7 of WO | | | |
| 2021/158918) | | | |
| As2Cpf1 (SEQ ID | AAUUUCUAC (SEQ ID NO: 75) | GUAGA | 5' TTTC |
| NO: 8 of WO | | | |
| 2021/158918) | | | |
| McCpf1 (SEQ ID NO: | GAAUUUCUAC (SEQ ID NO: | GUAGA | 5' TTTC |
| 9 of WO | 76 | | |
| 2021/158918) | | | |
| Lb3Cpf1 (SEQ ID | GAAUUUCUAC (SEQ ID NO: | GUAGA | 5' TTTC |
| NO: 10 of WO | 76) | | |
| 2021/158918) | | | |
| EcCpf1 (SEQ ID NO: | GAAUUUCUAC (SEQ ID NO: | GUAGA | 5' TTTC |
| 11 of WO | 76) | | |
| 2021/158918) | | | |
| SmCsm1 (SEQ ID NO: | GAAUUUCUAC (SEQ ID NO: | GUAGA | 5' TTTC |
| 12 of WO | 76) | | |
| 2021/158918) | | | |
| SsCsm1 (SEQ ID NO: | GAAUUUCUAC (SEQ ID NO: | GUAGA | 5' TTTC |
| 13 of WO | 76 | | |
| 2021/158918) | | | |
| MbCsm1 (SEQ ID NO: | GAAUUUCUAC (SEQ ID NO: | GUAGA | 5' TTTC |
| 14 of WO | 76) | | |
| 2021/158918) | | | |
| ART2 (SEQ ID NO: 2) | AAAUUUCUAC (SEQ ID NO: | GUAGA | 5' TTTN |
| | 77) | | or 5' |
| | | | NTTN |
| ART11 (SEQ ID NO: | UAAUUUCUAC (SEQ ID NO: | GUAGA | 5' TTTN |
| 11) | 70) | | or 5' |
| | | | NTTN |
| ART11* (SEQ ID NO: | UAAUUUCUAC (SEQ ID NO: | GUAGA | 5' TTTN |
| 36) | 70 | | or 5' |
| | | | NTTN |

¹It is understood that a “modulator sequence” listed herein may constitute the nucleotide sequence of a modulator nucleic acid. Alternatively, additional nucleotide sequences can be comprised in the modulator nucleic acid 5' and/or 3' to a “modulator sequence” listed herein.
²In the consensus PAM sequences, N represents A, C, G, or T. Where the PAM sequence is preceded by “5',” it means that the PAM is located immediately upstream of the target nucleotide sequence when using the non-target strand (i.e., the strand not hybridized with the spacer sequence) as the coordinate.

In certain embodiments, a guide nucleic acid, in the context of a type V-A CRISPR-Cas system, comprises a targeter stem sequence listed in Table 4. The same targeter stem sequences, as a portion of scaffold sequences, are bold-underlined in Table 3.

In certain embodiments, a guide nucleic acid is a single guide nucleic acid that comprises, from 5′ to 3′, a modulator stem sequence, a loop sequence, a targeter stem sequence, and a spacer sequence. In certain embodiments, the targeter stem sequence in the single guide nucleic acid is listed in Table 3 as a bold-underlined portion of scaffold sequence, and the modulator stem sequence is complementary (e.g., 100% complementary) to the targeter stem sequence. In certain embodiments, the single guide nucleic acid comprises, from 5′ to 3′, a modulator sequence listed in Table 3 as an underlined portion of a scaffold sequence, a loop sequence, a targeter stem sequence a bold-underlined portion of the same scaffold sequence, and a spacer sequence. In certain embodiments, an engineered, non-naturally occurring system comprises a single guide nucleic acid comprising a scaffold sequence listed in Table 3. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 3. In certain embodiments, the system further comprises a Cas protein (e.g., Cas nuclease) comprising the amino acid sequence set forth in the SEQ ID NO listed in the same line of Table 3. In certain embodiments, the system is useful for targeting, editing, or modifying a nucleic acid comprising a target nucleotide sequence close or adjacent to (e.g., immediately downstream of) a PAM listed in the same line of Table 3 when using the non-target strand (i.e., the strand not hybridized with the spacer sequence) as the coordinate.

In certain embodiments, a guide nucleic acid, e.g., dual gNA, comprises a targeter guide nucleic acid that comprises, from 5′ to 3′, a targeter stem sequence and a spacer sequence. In certain embodiments, the targeter stem sequence in the targeter nucleic acid is listed in Table 4. In certain embodiments, an engineered, non-naturally occurring system comprises the targeter nucleic acid and a modulator stem sequence complementary (e.g., 100% complementary) to the targeter stem sequence. In certain embodiments, the modulator nucleic acid comprises a modulator sequence listed in the same line of Table 4. In certain embodiments, the system further comprises a Cas protein (e.g., Cas nuclease) comprising an amino acid sequence at least 30%, 40%, 50%, 60%, 70%, 80%, 85%, 90%, 91%, 92%, 93%, 94%, 95%, 96%, 97%, 98%, 99%, or 100% identical to the amino acid sequence set forth in the SEQ ID NO listed in the same line of Table 4. In certain embodiments, the system further comprises a Cas protein (e.g., Cas nuclease) comprising the amino acid sequence set forth in the SEQ ID NO listed in the same line of Table 4. In certain embodiments, the system is useful for targeting, editing, or modifying a nucleic acid comprising a target nucleotide sequence close or adjacent to (e.g., immediately downstream of) a PAM listed in the same line of Table 4 when using the non-target strand (i.e., the strand not hybridized with the spacer sequence) as the coordinate.

A single guide nucleic acid, the targeter nucleic acid, and/or the modulator nucleic acid can be synthesized chemically or produced in a biological process (e.g., catalyzed by an RNA polymerase in an in vitro reaction). Such reaction or process may limit the lengths of the single guide nucleic acid, targeter nucleic acid, and/or modulator nucleic acid. In certain embodiments, a single guide nucleic acid is no more than 100, 90, 80, 70, 60, 50, 40, 30, or 25 nucleotides in length. In certain embodiments, a single guide nucleic acid is at least 20, 25, 30, 40, 50, 60, 70, 80, or 90 nucleotides in length. In certain embodiments, the single guide nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a targeter nucleic acid is no more than 100, 90, 80, 70, 60, 50, 40, 30, or 25 nucleotides in length. In certain embodiments, a targeter nucleic acid is at least 20, 25, 30, 40, 50, 60, 70, 80, or 90 nucleotides in length. In certain embodiments, the targeter nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, a modulator nucleic acid is no more than 100, 90, 80, 70, 60, 50, 40, 30, or 20 nucleotides in length. In certain embodiments, a modulator nucleic acid is at least 10, 15, 20, 25, 30, 40, 50, 60, 70, 80, or 90 nucleotides in length. In certain embodiments, the modulator nucleic acid is 10-100, 10-90, 10-80, 10-70, 10-60, 10-50, 10-40, 10-30, 10-20, 15-100, 15-90, 15-80, 15-70, 15-60, 15-50, 15-40, 15-30, 15-20, 20-100, 20-90, 20-80, 20-70, 20-60, 20-50, 20-40, 20-30, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length.

It is contemplated that the length of the duplex formed within the single guide nuclei acid or formed between the targeter nucleic acid and the modulator nucleic acid, e.g. in a dual gNA, may be a factor in providing an operative CRISPR system. In certain embodiments, the targeter stem sequence and the modulator stem sequence each consist of 4-10 nucleotides that base pair with each other. In certain embodiments, the targeter stem sequence and the modulator stem sequence each consist of 4-9, 4-8, 4-7, 4-6, 4-5, 5-10, 5-9, 5-8, 5-7, or 5-6 nucleotides that base pair with each other. In certain embodiments, the targeter stem sequence and the modulator stem sequence each consist of 4, 5, 6, 7, 8, 9, or 10 nucleotides. It is understood that the composition of the nucleotides in each sequence affects the stability of the duplex, and a C-G base pair confers greater stability than an A-U base pair. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of the base pairs are C-G base pairs.

In certain embodiments, the targeter stem sequence and the modulator stem sequence each consist of 5 nucleotides. As such, the targeter stem sequence and the modulator stem sequence form a duplex of 5 base pairs. In certain embodiments, 0-4, 0-3, 0-2, 0-1, 1-5, 1-4, 1-3, 1-2, 2-5, 2-4, 2-3, 3-5, 3-4, or 4-5 out of the 5 base pairs are C-G base pairs. In certain embodiments, 0, 1, 2, 3, 4, or 5 out of the 5 base pairs are C-G base pairs. In certain embodiments, the targeter stem sequence consists of 5′-GUAGA-3′ and the modulator stem sequence consists of 5′-UCUAC-3′. In certain embodiments, the targeter stem sequence consists of 5′-GUGGG-3′ and the modulator stem sequence consists of 5′-CCCAC-3′.

In certain embodiments, in a type V-A system, the 3′ end of the targeter stem sequence is linked by no more than 1, 2, 3, 4, 5, 6, 7, 8, 9, or 10 nucleotides to the 5′ end of the spacer sequence. In certain embodiments, the targeter stem sequence and the spacer sequence are adjacent to each other, directly linked by an internucleotide bond. In certain embodiments, the targeter stem sequence and the spacer sequence are linked by one nucleotide, e.g., a uridine. In certain embodiments, the targeter stem sequence and the spacer sequence are linked by two or more nucleotides. In certain embodiments, the targeter stem sequence and the spacer sequence are linked by 1, 2, 3, 4, 5, 6, 7, 8, 9, or 10 nucleotides.

In certain embodiments, the targeter nucleic acid further comprises an additional nucleotide sequence 5′ to the targeter stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 2 nucleotides. In certain embodiments, the additional nucleotide sequence is reminiscent to the loop or a fragment thereof (e.g., one, two, three, or four nucleotides at the 3′ end of the loop) in a crRNA of a corresponding single guide CRISPR-Cas system. It is understood that an additional nucleotide sequence 5′ to the targeter stem sequence can be dispensable. Accordingly, in certain embodiments, the targeter nucleic acid does not comprise any additional nucleotide 5′ to the targeter stem sequence.

In certain embodiments, the targeter nucleic acid or the single guide nucleic acid further comprises an additional nucleotide sequence containing one or more nucleotides at the 3′ end that does not hybridize with the target nucleotide sequence. The additional nucleotide sequence may protect the targeter nucleic acid from degradation by 3′-5′ exonuclease. In certain embodiments, the additional nucleotide sequence is no more than 100 nucleotides in length. In certain embodiments, the additional nucleotide sequence is no more than 90, 80, 70, 60, 50, 40, 30, 20, or 10 nucleotides in length. In certain embodiments, the additional nucleotide sequence is at least 1, 2, 3, 4, 5, 6, 7, 8, 9, 10, 15, 20, 25, 30, 35, 40, 45, or 50 nucleotides in length. In certain embodiments, the additional nucleotide sequence is 5-100, 5-50, 5-40, 5-30, 5-25, 5-20, 5-15, 5-10, 10-100, 10-50, 10-40, 10-30, 10-25, 10-20, 10-15, 15-100, 15-50, 15-40, 15-30, 15-25, 15-20, 20-100, 20-50, 20-40, 20-30, 20-25, 25-100, 25-50, 25-40, 25-30, 30-100, 30-50, 30-40, 40-100, 40-50, or 50-100 nucleotides in length.

In certain embodiments, the additional nucleotide sequence forms a hairpin with the spacer sequence. Such secondary structure may increase the specificity of guide nucleic acid or the engineered, non-naturally occurring system (see, Kocak et al. (2019) Nat. Biotech. 37:657-66). In certain embodiments, the free energy change during the hairpin formation is greater than or equal to −20 kcal/mol, −15 kcal/mol, −14 kcal/mol, −13 kcal/mol, −12 kcal/mol, −11 kcal/mol, or −10 kcal/mol. In certain embodiments, the free energy change during the hairpin formation is greater than or equal to −5 kcal/mol, −6 kcal/mol, −7 kcal/mol, −8 kcal/mol, −9 kcal/mol, −10 kcal/mol, −11 kcal/mol, −12 kcal/mol, −13 kcal/mol, −14 kcal/mol, or −15 kcal/mol. In certain embodiments, the free energy change during the hairpin formation is in the range of −20 to −10 kcal/mol, −20 to −11 kcal/mol, −20 to −12 kcal/mol, −20 to −13 kcal/mol, −20 to −14 kcal/mol, −20 to −15 kcal/mol, −15 to −10 kcal/mol, −15 to −11 kcal/mol, −15 to −12 kcal/mol, −15 to −13 kcal/mol, −15 to −14 kcal/mol, −14 to −10 kcal/mol, −14 to −11 kcal/mol, −14 to −12 kcal/mol, −14 to −13 kcal/mol, −13 to −10 kcal/mol, −13 to −11 kcal/mol, −13 to −12 kcal/mol, −12 to −10 kcal/mol, −12 to −11 kcal/mol, or −11 to −10 kcal/mol. In other embodiments, the targeter nucleic acid or the single guide nucleic acid does not comprise any nucleotide 3′ to the spacer sequence.

In certain embodiments, the modulator nucleic acid further comprises an additional nucleotide sequence 3′ to the modulator stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 1 nucleotide (e.g., uridine). In certain embodiments, the additional nucleotide sequence consists of 2 nucleotides. In certain embodiments, the additional nucleotide sequence is reminiscent to the loop or a fragment thereof (e.g., one, two, three, or four nucleotides at the 5′ end of the loop) in a crRNA of a corresponding single guide CRISPR-Cas system. It is understood that an additional nucleotide sequence 3′ to the modulator stem sequence can be dispensable. Accordingly, in certain embodiments, the modulator nucleic acid does not comprise any additional nucleotide 3′ to the modulator stem sequence.

It is understood that the additional nucleotide sequence 5′ to the targeter stem sequence and the additional nucleotide sequence 3′ to the modulator stem sequence, if present, may interact with each other. For example, although the nucleotide immediately 5′ to the targeter stem sequence and the nucleotide immediately 3′ to the modulator stem sequence do not form a Watson-Crick base pair (otherwise they would constitute part of the targeter stem sequence and part of the modulator stem sequence, respectively), other nucleotides in the additional nucleotide sequence 5′ to the targeter stem sequence and the additional nucleotide sequence 3′ to the modulator stem sequence may form one, two, three, or more base pairs (e.g., Watson-Crick base pairs). Such interaction may affect the stability of a complex comprising the targeter nucleic acid and the modulator nucleic acid.

The stability of a complex comprising a targeter nucleic acid and a modulator nucleic acid can be assessed by the Gibbs free energy change (ΔG) during the formation of the complex, either calculated or actually measured. Where all the predicted base pairing in the complex occurs between a base in the targeter nucleic acid and a base in the modulator nucleic acid, i.e., there is no intra-strand secondary structure, the ΔG during the formation of the complex correlates generally with the ΔG during the formation of a secondary structure within the corresponding single guide nucleic acid. Methods of calculating or measuring the ΔG are known in the art. An exemplary method is RNAfold (rna.tbi.univic.ac.at/cgi-bin/RNAWebSuite/RNAfold.cgi) as disclosed in Gruber et al. (2008) Nucleic Acids Res., 36 (Web Server issue): W70-W74. Unless indicated otherwise, the ΔG values in the present disclosure are calculated by RNAfold for the formation of a secondary structure within a corresponding single guide nucleic acid. In certain embodiments, the ΔG is lower than or equal to −1 kcal/mol, e.g., lower than or equal to −2 kcal/mol, lower than or equal to −3 kcal/mol, lower than or equal to −4 kcal/mol, lower than or equal to −5 kcal/mol, lower than or equal to −6 kcal/mol, lower than or equal to −7 kcal/mol, lower than or equal to −7.5 kcal/mol, or lower than or equal to −8 kcal/mol. In certain embodiments, the ΔG is greater than or equal to −10 kcal/mol, e.g., greater than or equal to −9 kcal/mol, greater than or equal to −8.5 kcal/mol, or greater than or equal to −8 kcal/mol. In certain embodiments, the ΔG is in the range of −10 to −4 kcal/mol. In certain embodiments, the ΔG is in the range of −8 to −4 kcal/mol, −7 to −4 kcal/mol, −6 to −4 kcal/mol, −5 to −4 kcal/mol, −8 to −4.5 kcal/mol, −7 to −4.5 kcal/mol, −6 to −4.5 kcal/mol, or −5 to −4.5 kcal/mol. In certain embodiments, the ΔG is about-8 kcal/mol, −7 kcal/mol, −6 kcal/mol, −5 kcal/mol, −4.9 kcal/mol, −4.8 kcal/mol, −4.7 kcal/mol, −4.6 kcal/mol, −4.5 kcal/mol, −4.4 kcal/mol, −4.3 kcal/mol, −4.2 kcal/mol, −4.1 kcal/mol, or −4 kcal/mol.

It is understood that the ΔG may be affected by a sequence in the targeter nucleic acid that is not within the targeter stem sequence, and/or a sequence in the modulator nucleic acid that is not within the modulator stem sequence. For example, one or more base pairs (e.g., Watson-Crick base pair) between an additional sequence 5′ to the targeter stem sequence and an additional sequence 3′ to the modulator stem sequence may reduce the ΔG, i.e., stabilize the nucleic acid complex. In certain embodiments, the nucleotide immediately 5′ to the targeter stem sequence comprises a uracil or is a uridine, and the nucleotide immediately 3′ to the modulator stem sequence comprises a uracil or is a uridine, thereby forming a nonconventional U-U base pair.

In certain embodiments, the modulator nucleic acid or the single guide nucleic acid comprises a nucleotide sequence referred to herein as a “5′ tail” positioned 5′ to the modulator stem sequence. In a naturally occurring type V-A CRISPR-Cas system, the 5′ tail is a nucleotide sequence positioned 5′ to the stem-loop structure of the crRNA. A 5′ tail in an engineered type V-A CRISPR-Cas system, whether single guide or dual guide, can be reminiscent to the 5′ tail in a corresponding naturally occurring type V-A CRISPR-Cas system.

Without being bound by theory, it is contemplated that the 5′ tail may participate in the formation of the CRISPR-Cas complex. For example, in certain embodiments, the 5′ tail forms a pseudoknot structure with the modulator stem sequence, which is recognized by the Cas protein (see, Yamano et al. (2016) Cell, 165:949). In certain embodiments, the 5′ tail is at least 3 (e.g., at least 4 or at least 5) nucleotides in length. In certain embodiments, the 5′ tail is 3, 4, or 5 nucleotides in length. In certain embodiments, the nucleotide at the 3′ end of the 5′ tail comprises a uracil or is a uridine. In certain embodiments, the second nucleotide in the 5′ tail, the position counted from the 3′ end, comprises a uracil or is a uridine. In certain embodiments, the third nucleotide in the 5′ tail, the position counted from the 3′ end, comprises an adenine or is an adenosine. This third nucleotide may form a base pair (e.g., a Watson-Crick base pair) with a nucleotide 5′ to the modulator stem sequence. Accordingly, in certain embodiments, the modulator nucleic acid comprises a uridine or a uracil-containing nucleotide 5′ to the modulator stem sequence. In certain embodiments, the 5′ tail comprises the nucleotide sequence of 5′-AUU-3′. In certain embodiments, the 5′ tail comprises the nucleotide sequence of 5′-AAUU-3′. In certain embodiments, the 5′ tail comprises the nucleotide sequence of 5′-UAAUU-3′. In certain embodiments, the 5′ tail is positioned immediately 5′ to the modulator stem sequence.

In certain embodiments, the single guide nucleic acid, the targeter nucleic acid, and/or the modulator nucleic acid are designed to reduce the degree of secondary structure other than the hybridization between the targeter stem sequence and the modulator stem sequence. In certain embodiments, no more than about 75%, 50%, 40%, 30%, 25%, 20%, 15%, 10%, 5%, 1%, or fewer of the nucleotides of the single guide nucleic acid other than the targeter stem sequence and the modulator stem sequence participate in self-complementary base pairing when optimally folded. In certain embodiments, no more than about 75%, 50%, 40%, 30%, 25%, 20%, 15%, 10%, 5%, 1%, or fewer of the nucleotides of the targeter nucleic acid and/or the modulator nucleic acid participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (sce e.g., A. R. Gruber et al., 2008, Cell 106 (1): 23-24; and PA Carr and GM Church, 2009, Nature Biotechnology 27 (12): 1151-62).

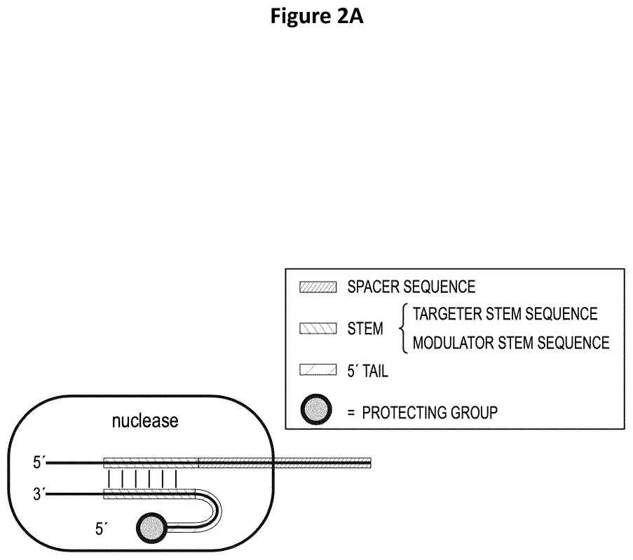

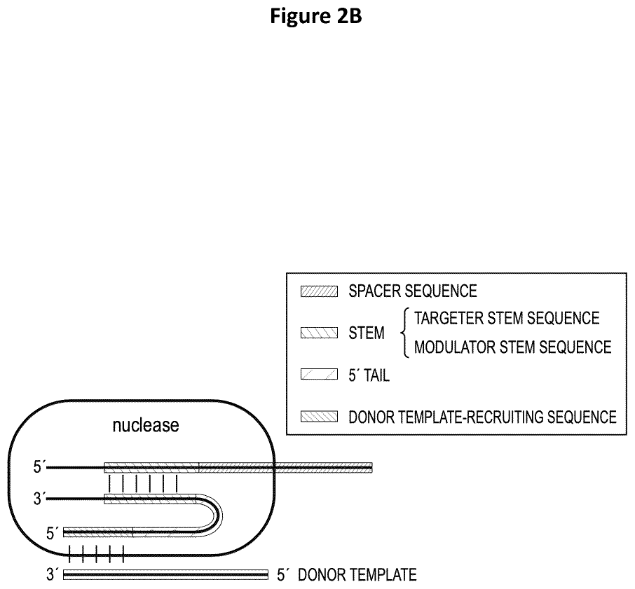

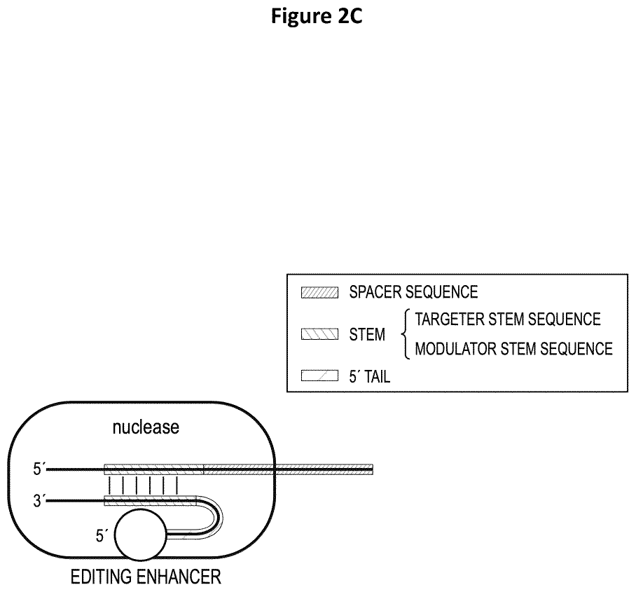

The targeter nucleic acid is directed to a specific target nucleotide sequence, and a donor template can be designed to modify the target nucleotide sequence or a sequence nearby. It is understood, therefore, that association of the single guide nucleic acid, the targeter nucleic acid, or the modulator nucleic acid with a donor template can increase editing efficiency and reduce off-targeting. Accordingly, in certain embodiments, the single guide nucleic acid or the modulator nucleic acid further comprises a donor template-recruiting sequence capable of hybridizing with a donor template (see FIG. 2B). Donor templates are described in the “Donor Templates” subsection of section II infra. The donor template and donor template-recruiting sequence can be designed such that they bear sequence complementarity. In certain embodiments, the donor template-recruiting sequence is at least 90% (e.g., at least 91%, at least 92%, at least 93%, at least 94%, at least 95%, at least 96%, at least 97%, at least 98%, or at least 99%) complementary to at least a portion of the donor template. In certain embodiments, the donor template-recruiting sequence is 100% complementary to at least a portion of the donor template. In certain embodiments, where the donor template comprises an engineered sequence not homologous to the sequence to be repaired, the donor template-recruiting sequence is capable of hybridizing with the engineered sequence in the donor template. In certain embodiments, the donor template-recruiting sequence is at least 20, 25, 30, 35, 40, 45, 50, 55, 60, 65, 70, 75, 80, 85, 90, 95, or 100 nucleotides in length. In certain embodiments, the donor template-recruiting sequence is positioned at or near the 5′ end of the single guide nucleic acid or at or near the 5′ end of the modulator nucleic acid. In certain embodiments, the donor template-recruiting sequence is linked to the 5′ tail, if present, or to the modulator stem sequence, of the single guide nucleic acid or the modulator nucleic acid through an internucleotide bond or a nucleotide linker.

In certain embodiments, the single guide nucleic acid or the modulator nucleic acid further comprises an editing enhancer sequence, which increases the efficiency of gene editing and/or homology-directed repair (HDR) (see FIG. 2C). Exemplary editing enhancer sequences are described in Park et al. (2018) Nat. Commun. 9:3313. In certain embodiments, the editing enhancer sequence is positioned 5′ to the 5′ tail, if present, or 5′ to the single guide nucleic acid or the modulator stem sequence. In certain embodiments, the editing enhancer sequence is 1-50, 4-50, 9-50, 15-50, 25-50, 1-25, 4-25, 9-25, 15-25, 1-15, 4-15, 9-15, 1-9, 4-9, or 1-4 nucleotides in length. In certain embodiments, the editing enhancer sequence is about 1, 2, 3, 4, 5, 6, 7, 8, 9, 10, 15, 20, 25, 30, 35, 40, 45, 50, or 55 nucleotides in length. The editing enhancer sequence is designed to minimize homology to the target nucleotide sequence or any other sequence that the engineered, non-naturally occurring system may be contacted to, e.g., the genome sequence of a cell into which the engineered, non-naturally occurring system is delivered. In certain embodiments, the editing enhancer is designed to minimize the presence of hairpin structure. The editing enhancer can comprise one or more of the chemical modifications disclosed herein.

The single guide nucleic acid, the modulator nucleic acid, and/or the targeter nucleic acid can further comprise a protective nucleotide sequence that prevents or reduces nucleic acid degradation. In certain embodiments, the protective nucleotide sequence is at least 5 (e.g., at least 10, at least 15, at least 20, at least 25, at least 30, at least 35, at least 40, at least 45, or at least 50) nucleotides in length. The length of the protective nucleotide sequence increases the time for an exonuclease to reach the 5′ tail, modulator stem sequence, targeter stem sequence, and/or spacer sequence, thereby protecting these portions of the single guide nucleic acid, the modulator nucleic acid, and/or the targeter nucleic acid from degradation by an exonuclease. In certain embodiments, the protective nucleotide sequence forms a secondary structure, such as a hairpin or a tRNA structure, to reduce the speed of degradation by an exonuclease (see, for example, Wu ct al. (2018) Cell. Mol. Life Sci., 75 (19): 3593-3607). Secondary structures can be predicted by methods known in the art, such as the online webserver RNAfold developed at University of Vienna using the centroid structure prediction algorithm (see, Gruber et al. (2008) Nucleic Acids Res., 36: W70). Certain chemical modifications, which may be present in the protective nucleotide sequence, can also prevent or reduce nucleic acid degradation, as disclosed in the “RNA Modifications” subsection infra.

A protective nucleotide sequence is typically located at the 5′ or 3′ end of the single guide nucleic acid, the modulator nucleic acid, and/or the targeter nucleic acid. In certain embodiments, the single guide nucleic acid comprises a protective nucleotide sequence at the 5′ end, at the 3′ end, or at both ends, optionally through a nucleotide linker. In certain embodiments, the modulator nucleic acid comprises a protective nucleotide sequence at the 5′ end, at the 3′ end, or at both ends, optionally through a nucleotide linker. In particular embodiments, the modulator nucleic acid comprises a protective nucleotide sequence at the 5′ end (see FIG. 2A). In certain embodiments, the targeter nucleic acid comprises a protective nucleotide sequence at the 5′ end, at the 3′ end, or at both ends, optionally through a nucleotide linker.

As described above, various nucleotide sequences can be present in the 5′ portion of a single nucleic acid or a modulator nucleic acid, including but not limited to a donor template-recruiting sequence, an editing enhancer sequence, a protective nucleotide sequence, and a linker connecting such sequence to the 5′ tail, if present, or to the modulator stem sequence. It is understood that the functions of donor template recruitment, editing enhancement, protection against degradation, and linkage are not exclusive to each other, and one nucleotide sequence can have one or more of such functions. For example, in certain embodiments, the single guide nucleic acid or the modulator nucleic acid comprises a nucleotide sequence that is both a donor template-recruiting sequence and an editing enhancer sequence. In certain embodiments, the single guide nucleic acid or the modulator nucleic acid comprises a nucleotide sequence that is both a donor template-recruiting sequence and a protective sequence. In certain embodiments, the single guide nucleic acid or the modulator nucleic acid comprises a nucleotide sequence that is both an editing enhancer sequence and a protective sequence. In certain embodiments, the single guide nucleic acid or the modulator nucleic acid comprises a nucleotide sequence that is a donor template-recruiting sequence, an editing enhancer sequence, and a protective sequence. In certain embodiments, the nucleotide sequence 5′ to the 5′ tail, if present, or 5′ to the modulator stem sequence is 1-90, 1-80, 1-70, 1-60, 1-50, 1-40, 1-30, 1-20, 1-10, 10-90, 10-80, 10-70, 10-60, 10-50, 10-40, 10-30, 10-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, or 80-90 nucleotides in length.

In certain embodiments, an engineered, non-naturally occurring system further comprises one or more compounds (e.g., small molecule compounds) that enhance HDR and/or inhibit NHEJ. Exemplary compounds having such functions are described in Maruyama et al. (2015) Nat Biotechnol. 33 (5): 538-42; Chu et al. (2015) Nat Biotechnol. 33 (5): 543-48; Yu et al. (2015) Cell Stem Cell 16 (2): 142-47; Pinder et al. (2015) Nucleic Acids Res. 43 (19): 9379-92; and Yagiz et al. (2019) Commun. Biol. 2:198. In certain embodiments, an engineered, non-naturally occurring system further comprises one or more compounds selected from the group consisting of DNA ligase IV antagonists (e.g., SCR7 compound, Ad4 E1B55K protein, and Ad4 E4orf6 protein), RAD51 agonists (e.g., RS-1), DNA-dependent protein kinase (DNA-PK) antagonists (e.g., NU7441 and KU0060648), B3-adrenergic receptor agonists (e.g., L755507), inhibitors of intracellular protein transport from the ER to the Golgi apparatus (e.g., brefeldin A), and any combinations thereof.

In certain embodiments, an engineered, non-naturally occurring system comprising a targeter nucleic acid and a modulator nucleic acid is tunable or inducible. For example, in certain embodiments, the targeter nucleic acid, the modulator nucleic acid, and/or the Cas protein can be introduced to the target nucleotide sequence at different times, the system becoming active only when all components are present. In certain embodiments, the amounts of the targeter nucleic acid, the modulator nucleic acid, and/or the Cas protein can be titrated to achieve desired efficiency and specificity. In certain embodiments, excess amount of a nucleic acid comprising the targeter stem sequence or the modulator stem sequence can be added to the system, thereby dissociating the complex of the targeter nucleic and modulator nucleic acid and turning off the system.

### C. gNA Modifications

Guide nucleic acids, including a single guide nucleic acid, a targeter nucleic acid, and/or a modulator nucleic acid, may comprise a DNA (e.g., modified DNA), an RNA (e.g., modified RNA), or a combination thereof. In certain embodiments, the single guide nucleic acid comprises a DNA (e.g., modified DNA), an RNA (e.g., modified RNA), or a combination thereof. In certain embodiments, the targeter nucleic acid comprises a DNA (e.g., modified DNA), an RNA (e.g., modified RNA), or a combination thereof. In certain embodiments, the modulator nucleic acid comprises a DNA (e.g., modified DNA), an RNA (e.g., modified RNA), or a combination thereof. Spacer sequences can be presented as DNA sequences by including thymidines (T) rather than uridines (U). It is understood that corresponding RNA sequences and DNA/RNA chimeric sequences are also contemplated. For example, where the spacer sequence is an RNA, its sequence can be derived from a DNA sequence disclosed herein by replacing each T with U. As a result, for the purpose of describing a nucleotide sequence, T and U are used interchangeably herein.

In certain embodiments engineered, non-naturally occurring systems comprising a targeter nucleic acid comprising: a spacer sequence designed to hybridize with a target nucleotide sequence and a targeter stem sequence; and a modulator nucleic acid comprising a modulator stem sequence complementary to the targeter stem sequence, and, optionally, a 5′ sequence, e.g., a tail sequence, wherein, in a single guide nucleic acid the targeter nucleic acid and the modulator nucleic acid are part of a single polynucleotide, and in a dual guide nucleic acid, the targeter nucleic acid and the modulator nucleic acid are separate nucleic acids; modifications can include one or more chemical modifications to one or more nucleotides or internucleotide linkages at or near the 3′ end of the targeter nucleic acid (dual and single gNA), at or near the 5′ end of the targeter nucleic acid (dual gNA), at or near the 3′ end of the modulator nucleic acid (dual gNA), at or near the 5′ end of the modulator nucleic acid (single and dual gNA), or combinations thereof as appropriate for single or dual gNA. In certain embodiments, the Cas nuclease is a type V-A Cas nuclease. Modulator and/or targeter nucleic sequences can include further sequences, as detailed in the Guide Nucleic Acids section, and modifications can be in these further sequences, as appropriate and apparent to one of skill in the art. In embodiments described in this section, below, in certain embodiments, guide nucleic acid is oriented from 5′ at the modulator nucleic acid to 3′ at the modulator stem sequence, and 5′ at the targeter stem sequence to 3′ at the targeter sequence (see, e.g., FIGS. 1A and 1B); in certain embodiments, as appropriate, guide nucleic acid is oriented from 3′ at the modulator nucleic acid to 5′ at the modulator stem sequence, and 3′ at the targeter stem sequence to 5′ at the targeter sequence.

The targeter nucleic acid may comprise a DNA (e.g., modified DNA), an RNA (e.g., modified RNA), or a combination thereof. The modulator nucleic acid may comprise a DNA (e.g., modified DNA), an RNA (e.g., modified RNA), or a combination thereof. In certain embodiments, the targeter nucleic acid is an RNA and the modulator nucleic acid is an RNA. A targeter nucleic acid in the form of an RNA is also called targeter RNA, and a modulator nucleic acid in the form of an RNA is also called modulator RNA. The nucleotide sequences disclosed herein are presented as DNA sequences by including thymidines (T) and/or RNA sequences including uridines (U). It is understood that corresponding DNA sequences, RNA sequences, and DNA/RNA chimeric sequences are also contemplated. For example, where a spacer sequence is presented as a DNA sequence, a nucleic acid comprising this spacer sequence as an RNA can be derived from the DNA sequence disclosed herein by replacing each T with U. As a result, for the purpose of describing a nucleotide sequence, T and U are used interchangeably herein.

In certain embodiments some or all of the gNA is RNA, e.g., a gRNA. In certain embodiments, 5-100%, 10-100%, 20-100%, 30-100%, 40-100%, 50-100%, 60-100%, 70-100%, 80-100%, 90-100%, 95-100%, 99-100%, 99.5-100% of the gNA is gRNA. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of gNA is RNA. In certain embodiments, 50% of the gNA is RNA. In certain embodiments, 70% of the gNA is RNA. In certain embodiments, 90% of the gNA is RNA. In certain embodiments, 100% of the gNA is RNA, e.g., a gRNA. In further embodiments, the remaining portion of the gNA that is not RNA comprises a modified ribonucleotide, a deoxyribonucleotide, a modified deoxyribonucleotide, or a synthetic, e.g., unnatural nucleotide, for example, not intended to be limiting, threose nucleic acid, locked nucleic acid, peptide nucleic acid, arabinonucleic acid, hexose nucleic acid, among others.

In certain embodiments, the targeter nucleic acid and/or the modulator nucleic acid are RNAs with one or more modifications in a ribose group, one or more modifications in a phosphate group, one or more modifications in a nucleobase, one or more terminal modifications, or a combination thereof. Exemplary modifications are disclosed in U.S. Pat. Nos. 10,900,034 and 10,767,175, U.S. Patent Application Publication No. 2018/0119140, Watts et al. (2008) Drug Discov. Today 13:842-55, and Hendel et al. (2015) NAT. BIOTECHNOL. 33:985.

In certain embodiments, a targeter nucleic acid, e.g., RNA, comprises at least one nucleotide at or near the 3′ end comprising a modification to a ribose, phosphate group, nucleobase, or terminal modification. In certain embodiments, the 3′ end of the targeter nucleic acid comprises the spacer sequence. In certain embodiments, the 3′ end of the targeter nucleic acid comprises the targeter stem sequence. Exemplary modifications are disclosed in Dang et al. (2015) Genome Biol. 16:280, Kocaz et al. (2019) Nature Biotech. 37:657-66, Liu et al. (2019) Nucleic Acids Res. 47(8): 4169-4180, Schubert et al. (2018) J. Cytokine Biol. 3(1): 121, Teng et al. (2019) Genome Biol. 20(1): 15, Watts et al. (2008) Drug Discov. Today 13(19-20): 842-55, and Wu et al. (2018) Cell Mol. Life. Sci. 75(19): 3593-607.

Modifications in a ribose group include but are not limited to modifications at the 2′ position or modifications at the 4′ position. For example, in certain embodiments, the ribose comprises 2′-O—C1-4alkyl, such as 2′-O-methyl (2′-OMe, or M). In certain embodiments, the ribose comprises 2′-O—C1-3alkyl-O-C1-3alkyl, such as 2′-methoxyethoxy (2′-O—CH2CH₂OCH3) also known as 2′-O-(2-methoxyethyl) or 2′-MOE. In certain embodiments, the ribose comprises 2′-O-allyl. In certain embodiments, the ribose comprises 2′-O-2,4-Dinitrophenol (DNP). In certain embodiments, the ribose comprises 2′-halo, such as 2′-F, 2′-Br, 2′-Cl, or 2′-I. In certain embodiments, the ribose comprises 2′-NH₂. In certain embodiments, the ribose comprises 2′-H (e.g., a deoxynucleotide). In certain embodiments, the ribose comprises 2′-arabino or 2′-F-arabino. In certain embodiments, the ribose comprises 2′-LNA or 2′-ULNA. In certain embodiments, the ribose comprises a 4′-thioribosyl.

Modifications can also include a deoxy group, for example a 2′-deoxy-3′-phosphonoacetate (DP), a 2′-deoxy-3′-thiophosphonoacetate (DSP).

Internucleotide linkage modifications in a phosphate group include but are not limited to a phosphorothioate(S), a chiral phosphorothioate, a phosphorodithioate, a boranophosphonate, a C₁₋₄alkyl phosphonate such as a methylphosphonate, a boranophosphonate, a phosphonocarboxylate such as a phosphonoacetate (P), a phosphonocarboxylate ester such as a phosphonoacetate ester, an amide, a thiophosphonocarboxylate such as a thiophosphonoacetate (SP), a thiophosphonocarboxylate ester such as a thiophosphonoacetate ester, and a 2′,5′-linkage having a phosphodiester or any of the modified phosphates above. Various salts, mixed salts and free acid forms are also included.

Modifications in a nucleobase include but are not limited to 2-thiouracil, 2-thiocytosine, 4-thiouracil, 6-thioguanine, 2-aminoadenine, 2-aminopurine, pseudouracil, hypoxanthine, 7-deazaguanine, 7-deaza-8-azaguanine, 7-deazaadenine, 7-deaza-8-azaadenine, 5-methylcytosinc, 5-methyluracil, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil, 5-allylcytosine, 5-aminoallyluracil, 5-aminoallyl-cytosine, 5-bromouracil, 5-iodouracil, diaminopurine, difluorotoluene, dihydrouracil, an abasic nucleotide, Z base, P base, Unstructured Nucleic Acid, isoguanine, isocytosine (see, Piccirilli et al. (1990) NATURE, 343:33), 5-methyl-2-pyrimidine (see, Rappaport (1993) BIOCHEMISTRY, 32:3047), x(A,G,C,T), and y(A,G,C,T).

Terminal modifications include but are not limited to polyethyleneglycol (PEG), hydrocarbon linkers (such as heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers, propanediol), spermine linkers, dyes such as fluorescent dyes (for example, fluoresceins, rhodamines, cyanines), quenchers (for example, dabcyl, BHQ), and other labels (for example biotin, digoxigenin, acridine, streptavidin, avidin, peptides and/or proteins). In certain embodiments, a terminal modification comprises a conjugation (or ligation) of the RNA to another molecule comprising an oligonucleotide (such as deoxyribonucleotides and/or ribonucleotides), a peptide, a protein, a sugar, an oligosaccharide, a steroid, a lipid, a folic acid, a vitamin and/or other molecule. In certain embodiments, a terminal modification incorporated into the RNA is located internally in the RNA sequence via a linker such as 2-(4-butylamidofluorescein) propane-1,3-diol bis(phosphodiester) linker, which is incorporated as a phosphodiester linkage and can be incorporated anywhere between two nucleotides in the RNA.

The modifications disclosed above can be combined in the targeter nucleic acid and/or the modulator nucleic acid that are in the form of RNA. In certain embodiments, the modification in the RNA is selected from the group consisting of incorporation of 2′-O-methyl-3′-phosphorothioate (MS), 2′-O-methyl-3′-phosphonoacetate (MP), 2′-O-methyl-3′-thiophosphonoacetate (MSP), 2′-halo-3′-phosphorothioate (e.g., 2′-fluoro-3′-phosphorothioate), 2′-halo-3′-phosphonoacctate (e.g., 2′-fluoro-3′-phosphonoacetate), and 2′-halo-3′-thiophosphonoacetate (e.g., 2′-fluoro-3′-thiophosphonoacetate).

In certain embodiments, modifications can include 2′-O-methyl (M), a phosphorothioate(S), a phosphonoacetate (P), a thiophosphonoacetate (SP), a 2′-O-methyl-3′-phosphorothioate (MS), a 2′-O-methyl-3′-phosphonoacetate (MP), a 2′-O-methyl-3′-thiophosphonoacetate (MSP), a 2′-deoxy-3′-phosphonoacetate (DP), a 2′-deoxy-3′-thiophosphonoacetate (DSP), or a combination thereof, at or near either the 3′ or 5′ end of either the targeter or modulator nucleic acid, as appropriate for single or dual gNA. In certain embodiments, modifications can include either a 5′ or a 3′ propanediol or C3 linker modification.

In certain embodiments, the modification alters the stability of the RNA. In certain embodiments, the modification enhances the stability of the RNA, e.g., by increasing nuclease resistance of the RNA relative to a corresponding RNA without the modification. Stability-enhancing modifications include but are not limited to incorporation of 2′-O-methyl, a 2′-O—C₁₋₄alkyl, 2′-halo (e.g., 2′-F, 2′-Br, 2′-Cl, or 2′-1), 2′MOE, a 2′-O—C₁₋₃alkyl-O—C₁₋₃alkyl, 2′-NH₂, 2′-H (or 2′-deoxy), 2′-arabino, 2′-F-arabino, 4′-thioribosyl sugar moiety, 3′-phosphorothioate, 3′-phosphonoacetate, 3′-thiophosphonoacetate, 3′-methylphosphonate, 3′-boranophosphate, 3′-phosphorodithioate, locked nucleic acid (“LNA”) nucleotide which comprises a methylene bridge between the 2′ and 4′ carbons of the ribose ring, and unlocked nucleic acid (“ULNA”) nucleotide. Such modifications are suitable for use as a protecting group to prevent or reduce degradation of the 5′ sequence, e.g., a tail sequence, modulator stem sequence (dual guide nucleic acids), targeter stem sequence (dual guide nucleic acids), and/or spacer sequence (see, the “Targeter and Modulator nucleic acids” subsection).

In certain embodiments, the modification alters the specificity of the engineered, non-naturally occurring system. In certain embodiments, the modification enhances the specificity of the engineered, non-naturally occurring system, e.g., by enhancing on-target binding and/or cleavage, or reducing off-target binding and/or cleavage, or a combination thereof. Specificity-enhancing modifications include but are not limited to 2-thiouracil, 2-thiocytosine, 4-thiouracil, 6-thioguanine, 2-aminoadenine, and pseudouracil. Within 10, 5, 4, 3, 2, or 1 nucleotide of the 3′ end, for example the 3′ end nucleotide, is modified

In certain embodiments, the modification alters the immunostimulatory effect of the RNA relative to a corresponding RNA without the modification. For example, in certain embodiments, the modification reduces the ability of the RNA to activate TLR7, TLR8, TLR9, TLR3, RIG-I, and/or MDA5.

In certain embodiments, the targeter nucleic acid and/or the modulator nucleic acid comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified nucleotides or internucleotide linkages. The modification can be made at one or more positions in the targeter nucleic acid and/or the modulator nucleic acid such that these nucleic acids retain functionality. For example, the modified nucleic acids can still direct the Cas protein to the target nucleotide sequence and allow the Cas protein to exert its effector function. It is understood that the particular modification(s) at a position may be selected based on the functionality of the nucleotide or internucleotide linkage at the position. For example, a specificity-enhancing modification may be suitable for a nucleotide or internucleotide linkage in the spacer sequence, the targeter stem sequence, or the modulator stem sequence. A stability-enhancing modification may be suitable for one or more terminal nucleotides or internucleotide linkages in the targeter nucleic acid and/or the modulator nucleic acid. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the targeter nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the targeter nucleic acid are modified. In certain embodiments, at least 1 (e.g., at least 2, at least 3, at least 4, or at least 5) terminal nucleotides or internucleotide linkages at or near the 5′ end and/or at least 1 (e.g., at least 2, at least 3, at least 4, or at least 5) terminal nucleotides or internucleotide linkages at or near the 3′ end of the modulator nucleic acid are modified. In certain embodiments, 5 or fewer (e.g., 1 or fewer, 2 or fewer, 3 or fewer, or 4 or fewer) terminal nucleotides or internucleotide linkages at or near the 5′ end and/or 5 or fewer (e.g., 1 or fewer, 2 or fewer, 3 or fewer, or 4 or fewer) terminal nucleotides or internucleotide linkages at or near the 3′ end of the modulator nucleic acid are modified. Selection of positions for modifications is described in U.S. Pat. Nos. 10,900,034 and 10,767,175. As used in this paragraph, where the targeter or modulator nucleic acid is a combination of DNA and RNA, the nucleic acid as a whole is considered as an RNA, and the DNA nucleotide(s) are considered as modification(s) of the RNA, including a 2′-H modification of the ribose and optionally a modification of the nucleobase.

It is understood that, in dual guide nucleic acid systems the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

### III. COMPOSITION AND METHODS FOR TARGETING, EDITING, AND/OR MODIFYING GENOMIC DNA

An engineered, non-naturally occurring system, such as disclosed herein, can be useful for targeting, editing, and/or modifying a target nucleic acid, such as a DNA (e.g., genomic DNA) in a cell or organism.

The present invention provides a method of cleaving a target nucleic acid (e.g., DNA) comprising the sequence of a preselected target sequence or a portion thereof, the method comprising contacting the target DNA with an engineered, non-naturally occurring system disclosed herein, thereby resulting in cleavage of the target DNA.

In addition, the present invention provides a method of binding a target nucleic acid (e.g., DNA) comprising the sequence of a preselected target sequence or a portion thereof, the method comprising contacting the target DNA with an engineered, non-naturally occurring system disclosed herein, thereby resulting in binding of the system to the target DNA. This method can be useful, e.g., for detecting the presence and/or location of the a preselected target gene, for example, if a component of the system (e.g., the Cas protein) comprises a detectable marker.

In addition, provided are methods of modifying a target nucleic acid (e.g., DNA) comprising the sequence of a preselected target sequence or a portion thereof, or a structure (e.g., protein) associated with the target DNA (e.g., a histone protein in a chromosome), the method comprising contacting the target DNA with an engineered, non-naturally occurring system disclosed herein, wherein the Cas protein comprises an effector domain or is associated with an effector protein, thereby resulting in modification of the target DNA or the structure associated with the target DNA. The modification corresponds to the function of the effector domain or effector protein. Exemplary functions described in the “Cas Proteins” subsection in Section I supra are applicable hereto.

An engineered, non-naturally occurring system can be contacted with the target nucleic acid as a complex. Accordingly, in certain embodiments, a method comprises contacting the target nucleic acid with a CRISPR-Cas complex comprising a targeter nucleic acid, a modulator nucleic acid, and a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

In certain embodiments, provided is a method of editing a human genomic sequence at one of a group of preselected target gene loci, the method comprising delivering an engineered, non-naturally occurring system disclosed herein into a human cell, thereby resulting in editing of the genomic sequence at the target gene locus in the human cell. In certain embodiments, provided herein is a method of detecting a human genomic sequence at one of a group of preselected target gene loci, the method comprising delivering the engineered, non-naturally occurring system disclosed herein into a human cell, wherein a component of the system (e.g., the Cas protein) comprises a detectable marker, thereby detecting the target gene locus in the human cell. In certain embodiments, provided herein is a method of modifying a human chromosome at one of a group of preselected target gene loci, the method comprising delivering the engineered, non-naturally occurring system disclosed herein into a human cell, wherein the Cas protein comprises an effector domain or is associated with an effector protein, thereby resulting in modification of the chromosome at the target gene locus in the human cell.

The CRISPR-Cas complex may be delivered to a cell by introducing a pre-formed ribonucleoprotein (RNP) complex into the cell. Alternatively, one or more components of the CRISPR-Cas complex may be expressed in the cell. Exemplary methods of delivery are known in the art and described in, for example, U.S. Pat. Nos. 8,697,359, 10,113,167, 10,570,418, 10,829,787, 11,118,194, and 11,125,739 and U.S. Patent Application Publication Nos. 2015/0344912, 2018/0119140, and 2018/0282763.

It is understood that contacting a DNA (e.g., genomic DNA) in a cell with a CRISPR-Cas complex does not require delivery of all components of the complex into the cell. For example, one or more of the components may be pre-existing in the cell. In certain embodiments, the cell (or a parental/ancestral cell thereof) has been engineered to express the Cas protein, and the single guide nucleic acid (or a nucleic acid comprising a regulatory element operably linked to a nucleotide sequence encoding the single guide nucleic acid), the targeter nucleic acid (or a nucleic acid comprising a regulatory element operably linked to a nucleotide sequence encoding the targeter nucleic acid), and/or the modulator nucleic acid (or a nucleic acid comprising a regulatory element operably linked to a nucleotide sequence encoding the modulator nucleic acid) are delivered into the cell. In certain embodiments, the cell (or a parental/ancestral cell thereof) has been engineered to express the modulator nucleic acid, and the Cas protein (or a nucleic acid comprising a regulatory element operably linked to a nucleotide sequence encoding the Cas protein) and the targeter nucleic acid (or a nucleic acid comprising a regulatory element operably linked to a nucleotide sequence encoding the targeter nucleic acid) are delivered into the cell. In certain embodiments, the cell (or a parental/ancestral cell thereof) has been engineered to express the Cas protein and the modulator nucleic acid, and the targeter nucleic acid (or a nucleic acid comprising a regulatory element operably linked to a nucleotide sequence encoding the targeter nucleic acid) is delivered into the cell.

In certain embodiments, the target DNA is in the genome of a target cell. Accordingly, the present invention also provides a cell comprising the non-naturally occurring system or a CRISPR expression system described herein. In addition, the present invention provides a cell whose genome has been modified by the CRISPR-Cas system or complex disclosed herein.

The target cells can be mitotic or post-mitotic cells from any organism, such as a bacterial cell (e.g., E. coli), an archacal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, or the like, a fungal cell (e.g., a yeast cell, such as S. cervisiae), an animal cell, a cell from an invertebrate animal (e.g. fruit fly, enidarian, echinoderm, nematode, etc.), a cell from a vertebrate animal (e.g., fish, amphibian, reptile, bird, mammal), a cell from a mammal, a cell from a rodent, or a cell from a human. The types of target cells include but are not limited to a stem cell (e.g., an embryonic stem (ES) cell, an induced pluripotent stem (iPS) cell, a germ cell), a somatic cell (e.g., a fibroblast, a hematopoietic cell, a T lymphocyte (e.g., CD8+ T lymphocyte), an NK cell, a neuron, a muscle cell, a bone cell, a hepatocyte, a pancreatic cell), an in vitro or in vivo embryonic cell of an embryo at any stage (e.g., a 1-cell, 2-cell, 4-cell, 8-cell; stage zebrafish embryo). Cells may be from established cell lines or may be primary cells (i.e., cells and cells cultures that have been derived from a subject and allowed to grow in vitro for a limited number of passages of the culture). For example, primary cultures are cultures that may have been passaged within 0 times, 1 time, 2 times, 4 times, 5 times, 10 times, or 15 times, but not enough times to go through the crisis stage. Typically, the primary cell lines are maintained for fewer than 10 passages in vitro. If the cells are primary cells, they may be harvest from an individual by any suitable method. For example, leukocytes may be harvested by apheresis, leukocytapheresis, or density gradient separation, while cells from tissues such as skin, muscle, bone marrow, spleen, liver, pancreas, lung, intestine, or stomach can be harvested by biopsy. The harvested cells may be used immediately, or may be stored under frozen conditions with a cryopreservative and thawed at a later time in a manner as commonly known in the art.

### A. Ribonucleoprotein (RNP) Delivery and “cas RNA” Delivery

An engineered, non-naturally occurring system disclosed herein can be delivered into a cell by suitable methods known in the art, including but not limited to ribonucleoprotein (RNP) delivery and “Cas RNA” delivery described below.

In certain embodiments, a CRISPR-Cas system including a single guide nucleic acid and a Cas protein, or a CRISPR-Cas system including a targeter nucleic acid, a modulator nucleic acid, and a Cas protein, can be combined into a RNP complex and then delivered into the cell as a pre-formed complex. This method is suitable for active modification of the genetic or epigenetic information in a cell during a limited time period. For example, where the Cas protein has nuclease activity to modify the genomic DNA of the cell, the nuclease activity only needs to be retained for a period of time to allow DNA cleavage, and prolonged nuclease activity may increase off-targeting. Similarly, certain epigenetic modifications can be maintained in a cell once established and can be inherited by daughter cells.

A “ribonucleoprotein” or “RNP,” as used herein, can refer to a complex comprising a nucleoprotein and a ribonucleic acid. A “nucleoprotein” as provided herein can refer to a protein capable of binding a nucleic acid (e.g., RNA, DNA). Where the nucleoprotein binds a ribonucleic acid it can be referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, or the like). In certain embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA.

To ensure efficient loading of the Cas protein, the single guide nucleic acid, or the combination of the targeter nucleic acid and the modulator nucleic acid, can be provided in excess molar amount (e.g., at least 2 fold, at least 3 fold, at least 4 fold, or at least 5 fold) relative to the Cas protein. In certain embodiments, the targeter nucleic acid and the modulator nucleic acid are annealed under suitable conditions prior to complexing with the Cas protein. In other embodiments, the targeter nucleic acid, the modulator nucleic acid, and the Cas protein are directly mixed together to form an RNP.

A variety of delivery methods can be used to introduce an RNP disclosed herein into a cell. Exemplary delivery methods or vehicles include but are not limited to microinjection, liposomes (see, e.g., U.S. Pat. No. 10,829,787,) such as molecular trojan horses liposomes that delivers molecules across the blood brain barrier (see, Pardridge et al. (2010) Cold Spring Harb. Protoc., doi: 10.1101/pdb.prot5407), immunoliposomes, virosomes, microvesicles (e.g., exosomes and ARMMs), polycations, lipid: nucleic acid conjugates, electroporation, cell permeable peptides (see, U.S. Pat. No. 11,118,194), nanoparticles, nanowires (see, Shalek et al. (2012) Nano Letters, 12:6498), exosomes, and perturbation of cell membrane (e.g., by passing cells through a constriction in a microfluidic system, see, U.S. Pat. No. 11,125,739). Where the target cell is a proliferating cell, the efficiency of RNP delivery can be enhanced by cell cycle synchronization (see, U.S. Pat. No. 10,570,418). In certain embodiments, an RNP is delivered into a cell by electroporation.

In certain embodiments, a CRISPR-Cas system is delivered into a cell in a “approach, i.e., delivering (a) a single guide nucleic acid, or a combination of a targeter nucleic acid and a modulator nucleic acid, and (b) an RNA (e.g., messenger RNA (mRNA)) encoding a Cas protein. The RNA encoding the Cas protein can be translated in the cell and form a complex with the single guide nucleic acid or combination of the targeter nucleic acid and the modulator nucleic acid intracellularly. Similar to the RNP approach, RNAs have limited half-lives in cells, even though stability-increasing modification(s) can be made in one or more of the RNAs. Accordingly, the “Cas RNA” approach is suitable for active modification of the genetic or epigenetic information in a cell during a limited time period, such as DNA cleavage, and has the advantage of reducing off-targeting.

The mRNA can be produced by transcription of a DNA comprising a regulatory element operably linked to a Cas coding sequence. Given that multiple copies of Cas protein can be generated from one mRNA, the single guide nucleic acid, or the targeter nucleic acid and the modulator nucleic acid are generally provided in excess molar amount (e.g., at least 5 fold, at least 10 fold, at least 20 fold, at least 30 fold, at least 50 fold, or at least 100 fold) relative to the mRNA. In certain embodiments, the targeter nucleic acid and the modulator nucleic acid are annealed under suitable conditions prior to delivery into the cells. In other embodiments, the targeter nucleic acid and the modulator nucleic acid are delivered into the cells without annealing in vitro.

A variety of delivery systems can be used to introduce an “Cas RNA” system into a cell. Non-limiting examples of delivery methods or vehicles include microinjection, biolistic particles, liposomes (see, e.g., U.S. Pat. No. 10,829,787) such as molecular trojan horses liposomes that delivers molecules across the blood brain barrier (see, Pardridge et al. (2010) Cold Spring Harb. Protoc., doi: 10.1101/pdb.prot5407), immunoliposomes, virosomes, polycations, lipid: nucleic acid conjugates, electroporation, nanoparticles, nanowires (see, Shalek et al. (2012) Nano Letters, 12:6498), exosomes, and perturbation of cell membrane (e.g., by passing cells through a constriction in a microfluidic system, see, U.S. Pat. No. 11,125,739). Specific examples of the “nucleic acid only” approach by electroporation are described in International (PCT) Publication No. WO 2016/164356.

In certain embodiments, the CRISPR-Cas system is delivered into a cell in the form of (a) a single guide nucleic acid or a combination of a targeter nucleic acid and a modulator nucleic acid, and (b) a DNA comprising a regulatory element operably linked to a Cas coding sequence. The DNA can be provided in a plasmid, viral vector, or any other form described in the “CRISPR Expression Systems” subsection. Such delivery method may result in constitutive expression of Cas protein in the target cell (e.g., if the DNA is maintained in the cell in an episomal vector or is integrated into the genome), and may increase the risk of off-targeting which is undesirable when the Cas protein has nuclease activity. Notwithstanding, this approach is useful when the Cas protein comprises a non-nuclease effector (e.g., a transcriptional activator or repressor). It is also useful for research purposes and for genome editing of plants.

### B. CRISPR Expression Systems

Also provided herein is a nucleic acid comprising a regulatory element operably linked to a nucleotide sequence encoding a guide nucleic acid disclosed herein. In certain embodiments, the nucleic acid comprises a regulatory element operably linked to a nucleotide sequence encoding a single guide nucleic acid; this nucleic acid alone can constitute a CRISPR expression system. In certain embodiments, the nucleic acid comprises a regulatory element operably linked to a nucleotide sequence encoding a targeter nucleic acid. In certain embodiments, the nucleic acid further comprises a nucleotide sequence encoding a modulator nucleic acid, wherein the nucleotide sequence encoding the modulator nucleic acid is operably linked to the same regulatory element as the nucleotide sequence encoding the targeter nucleic acid or a different regulatory element; this nucleic acid alone can constitute a CRISPR expression system.

In addition, the present invention provides a CRISPR expression system comprising: (a) a nucleic acid comprising a first regulatory element operably linked to a nucleotide sequence encoding a targeter nucleic acid and (b) a nucleic acid comprising a second regulatory element operably linked to a nucleotide sequence encoding a modulator nucleic acid.

In certain embodiments, a CRISPR expression system further comprises a nucleic acid comprising a third regulatory element operably linked to a nucleotide sequence encoding a Cas protein, such as a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

As used in this context, the term “operably linked” can mean that the nucleotide sequence of interest is linked to the regulatory element in a manner that allows for expression of the nucleotide sequence (e.g., in an in vitro transcription/translation system or in a host cell when the vector is introduced into the host cell).

The nucleic acids of a CRISPR expression system described above may be independently selected from various nucleic acids such as DNA (e.g., modified DNA) and RNA (e.g., modified RNA). In certain embodiments, the nucleic acids comprising a regulatory element operably linked to one or more nucleotide sequences encoding the guide nucleic acids are in the form of DNA. In certain embodiments, the nucleic acid comprising a third regulatory element operably linked to a nucleotide sequence encoding the Cas protein is in the form of DNA. The third regulatory element can be a constitutive or inducible promoter that drives the expression of the Cas protein. In other embodiments, the nucleic acid comprising a third regulatory element operably linked to a nucleotide sequence encoding the Cas protein is in the form of RNA (e.g., mRNA).

Nucleic acids of a CRISPR expression system can be provided in one or more vectors. The term “vector,” as used herein, can refer to a nucleic acid molecule capable of transporting another nucleic acid to which it has been linked. Conventional viral and non-viral based gene transfer methods can be used to introduce nucleic acids in cells, such as prokaryotic cells, eukaryotic cells, mammalian cells, or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Gene therapy procedures are known in the art and disclosed in Van Brunt (1988) BIOTECHNOLOGY, 6:1149; Anderson (1992) SCIENCE, 256:808; Nabel & Feigner (1993) TIBTECH, 11:211; Mitani & Caskey (1993) TIBTECH, 11:162; Dillon (1993) TIBTECH, 11:167; Miller (1992) NATURE, 357:455; Vigne, (1995) RESTORATIVE NEUROLOGY AND NEUROSCIENCE, 8:35; Kremer & Perricaudet (1995) BRITISH MEDICAL BULLETIN, 51:31; Haddada et al. (1995) CURRENT TOPICS IN MICROBIOLOGY AND IMMUNOLOGY, 199:297; Yu et al. (1994) GENE THERAPY, 1:13; and Doerfler and Bohm (Eds.) (2012) The Molecular Repertoire of Adenoviruses II: Molecular Biology of Virus-Cell Interactions. In certain embodiments, at least one of the vectors is a DNA plasmid. In certain embodiments, at least one of the vectors is a viral vector (e.g., retrovirus, adenovirus, or adeno-associated virus).

Certain vectors are capable of autonomous replication in a host cell into which they are introduced (e.g., bacterial vectors having a bacterial origin of replication and episomal mammalian vectors). Other vectors (e.g., non-episomal mammalian vectors and replication defective viral vectors) do not autonomously replicate in the host cell. Certain vectors, however, may be integrated into the genome of the host cell and thereby are replicated along with the host genome. A skilled person in the art will appreciate that different vectors may be suitable for different delivery methods and have different host tropism, and will be able to select one or more vectors suitable for the use.

The term “regulatory element,” as used herein, can refer to a transcriptional and/or translational control sequence, such as a promoter, enhancer, transcription termination signal (e.g., polyadenylation signal), internal ribosomal entry sites (IRES), protein degradation signal, or the like, that provide for and/or regulate transcription of a non-coding sequence (e.g., a targeter nucleic acid or a modulator nucleic acid) or a coding sequence (e.g., a Cas protein) and/or regulate translation of an encoded polypeptide. Such regulatory elements are described, for example, in Goeddel, GENE EXPRESSION TECHNOLOGY: METHODS IN ENZYMOLOGY, 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In certain embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (see, Takebe et al. (1988) MOL. CELL. BIOL., 8:466); SV40 enhancer; and the intron sequence between exons 2 and 3 of rabbit β-globin (see, O'Hare et al. (1981) PROC. NATL. ACAD. SCI. USA., 78:1527). It will be appreciated by those skilled in the art that the design of the expression vector can depend on factors such as the choice of the host cell to be transformed, the level of expression desired, etc. A vector can be introduced into host cells to produce transcripts, proteins, or peptides, including fusion proteins or peptides, encoded by nucleic acids as described herein (e.g., CRISPR transcripts, proteins, enzymes, mutant forms thereof, or fusion proteins thereof).

In certain embodiments, the nucleotide sequence encoding the Cas protein is codon optimized for expression in a prokaryotic cell, e.g., E. coli, eukaryotic host cell, e.g., a yeast cell (e.g., S. cerevisiae), a mammalian cell (e.g., a mouse cell, a rat cell, or a human cell), or a plant cell. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (IRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.or.jp/codon/and these tables can be adapted in a number of ways (see, Nakamura et al. (2000) NUCL. ACIDS RES., 28:292). Computer algorithms for codon optimizing a particular sequence for expression in a particular host cell, such as Gene Forge (Aptagen; Jacobus, Pa.), are also available. In certain embodiments, the codon optimization facilitates or improves expression of the Cas protein in the host cell.

### C. Donor Templates

Cleavage of a target nucleotide sequence in the genome of a cell by a CRISPR-Cas system or complex can activate DNA damage pathways, which may rejoin the cleaved DNA fragments by NHEJ or HDR. HDR requires a repair template, either endogenous or exogenous, to transfer the sequence information from the repair template to the target.

In certain embodiments, an engineered, non-naturally occurring system or CRISPR expression system further comprises a donor template. As used herein, the term “donor template” can refer to a nucleic acid designed to serve as a repair template at or near the target nucleotide sequence upon introduction into a cell or organism. In certain embodiments, the donor template is complementary to a polynucleotide comprising the target nucleotide sequence or a portion thereof. When optimally aligned, a donor template may overlap with one or more nucleotides of a target nucleotide sequences (e.g. about or more than about 1, 5, 10, 15, 20, 25, 30, 35, 40, or more nucleotides). The nucleotide sequence of the donor template is typically not identical to the genomic sequence that it replaces. Rather, the donor template may contain one or more substitutions, insertions, deletions, inversions or rearrangements with respect to the genomic sequence, so long as sufficient homology is present to support homology-directed repair. In certain embodiments, the donor template comprises a non-homologous sequence flanked by two regions of homology (i.e., homology arms), such that homology-directed repair between the target DNA region and the two flanking sequences results in insertion of the non-homologous sequence at the target region. In certain embodiments, the donor template comprises a non-homologous sequence 10-100 nucleotides, 50-500 nucleotides, 100-1,000 nucleotides, 200-2,000 nucleotides, or 500-5,000 nucleotides in length positioned between two homology arms.

Generally, the homologous region(s) of a donor template has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the donor template comprises a first homology arm homologous to a sequence 5′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 5′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the donor template sequence and a polynucleotide comprising a target nucleotide sequence are optimally aligned, the nearest nucleotide of the donor template is within about 1, 5, 10, 15, 20, 25, 50, 75, 100, 200, 300, 400, 500, 1000, 2000, 3000, 4000, or more nucleotides from the target nucleotide sequence.

In certain embodiments, the donor template further comprises an engineered sequence not homologous to the sequence to be repaired. Such engineered sequence can harbor a barcode and/or a sequence capable of hybridizing with a donor template-recruiting sequence disclosed herein.

In certain embodiments, the donor template further comprises one or more mutations relative to the genomic sequence, wherein the one or more mutations reduce or prevent cleavage, by the same CRISPR-Cas system, of the donor template or of a modified genomic sequence with at least a portion of the donor template sequence incorporated. In certain embodiments, in the donor template, the PAM adjacent to the target nucleotide sequence and recognized by the Cas nuclease is mutated to a sequence not recognized by the same Cas nuclease. In certain embodiments, in the donor template, the target nucleotide sequence (e.g., the seed region) is mutated. In certain embodiments, the one or more mutations are silent with respect to the reading frame of a protein-coding sequence encompassing the mutated sites.

The donor template can be provided to the cell as single-stranded DNA, single-stranded RNA, double-stranded DNA, or double-stranded RNA. In certain embodiments, the linear double-stranded DNA comprises covalently closed ends, for example as generated by a telomerase enzyme such as TelN or a suitable alternative. It is understood that a CRISPR-Cas system, such as a system disclosed herein, may possess nuclease activity to cleave the target strand, the non-target strand, or both. When HDR of the target strand is desired, a donor template having a nucleic acid sequence complementary to the target strand is also contemplated.

The donor template can be introduced into a cell in linear or circular form. If introduced in linear form, the ends of the donor template may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example, Chang et al. (1987) PROC. NATL. ACAD SCI USA, 84:4959; Nehls et al. (1996) SCIENCE, 272:886; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. For example, linearly covalently closed DNA, for example as generated by a telomerase enzyme like TelN, can be used as a donor template. As an alternative to protecting the termini of a linear donor template, additional lengths of sequence may be included outside of the regions of homology that can be degraded without impacting recombination.

A donor template can be a component of a vector as described herein, contained in a separate vector, or provided as a separate polynucleotide, such as an oligonucleotide, linear polynucleotide, or synthetic polynucleotide. In certain embodiments, the donor template is a DNA. In certain embodiments, a donor template is in the same nucleic acid as a sequence encoding the single guide nucleic acid, a sequence encoding the targeter nucleic acid, a sequence encoding the modulator nucleic acid, and/or a sequence encoding the Cas protein, where applicable. In certain embodiments, a donor template is provided in a separate nucleic acid. A donor template polynucleotide may be of any suitable length, such as about or at least about 50, 75, 100, 150, 200, 500, 1000, 2000, 3000, 4000, or more nucleotides in length.

A donor template can be introduced into a cell as an isolated nucleic acid. Alternatively, a donor template can be introduced into a cell as part of a vector (e.g., a plasmid) having additional sequences such as, for example, replication origins, promoters and genes encoding antibiotic resistance, that are not intended for insertion into the DNA region of interest. Alternatively, a donor template can be delivered by viruses (e.g., adenovirus, adeno-associated virus (AAV)). In certain embodiments, the donor template is introduced as an AAV, e.g., a pseudotyped AAV. The capsid proteins of the AAV can be selected by a person skilled in the art based upon the tropism of the AAV and the target cell type. For example, in certain embodiments, the donor template is introduced into a hepatocyte as AAV8 or AAV9. In certain embodiments, the donor template is introduced into a hematopoietic stem cell, a hematopoietic progenitor cell, or a T lymphocyte (e.g., CD8⁺ T lymphocyte) as AAV6 or an AAVHSC (see, U.S. Pat. No. 9,890,396). It is understood that the sequence of a capsid protein (VP1, VP2, or VP3) may be modified from a wild-type AAV capsid protein, for example, having at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to a wild-type AAV capsid sequence.

The donor template can be delivered to a cell (e.g., a primary cell) by various delivery methods, such as a viral or non-viral method disclosed herein. In certain embodiments, a non-viral donor template is introduced into the target cell as a naked nucleic acid or in complex with a liposome or poloxamer. In certain embodiments, a non-viral donor template is introduced into the target cell by electroporation. In other embodiments, a viral donor template is introduced into the target cell by infection. The engineered, non-naturally occurring system can be delivered before, after, or simultaneously with the donor template (see, International (PCT) Application Publication No. WO 2017/053729). A skilled person in the art will be able to choose proper timing based upon the form of delivery (consider, for example, the time needed for transcription and translation of RNA and protein components) and the half-life of the molecule(s) in the cell. In particular embodiments, where the CRISPR-Cas system including the Cas protein is delivered by electroporation (e.g., as an RNP), the donor template (e.g., as an AAV) is introduced into the cell within 4 hours (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150, 180, 210, or 240 minutes) after the introduction of the engineered, non-naturally occurring system.

In certain embodiments, the donor template is conjugated covalently to a modulator nucleic acid. Covalent linkages suitable for this conjugation are known in the art and are described, for example, in U.S. Pat. No. 9,982,278 and Savic et al. (2018) ELIFE 7:033761. In certain embodiments, the donor template is covalently linked to a modulator nucleic acid (e.g., the 5′ end of the modulator nucleic acid) through an internucleotide bond. In certain embodiments, the donor template is covalently linked to a modulator nucleic acid (e.g., the 5′ end of the modulator nucleic acid) through a linker.

In certain embodiments, the donor template can comprise any nucleic acid chemistry. In certain embodiments, the donor template can comprise DNA and/or RNA nucleotides. In certain embodiments, the donor template can comprise single-stranded DNA, linear single-stranded RNA, linear double-stranded DNA, linear double-stranded RNA, circular single-stranded DNA, circular single-stranded RNA, circular double-stranded DNA, or circular double-stranded RNA. In certain embodiments, the linear double-stranded DNA comprises covalently closed ends, for example as generated by a telomerase enzyme such as TelN or a suitable alternative. In certain embodiments, the donor template comprises a mutation in a PAM sequence to partially or completely abolish binding of the RNP to the DNA. In certain embodiments, the donor template is present at a concentration of at least 0.05, 0.01, 0.2, 0.3, 0.4, 0.5, 0.6, 0.7, 0.8, 0.9, 1, 1.25, 1.5, 1.75, 2, 3, or 4, and/or no more than 0.01, 0.2, 0.3, 0.4, 0.5, 0.6, 0.7, 0.8, 0.9, 1, 1.25, 1.5, 1.75, 2, 3, 4, or 5 μg μL⁻¹, for example 0.01-5 μg μL⁻¹. In certain embodiments, the donor template comprises one or more promoters. In certain embodiments, the donor template comprises a promoter that shares at least 70%, at least 75%, at least 80%, at least 85%, at least 90%, at least 95%, at least 99.5% sequence identity with any one of SEQ ID NOs: 78-85 of Table 5.

TABLE 5
Promoter sequences

| | SEQ | |
|---|---|---|
| | ID | |
| Name | NO | Sequence |
| CMV | 78 | CGTTACATAACTTACGGTAAATGGCCCGCCTGGCTGACCGCCCAACGACCCCC |
| | | GCCCATTGACGTCAATAATGACGTATGTTCCCATAGTAACGCCAATAGGGACT |
| | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTGATGCGGTTTTG |
| | | GCAGTACATCAATGGGCGTGGATAGCGGTTTGACTCACGGGGATTTCCAAGTC |
| | | TCCACCCCATTGACGTCAATGGGAGTTTGTTTTGGCACCAAAATCAACGGGAC |
| | | TTTCCAAAATGTCGTAACAACTCCGCCCCATTGACGCAAATGGGCGGTAGGCG |
| | | TGTACGGTGGGAGGTCTATATAAGCAGAGCT |
| SCP | 79 | GTACTTATATAAGGGGGTGGGGGCGCGTTCGTCCTCAGTCGCGATCGAACACT |
| | | CGAGCCGAGCAGACGTGCCTACGGACCG |
| CMV | 80 | CGTTACATAACTTACGGTAAATGGCCCGCCTGGCTGACCGCCCAACGACCCCC |
| e- | | GCCCATTGACGTCAATAATGACGTATGTTCCCATAGTAACGCCAATAGGGACT |
| SCP | | TTCCATTGACGTCAATGGGTGGAGTATTTACGGTAAACTGCCCACTTGGCAGT |
| | | ACATCAAGTGTATCATATGCCAAGTACGCCCCCTATTGACGTCAATGACGGTA |
| | | AATGGCCCGCCTGGCATTATGCCCAGTACATGACCTTATGGGACTTTCCTACT |
| | | TGGCAGTACATCTACGTATTAGTCATCGCTATTACCATGGTACTTATATAAGG |
| | | GGGTGGGGGCGCGTTCGTCCTCAGTCGCGATCGAACACTCGAGCCGAGCAGAC |
| | | GTGCCTACGGACCG |
| CMV | 81 | TCAATATTGGCCATTAGCCATATTATTCATTGGTTATATAGCATAAATCAATA |
| max | | TTGGCTATTGGCCATTGCATACGTTGTATCTATATCATAATATGTACATTTAT |
| | | ATTGGCTCATGTCCAATATGACCGCCATGTTGGCATTGATTATTGACTAGTTA |
| | | TTAATAGTAATCAATTACGGGGTCATTAGTTCATAGCCCATATATGGAGTTCC |
| | | GCGTTACATAACTTACGGTAAATGGCCCGCCTGGCTGACCGCCCAACGACCCC |
| | | CGCCCATTGACGTCAATAATGACGTATGTTCCCATAGTAACGCCAATAGGGAC |
| | | TTTCCATTGACGTCAATGGGTGGAGTATTTACGGTAAACTGCCCACTTGGCAG |
| | | TACATCAAGTGTATCATATGCCAAGTCCGCCCCCTATTGACGTCAATGACGGT |
| | | AAATGGCCCGCCTGGCATTATGCCCAGTACATGACCTTACGGGACTTTCCTAC |
| | | TTGGCAGTACATCTACGTATTAGTCATCGCTATTACCATGGTGATGCGGTTTT |
| | | GGCAGTACACCAATGGGCGTGGATAGCGGTTTGACTCACGGGGATTTCCAAGT |
| | | CTCCACCCCATTGACGTCAATGGGAGTTTGTTTTGGCACCAAAATCAACGGGA |
| | | CTTTCCAAAATGTCGTAATAACCCCGCCCCGTTGACGCAAATGGGCGGTAGGC |
| | | GTGTACGGTGGGAGGTCTATATAAGCAGAGGTCGTTTAGTGAACCGTCAGATC |
| | | ACTAGTAGCTTTATTGCGGTAGTTTATCACAGTTAAATTGCTAACGCAGTCAG |
| | | TGCTCGACTGATCACAGGTAAGTATCAAGGTTACAAGACAGGTTTAAGGAGGC |
| | | CAATAGAAACTGGGCTTGTCGAGACAGAGAAGATTCTTGCGTTTCTGATAGGC |
| | | ACCTATTGGTCTTACTGACATCCACTTTGCCTTTCTCTCCACAGGG |
| JET | 82 | GAATTCGGGCGGAGTTAGGGCGGAGCCAATCAGCGTGCGCCGTTCCGAAAGTT |
| | | GCCTTTTATGGCTGGGCGGAGAATGGGCGGTGAACGCCGATGATTATATAAGG |
| | | ACGCGCCGGGTGTGGCACAGCTAGTTCCGTCGCAGCCGGGATTTGGGTCGCGG |
| | | TTCTTGTTTGTGGATCCCTGTGATCGTCACTTGACA |
| CAG | 83 | ATCTCGACTAGTTATTAATAGTAATCAATTACGGGGTCATTAGTTCATAGCCC |
| | | ATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTA |
| | | ACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTG |
| | | ACGTCAATGACGGTAAATGGCCCGCCTGGCATTATGCCCAGTACATGACCTTA |
| | | TGGGACTTTCCTACTTGGCAGTACATCTACGTATTAGTCATCGCTATTACCAT |
| | | GGTCGAGGTGAGCCCCACGTTCTGCTTCACTCTCCCCATCTCCCCCCCCTCCC |
| | | CACCCCCAATTTTGTATTTATTTATTTTTTAATTATTTTGTGCAGCGATGGGG |
| | | GCGGGGGGGGGGGGGGGGCGCGCGCCAGGCGGGGCGGGGCGGGGCGAGGGGCG |
| | | GGGCGGGGCGAGGCGGAGAGGTGCGGCGGCAGCCAATCAGAGCGGCGCGCTCC |
| | | GAAAGTTTCCTTTTATGGCGAGGCGGCGGCGGCGGCGGCCCTATAAAAAGCGA |
| | | AGCGCGCGGCGGGCGGGGAGTCGCTGCGACGCTGCCTTCGCCCCGTGCCCCGC |
| | | TCCGCCGCCGCCTCGCGCCGCCCGCCCCGGCTCTGACTGACCGCGTTACTCCC |
| | | ACAGGTGAGCGGGCGGGACGGCCCTTCTCCTCCGGGCTGTAATTAGCGCTTGG |
| | | TTTAATGACGGCTTGTTTCTTTTCTGTGGCTGCGTGAAAGCCTTGAGGGGCTC |
| | | CGGGAGGGCCCTTTGTGCGGGGGGAGCGGCTCGGGGGGTGCGTGCGTGTGTGT |
| | | GTGCGTGGGGAGCGCCGCGTGCGGCTCCGCGCTGCCCGGCGGCTGTGAGCGCT |
| | | GCGGGCGCGGCGCGGGGCTTTGTGCGCTCCGCAGTGTGCGCGAGGGGAGCGCG |
| | | GCCGGGGGCGGTGCCCCGCGGTGCGGGGGGGGCTGCGAGGGGAACAAAGGCTG |
| | | CGTGCGGGGTGTGTGCGTGGGGGGGTGAGCAGGGGGTGTGGGCGCGTCGGTCG |
| | | GGCTGCAACCCCCCCTGCACCCCCCTCCCCGAGTTGCTGAGCACGGCCCGGCT |
| | | TCGGGTGCGGGGCTCCGTACGGGGCGTGGCGCGGGGCTCGCCGTGCCGGGCGG |
| | | GGGGTGGCGGCAGGTGGGGGTGCCGGGCGGGGGGGGGCCGCCTCGGGCCGGGG |
| | | AGGGCTCGGGGGAGGGGCGCGGCGGCCCCCGGAGCGCCGGCGGCTGTCGAGGC |
| | | GCGGCGAGCCGCAGCCATTGCCTTTTATGGTAATCGTGCGAGAGGGCGCAGGG |
| | | ACTTCCTTTGTCCCAAATCTGTGCGGAGCCGAAATCTGGGAGGCGCCGCCGCA |
| | | CCCCCTCTAGCGGGCGCGGGGCGAAGCGGTGCGGCGCCGGCAGGAAGGAAATG |
| | | GGCGGGGAGGGCCTTCGTGCGTCGCCGCGCCGCCGTCCCCTTCTCCCTCTCCA |
| | | GCCTCGGGGCTGTCCGCGGGGGGACGGCTGCCTTCGGGGGGGACGGGGCAGGG |
| | | CGGGGTTCGGCTTCTGGCGTGTGACCGGCGGCTCTAGAGCCTCTGCTAACCAT |
| | | GTTCATGCCTTCTTCTTTTTCCTACAGCTCCTGGGCAACGTGCTGGTTATTGT |
| | | GCTGTCTCATCATTTTGGCAAAGAATT |
| PGK | 84 | GGGGTTGGGGTTGCGCCTTTTCCAAGGCAGCCCTGGGTTTGCGCAGGGACGCG |
| | | GCTGCTCTGGGCGTGGTTCCGGGAAACGCAGCGGCGCCGACCCTGGGTCTCGC |
| | | ACATTCTTCACGTCCGTTCGCAGCGTCACCCGGATCTTCGCCGCTACCCTTGT |
| | | GGGCCCCCCGGCGACGCTTCCTGCTCCGCCCCTAAGTCGGGAAGGTTCCTTGC |
| | | GGTTCGCGGCGTGCCGGACGTGACAAACGGAAGCCGCACGTCTCACTAGTACC |
| | | CTCGCAGACGGACAGCGCCAGGGAGCAATGGCAGCGCGCCGACCGCGATGGGC |
| | | TGTGGCCAATAGCGGCTGCTCAGCAGGGCGCGCCGAGAGCAGCGGCCGGGAAG |
| | | GGGCGGTGCGGGAGGCGGGGTGTGGGGCGGTAGTGTGGGCCCTGTTCCTGCCC |
| | | GCGCGGTGTTCCGCATTCTGCAAGCCTCCGGAGCGCACGTCGGCAGTCGGCTC |
| | | CCTCGTTGACCGAATCACCGACCTCTCTCCCCAG |
| EF- | 85 | GAATTCAGGCTCCGGTGCCCGTCAGTGGGCAGAGCGCACATCGCCCACAGTCC |
| 1a | | CCGAGAAGTTGGGGGGAGGGGTCGGCAATTGAACCGGTGCCTAGAGAAGGTGG |
| | | CGCGGGGTAAACTGGGAAAGTGATGTCGTGTACTGGCTCCGCCTTTTTCCCGA |
| | | GGGTGGGGGAGAACCGTATATAAGTGCAGTAGTCGCCGTGAACGTTCTTTTTC |
| | | GCAACGGGTTTGCCGCCAGAACACAGGTAAGTGCCGTGTGTGGTTCCCGCGGG |
| | | CCTGGCCTCTTTACGGGTTATGGCCCTTGCGTGCCTTGAATTACTTCCACCTG |
| | | GCTGCAGTACGTGATTCTTGATCCCGAGCTTCGGGTTGGAAGTGGGTGGGAGA |
| | | GTTCGAGGCCTTGCGCTTAAGGAGCCCCTTCGCCTCGTGCTTGAGTTGAGGCC |
| | | TGGCCTGGGCGCTGGGGCCGCCGCGTGCGAATCTGGTGGCACCTTCGCGCCTG |
| | | TCTCGCTGCTTTCGATAAGTCTCTAGCCATTTAAAATTTTTGATGACCTGCTG |
| | | CGACGCTTTTTTTCTGGCAAGATAGTCTTGTAAATGCGGGCCAAGATCTGCAC |
| | | ACTGGTATTTCGGTTTTTGGGGCCGCGGGCGGCGACGGGGCCCGTGCGTCCCA |
| | | GCGCACATGTTCGGCGAGGCGGGGCCTGCGAGCGCGGCCACCGAGAATCGGAC |
| | | GGGGGTAGTCTCAAGCTGGCCGGCCTGCTCTGGTGCCTGGTCTCGCGCCGCCG |
| | | TGTATCGCCCCGCCCTGGGCGGCAAGGCTGGCCCGGTCGGCACCAGTTGCGTG |
| | | AGCGGAAAGATGGCCGCTTCCCGGCCCTGCTGCAGGGAGCTCAAAATGGAGGA |
| | | CGCGGCGCTCGGGAGAGCGGGCGGGTGAGTCACCCACACAAAGGAAAAGGGCC |
| | | TTTCCGTCCTCAGCCGTCGCTTCATGTGACTCCACGGAGTACCGGGCGCCGTC |
| | | CAGGCACCTCGATTAGTTCTCGAGCTTTTGGAGTACGTCGTCTTTAGGTTGGG |
| | | GGGAGGGGTTTTATGCGATGGAGTTTCCCCACACTGAGTGGGTGGAGACTGAA |
| | | GTTAGGCCAGCTTGGCACTTGATGTAATTCTCCTTGGAATTTGCCCTTTTTGA |
| | | GTTTGGATCTTGGTTCATTCTCAAGCCTCAGACAGTGGTTCAAAGTTTTTTTC |
| | | TTCCATTTCAGGTGTCGTGACATCATTTT |

### D. Efficiency and Specificity

An engineered, non-naturally occurring system can be evaluated in terms of efficiency and/or specificity in nucleic acid targeting, cleavage, or modification.

In certain embodiments, an engineered, non-naturally occurring system has high efficiency. For example, in certain embodiments, at least 1, 1.5, 2, 2.5, 3, 4, 5, 10, 15, 20, 25, 30, 35, 40, 45, 50, 55, 60, 65, 70, 75, 80, 85, 90, 95, 96, 97, 98, 99, or 100% of a population of nucleic acids having the target nucleotide sequence and a cognate PAM, when contacted with the engineered, non-naturally occurring system, is targeted, cleaved, or modified. In certain embodiments, the genomes of at least 5, 10, 15, 20, 25, 30, 35, 40, 45, 50, 55, 60, 65, 70, 75, 80, 85, 90, 95, 96, 97, 98, 99, or 100% of a population of cells, when the engineered, non-naturally occurring system is delivered into the cells, are targeted, cleaved, or modified.

It has been observed that for a given spacer sequence, the occurrence of on-target events and the occurrence of off-target events are generally correlated. For certain therapeutic purposes, lower on-target efficiency can be tolerated and low off-target frequency is more desirable. For example, when editing or modifying a proliferating cell that will be delivered to a subject and proliferate in vivo, tolerance to off-target events is low. Prior to delivery, it is possible to assess the on-target and off-target events, thereby selecting one or more colonies that have the desired edit or modification and lack any undesired edit or modification. Notwithstanding, the on-target efficiency may need to meet a certain standard to be suitable for therapeutic use. High editing efficiency in a standard CRISPR-Cas system allows tuning of the system, for example, by reducing the binding of the guide nucleic acids to the Cas protein, without losing therapeutic applicability.

In certain embodiments, when a population of nucleic acids having the target nucleotide sequence and a cognate PAM is contacted with the engineered, non-naturally occurring system disclosed herein, the frequency of off-target events (e.g., targeting, cleavage, or modification, depending on the function of the CRISPR-Cas system) is reduced. Methods of assessing off-target events were summarized in Lazzarotto et al. (2018) Nat Protoc. 13(11): 2615-42, and include discovery of in situ Cas off-targets and verification by sequencing (DISCOVER-seq) as disclosed in Wienert et al. (2019) Science 364(6437): 286-89; genome-wide unbiased identification of double-stranded breaks (DSBs) enabled by sequencing (GUIDE-seq) as disclosed in Kleinstiver et al. (2016) Nat. Biotech. 34:869-74; circularization for in vitro reporting of cleavage effects by sequencing (CIRCLE-seq) as described in Kocak et al. (2019) Nat. Biotech. 37:657-66. In certain embodiments, the off-target events include targeting, cleavage, or modification at a given off-target locus (e.g., the locus with the highest occurrence of off-target events detected). In certain embodiments, the off-target events include targeting, cleavage, or modification at all the loci with detectable off-target events, collectively.

In certain embodiments, genomic mutations are detected in no more than 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% of the cells at any off-target loci (in aggregate). In certain embodiments, the ratio of the percentage of cells having an on-target event to the percentage of cells having any off-target event (e.g., the ratio of the percentage of cells having an on-target editing event to the percentage of cells having a mutation at any off-target loci) is at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000. It is understood that genetic variation may be present in a population of cells, for example, by spontaneous mutations, and such mutations are not included as off-target events.

### E. Multiplexing

The method of targeting, editing, and/or modifying a genomic DNA disclosed herein can be conducted in multiplicity. For example, a library of targeter nucleic acids can be used to target multiple genomic loci; a library of donor templates can also be used to generate multiple insertions, deletions, and/or substitutions. The multiplex assay can be conducted in a screening method wherein each separate cell culture (e.g., in a well of a 96-well plate or a 384-well plate) is exposed to a different guide nucleic acid having a different targeter stem sequence and/or a different donor template. The multiplex assay can also be conducted in a selection method wherein a cell culture is exposed to a mixed population of different guide nucleic acids and/or donor templates, and the cells with desired characteristics (e.g., functionality) are enriched or selected by advantageous survival or growth, resistance to a certain agent, expression of a detectable protein (e.g., a fluorescent protein that is detectable by flow cytometry), etc.

In certain embodiments, the plurality of guide nucleic acids and/or the plurality of donor templates are designed for saturation editing. For example, in certain embodiments, each nucleotide position in a sequence of interest is systematically modified with each of all four traditional bases, A, T, G and C. In other embodiments, at least one sequence in each gene from a pool of genes of interest is modified, for example, according to a CRISPR design algorithm. In certain embodiments, each sequence from a pool of exogenous elements of interest (e.g., protein coding sequences, non-protein coding genes, regulatory elements) is inserted into one or more given loci of the genome.

It is understood that the multiplex methods suitable for the purpose of carrying out a screening or selection method, which is typically conducted for research purposes, may be different from the methods suitable for therapeutic purposes. For example, constitutive expression of certain elements (e.g., a Cas nuclease and/or a guide nucleic acid) may be undesirable for therapeutic purposes due to the potential of increased off-targeting. Conversely, for research purposes, constitutive expression of a Cas nuclease and/or a guide nucleic acid may be desirable. For example, the constitutive expression provides a large window during which other elements can be introduced. When a stable cell line is established for the constitutive expression, the number of exogenous elements that need to be co-delivered into a single cell is also reduced. Therefore, constitutive expression of certain elements can increase the efficiency and reduce the complexity of a screening or selection process. Inducible expression of certain elements of the system disclosed herein may also be used for research purposes given similar advantages. Expression may be induced by an exogenous agent (e.g., a small molecule) or by an endogenous molecule or complex present in a particular cell type (e.g., at a particular stage of differentiation). Methods known in the art, such as those described herein, can be used for constitutively or inducibly expressing one or more elements. For example, the specificity of CRISPR nucleases is at least partially dictated by the uniqueness of the spacer (in combination with spacer sequence's proximity to a requisite PAM) and its off-target score can be calculated with algorithms, such as crispr.mit.edu (Hsu et al. (2013) Nat. Biotech. 31:827-832). The highest possible score is 100, which shows probability for high specificity and few off targets. Because our SHS library targets intergenic regions, the algorithm for gRNA prediction should be able to make alignments with repeated regions and low-complexity sequences.

It is further understood that despite the need to introduce multiple elements—the single guide nucleic acid and the Cas protein; or the targeter nucleic acid, the modulator nucleic acid, and the Cas protein—these elements can be delivered into the cell as a single complex of pre-formed RNP. Therefore, the efficiency of the screening or selection process can also be achieved by pre-assembling a plurality of RNP complexes in a multiplex manner.

In certain embodiments, the method disclosed herein further comprises a step of identifying a guide nucleic acid, a Cas protein, a donor template, or a combination of two or more of these elements from the screening or selection process. A set of barcodes may be used, for example, in the donor template between two homology arms, to facilitate the identification. In specific embodiments, the method further comprises harvesting the population of cells; selectively amplifying a genomic DNA or RNA sample including the target nucleotide sequence(s) and/or the barcodes; and/or sequencing the genomic DNA or RNA sample and/or the barcodes that has been selectively amplified.

In addition, the present invention provides a library comprising a plurality of guide nucleic acids, such as a plurality of guide nucleic acids disclosed herein. In another aspect, the present invention provides a library comprising a plurality of nucleic acids each comprising a regulatory element operably linked to a different guide nucleic acid such as a different guide nucleic acid disclosed herein. These libraries can be used in combination with one or more Cas proteins or Cas-coding nucleic acids, such as disclosed herein, and/or one or more donor templates, such as disclosed herein, for a screening or selection method.

### F. Genomic Safe Harbors

Genome engineering is an area of research seeking to modify genes of living organisms to improve our understanding of gene function and to develop methods for genome engineering that treat genetic or acquired diseases, among many others. To modify the genome of target cells, skilled artisans use one or more available tools to introduce changes into the genome at targeted locations to modify the sequence of a target polynucleotide, e.g., a target gene, in desired ways, e.g., modulate gene expression, modulate gene sequences, remove gene sequences, introduce genes, e.g., exogenous DNA, e.g., transgenes, and the like. Efficient transgene insertion may be accomplished through non-precise methods including but not limited to viral vectors, such as, retroviral vectors, e.g., adeno-associate virus (AAV) and the like, or precise methods including but not limited to guided nucleases, such as, zinc finger nucleases (ZFNs), transcription activator-like effector nucleases (TALENs), homing endonucleases, e.g., restriction endonucleases, or nucleic acid-guided nuclease, e.g., CRISPR-cas, e.g., Cas9 and Cas12a and engineered versions thereof.

Exogenous genes, e.g., transgenes, inserted into the genome of a target human cell either randomly, e.g., through retroviral vectors, or in a targeted manner, e.g., through the action of a nucleic acid-guided nuclease, such as Cas, may interact with other genomic elements in unpredictable ways. Due to the complex transcriptional regulation of genes in mammalian cells through networks of cis and trans regulatory elements, such as proximal and distal enhancers, and multiple transcription factors, attempts to alter the default genomic architecture by integration of exogenous DNA, e.g., transgenes, or synthetic sequences can affect the expression of the transgene itself leading to complete attenuation or complete silencing, and/or the expression of both nearby and distant endogenous genes that can, e.g., compromise the safety checkpoints that healthy cells have including dysregulation of expression of key genes, such as oncogenes and tumor suppressor genes, that can alter cellular behavior in dramatic ways, i.e., promoting clonal expansion or malignant transformation of the host.

Gene integration next to regulatory elements of proto-oncogenes has been shown to cause oncogenic transformation, which is particularly important when engineering cells for therapeutic applications. Therefore, the identification of suitable target polynucleotide comprising a target nucleotide sequence in the human genome wherein the insertion of a transgene leads to suitable expression of the transgene without disruption of neighboring genes is desired. In particular, for gene and cell therapy applications, suitable target polynucleotide comprising a target nucleotide sequence in the human genome wherein the insertion of a transgene leads to sufficient expression of the transgene in a therapeutic cell e.g., a T cell, e.g., a CAR T cell; or precursor cell, e.g., a stem cell, such as a hematopoietic stem cell, without malignant transformation or any other disruption that would be harmful to an individual after implantation is desired.

Expression of exogenous genes, e.g., transgenes, in desired cell types and/or developmental/differentiation stages relies on integration into suitable target polynucleotide comprising a target nucleotide sequence that results in sufficient expression, to a degree sufficient for the intended purpose, from the candidate locus. Expression from a specific genomic site can be affected by many factors including but not limited to cell type and differentiation stage, as one or more components of the target polynucleotide get activated during differentiation while others get silenced, and changes in chromatin architecture. Therefore, the identification of suitable target polynucleotides comprising a target nucleotide sequence in the human genome wherein insertion of exogenous DNA, e.g., a transgene, leads to sufficient expression in the target human cell, and, in the case of stem cells, the expression is maintained at a sufficient level through (1) differentiation and (2) through clonal expansion is desired. The current disclosure provides significant advances in the ability engineer human genomes by providing compositions and methods for targeting and delivering exogenous genes, e.g., transgenes, to the suitable target polynucleotide comprising a target nucleotide sequence.

Provided herein are compositions and methods for genome engineering. Certain embodiments comprise compositions. Certain embodiments comprise composition for editing genomes. embodiments disclosed herein concern novel guide nucleic acids (gNAs), e.g., gRNAs, that are complementary to a target nucleotide sequence in a target polynucleotide. As used herein, a “target polynucleotide,” includes a polynucleotide in which a target nucleotide sequence is located. As used herein, a “target nucleotide sequence” includes a sequence to which a guide sequence can bind, e.g., has complementarity to, where binding between a target nucleotide sequence and a guide sequence may allow the activity of a nucleic acid-guided nuclease complex. Further embodiments disclosed herein concern novel gNAs, e.g., gRNAs, that are complementary to a target nucleotide sequence in a target polynucleotide into which insertion of exogenous DNA, e.g., a transgene, doesn't negatively affect the cell, e.g., significantly affect the expression of one or more endogenous genes or result in a malignant transformation of the cell. In further embodiments disclosed herein, gene expression demonstrated in the human target cell is maintained through differentiation of the human target cell and/or through proliferation in the one or more progeny cells at a level sufficient for the ultimate use of the cells. Certain embodiments disclosed herein concern novel nucleic acid-guided nuclease complexes, e.g., RNPs, such as Cas bound to a gNA, that are complementary to a target nucleotide sequence within a target polynucleotide and hydrolyze the phosphodiester back bone (also referred as cleave or cut) in at least one position on at least one strand of the target polynucleotide. Certain embodiments disclosed herein concern methods for selecting and using gNAs, e.g., gRNAs, for genome engineering. Certain embodiments concern methods for using gNAs that are complementary to a target nucleotide sequence within a target polynucleotide, synthesizing the gNA and nucleic-acid-guided nuclease, and/or combining the nucleic guided nuclease with the gNA to form a nucleic acid-guided nuclease complex, e.g., RNP. Certain embodiments disclosed herein concern methods. Certain embodiments disclosed herein concern methods for engineering genomes. Certain embodiments disclosed herein concern methods where a nucleic acid-guided nuclease complex, e.g., RNP, is introduced, e.g., transfected, into a human target cell along with a donor template, e.g., an exogenous DNA, e.g., a transgene, in which the nucleic-acid guided nuclease cleaves the backbone at a least one position in at least one of the strands of the target polynucleotide and the donor template is used to repair the cleaved target polynucleotide, introducing at least a portion of the donor template into the target polynucleotide. As used herein, “exogenous DNA” or a “transgene” includes any gene, natural or synthetic, which is introduced into the genome of an organism or cell to which it is not endogenous. The transgene may or may not retain the ability to be expressed and/or produce RNA or protein in the human target cell. The transgene may or may not alter the resulting phenotype of the human target cell. Certain embodiments include human target cells, e.g., a eukaryotic cell, e.g., a mammalian cell, such as a human cell, for example a stem cell or an immune cell, generated through a method where the nucleic acid-guided nuclease complex, e.g., RNP, is introduced, e.g., transfected, into a human target cell along with a donor template, e.g., as an exogenous DNA or a transgene, such as a chimeric antigen receptor (CAR), in which the nucleic-acid guided nuclease cleaves at or near a targets sequence in a target polynucleotide and the donor template is used to repair the cleaved target polynucleotide introducing at least a portion of the donor template into the target polynucleotide. Certain embodiments disclosed herein include promoter sequences adjacent to an exogenous gene, e.g., a transgene; in certain cases, constructs including the promoter, when introduced into a target polynucleotide of a human target cell, e.g., an immune cell or a stem cell, maintain sufficient gene expression in the edited human target cell for the intended purpose of the cell or its progeny. In certain embodiments, the human target cell is viable after introduction of the exogenous DNA.

As used herein, a “human target cell” includes a cell into which an exogenous product, e.g., a protein, a nucleic acid, or a combination thereof, has been introduced. In certain cases, a human target cell may be used to produce a gene product from an exogenous DNA, e.g., a transgene, such as an exogenous protein, e.g., a CAR. In certain cases, a human target cell may comprise a target nucleotide sequence within target polynucleotide wherein a nucleic acid-guided nuclease hybridizes and cleaves at a site of cleavage at one or more positions on one or more strands of the target polynucleotide at or near the target nucleotide sequence.

As used herein, a “site of cleavage” includes the location or locations at which a nucleic acid-guided nuclease complex will hydrolyze the phosphodiester backbone of a single-stranded or double-stranded target polynucleotide, after binding at a target nucleotide sequence in the target polynucleotide. In certain cases in which the target polynucleotide of a nucleic acid-guided nuclease complex is double stranded, binding of the nucleic acid-guided nuclease complex to a target nucleotide sequence within the target polynucleotide can result in hydrolysis of one of the strands of the target polynucleotide at or near the target nucleotide sequence, resulting in strand cleavage. In such a case, the nucleic acid-guided nuclease complex can cleave either strand of the target polynucleotide. In certain cases, binding of the nucleic acid-guided nuclease complex to a target nucleotide sequence within a target polynucleotide can result in hydrolysis of both strands of the target polynucleotide at or near the target nucleotide sequence, resulting in cleavage of both strands. The sites of cleavage can be the same for both strands, resulting in a blunt end, or the sites of cleavage for each strand can be offset resulting in single strand overhangs, e.g., sticky ends. In certain cases, mismatches at or near the site of cleavage may or may not affect the cleavage efficiency of the nucleic acid-guided nuclease complex.

In certain cases, uncontrolled gene integration next to regulatory elements of proto-oncogenes has been shown to cause oncogenic transformation, which is particularly important

when engineering cells for therapeutic applications. Therefore, it is desired to identify suitable target polynucleotides comprising target nucleotide sequences that result in safe, stable integration of exogenous DNA with sufficient expression in a human target cell and its resultant progeny.

Exemplary characteristics of a target nucleotide sequence that can demonstrate predictable function without potentially harmful alterations in human target cell genomic activity include one or more of (1) >150 kb, for example, >200, such as >250, and in some cases >300 kb away from a known cancer-related gene, (2) >150 kb, for example, >200, such as >250, and in some cases >300 kb away from any miRNA/other functional small RNA, (3) >10 kb, for example, >20, such as >30, and in some cases >50 kb away from any 5′ gene end, (4) >10 kb, for example, >20, such as >30, and in some cases >50 kb away from any replication origin, (5) >10 kb, for example, >20, such as >30, and in some cases >50 kb away from any ultra-conserved element, (6) demonstrating low transcriptional activity, (7) outside of a copy number variable region, (8) located in open chromatin, and (9) unique, i.e., 1 copy per genome.

In certain embodiments, provided herein are compositions. In certain embodiments, provided herein are compositions for engineering a human target cell at suitable target nucleotide sequences within a target polynucleotide of the human target cell.

In certain embodiments, a suitable target polynucleotide that comprises a target nucleotide sequence has at least one of the exemplary characteristics. In certain embodiments, a suitable target polynucleotide that comprises a target nucleotide sequence has at least two of the exemplary characteristics. In certain embodiments, a suitable target polynucleotide that comprises a target nucleotide sequence has at least three of the exemplary characteristics. In certain embodiments, a suitable target polynucleotide that comprises a target nucleotide sequence has at least four of the exemplary characteristics. In certain embodiments, a suitable target polynucleotide that comprises a target nucleotide sequence has at least five of the exemplary characteristics. In certain embodiments, a suitable target polynucleotide that comprises a target nucleotide sequence has at least six of the exemplary characteristics. In certain embodiments, a suitable target polynucleotide that comprises a target nucleotide sequence has at least seven of the exemplary characteristics. In certain embodiments, a suitable target polynucleotide that comprises a target nucleotide sequence has at least eight of the exemplary characteristics. In certain embodiments, a suitable target polynucleotide that comprises a target nucleotide sequence has all the exemplary characteristics.

In certain embodiments, a suitable target polynucleotide is >10 kb, for example, >20, such as >30, and in some cases >50 kb away from any 5′ gene end. In certain embodiments, a suitable target polynucleotide is >10 kb, for example, >20, such as >30, and in some cases >50 kb away from any 5′ gene end and further comprises at one additional exemplary characteristic. In certain embodiments, a suitable target polynucleotide is >10 kb, for example, >20, such as >30, and in some cases >50 kb away from any 5′ gene end and further comprises at least two additional exemplary characteristics. In certain embodiments, a suitable target polynucleotide is >10 kb, for example, >20, such as >30, and in some cases >50 kb away from any 5′ gene end and further comprises at least three additional exemplary characteristics. In certain embodiments, a suitable target polynucleotide is >10 kb, for example, >20, such as >30, and in some cases >50 kb away from any 5′ gene end and further comprises at least four additional exemplary characteristics. In certain embodiments, a suitable target polynucleotide is >10 kb, for example, >20, such as >30, and in some cases >50 kb away from any 5′ gene end and further comprises at least five additional exemplary characteristics. In certain embodiments, a suitable target polynucleotide is >10 kb, for example, >20, such as >30, and in some cases >50 kb away from any 5′ gene end and further comprises at least six additional exemplary characteristics. In certain embodiments, a suitable target polynucleotide is >10 kb, for example, >20, such as >30, and in some cases >50 kb away from any 5′ gene end and further comprises at least seven additional exemplary characteristics. In certain embodiments, a suitable target polynucleotide is >10 kb, for example, >20, such as >30, and in some cases >50 kb away from any 5′ gene end and further comprises all eight additional exemplary characteristics.

In certain embodiments, a suitable target polynucleotide is >150 kb, for example, >200, such as >250, and in some cases >300 kb away from a known cancer-related gene. In certain embodiments, a suitable target polynucleotide is >150 kb, for example, >200, such as >250, and in some cases >300 kb away from a known cancer-related gene and further comprises at one additional exemplary characteristic. In certain embodiments, a suitable target polynucleotide is >150 kb, for example, >200, such as >250, and in some cases >300 kb away from a known cancer-related gene and further comprises at least two additional exemplary characteristics. In certain embodiments, a suitable target polynucleotide is >150 kb, for example, >200, such as >250, and in some cases >300 kb away from a known cancer-related gene and further comprises at least three additional exemplary characteristics. In certain embodiments, a suitable target polynucleotide is >150 kb, for example, >200, such as >250, and in some cases >300 kb away from a known cancer-related gene and further comprises at least four additional exemplary characteristics. In certain embodiments, a suitable target polynucleotide is >150 kb, for example, >200, such as >250, and in some cases >300 kb away from a known cancer-related gene and further comprises at least five additional exemplary characteristics. In certain embodiments, a suitable target polynucleotide is >150 kb, for example, >200, such as >250, and in some cases >300 kb away from a known cancer-related gene and further comprises at least six additional exemplary characteristics. In certain embodiments, a suitable target polynucleotide is >150 kb, for example, >200, such as >250, and in some cases >300 kb away from a known cancer-related gene and further comprises at least seven additional exemplary characteristics. In certain embodiments, a suitable target polynucleotide is >150 kb, for example, >200, such as >250, and in some cases >300 kb away from a known cancer-related gene and further comprises all eight additional exemplary characteristics.

In certain embodiments, a suitable target polynucleotide is >150 kb, for example, >200, such as >250, and in some cases >300 kb away from a known cancer-related gene, and >10 kb, for example, >20, such as >30, and in some cases >50 kb away from any 5′ gene end. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least one additional exemplary characteristic. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least two additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least three additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least four additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least five additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises at least six additional exemplary characteristics. In certain embodiments, a suitable target polynucleotide is >150 kb, for example, >200, such as >250, and in some cases >300 kb away from a known cancer-related gene, >10 kb, for example, >20, such as >30, and in some cases >50 kb away from any 5′ gene end, and further comprises all seven additional exemplary characteristics.

In a preferred embodiment, a suitable target polynucleotide is >10 kb, for example, >20, such as >30, and in some cases >50 kb away from any 5′ gene end and >150, for example, >200, such as >250, and in some cases >300 kb away from a known cancer-related gene. In certain embodiments, a suitable target polynucleotide comprising a target

nucleotide sequence, e.g., for transgene insertion, may comprise any one of SEQ ID NOs: 2020-2043 of Table 6. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2043. In a preferred embodiment, a suitable target polynucleotide comprising a target nucleotide sequence is at least 98% identical to any one of SEQ ID NOs: 2020-2043. In a more preferred embodiment, a suitable target polynucleotide comprising a target nucleotide sequence is at least 99% identical to any one of SEQ ID NOs: 2020-2043.

In certain embodiments, a suitable target polynucleotide comprising a target nucleotide sequence, e.g., for transgene insertion, may comprise any one of SEQ ID NOs: 2020-2042 of Table 6. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2042. In a preferred embodiment, a suitable target polynucleotide comprising a target nucleotide sequence is at least 98% identical to any one of SEQ ID NOs: 2020-2042. In a more preferred embodiment, a suitable target polynucleotide comprising a target nucleotide sequence is at least 99% identical to any one of SEQ ID NOs: 2020-2042.

In certain embodiments, a suitable target polynucleotide comprising a target nucleotide sequence, e.g., for transgene insertion, may comprise any one of SEQ ID NOs: 2020-2041 and 2043 of Table 6. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2041 and 2043. In a preferred embodiment, a suitable target polynucleotide comprising a target nucleotide sequence is at least 98% identical to any one of SEQ ID NOs: 2020-2041 and 2043. In a more preferred embodiment, a suitable target polynucleotide comprising a target nucleotide sequence is at least 99% identical to any one of SEQ ID NOs: 2020-2041 and 2043.

In certain embodiments, a suitable target polynucleotide comprising a target nucleotide sequence, e.g., for transgene insertion, may comprise any one of SEQ ID NOs: 2020-2041 of Table 6. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to any one of SEQ ID NOs: 2020-2041. In a preferred embodiment, a suitable target polynucleotide comprising a target nucleotide sequence is at least 98% identical to any one of SEQ ID NOs: 2020-2041. In a more preferred embodiment, a suitable target polynucleotide comprising a target nucleotide sequence is at least 99% identical to any one of SEQ ID NOs: 2020-2041.

In certain embodiments, a suitable target polynucleotide comprising a target nucleotide sequence, e.g., for transgene insertion, may comprise at least a portion of, for example, nucleotides 1-495, 1-490, 1-485, 1-480, 1-475, 1-470, 1-465, 1-460, 1-455, 1-450, 1-445, 1-440, 1-435, 1-430, 1-425, 1-420, 1-415, 1-410, 1-405, or 1-400, of any one of SEQ ID NOs: 2020-2030 of Table 6. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to the portion of any one of SEQ ID NOs: 2020-2030.

In certain embodiments, a suitable target polynucleotide comprising a target nucleotide sequence, e.g., for transgene insertion, may comprise at least a portion of, for example, nucleotides 5-500, 10-500, 15-500, 20-500, 25-500, 30-500, 35-500, 40-500, 45-500, 50-500, 55-500, 60-500, 65-500, 70-500, 75-500, 80-500, 85-500, 90-500, 95-500, or 100-500, of any one of SEQ ID NOs: 2031-2041 of Table 6. In certain embodiments, a suitable target polynucleotide comprising a target nucleotide sequence is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or completely identical to the portion of any one of SEQ ID NOs: 2031-2041.

TABLE 6
suitable target polynucleotides comprising a target
nucleotide sequence for transgene insertion

| SEQ ID NO | Sequence |
|---|---|
| 2020 | GCCTCCCAAAGTGCTGAGATTATGGGCATGAGCCACCGCACCTGGCCCTGAC |
| | AAGAACCTTTGAGTTAGGTATAATGGTTCACCCCAATTTATAGATAATGAAC |
| | CCAAGTCACAGGGGAAGTGAAGTCAGTTGCCTAAGGTCAGACAGCAGTAAAT |
| | GGTTCTCTGACCCTAACTCCACTGCCTCCCTCTCATAAAAACACTGGGTGGT |
| | TACAGTGGGCCCACCTGGAGAAGTCAAGCTATTCTCTCCATCTCAAGAACAT |
| | TAATTTAATCATCCTTTTTACCATATAAGATAACATCTTCACAGGTTCTGAG |
| | GATGAGAATGTTGACATCTTTGGGTGGTCGTTATTCAGCCTATCACAGGTAT |
| | CCAGGGAAGAAAAAAGGAATTTCCAAAAAGAGAAAATACGAACATTGGGAAG |
| | GCTAATTACAGATGGTGACTACTGAAGGGTTAGTCAGAAGCATAATGGAGGC |
| | AGTGATGAGATGACAGCACAGATGCATGACTCTAGTCCCAGCAACTCCTAAA |
| | AGGTAAAGAAATGTATCCTGCCACCCTCAGCTTCTTTGGGGTGTCCTCATAA |
| | AAGAGAGGCAGTAAAGCAGAATCAGAGTCAGATAGAGAGGTTGTAAGAAGAG |
| | AAGCAGAGGTGAGTAAGCTGTGTTTCAAACCCAGAGTCAAGGCTCTTGCCCC |
| | TCTGCGGTGCTGCCGAAGCCCAGGGTGGGTGGGGACTGACATGCAACTCAGG |
| | TACTGTGTGGCAGACTTTGTGCCTTGGCATGAAACTATGCCTGCCCACAGGA |
| | AGGGGCACCATTTTCTCATTAGCTCAAAGAGACTTCTGCTGGCCAATTCCTG |
| | TCTTCTCAATACTGCAGCTCTCCAGAGACAACACTGTTCTCTATTCTCCTGT |
| | AAGTGAGGCAGAGCCTGGCAGTACCCTCTATGCCACCTCTCACTAGTACAGG |
| | TTAGCACTCAGGGTGGCCCACTGGTGTGTGTCTCAGCTGCTGGTGTGCGTGC |
| | TGGTGCAGGTAC |
| 2021 | TGGGCTGAGGGTTGTGGCTGGATCTCTTTGCATTGCCACATCCACAACAGAA |
| | TTTTGAGAAGTCCGAGAATTCTAAATTGGAGCCTGACCTTCTTCATAATAGT |
| | ATATTTGTCAAGGTAGGAGGATAAAACATTTTATTGAACAGTTTGCTAAGCT |
| | GATTTAAAATTTTCCAGCATTTAGCTATATGGTATATGGACCTCCACATGTA |
| | TGATTTCATTTATATTAAATGTCCAGAATAGACAAATCTATAATGACAATAA |
| | AGAGATTAGTAGTTGCCAGAGGCTGGGAGGAGGGGAGAAACAATGAGTGATT |
| | GCGGACGGGTGTGGGGTTTCTTTCTGGGGCGATAACAGTGTCCTGGAATTCA |
| | ATAGTGATAATGGATGCACACTGTGAATATACTAAAAGCCACTCACACTTTA |
| | AAAGTGTGGGTTTTATGGTAATTTGAATGATATATCAAGCTATCACCAAAAA |
| | TACACAATGGGAGTTCAGAAATGCCACCCCAAACTATGATGATTTGACATGC |
| | TCATTACTTTGAACTGATGTCACTTGGGGAAAAACAGATGCAGGCAGAGACT |
| | TTCTCTGAGATCTGCTTATCTGCCTAAGACAGATCAAGGGATCCTCCAAAAG |
| | GAACTCAATTGTCATGAATCCCCTCCCCTGGAACCTTATCAACCAGGGACAA |
| | TGAACTTAGATCAGAGAGGGGGAGACTGGAGGTTGACATCATGCCTAGACAG |
| | CCACCTCTTCTTCTGAGGGCTGCTCCAAGAGAACCTTTATTACTTGAGAGGC |
| | TTCTCATTTGCATAACAAGAAACCTTTGTTCACCATACACTTCCTCCCCTCA |
| | TATTCTCATAACTGGTGTCACCACCACCCACGCAGAAGTCCAAAGCCTCTAT |
| | TCCCTTCTGTACCTCAGGGTGCTATATAAGCTTCAATCATCTGACCCTTCTT |
| | TGAATCTCATATTTTGTGGGCTTGCATGGGTATGTACATAATTAAAAATGGA |
| | TTTCCTCTTGTT |
| 2022 | ATTTACACACATGCCACAGACAGAAACATTTTAATAGACCTTTGCTTATGGA |
| | AAAGTAAAGCAAAAATGTAATTCTAGAAGGGAGAAATTTTAGTCAATTAGAA |
| | AATAAGATGGTCAGGCATTGTAGCTCTCATGTGTAATCCCAGTGCTTTGGAA |
| | GGTTGAGGCGAGAGGATTGCTTGAGACCAGGAGTTTGCGACCAGCCTAGACA |
| | ACATAGCTGGTCATATAAAAAAACTTCAAAAAAATTAGCTAGCTGTAGAGCT |
| | TTCTGCCTATATTTCCAGCTACTCAAGGATGAGGCAAAAGAATCCCTTAAGC |
| | CCAGGAGGTTGAGGTTGCAGTGAACTGTAATTGCACCACCACACTCTAGCCT |
| | GGGTAACAGAGCAAGGTCCCATCTCCTAAAAAAAAAAGAAAGGAAAATAAAA |
| | AGAAAATAAACTATTCTCCATAATAATGTAGACAGCAATCCTCACTGTGAAC |
| | CAGAAGGAACCTCGGCAAATTTTTTAGACATCAATGGGATTTCACTATCAGC |
| | TGAGAGTGTTCCCTTTTTAGCATGGCAAGCTGTTTCCTGAAGCAATAGAGAG |
| | AAGCAAGACCAAGGAAAAATCTAGAAAGAGCCTCTCTGTAGAAAAGCAGAGC |
| | AATGATCTCTAATCACAATGCTATCAAATATTCCAGGCTAAATTTTCCTTTA |
| | TAGCATTAAAATTTTCCTCACATCCACAAGATTCCAATAGTTTTCTTAATGC |
| | CATAGCCTGGTGTCTATTCTGCCTTGTGGATTCCCATAATGCAAAATGCCAT |
| | TAAAAAAGGAACAGACCATGAGAAGTGGGCCTCCGAAGCACATGAAGCTTGG |
| | TATCATCAGAAAGATAAGGGGCAACAGTCAGGAATAATTGTTGGGACATTTA |
| | ATAAGTCCCTGGAAATTCCTAGAAACATAATTTTTTTTTGAGTCTAAGATGC |
| | TATCATTTTAAGGTGCACCATTATTTTATTTGCTACAATGTAGAAAACAATA |
| | ACACTGCCAATT |
| 2023 | TGATTAGGTAAAATATCAGAGACACAAATCAGGTTAAATTGATTTTTTATTG |
| | TAATTACATTTAAAATTTTAGAATTCATCAGTAGGTATGAACAAACATATAC |
| | ATACATATATATAATTTATATTATAAGTTTATTATTTATACTATACATTATA |
| | AAAATAACTGAGAGATAAACTTTCGTTTATCCTTAATGCTAAAATAATTCAT |
| | TTACCTTGGAGAGATCAGAACTCTGTCCATTTCCCCTACATAAAAACTAGAG |
| | AGTACTATTGCTTTCTCTTTCTCGGGCTTACTCTGGTCTCATAGAATATGCA |
| | TTTTCATTTTTTTTCAACAGAATATCCGTGGATAGCTAAAATTTCTGCTTCC |
| | TTTGTCAACATTTGTATTTCCCCAGTGGACATTTCTGCAAAATTTATTTTCA |
| | TTTCTTTGTTACCAGAGAAACTCTGTTGGTCAAGTTCAATAGCATCCTCAGC |
| | ATAATTTCAGAAGGAAATTACAGGGAGCAATTGAAGTCCATCACTTTCTTGG |
| | AGGGGAAATATTAACACCCTCACCTCTTGCTCCCAATATTAGGTGGTAGGCA |
| | GGAGTGAGTTACTCATTTTCTGAAGGAGCAGTAACTCTTTGGACCCCTCGAG |
| | TCACTTGGTAAATAAACTCTAGCACTGCCCCGAAGAGTGCCTCAGAGATTTC |
| | AAGGAATAAATGCTTTAAAGGTAGGAAAATGCTAAGAAACACCATCATATAA |
| | GTGAGTTATTTCCAATTTTATTTTAAATACAGCCATATATTATTACATACAG |
| | CCACACATTATTAAATAATGTATTAATACATTATTATTAAATACAGCCATAT |
| | ATATGTATATATGTGTGTGTGTATATATATACATATATATGTAAGTATGTAG |
| | CTGCTATACCCTCCTGAAGCAATGAATGTAGCTGCTATACCCTCCAGAAGCA |
| | ATGATACCCTCCAGAGGTGATAACAGATACAAGTAACAACCACACTCTCTGG |
| | TTTTGACAACCA |
| 2024 | CAGAGAGCTTCCAAGGCATTATCCCATCCAAAGGGTAAAGAGGCTGGGATAT |
| | TTATCGACTAGCTCCCATTCTTCACTGGCTGTAACTTGTCCACGTCTCACAG |
| | CTGTAACTCCCTTGTATTCCCTACCTATCTGGTGTGAGGACCAAGCTTGTGT |
| | CTGTGGATAGAGAAAGCCCTAAAGCAGAAAGTCTAGGTGCTTGCACAAAAAG |
| | ATCATCTGCACAGAATGATGATCAAGAGATGTGAGTGGGGCACCACAACATT |
| | TACCTCAGGAATCTCTGTTCAGGACTCAGCTTTGGTCTCAAACCTTGGGAAG |
| | CTTATACACTGAGGCAGTGTTAGGATCTCTTTTCTCTGCCTTCCTGTGCTTT |
| | TAAGTGTATTTCACTGTTTTTGATCCCTTGTCTGCCCCTTATATTTGACTAT |
| | CAGGCTCTTGAAGGTCTATTACACTTACTCATTGTTTTTACCCCCTGTTCCT |
| | ATCTCAGTGCCCAACACAGAGCTGACAGTTAATATATGTTGGTTGGATGCAT |
| | GTGTGGGTATCTTATCTTTTTATCCTTTAAAAGACCTCACACGTAGATGAAA |
| | ATTTTAAAATCATTAATTCAATCATCAATTCAATTCAATCATCTTTTTATCC |
| | TTTAAAAGACCTCACACATTGATGAAAATTTTAAAATCATTAATTCAATTGA |
| | AGAGGCCTTGTGATTGACATGAGTATAAATTGGACCATTATTAACTTCAAAC |
| | TAATTCTACTATGCCAGAAACCATGCCTGAAGTATTAAAACATCACGTTAAA |
| | AAACAAAAGACAAAAAAAAAACTTATCTAAAAAATTACATTAAATAAAATAG |
| | ACCAAAGGTAAATCTTACTCAAGTTTTCAGGAAAAAAAAATTGTTTTCTATA |
| | CTCTTTTCTCACCTATTCTTCCTTGTCACAGAGAAGCAATTATTATATTAGA |
| | CTTTCCTTTTTCAATGTGTAGATGACATCATATGATTTAAATTTTTTATGTA |
| | TTTCTCTTGCAA |
| 2025 | ATCAGCAGCAGAGGCTGCAGAACAGCGGATATTAGTGAAAAGCAAATGTTGC |
| | TGTCTGATCGTTCCTGTGGAAGTTTTGTCTCAGAGGAGTACCCGGCCGTGTG |
| | AGGTGTCAGTCTGCCCCTACTCGGGGGTGCCTCCCAGTTAGGCTACTCAGGG |
| | GTCAGGGACCCACTTGAGGAGGCAGTCTGTCTGTTCTCAGATCTCAAGCTGT |
| | GTGCTGGGAGAACCACTACTCTCTTCAAAGCTGTCAGACAGGGACATTTAAG |
| | TCTGCAGAGGTTTCTGCTGCCTTTTGTTGGGCTATGCCCTGCCCCCAGAGGT |
| | GGAGTCTACAGAGACAGGCAGGCCTTGAGCTGCAGTGGGCTCCACCCAGTTC |
| | GAGCTTCCTGGCTGCTTTGTTTACCTACAATGGTGGGCTCCCCTCCCCCAGC |
| | CTTGCTGCTGCCTTGCAGTTTGATCTCAGACTGCTGTGCTAGCAATGAGCGA |
| | GGCTCCATGGGCGTAGGACCCTCCGAGCCAGGTGGGATACAATCTTCTAGTT |
| | TGCTGTTTGCTAGGACCATTGGAAAAGCACAGTATTAGGGTGGGAGTGACCC |
| | GATTTTCCAGGTGCTGTCTGTCACCCCTTTCCTTGGCTAGGAAAGGGAATTC |
| | CCTGACCCCTTGCGCTTCCTGGGTGAGGTGATGCCTTGCCCTGCTTCGGCTC |
| | ATGCTCAGTGCACTGCACCCACTGTCTTGCACCCACTGTCCGACAATCCCCA |
| | GTGTGATGAACCCGGTACCTCAGTTGGAAATGCAGAAATCATTCATCTTCTG |
| | AGTCACTCACGCTGGGAGCTGTAGACTGGAGCTGTTCCTATTCGGCCATCTA |
| | CATGTTCTTTCTTCCCTCATCATCACTTCTTTACTTCTTTTATTTCACTTCT |
| | GGCTTTCTGTCCTCCCACGCTGAGGAAGACTGATTTGGTGGACATGTATTTA |
| | TTCTGCTGAGTACCAGTTGATGTGGAAGTAGTTGTTTTATAGTCAACATGTT |
| | TTTATGACTAAT |
| 2026 | GAGTGATGTCTAATCACAATCTGTGATAGGTATTTGCTTTAAGGTGCATCTA |
| | ATAACATGACAGTGATTTTCATCTCATATAACCTTCATTAACTCTGGTTCCC |
| | TGCTAAGATAAAGCCTTCCCTATAAGCCAACTGAGAATACTGTAGTCAGAAT |
| | TTACAGGTACTTCCCATTGTGGTTGTTCACCTTATTTGTGCCAGTTTTTCTT |
| | CTTCTTTATTCATACCTTTTGCCATGTGAATTTGCATTTCTTCTGGGTTGGA |
| | GTCAAGTATATATTTATCCTTTTTACCTTTGACTCTGAGGCTGGCCAAAGGA |
| | ATAAGGTGGATGTGACAAGGTACAATTTCTGAGCCTAGCCCTTAGAGGCCTT |
| | CCATGTTTCCACTTGTTCTCTTGCACTTGCGACGTTGCTGTCAAAAGAACAT |
| | GCAATGGCTAGCTAGCAGCCTGTGCACCTGCAGTGAGAACCAGAGCCACCCA |
| | GTTGCTGCAGCCTGAGACCAAGCTGCTCAGCTAAGCATAGCTTAGATCACCA |
| | TTGAGTTCTGAGGTGGTTTGTCATACAGCAATGGCAATCAGATATATCCACA |
| | CAAATATAATTTTAGTTTATATTTTTGTTACTGCAGTTCTCATCTTATTCTG |
| | AGGATACGTGACAAAATAATTCTTTCAAAAATATTGATGCTGTGCCAGATTA |
| | CTATTTTGAATGAATTATTAGACAAATACTTCATATGTATCTTATTATGTGG |
| | GTTTACACATTATTTATCTTATTGATTTAACTTCAAAACTAAACTTTAGTTT |
| | AGCTCTTGGGCCCTATCTGGGAAAGGGTCATCTTTTAATCACCATTAAATCA |
| | CTGAAGTCATCAGTTTATTCAAAGTACTCTGCACAAAATTAGCATTCTTTAG |
| | TGGTTGTGAAATAAATAGACTTTAAACTTATCATTAATATTCCCAATGGTAC |
| | TATGGGGGAGGCAAAATTTTCTATCTTCTTAGTGGTTTTTTTTTTTTTGGCT |
| | AGGGCTAAGGAT |
| 2027 | ACGCACCTGAGAAATGTGTTAAGGATTAAGATGCTAGTGCTAGATGTTTGAT |
| | TTTCTGAATCGAACCACTATTGGTGAGATCCAGAAGCTCAAAGACATGATAT |
| | ACCCACCTTCAAATAATGTTTATGTAGGTAATCTATTCTCAGGATTTATAGA |
| | CACTGCTGTTAAGACCTATTGTCATTGGGGTAAAAAAAAATCCTTATTATAT |
| | TATACAAATTATTATATACTATTATATTATAGAAATTATATTTCTATTAAAT |
| | AGCTTGTGTAGAAAGTAACCATATATAGTTAGAAAAACACTGATCTCAAGAA |
| | CAGGATTTTAGATTTGACTCTGACAATTTCTGTTCGGTCTTGTATAAATGTA |
| | TCAATTTAGATTTAGGGCTTTATTTTCTAATCCATAAAATGTGTAGCATACT |
| | TCTGCTAGCTATACATTTACTGAAGTTATTATTTTAAACTATTTTTATTTTC |
| | ATTTTTTTGTTTTGAGTTATAATCATAATTAATGGATTCAAGTGACAGAGAA |
| | AAGAAAGTAATTAGTCATCTTTTTTCAGAATACAGTCTTTGTTCTGAAGGTA |
| | TTTCGTATGAATCAAGTTTCAAATCTTCAGATAAATTTTCACCTTGCCAATG |
| | TGCTTTCTGCTCTAAATCATTCCTGAATTTTGCTATGATTTTTCTTTCTTAT |
| | AAAATCTTGACACTAAATTGTCAGGAGATATACATATATGTATATATGTAAA |
| | ATATATATATCATATATAAATATATATAAATTTTGAGTTAAAGTACTATTAC |
| | AGTATTCAATTCTACCAGTAATTCTAATAGTATGAAAATAAAGTCACCAGTT |
| | GAAGTAAGACCTACTGACACCTTCTATTATATTTCGATAATTCTATTTGAAA |
| | CTAATTATATAGTAGGACATTTTCATTGTTTTCAGTATTAACTGGCACTCAT |
| | GTAGATATTGCAGGCCAAATTTTACCTCTACCTTTTGGAATTTTCTGGGGTA |
| | GACTTGAGAATT |
| 2028 | TACATGTGTAAACAGTTTTAGCGTAGATTTCCTCGCACTTTTAAATTTTGGA |
| | TTCTTAATTTCCCTGTCCCCCCTGCCCCCCCCCCAAAAAAAACCTGCTAACG |
| | TTTAAACGAACACAGTTTGGGAAATCTGCGTTAAGTCCTTCGTGGGAGTGGG |
| | GTTGCTCAGCTCACAGTAGGCCACGAACCTGAATTTTCTCTTGTCTGCTGCC |
| | CCCTTTTGATAGATGGAGGGAAGAGCAGGCTTCCAGTGCAATGGACAGAAGA |
| | GGGAGCCTGCAAGTTGGTAACAGAGTCTATTAGGGAAAGAGAGAGTCACTTG |
| | AATCCTCAGAGCTGCTCCTGTCAACTGCTTTGTGCAGTTTTTGTGACTTATT |
| | AGCTGCTTGTTTGCACTCTATCTACGCCTGCCCAGGTGTGTTTGGGCCCTAG |
| | AGCGAAGGGAGCACAGGCGTTCATTTAGAAACTTATCCCTCCGTCCAAATAT |
| | TGGATGCTTACCATGTGCCTGGTGCAATGCAGGGTGATACAAAGAGGAAGAT |
| | AAGTGAGGCATTCTTATCGAAGGACCAGACACTCTTCCAGCCTGACTATATT |
| | CATTACACTCGTGCCTGACCTTTCTTTGACTCTAAGATTCTTCCTTTCTAAA |
| | TGTGAATCTTAAAGACTGAAGTCTTTGATCTAAGACTGCTTTCTTATCACAT |
| | CACATCCAACAACCAACTTTTCACAGCTTCCCAGATCCCAAATTCTGTTTAG |
| | CAAGGACACTTGGATTTTTTTGTTTTTTGTTATAAATGACCTCTTCAGGTTC |
| | ATATTTTCACTATGTCCAGAATTCTTATTTTATTCTGTTTTGTGCTGACATT |
| | GGAGGCAGAGTCTGTGTCACAGAATACACCACTAGGGGTTACCCTGGACATG |
| | GAAGGGTATTCACTCGGGGAAGAAATTTTAATGGAATTTTTAATATCTAGAG |
| | CTGTCATTATCCTGTGATGGTTCACAAGAAATGGAACACTTAAAAATTTCTA |
| | CAGAAAAAAAGG |
| 2029 | GCCACAAATTTGTTTTCTGTATCTGTAGATTTGCATTTTTTTCCGAACATCT |
| | CATATGAATAGAATCACAAAATTTGTGTATTTTGTGCCAAACTTCTTTCACT |
| | TAGCATACTGATTTCAAAATTGATCCAACTTATAGCATATATCAGTACTTTA |
| | TTCCTTTTTAGGGCAAAGAAATCTTCCATTACACGGATACCCCACATTTTAT |
| | TTCTCTACCCATCGCTTGCTGGGCATGAGTTGTTTGTGACAAATATTCATAT |
| | ACATATTCTTGTGTGGACATATGTTTTCGCTTCTCTTGGGTATATATCTAGG |
| | AGTAGGATTGCTGGGTCATATGGTAAGTCTCTATTTAATGGTTTAGACTCAG |
| | TACTTTGTTTTCTGCCTTTCCACAGCTCAGTTTCATAAAGAGGCAGGAGCCT |
| | TTTGTTCAGGGCTCCTTGGCAGTAAGGTAATTTCTTCTTCTGCATTGTATCC |
| | AGCTGACCCTTGCTCAGTGCTGTTCTTTGGGGGAAAGATGGAATGCTGGGAA |
| | GCCAGCACCTCTTATTCCTTCTAGCTAACACTTTTACAGTGACGGATATAAT |
| | AGATATCTTCAACTAGTATTGTTGAATTATCTCCCTGATGCTGTCCAATTTT |
| | GCTTCATATATTTTGGGGCTCTGTTATTAGGTATGCATATATAGTCATTATT |
| | GTTATATCTTTGTGGTGGTGTGGCCTTTTTATTATTTTAGCACTTTTATATC |
| | TTTACCTCTAATAACGTTTTTAAAAATTGAACGTTGATTTTGTCTGATGTTA |
| | GTACAACCACTTCAGCTTCTTTGTAGTTGCTGTTTGCATGACATATCTTTCT |
| | CCATTCTTTTACTTTCAATCTATTTGTATCTCTGGGTCTAAAATGTGTAGAT |
| | AGCACATAGTTGAATCTTTTAAAAAATACATTTTACAATCTCTGATTTTTAT |
| | TGGAATGTTTAATCCATCCACATTTAATGTTACGATTGATGGAGCTGGACTT |
| | ATTTCTGCCATA |
| 2030 | AACACAGAGCTAAAACCAAGTAAGAGGCGATTCTCCAAAAGCACTTCCTCAG |
| | CAAACAGCATATCTATTGTGTGTGGGTTCTTTAATTGGCTGAGAACTGAATT |
| | TCACCTTTGGCATTAAAGAGAAGTGTTTATTTTTACTGTCTTCACTGTTTTA |
| | ATGTTTAAACAAAATCTAAATACTGAGGTGAACTCTATCATAAAACAAGTGA |
| | AACGGCAACATAGGTTGATCCAGAAAGAAGCAAATTCCAGCATGGGGGCAC |
| | TACATGTTTCAGCTCATCAGTTATCTGAATCTTATGGCTCTAAAGATGGATG |
| | GATGAGAATACATAGGCAGAAGCTTCCTGGTGAGGCTGGTATGATTCTGTTG |
| | TCCTATCTTCAACACTATCCTTCTACCTTCAGGGTTGCTGTTGTAGGTTTTA |
| | TTTCTTTGGCTTCTGTTGCCAGTAATGGAAAAGGACCACATGGAAGACTGTA |
| | TTTATGTACATCATGTCCAAACAGAATATCCTATAATAGTGAATCTTGGAAG |
| | AAAGCTTGAGAGATGTGGCCCAGCGCGGTGGCTCACACCTGTAATCCCAGCA |
| | CTTTGGGAGACTGAGGTGGGCTGATCACGAGGTCAGGAGTTCGAGACCAGTG |
| | TGACCAACATGGTGAAACCCCATCTCTACTAAAAAGACAAAAATTAGCCGGG |
| | CCTGGTGGTGTTGCACCCGTAATCCCAGCTACCCAGGAGGCTGAGGCAGGAG |
| | AATTGCTTGAACCCAGGAGGCAGAGGTTGCAGTGAGCCAAGATCGTACCACT |
| | GCACTCCAGCCCTCCAGCCTGGACAACAGAGCAAGACTCTGTCTCAAAAGAA |
| | AAAAAAAATACCAGTTTGAGAGATGTATGTGAGGACTGATTACCGAAAGCGA |
| | AAGGGTTTAGTACATCTCATGAGAACAGAGCAGTCACAAGTGATATAAACCA |
| | AACTCCCTTGGAAATTTGTAATCTATCAACTTCTTTATTTAAAGAGAATAGG |
| | AGGTTTACTGTG |
| 2031 | ACTCCCACTCCTACTAATTACAGCTTGTGTGTCCTTCAGTCATTCACTTCCC |
| | TTCACATGACCAGCCCAGCAGAAATGAACTACCAGGAACATGAGCTCAGAGC |
| | GATGGGCTGGCCACCTGCCAAGCACCTCTGAATGGAAAGAGCAGAATTTTGC |
| | ATTGCCTGCCATGCCACGTGGAGCAGGCCCTGGGTGGCTCTTTAGGGGATGG |
| | GTGTGGACTCCCACAACAAAACCAAGGGCCATATTCAAAGTTAAAAGCTCTG |
| | CCATAGATGGTATTTGTTGAGGCTGTGTGTGGTAGCTCATGCATGTATGCCC |
| | AACACTTTAGGAGGCTGAGGTGGGAAGATCACTTGAGGCTGGGAGTTCAAGT |
| | CTAGCCTAGGCAAGATAGTGAGATCCCTTCTCTAAAAAAGATAAAATATTAA |
| | CTGGGCATCATGGACGTGCCTGTAGCCCCAGCTACTGGGGAGGCTGAGGCAG |
| | GAGGATGGCTTGAGTCCAGGAGTTTGAGACTGCAGTGAGCTGTGATTGCACC |
| | ATTGCTCCCTAGCCCGGGTGACAGAACAAGACTTTTATTTCTTTAAAAAAAA |
| | AAAAAAAAAAGAAGGTGTTTACTGCAGTTGCTTTATTAAAAAAAAAGTAAAT |
| | GAATGTTCTGACTGTTCTACTTTTGAAAATAAGTGGCAAGGAATTAGAACTG |
| | TATCTTTCAGCAACAAAATGTACACTGTGGTTCCATGTCACAGCCAGGAATG |
| | GAGTCAGATGTCTCAGACCAGAATCACAGCTCTGCCACCTCCTGTGACATGG |
| | ACTTGCTAAGCTACCTTGACTCTCTGGAGCTCACTATGCCCATCAATAACAA |
| | GAAATAAATAAATCCGTCCTGTAAGGTTGTCAGGAGAAACAAATGAGGCACT |
| | ATATGTGGAAGTTCCTGGAATAGTGACCAGCACAGAGGACGTCTCAAAGAAA |
| | GATTTGCTGAACCCCAAAAGACAGGAGGACTGGAGGAACAACAAAGAGACAG |
| | GAAAGCTAGCAT |
| 2032 | AATTCATAGCCCAGCCAAGGAACTTAGAAGAGTAGAGGGAAGTCATTTTTCA |
| | CTCCCCTACAAGAACATTCTGCTGTAAAGAGGAGCTAGAAATAATTTTTGTT |
| | TTAAATTCAACCAAACATAGGGATAATTCTGAAATTTGGAACCAAAAGAATT |
| | ATAAGTACACTACTGGTGAATTTGTGCTTATCTGAAATCTACACATGTAGCT |
| | GTCTTTATGTATCTCTGTATATCGATGTTTTTCTATATATATAATCAGTGAA |
| | GTAAGATATCTAGTCATTCATTTACTCACCAAGTGATTGCAGTGGGGTGACA |
| | GGGACAGTGGGGGGTGTGGTGGCGGGTTGCCAGAGCATGAGGAGTATGCAAT |
| | AGAATCTAAGAAATCATACCTACCTGGCCAGGCACAGTTGCTCATGCCTGTA |
| | ATCCCAGCACTTTGGGAGGCAGAGGCAGGCGGATCACTTGAGGTCAGGAGTT |
| | CCAGACCAGCCTGGCCAACATGGTGAAATCCCATCTCTACTAAAAATACAAA |
| | AAATACAAAAAATTAGCTGGGTGTGGTGGCACATGCCTGTAATCCTGGCTAC |
| | TCTGGAGGCTGAGGCAGGAGAATGGCTTGAACCTGGGAGGCAGAGGCTGCAG |
| | TGAGCTGAAATTGTACTACTGCACTCCAGCCTGGGTGACAGAGTGAGACTCC |
| | ATCTCAAAAAAAAAAAAAAAAAAAAAAAATCAGACCTGCCTTCCATGAGCTC |
| | ATGGTATACTTGAATCTCCATAGGCTAGTTATTCAGGAGGGTATGTAATGTA |
| | ACTCAACAATGCACAATTACTTAAATTCGCTCAGGAGAATTACCTCATTTTG |
| | CCCAACTTGTTACTGTGAAAAAAAAAAAAGAAAGAAAATTTCAGGACCTTCC |
| | AAATTTATTATGCCAAAGGGAAAAGTCAAGCCCTGGAAACCAAGTCATGTAA |
| | CACGGCTGTTTTTCTTCTCTGGTGCATGACTGTTGCTTCCTGATCTTTTTGT |
| | TGATGTTATACA |
| 2033 | CATATAAATTAAATATTTATGTTATATTGAAGGAATACTTTTAGACTTGTTT |
| | AAACACAAATCTTTAAAAATTACATATCACTCTTGCATGTACATAAAAAATG |
| | AAAATATAGGCAATTAAATTAAGAGAGGTCTACAGTGTCTTTACATCAAGTC |
| | TGACTCTACTGAGTCCCTTTTTGACTCAGAGTCATTAATATATTGTTTTTTT |
| | CCAGTAATAATGTAGTGATGCAGCCTGTCTTCAAAGACTGCTCTACTATTGA |
| | CTCAGATTTTCTCCCAAGCCATTGATACTAGTTTTGAAGCTGATGCTTTTTA |
| | AATCTTGCTGTCAGACTTACGGGAAGGTTTTCATACAACAGGGCTCATATTC |
| | TTTCCTCAAATTATCCTTACATGTAAATGTTCAGAATGTCGAGATGATACAT |
| | AGGCCAGTTATGCCACTGTGAATATCTACCAAGGTCACATGTGTAATGAACA |
| | AAGACAGCTATTTCTGCTGCTGGCTGGCAGTGATTTGCAAGATTTTGTTGAC |
| | TGTAGGACATATCCTACTTCAATGATGTTAAAATGTGAACAAATATGCACTT |
| | CAGACTTTGTAAAATGTAGCACAGCACTTACAGAGCACACTAGGCTTCTGGC |
| | ACTCGCATAAAATGAAGACTTGGAGTTTTAGCTGAGTACTAAAGGAGGACCA |
| | TCCTCCCACCGAAGGATGAAGAATTTAAGGATATGTAAGTTGAGCTGTACTT |
| | ATGTTCATCTGTGATTTTTACAAGTCACTTATTGCTACATGTATCCTTTAAA |
| | TATGCGTTGTCCTTCCTCCTAAAATGGTTTCACCATAATAAGTGAAATGTCA |
| | GCTTGTCACATTAAATTATAAATTATAAATTACCATCACCTTAGTCCTCTAC |
| | ATATCCTTCAACTTCATTATGACACTGTCCTTCAGAGATAAGGAACAGAAAG |
| | GCTTTAATGAAAACTTCAGCTAATGTAATAATTAGGGAAGGATGAGCTAATT |
| | AAGAAACATACA |
| 2034 | CAAAGTCTCCCTAGAGGGCAAAATTGTCCCCATTGAAGACCACTGGGTTAGA |
| | TAGAAACTTACATCTCACACATGGAGAGTCCAGGCTGGCATGGTCGCTCTGC |
| | TGTGCACTGGGAGCCCAGGTTCCTCCTCGCTTTGCAAATTGTACAAGCTGCC |
| | CTCATCACCTGGATGCCTACATCTCACTTAAGAGTCTCAGTTCTAGGAGGGC |
| | ACAGACAATGGTGTACTGGTAAACAGACTCTGTTAAAAAAAAAAAAAAAAAA |
| | AACCAACACAATCAGGAACATTTTTTAAAAGCCCAGATTTGTAGTGTTTGCA |
| | GATTCTTATGTTTTAAATACTCCTGCCATGGCTGATGTGAAACTACCAACAG |
| | TTTAACAACTGGCTTACTAAATTTCTGAATATTTACCATTTGTCCCTTGTAA |
| | GACAGTATTAGTGGGCTGCAGTATATCAACAGAGAAAGGGAAGGAAAAGATA |
| | CAACCTTTTGTTGAAGGACAAAATGACATTTCACTTTTCTTCAGCCCCACTG |
| | GCCAAAACTTAGTCCCATGTTCACCTTAGCTGCAGGGGAGGCTGAAATGCAG |
| | TGTTTATTCTAAACAACCATGTATCCAGCCACAATACCAGGGGAATTTATCA |
| | CCAAGAGAAAGAGAGAGAGAATATCTAGTGCTTGAAAATTATCAGTCTCTGC |
| | CACAATTTTATTTAAAAAATAACCAGAAAAATGAGAGTGAATTTTATCTGAG |
| | AGGATCTTAGAAATCTCAGCATCGAGAAGGTAATAAATAAAGAGAGATAAGT |
| | CACAGACTTCCTGCGACAGTCAAGAATTCCCCATGCAGATGACACCCCAGGA |
| | GATGCCGGGTGATTGTTCTTACAATTTCTTCAGTTGAAGGTAAATGTGGCAC |
| | TAGCCATTTATTCTTTTAGCTCACGTTGTTTGAAGTGCATCGCCTATGTACT |
| | TCACCCTTTGGACTCACTAGAAAACAAAGAGAATTTTGGAATTAGAAGAGGC |
| | TTAATAATGTTA |
| 2035 | AAATATAAATAAAACATTTCTTTTGGAAATTTTATAATTCAAGCTAATTTAA |
| | AATTATGTAAACCTCTATCTTTCATGTAATCTTCTTCCTTCTTTTAAAACAA |
| | CATTTTTTTGGTGGTCATCTGTTCGGGAGAAAATGAAATTTTCTGTGGATAA |
| | GCAGATATTCTTCACGGAGAAAGCTAACATTCTGCATTCCTCTATTTTAAAA |
| | GTGGAAAACATAGTCCTGTTATTTGTATTTAGATGTATTTCTCACCAAAGAG |
| | TGCCAGGCTGGATTACAGAAGATCTATATTCTGATCTTGTCCTTTTTCTTTG |
| | CAAGCCTGAGGAATTGTCCAGACACAGAATTCCCTAGATCCCCAGATTTCTC |
| | ACCTATAATATGAAGGGTTGAAAGAGAGGTCTCAATCGGCTTTGAATTTTCT |
| | GTTCTATACTTCTGCACCACCACTGTAGCACTGACAATTGCATGAAAATATT |
| | AAGCTCTATTATGTTTTCAGTACTATCCTTAGCTTCTTTAAAAAATTAGTCT |
| | AGCTGTGTTTGTAAATAAATGATGTCACTGGAAAAATGGTTTCATACCATTG |
| | TTGTCAATAGTTGAATGTGGCTTGCCCTCAGGAACAATGCATTCTTCAATAA |
| | TATGGAGGATGGAAGGTGTATAAGGACTCAGATAGCTATTATTCTCATTTGC |
| | CCATGATCCTTTCATATCCCCGCCTCTGGTTTAGCATTCTCTTTCTTCCAGG |
| | GGAATTTCTCCCCCATTCCATGCATTCTAGTAGAATTTTTTATCACAGTAGA |
| | TTGTCCTGCCCTGCCACAGAAATGGGCATTTGACACAGTGGCCACAAAGATT |
| | GGTCTAAGCAGTAGGCCTGTGACCCAAGGTAGGCCAATTAGAGTTTTCTGTA |
| | GAATTTTTTAGATTCAAAGTGTATGTGTGTGGGGGGGATGACTCTTCTTGAA |
| | TTTTATATTAGGATGCATGCCAGAAATTGTTGAAAGGTCTTTAATGTACCAT |
| | GTACAGGAAGCT |
| 2036 | CACCTATAAGAGGAAATATACTTATGTCTAGGTGGACTCCAATGTGTCTGTT |
| | TACTGATACTTATTTATTCATTATTTTCAAGTAAAATGTAGAAGTGAATAAC |
| | TTAAGAGAATAACTATTTTTATGAGAGAAAAATACCCACTTTCTTTTTTATT |
| | ACTTTGTTCCTCTAGAGGTTCATGAATAATATATTGAACATGTGAGGAGTGA |
| | GGCCTGTCTAGCTCTTTTCCTAACATCTTCCACTCCTGTGGCCTCTTATTAG |
| | GTACCTTTCTCAGTGAAGATATACAATAAGAATTTTGCATGCTTATTGGGAA |
| | TTTATCTGTGAAAAATCACTCAAATGTCATTAAGTCTTTTCTGATAAACCTT |
| | AATCATCCAACAACCAGAGTTTTTCTTAAAATAGCTGTTGCTCTAGAAGAAT |
| | ACCATAGAATGAAGTTGCTTCCTAGCATGGCAGTCAAGGATCCTGGTTCCAA |
| | GTATGAGCTCTGAAGAAGATAGACTATGTTCACCGCTTACTATAGCTGAGTG |
| | CCCTTGGACAATTCATTTAAACTGCCCCTAATTTTCTTCCATCATCTGTAAA |
| | ATGAATGTAATAATAGCTCTTAATGAGTATTAAATTAGATAATAAGGGCACT |
| | GGCATTTATTAAGAACTTAATAAATGTTAGCTTTTGTTATTTCACATTTTTC |
| | CTTGATCACTCCTACCAGGAATAAAATTCTGGGAGGGTATAAGTAGGTAGTG |
| | AAGTGCTAACTGGTCTGGTTAATTGTTAGAGTTCTGTTAAAAAAAAGTTATT |
| | TGAAAAAAGTATTTTGGAGCTAGGATCTAATTTATTAATATATCTGGATTTT |
| | CTTTTTCAATTTTGGTGTCCATTATTCACATAAGTAATTGTGGTTTTGCTAT |
| | ATTTTTTCCTCCTGAAAAATTATGGCTATACAACTAACTTTATTGTATACTG |
| | AATTTTGGAATTTTTTAGGATTTGATGTTCTTACTGGGGAGAGGATTTTGAA |
| | TTATTTAACCAC |
| 2037 | AACAAGAGGAAAGCATACAAATTTATTTAATACATGTTTTATGTGGCACAGG |
| | AGCCCTCATAAAGTAATAAAAAATCCCCAAACACAGTTAGAGCTGAACATTT |
| | ATATACTAATCTGGACAAAACATTTATATACTGCGTGGACAAAGAGCAGTAA |
| | ATTGTGAAAATGGAACAAGGCAAGGGGGCTTAGACTACAGTAGTTAATCATC |
| | AAGAAGTGACAAAAAAAAATAAGGGTTAGTTAATAAGATTTGTTTAAGCAGA |
| | TTTCTCCCAGCTTTAGCTCTCTGTCTCTGGTGATCAGAATGCACTCCTTCCT |
| | TCAGACTCAGTGAGCACATATTCCACACGGAAGATTTCTTCCCTAGCTTTTA |
| | GGAAATCCAGAGAACCCTTTTTGTATCTGTTGTTTTTTTTTTTTTTTAAATG |
| | TCTTGTCTTTAACTCAAAACAATTTATGTGCCAGGATGACATATCTTTGGAT |
| | AATGTGTTCTGAACTCCTTCAGTACATACGTATATAAATTAAAGCAAATATT |
| | TTTTATGATAAGCTGGCATAATAGTTTCATAATTTAATCACTGATTTAAAAA |
| | TTTAATTAAAATTATTTTTTAATATTTTGTGTAATAATTTTTGAGGAGTATC |
| | TTTTGTGCTTAATGAGTGGCAGATGACACCCATGTTCTTAGCAGCATCATTC |
| | ACAATAGCTAAAAGATAGGAACAACTGCGTATTGATGGATGAATGGATAAGC |
| | AAAATGAGGTATATACATATAAGGGAATATTCTTCATCCTTAAAAAGGAAGG |
| | AAATTCTGACATATGCTACAACAAGGTTGAACCTCTAAGGACATTATGCTAA |
| | ATGAAATAAACCAGTCTCAAAAAGACAAATACTATGTGATTCCAGATACATA |
| | AGGCACCTAGAGACAAACTGATAGAGACAGAAAGTAGAATGAGTGATTACCA |
| | GGGGTTGTGAGAGGAAAAAAGAGAGGGTTGTTTGATACAGAGTTTCAGTTTT |
| | GCAAGATAAAAG |
| 2038 | AACAGGAGAAAAGCGTACAAGTTTATTAAATAGAAGTTTTGCAGCCGGGCGC |
| | GCTGGCTCACGCTTGTAATCCTGGCACTTTGGGAGGCCGAGGCGGGCAGATC |
| | ACGAGGTCAGGAGATCGAGACCACGGTGAAACCCCGTCTCTACTAAAAATAC |
| | AACAAATTAGCCAGGCGTGGTAGCGAGGCAGGAGAATGGTGTGAACCCGGGA |
| | GGCGGAGCTTGCCTCTGCACTCCAGATCATGCCACTGCACTCCAGCCTGGGT |
| | GACAGACCAAGACTCCGTCTCAAAAAAAAAAAAAAAAAAAAAAAAAAAAAAG |
| | AAGTTTTGCATGACATGGGAACCCTCATAAAAAAAGTGAAGTCCCAAAAAAG |
| | TGGCAAAATCTAAATGCTTTTATATTATGTTGACGAAAGAGGGGCAATTGTG |
| | GAAAAGTAACTAAATTATGAGGGTTAGGCTAACAGAAGATAAAAATTATTTT |
| | AACAAGTTCTGTTTGTATAAAATTTTCTCAATTTCAGCTACCCATCCTTGAT |
| | GATTAGAATGTTGCATTCCTTCTGGTATACAAGGAACATCTTCCATATGGGG |
| | GTTTTATCTTCTGCTTTCAGAAAAAAAAAAATAACTCTGTGTGTGGTAGGAT |
| | GAAGGTGTCAGAACATTCTTCTTGCACCTGCTGGTTGCTATCTTTTTAAACT |
| | GCATTTGTTTCAAAACAATCCTTATGACAAAGGGGTGTATTTTGGGGTGGCA |
| | TATTCTGTCACCCGTCAATATCAAAGTGGTTTTTGAGTTTGTGCTCATCTTC |
| | TTTCCTTATTCTGTTCCTTGTAAGGTAAGACTAAATATAATGGAATTTGCCG |
| | TCACATGTCTCTTATATGTAGTGAGTTTTAACAGGCATTCCAGGAAATGCCA |
| | TATGGTCTTTTAGCTTGGAAATTATTTTAGAAAACGATAAAATCTTTAGTGT |
| | GAAGTTATTTCCCAGATATGTATCGCTAAAATTATCATTACAGGTGCTCTAG |
| | GTAATATGTTTG |
| 2039 | TTAGTACTTCCATCCCTTTCCTGGCTGCTCTAACTTTACAGGTACTTGTAAG |
| | TGGCAATTAAGCACTTTTTCCTAATTCCAGAGTCTTGCCCCACTTCAGAGCA |
| | ACATAGAGTGGCCTAGACAGGCTGAGGTACTTTGCCGCTTCAGTCATCATTA |
| | ATCTATGGTATTTACTGATGAGAAGTAAAGTGGTAGAAGAAAAAAAAATTTT |
| | CTGTTATCCTGGGCACTTGGAAATGAATGTATTCTCACAATCTGTTCTCAAA |
| | ACAACTTACTGATTCTGGGGTTCTGGAAGCTCTGATGTGCAGGTGAGCCTTT |
| | TAAATTCCTCACTGTTGGAGCTCCTATCTAGGACTCACTGGCTGGATGAAAA |
| | CGGTTCTTTTTATTGCTTTCTGAATGTCTGCTAGACAGGCGTAAGCAACACC |
| | TTATATCTGCCTTCTGAAAAAGGTAAAAGAACTGGGACCCATCCACCATGCT |
| | GGACAGCTCGGCAGTGGCAGTGGCCTCCCCCAGACCCTGTTCCGAGTGCTCC |
| | ACCAACAAACTCACCAGCAGTCAGAGTCTAGCCTCTCCCCAAACTTCACCTT |
| | CATCACAATTCATTTTAAGCCCTTCCACAACCCAATCAACTCTAGATCTACT |
| | TAATGGATAATAATTTGATCTCATGCAAACTGCACTTTCCTCTTCTCAGAAT |
| | GATCCTTCTACCCCTTAATTAAACATTTGAGAGTGAAAGAAGAGAAAATTCG |
| | GGTTCAAAGATTGGTAAGTCTAAGAAACCTAAGGAAAAGGAGTTAGTAAACA |
| | TGTTAATCAAAGAGTGAGCACTTTTCGGAAGCGCAACATTCAGATACCTTTC |
| | TTGATTGGATTCCAGAAGACTATTTCTGGGAAGAGGAGATTTGCATTTTTCT |
| | AAAGTCTTCTACCCACAGCCTAACCACCCTAGGGCTTTGAAATATTTTTTTT |
| | CTGATGTGCAGTCATAATTGAATAAATAAAATGATTCCTGATCATTTCTTCT |
| | CTTCAGCTTTAT |
| 2040 | TATTCCTGTATTTCTATTGTACTTTTTTGCATTAAGAAACATTTTCCAATGT |
| | AACATTTTAATAGATTTTTCACTATTTGTTGAGTTATTTTTGAGTGGTTGTA |
| | CTTGAGCTTGCCATCTATGTCTTAACTTCAGATTTGTACTAACTTAATTCCA |
| | GGGAGATATAGAAGCATTATTCCTACATAGCTCTATATCAACCCCCTTTTCC |
| | TGTGGTATTATTGTTATACAAGGTACACCATATATGTTACAAATACAATTAT |
| | TTATAGTTATAATTATTACTTTAAATATATCATTTATGTCTATTAAAGAAGC |
| | TGAGAGCAGAGAGGAGATAAAGTATATATTTATAGAATTTGTTATATTAAGC |
| | TTCTTATTTGTCATTCTGATTCTCTTTGTTCTGGTGGACTTGAGTAAATATG |
| | TGATGTTATTTCATTATGCACACACAGCTTTGCTCCTTGTCATTTTATTTAT |
| | GCTGTCTTTCTCAAGTATATTGCATTTAAATACATTATAGGACCAACAATTC |
| | AAATATATTTATGTTGTGTTATACAATTGCTTTTTAAAATCAGTTAAGATAG |
| | ATGGGATATGCACTGATAGTATGGTTTTTAAAATTATACTTTAAGTTCTGGG |
| | TTACATATGCAGAACATGCTGTTTGGTTACATAGGTATACACGTGCCATGGT |
| | GGTTTGCTGCACCCATCAACCCACCACCTACATTAGGTATTTCTCCTAATGT |
| | TATCTGTCCTCTGGCCTCCAACCCCCCGACATGCCCCAGTGTGTGATGTTCC |
| | CCTCCCTGTGTCCATGTGTTCTCATTGTTCAACTCCCACTTATGAGTGAGAA |
| | CATGTGGTGTTTGGTTTTCTGATCTTGTGATAGTTTCCTGAGAATGATGGTT |
| | TCCAGCTTCATCCATGTCCCTGAAAAAGATATGAACTCATCCTAGACAATAA |
| | TTCAAACACACACACACACACACACACACACACACACACACACACGCAAATG |
| | GCACTAGTATCT |
| 2041 | TCCAGAAAACATAACAATTCAGAACATATATTTAATCCCTCCTCAATCCAGA |
| | TCCTTGTTGAAACAATGAAAGAGTACAATATACTGCCATGAAAAGTACTGAG |
| | AAAAGTCTACAGATAGTGACATGGAAGAAAAGAAAAAATATTAAATAGATCA |
| | AACTAGTTATATAATTTGTATCTCATTTCTGTAAAATAAATTTAACATTTAT |
| | AAGTGTATTAGTTTGTTCTCACATTGCTATAATAAAATACCTGAGACTGGGT |
| | AATTAAAAAAAAAAACAGATTTAATTGGCACACAGTTCTATAGGCTGTACAG |
| | AGAAAACAGTGGCTTCTGCTTCTGGGGAGGTTTCAGGAAACTTCCAATCATG |
| | ATGGAAGCCGAAGGGGAAGCAGACACATCTTACGTGGCCAGAGCAGGAGCAC |
| | AAGTGTGAAGGGAAGTGTCTGTTCATATTCTTCACTCACTTTTTAATGGGGT |
| | TGTTTGTTTTTTTCTTAGAAATTTAAGTTCCTTGTAGATTCTGGATATTAGG |
| | CCTTTGTCAGATGGATAGATTGCAAAAATGTTCTCCCATTCTGCAGGTTGCC |
| | TGTTCACTTTGATGATAGTTTCTTTTGCTGAGCAGAAGCTCTTTAGTTTAAT |
| | TTTGCAGGGACATGGATGAAGCTGGAAACCATTATCTTCAGTAGACTAACTG |
| | TTAACAGGAACAGAAAACCAAAAACAAACAAAAGCATGAAGAGGGAAGTGTC |
| | ACCCACATGAGAACTCACTATTGTGATGACAACACCAAGGGGAATGGTGTTA |
| | AACCATGAGAACCGGCCCCCATGATCCAATCACTTCCCACCAGGCCCCACCT |
| | CCAATACTGGATATTACAATTCAACAAGAGATTTGGGCAGGAATACAGATCC |
| | AAACCATATCAGTAAATATAATAAATATATATTAATAAATATGTAAATATAT |
| | GTATGCAAGTTAACAAATGAACCAGTTGGTATGTAAGTATGTATATAAAGGA |
| | CCATAGCAGTTA |
| 2042 | CTGAATACTAGAGGAGCAAGTACAACAAATGGAAAATGGGATCAAGTATGAG |
| | TGAGAGTTGCTAAGATGCCTGGTAGGGATGCAAAGGGGTAGAGAGCCTGGGG |
| | AGAGAGGGTGAGGGAGGGAAGCACTGGTTTCTCAAGCAAAAGCTAAAATTTT |
| | TCTATTAAGATTTAACCTGATGCTACACTTTGGTGGTGCAGCAAGGGTCTCA |
| | AATGGTATAAAACTCAGGTGATCATGCTTTATGTCTGTCTCTAGAAAAATGC |
| | TCCAAAAATGATAAGTAGTGATAATCCGCAGTCTCGTTGCATAAAATCAGCC |
| | CCAGGTGAATGACTAAGCTCCATTTCCCTACCCCACCCTTATTACAATAACC |
| | TCGACACCAACTCTAGTCCGTGGGAAGATAAACTAATCGGAGTCGCCCCTCA |
| | AATCTTACAGCTGCTCACTCCCCTGCAGGGCAACGCCCAGGGACCAAGTTAG |
| | CCCCTTAAGCCTAGGCAAAAGAATCCCGCCCATAATCGAGAAGCGACTCGAC |
| | ATGGAGGCGATGACGAGATCACGCGAGGAGGAAAGGAGGGAGGGCTTCTTCC |
| | AGGCCCAGGGCGGTCCTTACAAGACGGGAGGCAGCAGAGAACTCCCATAAAG |
| | GTATTGCGGCACTCCCCTCCCCCTGCCCAGAAGGGTGCGGCCTTCTCTCCAC |
| | CTCCTCCACCGCAGCTCCCTCAGGATTGCAGCTCGCGCCGGTTTTTGGAGAA |
| | CAAGCGCCTCCCACCCACAAACCAGCCGGACCGACCCCCGCTCCTCCCCCAC |
| | CCCCACGAGTGCCTGTAGCAGGTCGGGCTTGTCTCGCCCTTCAGGCGGTGGG |
| | AACCCGGGGCGGAGCCGCGGCCGCCGCCATCCAGAAGTCTCGGCCGGCAGCC |
| | CGCCCCCGCCTCCAGCGCGCGCTTCCTGCCACGTTGCGCAGGGGCGCGGGGC |
| | CAGACACTGCGGCGCTCGGCCTCGGGGAGGACCGTACCAACGCCCGCCTCCC |
| | CGCCACCCCCGCGCCCCGCGCAGTGGTTTCGCTCATGTGAGACTCGAGCCAG |
| | TAGCA |
| 2043 | GCCCTGCCAGGACGGGGCTGGCTACTGGCCTTATCTCACAGGTAAAACTGAC |
| | GCACGGAGGAACAATATAAATTGGGGACTAGAAAGGTGAAGAGCCAAAGTTA |
| | GAACTCAGGACCAACTTATTCTGATTTTGTTTTTCCAAACTGCTTCTCCTCT |
| | TGGGAAGTGTAAGGAAGCTGCAGCACCAGGATCAGTGAAACGCACCAGACGG |
| | CCGCGTCAGAGCAGCTCAGGTTCTGGGAGAGGGTAGCGCAGGGTGGCCACTG |
| | AGAACCGGGCAGGTCACGCATCCCCCCCTTCCCTCCCACCCCCTGCCAAGCT |
| | CTCCCTCCCAGGATCCTCTCTGGCTCCATCGTAAGCAAACCTTAGAGGTTCT |
| | GGCAAGGAGAGAGATGGCTCCAGGAAATGGGGGTGTGTCACCAGATAAGGAA |
| | TCTGCCTAACAGGAGGTGGGGGTTAGACCCAATATCAGGAGACTAGGAAGGA |
| | GGAGGCCTAAGGATGGGGCTTTTCTGTCACCAATCCTGTCCCTAGTGGCCCC |
| | ACTGTGGGGTGGAGGGGACAGATAAAAGTACCCAGAACCAGAGCCACATTAA |
| | CCGGCCCTGGGAATATAAGGTGGTCCCAGCTCGGGGACACAGGATCCCTGGA |
| | GGCAGCAAACATGCTGTCCTGAAGTGGACATAGGGGCCCGGGTTGGAGGAAG |
| | AAGACTAGCTGAGCTCTCGGACCCCTGGAAGATGCCATGACAGGGGGCTGGA |
| | AGAGCTAGCACAGACTAGAGAGGTAAGGGGGGTAGGGGAGCTGCCCAAATGA |
| | AAGGAGTGAGAGGTGACCCGAATCCACAGGAGAACGGGGTGTCCAGGCAAAG |
| | AAAGCAAGAGGATGGAGAGGTGGCTAAAGCCAGGGAGACGGGGTACTTTGGG |
| | GTTGTCCAGAAAAACGGTGATGATGCAGGCCTACAAGAAGGGGAGGCGGGAC |
| | GCAAGGGAGACATCCGTCGGAGAAGGCCATCCTAAGAAACGAGAGATGGCAC |
| | AGGCCCCAGAAGGAGAAGGAAAAGGGAACCCA |

In certain cases, expression of an exogenous DNA, e.g., transgene, inserted in a target polynucleotide at or near a target nucleotide sequence may depend on cell type and differentiation stage, as one or more components of a target polynucleotide get activated during differentiation while others get silenced, which may or may not be correlated with rearrangements of the chromatin architecture reorganization during differentiation. To overcome this, in certain embodiments, additional to the exemplary characteristics described above, a suitable target polynucleotide comprising a target nucleotide sequence demonstrates suitable expression of an inserted exogenous DNA, e.g., transgene, throughout differentiation and clonal expansion.

### IV. PHARMACEUTICAL COMPOSITIONS

Provided herein is a composition (e.g., pharmaceutical composition) comprising a guide nucleic acid, an engineered, non-naturally occurring system, or a eukaryotic cell, such as a guide nucleic acid, an engineered, non-naturally occurring system, or a eukaryotic cell, disclosed herein. In certain embodiments, the composition comprises an RNP comprising a guide nucleic acid, such as a guide nucleic acid disclosed herein, and a Cas protein (e.g., Cas nuclease). In certain embodiments, the composition comprises a single guide nucleic acid, such as a single guide nucleic acid disclosed herein. In certain embodiments, the composition comprises an RNP comprising the single guide nucleic acid, and a Cas protein (e.g., Cas nuclease). In certain embodiments, the composition comprises an RNP comprising the targeter nucleic acid, the modulator nucleic acid, and a Cas protein (e.g., Cas nuclease). In certain embodiments, the composition comprises a complex of a targeter nucleic acid and a modulator nucleic acid, such as a complex of a targeter nucleic acid and a modulator nucleic acid disclosed herein. In certain embodiments, the composition comprises an RNP comprising the targeter nucleic acid, the modulator nucleic acid, and a Cas protein (e.g., Cas nuclease).

In certain embodiments provided herein is a method of producing a composition, the method comprising incubating a single guide nucleic acid, such as a single guide nucleic acid disclosed herein, with a Cas protein, thereby producing a complex of the single guide nucleic acid and the Cas protein (e.g., an RNP). In certain embodiments, the method further comprises purifying the complex (e.g., the RNP).

In certain embodiments, provided is a method of producing a composition, the method comprising incubating a targeter nucleic acid and a modulator nucleic acid, such as a targeter nucleic acid and a modulator nucleic acid disclosed herein, under suitable conditions, thereby producing a composition (e.g., pharmaceutical composition) comprising a complex of the targeter nucleic acid and the modulator nucleic acid. In certain embodiments, the method further comprises incubating the targeter nucleic acid and the modulator nucleic acid with a Cas protein (e.g., the Cas nuclease that the targeter nucleic acid and the modulator nucleic acid are capable of activating or a related Cas protein), thereby producing a complex of the targeter nucleic acid, the modulator nucleic acid, and the Cas protein (e.g., an RNP). In certain embodiments, the method further comprises purifying the complex (e.g., the RNP).

For therapeutic use, a guide nucleic acid, an engineered, non-naturally occurring system, a CRISPR expression system, or a cell comprising such system or modified by such system disclosed herein is combined with a pharmaceutically acceptable carrier. The term “pharmaceutically acceptable” as used herein can refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit-to-risk ratio.

The term “pharmaceutically acceptable carrier” as used herein includes buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA (1975). Pharmaceutically acceptable carriers include buffers, solvents, dispersion media, coatings, isotonic and absorption delaying agents, or the like, that are compatible with pharmaceutical administration. The use of such media and agents for pharmaceutically active substances is known in the art.

In certain embodiments, a pharmaceutical composition disclosed herein comprises a salt, e.g., NaCl, MgCl2, KCl, MgSO4, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), MES sodium salt, 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; or the like. For example, in certain embodiments, a subject composition comprises a subject DNA-targeting RNA, e.g., gRNA, and a buffer for stabilizing nucleic acids.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) Bioeng. Transl. Med. 1:10-29). In certain embodiment, the pharmaceutical composition comprises an inorganic nanoparticle. Exemplary inorganic nanoparticles include, e.g., magnetic nanoparticles (e.g., Fe3MnO2) or silica. The outer surface of the nanoparticle can be conjugated with a positively charged polymer (e.g., polyethylenimine, polylysine, polyserine) which allows for attachment (e.g., conjugation or entrapment) of payload. In certain embodiment, the pharmaceutical composition comprises an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating. In certain embodiment, the pharmaceutical composition comprises a liposome, for example, a liposome disclosed in International (PCT) Application Publication No. WO 2015/148863.

In certain embodiments, the pharmaceutical composition comprises a targeting moiety to increase target cell binding or update of nanoparticles and liposomes. Exemplary targeting moieties include cell specific antigens, monoclonal antibodies, single chain antibodies, aptamers, polymers, sugars, and cell penetrating peptides. In certain embodiments, the pharmaceutical composition comprises a fusogenic or endosome-destabilizing peptide or polymer.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly(2-hydroxyethyl-inethacrylate), ethylene vinyl acetate, or poly-D(−)-3-hydroxybutyric acid. Sustained release compositions may also include liposomes that can be prepared by any of several methods known in the art.

A pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound (e.g., the guide nucleic acid, engineered, non-naturally occurring system, or CRISPR expression system disclosed herein) may be coated in a material to protect the compound from the action of acids and other natural conditions that may inactivate the compound.

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution. In certain embodiments, the pharmaceutical composition is lyophilized, and then reconstituted in buffered saline, at the time of administration.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the guide nucleic acid, engineered, non-naturally occurring system, or CRISPR expression system disclosed herein is employed in the pharmaceutical compositions of the invention. The compositions disclosed herein are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for case of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions disclosed herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### V. THERAPEUTIC USES

Guide nucleic acids, engineered, non-naturally occurring systems, and the CRISPR expression systems, e.g., as disclosed herein, are useful for targeting, editing, and/or modifying the genomic DNA in a cell or organism. These guide nucleic acids and systems, as well as a cell comprising one of the systems or a cell whose genome has been modified by one of the systems, can be used to treat a disease or disorder in which modification of genetic or epigenetic information is desirable. Accordingly, provided herein is a method of treating a disease or disorder, the method comprising administering to a subject in need thereof a guide nucleic acid, a non-naturally occurring system, a CRISPR expression system, or a cell disclosed herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treatment”, “treating”, “treat”, “treated”, or the like, as used herein, can refer to obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e.g., in a human, and includes: (a) inhibiting the disease, i.e., arresting its development; and (b) relieving the disease, i.e., causing regression of the disease. It is understood that a disease or disorder may be identified by genetic methods and treated prior to manifestation of any medical symptom.

For minimization of toxicity and off-target effect, it can be important to control the concentration of the CRISPR-Cas system delivered. Optimal concentrations can be determined by testing different concentrations in a cellular, tissue, or non-human eukaryote animal model and using deep sequencing to analyze the extent of modification at potential off-target genomic loci. The concentration that gives the highest level of on-target modification while minimizing the level of off-target modification is generally selected for ex vivo or in vivo delivery.

It is understood that the guide nucleic acid, the engineered, non-naturally occurring system, and the CRISPR expression system disclosed herein can be used to treat any suitable disease or disorder that can be improved by the system in a cell.

For therapeutic purposes, certain methods disclosed herein is particularly suitable for editing or modifying a proliferating cell, such as a stem cell (e.g., a hematopoietic stem cell), a progenitor cell (e.g., a hematopoietic progenitor cell or a lymphoid progenitor cell), or a memory cell (e.g., a memory T cell). Given that such cell is delivered to a subject and will proliferate in vivo, tolerance to off-target events is low. Prior to delivery, however, it is possible to assess the on-target and off-target events, thereby selecting one or more colonies that have the desired edit or modification and lack any undesired edit or modification. Therefore, lower editing or modifying efficiency can be tolerated for such cell. The engineered, non-naturally occurring system of the present invention has the advantage of increasing or decreasing the efficiency of nucleic acid cleavage by, for example, adjusting the hybridization of dual guide nucleic acids. As a result, it can be used to minimize off-target events when creating genetically engineered proliferating cells.

In certain embodiments, the guide nucleic acid, the engineered, non-naturally occurring system, and/or the CRISPR expression system disclosed herein can be used to engineer an immune cell. Immune cells include but are not limited to lymphocytes (e.g., B lymphocytes or B cells, T lymphocytes or T cells, and natural killer cells), myeloid cells (e.g., monocytes, macrophages, eosinophils, mast cells, basophils, and granulocytes), and the stem and progenitor cells that can differentiate into these cell types (e.g., hematopoietic stem cells, hematopoietic progenitor cells, and lymphoid progenitor cells). The cells can include autologous cells derived from a subject to be treated, or alternatively allogenic cells derived from a donor.

In certain embodiments, the immune cell is a T cell, which can be, for example, a cultured T cell, a primary T cell, a T cell from a cultured T cell line (e.g., Jurkat, SupTi), or a T cell obtained from a mammal, for example, from a subject to be treated. If obtained from a mammal, the T cell can be obtained from numerous sources, including but not limited to blood, bone marrow, lymph node, the thymus, or other tissues or fluids. T cells can also be enriched or purified. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), tumor infiltrating lymphocytes (TILs), memory T cells (e.g., central memory T cells and effector memory T cells), regulatory T cells, naive T cells, or the like.

In certain embodiments, an immune cell, e.g., a T cell, is engineered to express an exogenous gene. For example, in certain embodiments, an engineered CRISPR system disclosed herein may catalyze DNA cleavage at the gene locus, allowing for site-specific integration of the exogenous gene at the gene locus by HDR.

In certain embodiments, an immune cell, e.g., a T cell, is engineered to express a chimeric antigen receptor (CAR), i.e., the T cell comprises an exogenous nucleotide sequence encoding a CAR. As used herein, the term “chimeric antigen receptor” or “CAR” includes any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T cell signaling molecules, e.g. a T cell costimulatory domain (e.g., from CD28, CD137, OX40, ICOS, or CD27) in tandem with a T cell triggering domain (e.g. from CD3° C.). A T cell expressing a chimeric antigen receptor is referred to as a CAR T cell. Exemplary CAR T cells include CD19 targeted CTL019 cells (see, Grupp et al. (2015) BLOOD, 126:4983), 19-282 cells (see, Park et al. (2015) J. CLIN. ONCOL., 33:7010), and KTE-C19 cells (see, Locke et al. (2015) BLOOD, 126:3991). Additional exemplary CAR T cells are described in U.S. Pat. Nos. 7,446,190, 8,399,645, 8,906,682, 9,181,527, 9,272,002, 9,266,960, 10,253,086, 10,640,569, and 10,808,035, and International (PCT) Publication Nos. WO 2013/142034, WO 2015/120180, WO 2015/188141, WO 2016/120220, and WO 2017/040945. Exemplary approaches to express CARs using CRISPR systems are described in Hale et al. (2017) MOL THER METHODS CLIN DEV., 4:192, MacLcod et al. (2017) MOL THER, 25:949, and Eyquem et al. (2017) NATURE, 543:113.

In certain embodiments, an immune cell, e.g., a T cell, binds an antigen, e.g., a cancer antigen, through an endogenous T cell receptor (TCR). In certain embodiments, an immune cell, e.g., a T cell, is engineered to express an exogenous TCR, e.g., an exogenous naturally occurring TCR or an exogenous engineered TCR. T cell receptors comprise two chains referred to as the α- and β-chains, that combine on the surface of a T cell to form a heterodimeric receptor that can recognize MHC-restricted antigens. Each of α- and β-chain comprises a constant region and a variable region. Each variable region of the α- and β-chains defines three loops, referred to as complementary determining regions (CDRs) known as CDR₁, CDR₂, and CDR₃ that confer the T cell receptor with antigen binding activity and binding specificity.

In certain embodiments, a CAR or TCR binds a cancer antigen selected from B-cell maturation antigen (BCMA), mesothelin, prostate specific membrane antigen (PSMA), prostate stem cell antigen (PSCA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD70, CD74, CD123, CD133, CD138, epithelial glycoprotein2 (EGP 2), epithelial glycoprotein-40 (EGP-40), epithelial cell adhesion molecule (EpCAM), receptor-type tyrosine-protein kinase (FLT3), folate-binding protein (FBP), fetal acetylcholine receptor (AChR), folate receptor-a and β (FRa and β), Ganglioside G2 (GD2), Ganglioside G3 (GD3), epidermal growth factor receptor 2 (HER-2/ERB2), epidermal growth factor receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1; e.g., a truncated MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), and Trail Receptor (TRAIL-R).

Genetic loci suitable for insertion of a CAR- or exogenous TCR-encoding sequence include but are not limited to safe harbor loci (e.g., the AAVS1 locus) TCR subunit loci (e.g., the TCRα constant (TRAC) locus, the TCRβ constant 1 (TRBC1) locus, and the TCRβ constant 2 (TRBC2) locus). It is understood that insertion in the TRAC locus reduces tonic CAR signaling and enhances T cell potency (see, Eyquem et al. (2017) NATURE, 543:113). Furthermore, inactivation of the endogenous TRAC, TRBC1, or TRBC2 gene may reduce a graft-versus-host disease (GVHD) response, thereby allowing use of allogeneic T cells as starting materials for preparation of CAR T cells. Accordingly, in certain embodiments, an immune cell, e.g., a T cell, is engineered to have reduced expression of an endogenous TCR or TCR subunit, e.g., TRAC, TRBC1, and/or TRBC2. The cell may be engineered to have partially reduced or no expression of the endogenous TCR or TCR subunit. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the endogenous TCR or TCR subunit relative to a corresponding unmodified or parental cell. In certain embodiments, the immune cell, e.g., a T cell, is engineered to have no detectable expression of the endogenous TCR or TCR subunit. Exemplary approaches to reduce expression of TCRs using CRISPR systems are described in U.S. Pat. No. 9,181,527, Liu et al. (2017) CELL RES, 27:154, Ren et al. (2017) CLIN CANCER RES, 23:2255, Cooper et al. (2018) LEUKEMIA, 32:1970, and Ren et al. (2017) ONCOTARGET, 8:17002.

It is understood that certain immune cells, such as T cells, also express major histocompatibility complex (MHC) or human leukocyte antigen (HLA) genes, and inactivation of these endogenous gene may reduce an immune response, thereby allowing use of allogeneic T cells as starting materials for preparation of CAR T cells. Accordingly, in certain embodiments, an immune cell, e.g., a T-cell, is engineered to have reduced expression of one or more endogenous class I or class II MHCs or HLAs (e.g., beta 2-microglobulin (B2M), class II major histocompatibility complex transactivator (CIITA)). The cell may be engineered to have partially reduced or no expression of an endogenous MHC or HLA. For example, in certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous MHC (e.g., B2M, CIITA) relative to a corresponding unmodified or parental cell. In certain embodiments, the immune cell, e.g., a T cell, is engineered to have no detectable expression of an endogenous MHC (e.g., B2M, CIITA). In certain cases, a cell may be engineered to have expression of, e.g., HLA-E and/or HLA-G, in order to avoid attack by natural killer (NK) cells. Exemplary approaches to reduce expression of MHCs using CRISPR systems are described in Liu et al. (2017) CELL RES, 27:154, Ren et al. (2017) CLIN CANCER RES, 23:2255, and Ren et al. (2017) ONCOTARGET, 8:17002.

Other genes that may be inactivated include but are not limited to CD3, CD52, and deoxycytidine kinase (DCK). For example, inactivation of DCK may render the immune cells (e.g., T cells) resistant to purine nucleotide analogue (PNA) compounds, which are often used to compromise the host immune system in order to reduce a GVHD response during an immune cell therapy. In certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous CD52 or DCK relative to a corresponding unmodified or parental cell.

It is understood that the activity of an immune cell (e.g., T cell) may be enhanced by inactivating or reducing the expression of an immune suppressor such as an immune checkpoint protein. Accordingly, in certain embodiments, an immune cell, e.g., a T cell, is engineered to have reduced expression of an immune checkpoint protein. Exemplary immune checkpoint proteins expressed by wild-type T cells include but are not limited to PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. The cell may be modified to have partially reduced or no expression of the immune checkpoint protein. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the immune checkpoint protein relative to a corresponding unmodified or parental cell. In certain embodiments, the immune cell, e.g., a T cell, is engineered to have no detectable expression of the immune checkpoint protein. Exemplary approaches to reduce expression of immune checkpoint proteins using CRISPR systems are described in International (PCT) Publication No. WO 2017/017184, Cooper et al. (2018) LEUKEMIA, 32:1970, Su et al. (2016) ONCOIMMUNOLOGY, 6: c1249558, and Zhang et al. (2017) FRONT MED, 11:554.

The immune cell can be engineered to have reduced expression of an endogenous gene, e.g., an endogenous genes described above, by gene editing or modification. For example, in certain embodiments, an engineered CRISPR system disclosed herein may result in DNA cleavage at a gene locus, thereby inactivating the targeted gene. In other embodiments, an engineered CRISPR system disclosed herein may be fused to an effector domain (e.g., a transcriptional repressor or histone methylase) to reduce the expression of the target gene.

The immune cell can also be engineered to express an exogenous protein (besides an antigen-binding protein described above) at the locus of a human ADORA2A, B2M, CD52, CIITA, CTLA4, DCK, FAS, HAVCR2, LAG3, PDCD1, PTPN6, TIGIT, TRAC, TRBC1, TRBC2, CARD11, CD247, IL7R, LCK, or PLCG1 gene.

In certain embodiments, an immune cell, e.g., a T cell, is modified to express a dominant-negative form of an immune checkpoint protein. In certain embodiments, the dominant-negative form of the checkpoint inhibitor can act as a decoy receptor to bind or otherwise sequester the natural ligand that would otherwise bind and activate the wild-type immune checkpoint protein. Examples of engineered immune cells, for example, T cells containing dominant-negative forms of an immune suppressor are described, for example, in International (PCT) Publication No. WO 2017/040945.

In certain embodiments, an immune cell, e.g., a T cell, is modified to express a gene (e.g., a transcription factor, a cytokine, or an enzyme) that regulates the survival, proliferation, activity, or differentiation (e.g., into a memory cell) of the immune cell. In certain embodiments, the immune cell is modified to express TET2, FOXO1, IL-12, IL-15, IL-18, IL-21, IL-7, GLUT1, GLUT3, HK1, HK2, GAPDH, LDHA, PDK1, PKM2, PFKFB3, PGK1, ENO1, GYS1, and/or ALDOA. In certain embodiments, the modification is an insertion of a nucleotide sequence encoding the protein operably linked to a regulatory element. In certain embodiments, the modification is a substitution of a single nucleotide polymorphism (SNP) site in the endogenous gene. In certain embodiments, an immune cell, e.g., a T cell, is modified to express a variant of a gene, for example, a variant that has greater activity than the respective wild-type gene. In certain embodiments, the immune cell is modified to express a variant of CARD11, CD247, IL7R, LCK, or PLCG1. For example, certain gain-of-function variants of IL7R were disclosed in Zenatti et al., (2011) NAT. GENET. 43 (10): 932-39. The variant can be expressed from the native locus of the respective wild-type gene by delivering an engineered system described herein for targeting the native locus in combination with a donor template that carries the variant or a portion thereof.

In certain embodiments, an immune cell, e.g., a T cell, is modified to express a protein (e.g., a cytokine or an enzyme) that regulates the microenvironment that the immune cell is designed to migrate to (e.g., a tumor microenvironment). In certain embodiments, the immune cell is modified to express CA9, CA12, a V-ATPase subunit, NHE1, and/or MCT-1.

### A. Gene Therapies

It is understood that the engineered, non-naturally occurring system and CRISPR expression system, e.g., as disclosed herein, can be used to treat a genetic disease or disorder, i.e., a disease or disorder associated with or otherwise mediated by an undesirable mutation in the genome of a subject.

Exemplary genetic diseases or disorders include age-related macular degeneration, adrenoleukodystrophy (ALD), Alagille syndrome, alpha-1-antitrypsin deficiency, argininemia, argininosuccinic aciduria, ataxia (e.g., Friedreich ataxia, spinocerebellar ataxias, ataxia telangiectasia, essential tremor, spastic paraplegia), autism, biliary atresia, biotinidase deficiency, carbamoyl phosphate synthetase I deficiency, carbohydrate deficient glycoprotein syndrome (CDGS), a central nervous system (CNS)-related disorder (e.g., Alzheimer's disease, amyotrophic lateral sclerosis (ALS), canavan disease (CD), ischemia, multiple sclerosis (MS), neuropathic pain, Parkinson's disease), Bloom's syndrome, cancer, Charcot-Marie-Tooth disease (e.g., peroncal muscular atrophy, hereditary motor sensory neuropathy), congenital hepatic porphyria, citrullinemia, Crigler-Najjar syndrome, cystic fibrosis (CF), Dentatorubro-Pallidoluysian Atrophy (DRPLA). diabetes insipidus, Fabry, familial hypercholesterolemia (LDL receptor defect), Fanconi's anemia, fragile X syndrome, a fatty acid oxidation disorder, galactosemia, glucose-6-phosphate dehydrogenase (G6PD), glycogen storage diseases (e.g., type I (glucose-6-phosphatase deficiency, Von Gierke II (alpha glucosidase deficiency, Pompe), III (debrancher enzyme deficiency, Cori), IV (brancher enzyme deficiency, Anderson), V (muscle glycogen phosphorylase deficiency, McArdle), VII (muscle phosphofructokinase deficiency, Tauri), VI (liver phosphorylase deficiency, Hers), IX (liver glycogen phosphorylase kinase deficiency)), hemophilia A (associated with defective factor VIII), hemophilia B (associated with defective factor IX), Huntington's disease, glutaric aciduria, hypophosphatemia, Krabbe, lactic acidosis, Lafora disease, Leber's Congenital Amaurosis, Lesch Nyhan syndrome, a lysosomal storage disease, metachromatic leukodystrophy disease (MLD), mucopolysaccharidosis (MPS) (e.g., Hunter syndrome, Hurler syndrome, Maroteaux-Lamy syndrome, Sanfilippo syndrome, Scheie syndrome, Morquio syndrome, other, MPSI, MPSII, MPSIII, MSIV, MPS 7), a muscular/skeletal disorder (e.g., muscular dystrophy, Duchenne muscular dystrophy), myotonic Dystrophy (DM), neoplasia, N-acetylglutamate synthase deficiency, ornithine transcarbamylase deficiency, phenylketonuria, primary open angle glaucoma, retinitis pigmentosa, schizophrenia, Severe Combined Immune Deficiency (SCID), Spinobulbar Muscular Atrophy (SBMA), sickle cell anemia, Usher syndrome, Tay-Sachs disease, thalassemia (e.g., B-Thalassemia), trinucleotide repeat disorders, tyrosinemia, Wilson's disease, Wiskott-Aldrich syndrome, X-linked chronic granulomatous disease (CGD), X-linked severe combined immune deficiency, and xeroderma pigmentosum.

Additional exemplary genetic diseases or disorders and associated information are available on the world wide web at kumc.cdu/gec/support, genome.gov/10001200, and ncbi.nlm.nih.gov/books/NBK22183/. Additional exemplary genetic diseases or disorders, associated genetic mutations, and gene therapy approaches to treat genetic diseases or disorders are described in International (PCT) Publication Nos. WO 2013/126794, WO 2013/163628, WO 2015/048577, WO 2015/070083, WO 2015/089354, WO 2015/134812, WO 2015/138510, WO 2015/148670, WO 2015/148860, WO 2015/148863, WO 2015/153780, WO 2015/153789, and WO 2015/153791, U.S. Pat. Nos. 8,383,604, 8,859,597, 8,956,828, 9,255,130, and 9,273,296, and U.S. Patent Application Publication Nos. 2009/0222937, 2009/0271881, 2010/0229252, 2010/0311124, 2011/0016540, 2011/0023139, 2011/0023144, 2011/0023145, 2011/0023146, 2011/0023153, 2011/0091441, 2012/0159653, and 2013/0145487.

### B. Immune Cell Engineering

It is understood that the engineered, non-naturally occurring systems comprising ssODNs disclosed herein can be used to engineer an immune cell. Immune cells include but are not limited to lymphocytes (e.g., B lymphocytes or B cells, T lymphocytes or T cells, and natural killer cells), myeloid cells (e.g., monocytes, macrophages, eosinophils, mast cells, basophils, and granulocytes), and the stem and progenitor cells that can differentiate into these cell types (e.g., hematopoietic stem cells, hematopoietic progenitor cells, and lymphoid progenitor cells). The cells can include autologous cells derived from a subject to be treated, or alternatively allogenic cells derived from a donor.

It is understood that CRISPR systems comprising ssODNs disclosed herein can be used to treat any disease or disorder that can be improved by editing or modifying a target sequence; exemplary genes containing target sequences to be modified for therapeutic purposes include ADORA2A, ALPNR, B2M, BBS1, CALR, CARD11, CD3E, CD3G, CD38, CD40LG, CD52, CD58, CD247, CIITA, COL17A1, CSF1R, CSF2, CTLA4, DCK, DEFB134, DHODH, ERAP1, ERAP2, FAS, mir-101-2, HAVCR2 (also called TIM3), IFNGR1, IFNGR2, IL7R, JAK1, JAK2, LAG3, LCK, LCK1, MLANA, MVD, PDCD1 (also called PD-1), PLCG1, PLK1, PSMB5, PSMB8, PSMB9, PTCD2, PTPN1, PTPN6, PTPN11, RFX5, RFXAP, RPL23, RXANK, SOX10, SRP54, STAT1, Tap1, TAP2, TAPBP, TGFBR2, TIGIT, TIM3, TRAC, TRBC1, TRBC1+2, TRBC2, TUBB, TWF1, and/or U6 gene in a cell.

In certain embodiments, the immune cell is a T cell, which can be, for example, a cultured T cell, a primary T cell, a T cell from a cultured T cell line (e.g., Jurkat, SupTi), or a T cell obtained from a mammal, for example, from a subject to be treated. If obtained from a mammal, the T cell can be obtained from numerous sources, including but not limited to blood, bone marrow, lymph node, the thymus, or other tissues or fluids. T cells can also be enriched or purified. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), tumor infiltrating lymphocytes (TILs), memory T cells (e.g., central memory T cells and effector memory T cells), regulatory T cells, naive T cells, and the like.

In certain embodiments, an immune cell, e.g., a T cell, is engineered to express an exogenous gene. For example, in certain embodiments, an engineered CRISPR system disclosed herein may be used to engineer an immune cell to express an exogenous gene. For example, in certain embodiments, the guide nucleic acid, the engineered, non-naturally occurring system, and the CRISPR expression system disclosed herein may be used to engineer an immune cell to express an exogenous gene at the locus of a human ADORA2A, ALPNR, B2M, BBS1, CALR, CARD11, CD3E, CD3G, CD38, CD40LG, CD52, CD58, CD247, CIITA, COL17A1, CSF1R, CSF2, CTLA4, DCK, DEFB 134, DHODH, ERAP1, ERAP2, FAS, mir-101-2, HAVCR2 (also called TIM3), IFNGR1, IFNGR2, IL7R, JAK1, JAK2, LAG3, LCK, LCK1, MLANA, MVD, PDCD1 (also called PD-1), PLCG1, PLK1, PSMB5, PSMB8, PSMB9, PTCD2, PTPN1, PTPN6, PTPN11, RFX5, RFXAP, RPL23, RXANK, SOX10, SRP54, STAT1, Tap1, TAP2, TAPBP, TGFBR2, TIGIT, TIM3, TRAC, TRBC1, TRBC1+2, TRBC2, TUBB, TWF1, and/or U6 gene.

For example, in certain embodiments, an engineered CRISPR system comprising ssODNs disclosed herein may catalyze DNA cleavage at a gene locus, allowing for site-specific integration of the exogenous gene at the gene locus by HDR, while decreasing off-target effects by incorporating wild-type gene back into off-target cleaved sites by HDR.

In certain embodiments, an immune cell, e.g., a T cell, is engineered to express a chimeric antigen receptor (CAR), i.e., the T cell comprises an exogenous nucleotide sequence encoding a CAR. As used herein, the term “chimeric antigen receptor” or “CAR” includes any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T cell signaling molecules, e.g. a T cell costimulatory domain (e.g., from CD28, CD137, OX40, ICOS, or CD27) in tandem with a T cell triggering domain (e.g. from CD3≈i,àç). A T cell expressing a chimeric antigen receptor is referred to as a CAR T cell. Exemplary CAR T cells include CD19 targeted CTL019 cells (see, Grupp et al. (2015) BLOOD, 126:4983), 19-28z cells (see, Park et al. (2015) J. CLIN. ONCOL., 33:7010), and KTE-C19 cells (see, Locke et al. (2015) BLOOD, 126:3991). Additional exemplary CAR T cells are described in U.S. Pat. Nos. 8,399,645, 8,906,682, 7,446,190, 9,181,527, 9,272,002, and 9,266,960, U.S. Patent Publication Nos. 2016/0362472, 2016/0200824, and 2016/0311917, and International (PCT) Publication Nos. WO2013/142034, WO2015/120180, WO2015/188141, WO2016/120220, and WO2017/040945. Exemplary approaches to express CARs using CRISPR systems are described in Hale et al. (2017) MOL THER METHODS CLIN DEV., 4:192, MacLcod et al. (2017) MOL THER, 25:949, and Eyquem et al. (2017) NATURE, 543:113.

In certain embodiments, an immune cell, e.g., a T cell, binds an antigen, e.g., a cancer antigen, through an endogenous T cell receptor (TCR). In certain embodiments, an immune cell, e.g., a T cell, is engineered to express an exogenous TCR, e.g., an exogenous naturally occurring TCR or an exogenous engineered TCR. T cell receptors comprise two chains referred to as the (E±- and (E≤-chains, that combine on the surface of a T cell to form a heterodimeric receptor that can recognize MHC-restricted antigens. Each of (E±- and (E≤-chain comprises a constant region and a variable region. Each variable region of the (E±- and (E≤-chains defines three loops, referred to as complementary determining regions (CDRs) known as CDR1, CDR2, and CDR3 that confer the T cell receptor with antigen binding activity and binding specificity.

In certain embodiments, a CAR or TCR binds a cancer antigen selected from B-cell maturation antigen (BCMA), mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD70, CD74, CD123, CD133, CD138, epithelial glycoprotein2 (EGP 2), epithelial glycoprotein-40 (EGP-40), epithelial cell adhesion molecule (EpCAM), receptor-type tyrosine-protein kinase (FLT3), folate-binding protein (FBP), fetal acetylcholine receptor (AChR), folate receptor-(E± and (E≤(FR(E± and (E≤), Ganglioside G2 (GD2), Ganglioside G3 (GD3), epidermal growth factor receptor 2 (HER-2/ERB2), epidermal growth factor receptor vIII (EGFRvIII), ERB3, ERB4, human telomerase reverse transcriptase (hTERT), Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1; e.g., a truncated MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), and Trail Receptor (TRAIL-R).

Genetic loci suitable for insertion of a CAR- or exogenous TCR-encoding sequence include but are not limited to safe harbor loci (e.g., the AAVS1 locus), TCR subunit loci (e.g., the TCR(E± constant (TRAC) locus), and other loci associated with certain advantages (e.g., the CCR5 locus, the inactivation of which may prevent or reduce HIV infection). It is understood that insertion in the TRAC locus reduces tonic CAR signaling and enhances T cell potency (see, Eyquem et al. (2017) NATURE, 543:113). Furthermore, inactivation of the endogenous TRAC gene may reduce a graft-versus-host disease (GVHD) response, thereby allowing use of allogeneic T cells as starting materials for preparation of CAR-T cells. Accordingly, in certain embodiments, an immune cell, e.g., a T cell, is engineered to have reduced expression of an endogenous TCR or TCR subunit, e.g., TCR(E± subunit constant (TRAC). The cell may be engineered to have partially reduced or no expression of the endogenous TCR or TCR subunit. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the endogenous TCR or TCR subunit relative to a corresponding unmodified or parental cell. In certain embodiments, the immune cell, e.g., a T cell, is engineered to have no detectable expression of the endogenous TCR or TCR subunit. Exemplary approaches to reduce expression of TCRs using CRISPR systems are described in U.S. Pat. No. 9,181,527, Liu et al. (2017) CELL RES, 27:154, Ren et al. (2017) CLIN CANCER RES, 23:2255, Cooper et al. (2018) LEUKEMIA, 32:1970, and Ren et al. (2017) ONCOTARGET, 8:17002.

It is understood that certain immune cells, such as T cells, also express major histocompatibility complex (MHC) or human leukocyte antigen (HLA) genes, and inactivation of these endogenous gene may reduce a GVHD response, thereby allowing use of allogeneic T cells as starting materials for preparation of CAR-T cells. Accordingly, in certain embodiments, an immune cell, e.g., a T-cell, is engineered to have reduced expression of one or more endogenous class I or class II MHCs or HLAs (e.g., beta 2-microglobulin (B2M), class II major histocompatibility complex transactivator (CIITA), HLA-E, and/or HLA-G). The cell may be engineered to have partially reduced or no expression of an endogenous MHC or HLA. For example, in certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous MHC (e.g., B2M, CIITA, HLA-E, or HLA-G) relative to a corresponding unmodified or parental cell. In certain embodiments, the immune cell, e.g., a T cell, is engineered to have no detectable expression of an endogenous MHC (e.g., B2M, CIITA, HLA-E, or HLA-G). Exemplary approaches to reduce expression of MHCs using CRISPR systems are described in Liu et al. (2017) CELL RES, 27:154, Ren et al. (2017) CLIN CANCER RES, 23:2255, and Ren et al. (2017) ONCOTARGET, 8:17002.

Other genes that may be inactivated to reduce a GVHD response include but are not limited to CD3, CD52, and deoxycytidine kinase (DCK). For example, inactivation of DCK may render the immune cells (e.g., T cells) resistant to purine nucleotide analogue (PNA) compounds, which are often used to compromise the host immune system in order to reduce a GVHD response during an immune cell therapy. In certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous CD52 or DCK relative to a corresponding unmodified or parental cell.

In certain embodiments, an immune cell, e.g., a T cell, is engineered to have reduced expression of an endogenous gene. For example, in certain embodiments, an engineered CRISPR system disclosed herein may be used to engineer an immune cell to have reduced expression of an endogenous gene. For example, in certain embodiments, an engineered CRISPR system disclosed herein may result in DNA cleavage at a gene locus, thereby inactivating the targeted gene. In other embodiments, an engineered CRISPR system disclosed herein may be fused to an effector domain (e.g., a transcriptional repressor or histone methylase) to reduce the expression of the target gene.

It is understood that the activity of an immune cell (e.g., T cell) may be enhanced by inactivating or reducing the expression of an immune suppressor such as an immune checkpoint protein. Accordingly, in certain embodiments, an immune cell, e.g., a T cell, is engineered to have reduced expression of an immune checkpoint protein. Exemplary immune checkpoint proteins expressed by wild-type T cells include but are not limited to PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. The cell may be modified to have partially reduced or no expression of the immune checkpoint protein. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the immune checkpoint protein relative to a corresponding unmodified or parental cell. In certain embodiments, the immune cell, e.g., a T cell, is engineered to have no detectable expression of the immune checkpoint protein. Exemplary approaches to reduce expression of immune checkpoint proteins using CRISPR systems are described in International (PCT) Publication No. WO2017/017184, Cooper et al. (2018) LEUKEMIA, 32:1970, Su et al. (2016) ONCOIMMUNOLOGY, 6: e1249558, and Zhang et al. (2017) FRONT MED, 11:554.

The immune cell can also be engineered to express an exogenous protein (besides an antigen-binding protein described above) at the locus of a human ADORA2A, ALPNR, B2M, BBS1, CALR, CARD11, CD3E, CD3G, CD38, CD40LG, CD52, CD58, CD247, CIITA, COL17A1, CSF1R, CSF2, CTLA4, DCK, DEFB134, DHODH, ERAP1, ERAP2, FAS, mir-101-2, HAVCR2 (also called TIM3), IFNGR1, IFNGR2, IL7R, JAK1, JAK2, LAG3, LCK, LCK1, MLANA, MVD, PDCD1 (also called PD-1), PLCG1, PLK1, PSMB5, PSMB8, PSMB9, PTCD2, PTPN1, PTPN6, PTPN11, RFX5, RFXAP, RPL23, RXANK, SOX10, SRP54, STAT1, Tap1, TAP2, TAPBP, TGFBR2, TIGIT, TIM3, TRAC, TRBC1, TRBC1+2, TRBC2, TUBB, TWF1, and/or U6 gene.

In certain embodiments, an immune cell, e.g., a T cell, is modified to express a dominant-negative form of an immune checkpoint protein. In certain embodiments, the dominant-negative form of the checkpoint inhibitor can act as a decoy receptor to bind or otherwise sequester the natural ligand that would otherwise bind and activate the wild-type immune checkpoint protein. Examples of engineered immune cells, for example, T cells containing dominant-negative forms of an immune suppressor are described, for example, in International (PCT) Publication No. WO2017/040945.

In certain embodiments, an immune cell, e.g., a T cell, is modified to express a gene (e.g., a transcription factor, a cytokine, or an enzyme) that regulates the survival, proliferation, activity, or differentiation (e.g., into a memory cell) of the immune cell. In certain embodiments, the immune cell is modified to express TET2, FOXO1, IL-12, IL-15, IL-18, IL-21, IL-7, GLUT1, GLUT3, HK1, HK2, GAPDH, LDHA, PDK1, PKM2, PFKFB3, PGK1, ENO1, GYS1, and/or ALDOA. In certain embodiments, the modification is an insertion of a nucleotide sequence encoding the protein operably linked to a regulatory element. In certain embodiments, the modification is a substitution of a single nucleotide polymorphism (SNP) site in the endogenous gene. In certain embodiments, an immune cell, e.g., a T cell, is modified to express a variant of a gene, for example, a variant that has greater activity than the respective wild-type gene. In certain embodiments, the immune cell is modified to express a variant of CARD11, CD247, IL7R, LCK, or PLCG1. For example, certain gain-of-function variants of IL7R were disclosed in Zenatti et al., (2011) NAT. GENET. 43(10): 932-39. The variant can be expressed from the native locus of the respective wild-type gene by delivering an engineered system described herein for targeting the native locus in combination with a donor template that carries the variant or a portion thereof.

In certain embodiments, an immune cell, e.g., a T cell, is modified to express a protein (e.g., a cytokine or an enzyme) that regulates the microenvironment that the immune cell is designed to migrate to (e.g., a tumor microenvironment). In certain embodiments, the immune cell is modified to express CA9, CA12, a V-ATPase subunit, NHE1, and/or MCT-1.

In certain embodiments, provided is a method for treatment of a disease, e.g., a cancer, by administering to a subject suffering from the disease an effective amount of T cells modified to express a CAR specific to the disease using the modified guide nucleic acids and CRISPR-Cas systems described herein, e.g., in sections IA, IAI, IB, IC, and IVB. In certain embodiments, the T cells are autologous cells removed from the subject, treated to modify genomic DNA to express CAR, expanded, and administered to the subject; in certain embodiments, the T cells are allogeneic T cells that have been treated to modify genomic DNA to express CAR. In certain embodiments, the disease is a blood cancer, such as leukemia or lymphoma; in certain embodiments the disease is a solid tumor cancer.

### VI. KITS

It is understood that the guide nucleic acid, the engineered, non-naturally occurring system, the CRISPR expression system, and/or a library disclosed herein can be packaged in a kit suitable for use by a medical provider. Accordingly, in another aspect, the invention provides kits containing any one or more of the elements disclosed in the above systems, libraries, methods, and compositions. In certain embodiments, the kit comprises an engineered, non-naturally occurring system as disclosed herein and instructions for using the kit. The instructions may be specific to the applications and methods described herein. In certain embodiments, one or more of the elements of the system are provided in a solution. In certain embodiments, one or more of the elements of the system are provided in lyophilized form, and the kit further comprises a diluent. Elements may be provided individually or in combinations, and may be provided in any suitable container, such as a vial, a bottle, a tube, or immobilized on the surface of a solid base (e.g., chip or microarray). In certain embodiments, the kit comprises one or more of the nucleic acids and/or proteins described herein. In certain embodiments, the kit provides all elements of the systems of the invention.

In certain embodiments of a kit comprising the engineered, non-naturally occurring dual guide system, the targeter nucleic acid and the modulator nucleic acid are provided in separate containers. In other embodiments, the targeter nucleic acid and the modulator nucleic acid are pre-complexed, and the complex is provided in a single container.

In certain embodiments, the kit comprises a Cas protein or a nucleic acid comprising a regulatory element operably linked to a nucleic acid encoding a Cas protein provided in a separate container. In other embodiments, the kit comprises a Cas protein pre-complexed with the single guide nucleic acid or a combination of the targeter nucleic acid and the modulator nucleic acid, and the complex is provided in a single container.

In certain embodiments, the kit further comprises one or more donor templates provided in one or more separate containers. In certain embodiments, the kit comprises a plurality of donor templates as disclosed herein (e.g., in separate tubes or immobilized on the surface of a solid base such as a chip or a microarray), one or more guide nucleic acids disclosed herein, and optionally a Cas protein or a regulatory element operably linked to a nucleic acid encoding a Cas protein as disclosed herein. Such kits are useful for identifying a donor template that introduces optimal genetic modification in a multiplex assay. The CRISPR expression systems as disclosed herein are also suitable for use in a kit.

In certain embodiments, a kit further comprises one or more reagents and/or buffers for use in a process utilizing one or more of the elements described herein. Reagents may be provided in any suitable container and may be provided in a form that is usable in a particular assay, or in a form that requires addition of one or more other components before use (e.g., in concentrate or lyophilized form). A buffer may be a reaction or storage buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In certain embodiments, the buffer has a pH from about 7 to about 10. In certain embodiments, the kit further comprises a pharmaceutically acceptable carrier. In certain embodiments, the kit further comprises one or more devices or other materials for administration to a subject.

### VII. EMBODIMENTS

In embodiment 1 provided herein is a composition comprising: (A) a double-stranded DNA polynucleotide; and (B) a polypeptide comprising a nuclear localization signal (NLS) bound to the polynucleotide.

In embodiment 2 provided herein is the composition of embodiment 1, wherein the double-stranded DNA polynucleotide comprises a plasmid.

In embodiment 3 provided herein is the composition of embodiment 1, wherein the double-stranded DNA polynucleotide comprises a linear double-stranded DNA polynucleotide with covalently closed ends.

In embodiment 4 provided herein is the composition of any one of embodiments 1 through 3, wherein the double-stranded DNA polynucleotide comprises a donor template (D).

In embodiment 5 provided herein is the composition of any one of embodiments 1 through 4, wherein the polypeptide comprises a nucleic acid-guided nuclease complex comprising: (1) a nucleic acid-guided nuclease; and (2) a guide nucleic acid (gNA).

In embodiment 6 provided herein is the composition of embodiment 5, wherein the double-stranded DNA polynucleotide comprises a first PAM (P1) recognized by a nucleic acid-guided nuclease and a first target nucleotide sequence (T1) adjacent to but not within the donor template (D).

In embodiment 7 provided herein is the composition of embodiment 6, wherein the double-stranded DNA polynucleotide further comprises a second PAM (P2) recognized by a nucleic acid-guided nuclease and a second target nucleotide sequence (T2) adjacent to but not within the donor template (D).

In embodiment 8 provided herein is the composition of embodiment 6 or 7, wherein the first PAM and the first target nucleotide sequence are oriented 5′ P1+T1+D+ 3′.

In embodiment 9 provided herein is the composition of embodiment 6 or 7, wherein the first PAM and the first target nucleotide sequence are oriented 5′ T1-P1-D+ 3′.

In embodiment 10 provided herein is the composition of any one of embodiments 7 through 9, wherein the second PAM and the second target nucleotide sequence are oriented 5′ D+P2+T2+ 3′.

In embodiment 11 provided herein is the composition of any one of embodiments 7 through 9, wherein the second suitable PAM and the second target nucleotide sequence are oriented 5′ D+T2−P2− 3′.

In embodiment 12 provided herein is the composition of embodiment 7, wherein the first and second PAM target nucleotide sequences are oriented 5′ T1−P1−D+P2+T2+ 3′.

In embodiment 13 provided herein is the composition of embodiment 7, wherein the first and second PAM target nucleotide sequences are oriented 5′ P1+T1+D+T2−P2− 3′.

In embodiment 14 provided herein is the composition of embodiment 5, wherein the nucleic acid-guided nuclease comprises an engineered, non-naturally occurring nuclease.

In embodiment 15 provided herein is the composition of any one of embodiments 5 through 14, wherein the nucleic acid-guided nuclease complex comprises a Class 1 or a Class 2 nucleic acid-guided nuclease complex.

In embodiment 16 provided herein is the composition of embodiment 15, wherein the nucleic acid-guided nuclease complex comprises a Type II or a Type V nucleic acid-guided nuclease.

In embodiment 17 provided herein is the composition of embodiment 16, wherein the nucleic acid-guided nuclease comprises a Type V-A, V-B, V-C, V-D, or V-E nucleic acid-guided nuclease.

In embodiment 18 provided herein is the composition of embodiment 17, wherein the nucleic acid-guided nuclease comprises a Type V-A nucleic acid-guided nuclease.

In embodiment 19 provided herein is the composition of embodiment 18, nucleic acid-guided nuclease comprises a MAD nuclease, an ART nuclease, or an ABW nucleic acid-guided nuclease.

In embodiment 20 provided herein is the composition of embodiment 19, wherein the nucleic acid-guided nuclease comprises an amino acid sequence at least 80, 85, 90, 95, 99, or 100% identical to an amino acid sequence of a MAD, ART, or ABW nucleic acid-guided nuclease.

In embodiment 21 provided herein is the composition of embodiment 19, wherein the nucleic acid-guided nuclease comprises MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20.

In embodiment 22 provided herein is the composition of embodiment 19, wherein the nucleic acid-guided nuclease comprises ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35.

In embodiment 23 provided herein is the composition of embodiment 19, wherein the nucleic acid-guided nuclease comprises an amino acid sequence at least 80, 85, 90, 95, 99, or 100% identical to the amino acid sequence of MAD2, MAD7, ART2, ART11, or ART11*.

In embodiment 24 provided herein is the composition of embodiment 19, wherein the nucleic acid-guided nuclease comprises an amino acid sequence that is at least 80, 85, 90, 95, 99, or 100% identical to the amino acid sequence of SEQ ID NO: 37.

In embodiment 25 provided herein is the composition of any one of embodiments 6 through 24, wherein the first PAM (P1) is a PAM recognized by a Type V nucleic acid-guided nuclease.

In embodiment 26 provided herein is the composition of embodiment 25, wherein the PAM comprises a sequence of CTTN.

In embodiment 27 provided herein is the composition of any one of embodiments 7 through 26, wherein the second PAM (P2) is a PAM recognized by a Type V nucleic acid-guided nuclease.

In embodiment 28 provided herein is the composition of any one of embodiments 5 through 27, wherein the gNA is an engineering, non-naturally occurring gNA.

In embodiment 29 provided herein is the composition of any one of embodiments 5 through 28, wherein the gNA comprises a single polynucleotide.

In embodiment 30 provided herein is the composition of any one of embodiments 5 through 28, wherein the gNA comprises a dual gNA comprising a targeter nucleic acid and a modulator nucleic acid, wherein the targeter nucleic acid and the modulator nucleic acid are separate polynucleotides.

In embodiment 31 provided herein is the composition of embodiment 30, wherein the dual gNA is capable of binding to and activating a nucleic acid-guided nuclease, that, in a naturally occurring system, is activated by a single crRNA in the absence of a tracrRNA.

In embodiment 32 provided herein is the composition of any one of embodiments 28 through 31, wherein the gNA comprises a heterologous spacer sequence that shares complementarity with a first target sequence in the double-stranded DNA polynucleotide and a second target sequence in a human genome.

In embodiment 33 provided herein is the composition of any one of embodiments 28 through 31, wherein the gNA comprises a heterologous spacer sequence that does not share complementarity to a target sequence in a human genome.

In embodiment 34 provided herein is the composition of any one of embodiments 1 through 31, wherein the nucleic acid-guided nuclease comprises at least 4 NLS.

In embodiment 35 provided herein is the composition of embodiment 34, wherein the nucleic acid-guided nuclease comprises one N-terminal and three C-terminal NLS.

In embodiment 36 provided herein is the composition of embodiment 34, wherein the nucleic acid-guided nuclease comprises five or more N-terminal NLS.

In embodiment 37 provided herein is the composition of any one of embodiments 1 through 35, wherein the NLS comprise any one of SEQ ID NOs: 40-56.

In embodiment 38 provided herein is the composition of embodiment 37, wherein the NLS comprise SEQ ID NOs: 40, 51, and 56.

In embodiment 39 provided herein is the composition of any one of embodiments 1 through 38, further comprising at least one of (1) a buffer; (2) a RNP stabilizer.

In embodiment 40 provided herein is the composition of embodiment 39, wherein the buffer is magnesium deficient.

In embodiment 41 provided herein is the composition of embodiment 39 or 40, wherein the RNP stabilizer comprises a peptide, poly-L-glutamic acid (PGA), or a single-stranded oligodeoxynucleotide (ssODN).

In embodiment 42 provided herein is a composition comprising a polynucleotide comprising: (A) a donor template (D); and (B) a first PAM (P1) recognized by a Type V nucleic acid-guided nuclease and a first target nucleotide sequence (T1) adjacent to but not within the donor template (D).

In embodiment 43 provided herein is the composition of embodiment 42, wherein the polynucleotide comprises double-stranded DNA.

In embodiment 44 provided herein is the composition of embodiment 42 or 43, wherein the polynucleotide comprises circular DNA.

In embodiment 45 provided herein is the composition of any one of embodiments 42 through 44, wherein the polynucleotide is a plasmid.

In embodiment 46 provided herein is the composition of any one of embodiments 42 through 45, wherein the polynucleotide further comprises a second PAM (P2) recognized by a Type V nucleic acid-guided nuclease and a second target nucleotide sequence (T2) adjacent to but not within the donor template (D).

In embodiment 47 provided herein is the composition of any one of embodiments 42 through 46, wherein the first PAM and the first target nucleotide sequence are oriented 5′ P1+T1+D+ 3′.

In embodiment 48 provided herein is the composition of any one of embodiments 42 through 46, wherein the first PAM and the first target nucleotide sequence are oriented 5′ T1−P1−D+ 3′.

In embodiment 49 provided herein is the composition of any one of embodiments 46 through 48, wherein the second PAM and the second target nucleotide sequence are oriented 5′ D+P2+T2+ 3′.

In embodiment 50 provided herein is the composition of any one of embodiments 46 through 48, wherein the second suitable PAM and the second target nucleotide sequence are oriented 5′ D+T2−P2− 3′.

In embodiment 51 provided herein is the composition of embodiment 46, wherein the first and second PAM target nucleotide sequences are oriented 5′ T1−P1−D+P2+T₂+ 3′.

In embodiment 52 provided herein is the composition of any one of embodiments 42 through 51, wherein the polynucleotide further comprises a selectable marker and/or a replication origin.

In embodiment 53 provided herein is the composition of any one of embodiments 1 through 52, wherein the donor template comprises a first sequence encoding a first polypeptide comprising a first CAR or portion thereof.

In embodiment 54 provided herein is the composition of embodiment 53, wherein the donor template comprises a second sequence encoding a second polypeptide comprising a second CAR or portion thereof.

In embodiment 55 provided herein is the composition of embodiment 54, wherein the second sequence encoding a second polypeptide comprising a second CAR or portion thereof is different from the first sequence encoding a first polypeptide comprising a first CAR or portion thereof.

In embodiment 56 provided herein is the composition of embodiment 54 or 55, wherein the first and second polypeptides are the same polypeptide.

In embodiment 57 provided herein is the composition of embodiment 56, wherein the first and second polypeptides are linked by one or more amino acids.

In embodiment 58 provided herein is the composition of embodiment 54 or 55, wherein the first and second polypeptides are separate polypeptides.

In embodiment 59 provided herein is the composition of any one of embodiments 53 through 58, wherein the first and/or second CARs or portions thereof binds to a binding partner comprising B7H3, BCMA, GPRC5D, CD8, CD8a, CD19, CD20, CD22, CD28, 4-1BB, or CD3zeta or portion thereof.

In embodiment 60 provided herein is the composition of any one of embodiments 53 through 59, wherein the first or second polypeptide comprise a polypeptide that is at least 60, 70, 75, 80, 85, 90, 95, 96, 97, 98, 99, 99.5, or 100% identical to any one of the amino acid sequences of SEQ ID NOs: 86-124.

In embodiment 61 provided herein is a composition comprising a plurality of polynucleotides of any one of embodiments 42 through 60, wherein, for each integer x, the polynucleotide comprises: (A) a donor template (D)ₓ; (B) a first suitable PAM (P1)ₓ and a first target nucleotide sequence (T1)ₓ adjacent to but not within the donor template (D)ₓ; and (C) a second suitable PAM (P2)ₓ and a first target nucleotide sequence (T2)ₓ adjacent to but not within the donor template (D)ₓ.

In embodiment 62 provided herein is the composition of embodiment 61, wherein
(D)ₓ comprises a polynucleotide encoding a polypeptide comprising a CAR or portion thereof that binds a binding partner comprising B7H3, BCMA, GPRC5D, CD8, CD8a, CD19, CD20, CD22, CD28, 4-1BB, or CD3zeta or a portion thereof.

In embodiment 63 provided herein is the composition of embodiment 62, wherein
(D)ₓ comprises a polynucleotide encoding a CAR or portion thereof that comprises a polypeptide at least 60, 70, 75, 80, 85, 90, 95, 96, 97, 98, 99, 99.5, or 100% identical to any one of the amino acid sequences of SEQ ID NOs: 86-124.

In embodiment 64 provided herein is the composition of any one of embodiments 61 through 63, wherein the number of different integers x is at least 2, 3, 4, 5, 6, 7, 8, or 9 and/or no more than 10, 9, 8, 6, 5, 4, or 3.

In embodiment 65 provided herein is the composition of embodiment 64, wherein the number of different integers x is 2-10.

In embodiment 66 provided herein is the composition of embodiment 65, wherein the number of different integers x is 2-5.

In embodiment 67 provided herein is a cell comprising a composition comprising of any one of the proceeding, a progeny of a cell comprising a composition of any one of the proceeding embodiments, or a progeny of a cell comprising one or more genetic modifications, wherein the one or more genetic modifications were generated after contacting the cell with a composition of any one of the proceeding embodiments.

In embodiment 68 provided herein is the cell of embodiment 67, wherein the cell is a human cell.

In embodiment 69 provided herein is the cell of embodiment 68, wherein the human cell is an immune cell or a stem cell.

In embodiment 70 provided herein is the cell of embodiment 68, wherein the human cell is an immune cell comprising a neutrophil, cosinophil, basophil, mast cell, monocyte, macrophage, dendritic cell, natural killer cell, or a lymphocyte.

In embodiment 71 provided herein is the cell of embodiment 68, wherein the human cell is a T cell.

In embodiment 72 provided herein is the cell of embodiment 68, wherein the human cell is a stem cell that is a human pluripotent, multipotent stem cell, embryonic stem cell, induced pluripotent stem cell, hematopoietic stem cell, CD34+ cell.

In embodiment 73 provided herein is the cell of embodiment 68, wherein the human cell is an induced pluripotent stem cell.

In embodiment 74 provided herein is the cell of any one of embodiments 68 through 73, wherein the cell is a cell demonstrating reduced immunogenicity when placed in an allogeneic host.

In embodiment 75 provided herein is the cell of embodiment 74, wherein the cell is non-immunogenic when placed in an allogeneic host.

In embodiment 76 provided herein is a method for preparing a linearized polynucleotide comprising contacting a polynucleotide of any one of embodiments 42 through 66 with a nucleic acid-guided nuclease complex, wherein the nucleic acid-guided nuclease complexes binds to a target site on the polynucleotide and generates at least one strand break.

In embodiment 77 provided herein is a method for engineering a genome of a cell comprising delivering to the cell a composition comprising: (A) a polynucleotide of any one of embodiments 42 through 66; and (B) a nucleic acid-guided nuclease system comprising (1) a nucleic acid-guided nuclease, and (2) a gNA.

In embodiment 78 provided herein is a method of introducing a plurality of exogenous nucleic acids into the genome of a target cell comprising contacting the target cell with a composition comprising: (A) a plurality of polynucleotides, wherein, for each integer x, the DNA polynucleotide comprises: (1) a donor template (D)x; (2) a first suitable PAM (P1)x and a first target nucleotide sequence (T1)x adjacent to the 5′ end of (D)x but not within (D)x; and (3) a second suitable PAM (P2)x and a first target nucleotide sequence (T2)x adjacent to the 3′ end of (D)x but not within (D)x; and (4) a first homology arm (HA1)x between (P1)x (T1)x and (D)x and a second homology arm (HA2)x between (P2)x(T2)x and (D)x, where (HA1)x and (HA2)x are capable of initiating host cell mediated recombination of at least a portion of (D)x at a target site (TS)x selected from a plurality of target sites of the genome of the target cell; and (B) for each target site (TS)x, a plurality of nucleic acid-guided nuclease complexes (N)x capable of cleaving at (TS)x and at least one of (T1)x and (T2)x, wherein cleaving of (TS)x and at least one of (T1)x and (T2)x results in homologous recombination of at least a portion of (D)x at (TS)x.

In embodiment 79 provided herein is a method of introducing a plurality of exogenous nucleic acids into the genome of a target cell comprising contacting the target cell with a composition comprising: (A) a plurality of polynucleotides, wherein, for each integer x, the DNA polynucleotide comprises: (1) a donor template (D)x, wherein at least one donor template comprises a polynucleotide encoding for a first CAR; (2) a first suitable PAM (P1)x and a first target nucleotide sequence (T1)x adjacent to the 5′ end of (D)x but not within (D)x; and (3) a second suitable PAM (P2)x and a first target nucleotide sequence (T2)x adjacent to the 3′ end of (D)x but not within (D)x; and (4) a first homology arm (HA1)x between (P1)x(T1)x and (D)x and a second homology arm (HA2)x between (P2)x(T2)x and (D)x, where (HA1)x and (HA2)x are capable of initiating host cell mediated recombination of at least a portion of (D)x at a target site (TS)x selected from a plurality of target sites of the genome of the target cell; and (B) for each target site (TS)x, a plurality of nucleic acid-guided nuclease complexes (N)x capable of cleaving at (TS)x and at least one of (T1)x and (T1)x, wherein cleaving of (TS)x and at least one of (T1)x and (T1)x results in homologous recombination of at least a portion of (D)x at (TS)x.

In embodiment 80 provided herein is a composition comprising: (A) a linear double-stranded DNA polynucleotide with covalently closed ends; and (B) a polypeptide comprising a nuclear localization signal (NLS) bound to the polynucleotide.

In embodiment 81 provided herein is the composition of embodiment 80, wherein the double-stranded DNA polynucleotide comprises a donor template (D).

In embodiment 82 provided herein is the composition of embodiment 80 or 81, wherein the polypeptide comprises a nucleic acid-guided nuclease complex comprising: (1) a nucleic acid-guided nuclease; and (2) a guide nucleic acid (gNA).

In embodiment 83 provided herein is the composition of embodiment 82, wherein the double-stranded DNA polynucleotide comprises a first PAM (P1) recognized by a nucleic acid-guided nuclease and a first target nucleotide sequence (T1) adjacent to but not within the donor template (D).

In embodiment 84 provided herein is the composition of embodiment 83, wherein the double-stranded DNA polynucleotide further comprises a second PAM (P2) recognized by a nucleic acid-guided nuclease and a second target nucleotide sequence (T2) adjacent to but not within the donor template (D).

In embodiment 85 provided herein is the composition of embodiment 83 or 84, wherein the first PAM and the first target nucleotide sequence are oriented 5′ P1+T1+D+ 3′.

In embodiment 86 provided herein is the composition of embodiment 83 or 84, wherein the first PAM and the first target nucleotide sequence are oriented 5′ T1−P1−D+ 3′.

In embodiment 87 provided herein is the composition of any one of embodiments 84 through 86, wherein the second PAM and the second target nucleotide sequence are oriented 5′ D+P2+T2+ 3′.

In embodiment 88 provided herein is the composition of any one of embodiments 84 through 86, wherein the second suitable PAM and the second target nucleotide sequence are oriented 5′ D+T2−P2− 3′.

In embodiment 89 provided herein is the composition of embodiment 84, wherein the first and second PAM target nucleotide sequences are oriented 5′ T1−P1−D+P2+T2+ 3′. In embodiment 90 provided herein is the composition of embodiment 84, wherein the first and second PAM target nucleotide sequences are oriented 5′ P1+T1+D+T2−P2− 3′.

In embodiment 91 provided herein is a composition comprising a polynucleotide comprising: (A) linear double stranded DNA donor template (D) with covalently closed ends; (B) a first PAM (P1) recognized by a Type V nucleic acid-guided nuclease and a first target nucleotide sequence (T1) adjacent to but not within the donor template (D); and, optionally, (C) a second PAM (P2) recognized by a Type V nucleic-acid guided nuclease and a second target nucleotide sequence (T2) adjacent to but not within the donor template (D).

In embodiment 92 provided herein is the composition of embodiment 91, wherein the first PAM and the first target nucleotide sequence are oriented 5′ P1+T1+D+ 3′.

In embodiment 93 provided herein is the composition of embodiment 91, wherein the first PAM and the first target nucleotide sequence are oriented 5′ T1−P1−D+ 3′.

In embodiment 94 provided herein is the composition of embodiment 92 or 93, wherein the second PAM and the second target nucleotide sequence are oriented 5′ D+P2+T2+ 3′

In embodiment 95 provided herein is the composition of embodiment 92 or 93, wherein the second suitable PAM and the second target nucleotide sequence are oriented 5′ D+T2−P2− 3′.

In embodiment 96 provided herein is the composition of embodiment 91, wherein the first and second PAM target nucleotide sequences are oriented 5′ T1−P1−D+P2+T2+ 3′.

In embodiment 97 provided herein is the composition of embodiment 91, wherein the first and second PAM target nucleotide sequences are oriented 5′ P1+T1+D+T2−P2− 3′.

In embodiment 98 provided herein is the composition of any one of embodiments 91 through 97, further comprising a polypeptide comprising a nuclear localization signal (NLS) bound to the polynucleotide.

In embodiment 98 provided herein is a method comprising generating a linear double stranded DNA polynucleotide with covalently closed ends wherein the polynucleotide comprises: (1) a linear double stranded DNA donor template (D) with covalently closed ends; (2) a first PAM (P1) recognized by a Type V nucleic acid-guided nuclease and a first target nucleotide sequence (T1) adjacent to but not within the donor template (D); and, optionally, (3) a second PAM (P2) recognized by a Type V nucleic-acid guided nuclease and a second target nucleotide sequence (T2) adjacent to but not within the donor template (D), wherein the polynucleotide is generated from a circular double stranded DNA polynucleotide comprising protelomerase recognition sites flanking the donor template, the PAMs and the target nucleotide sequences and, optionally, comprises a polypeptide comprising a nuclear localization signal (NLS) bound to the polynucleotide.

In embodiment 100 provided herein is a method comprising contacting a cell with a composition of any one of embodiments 80 through 98 or a polynucleotide generated by a method of embodiment 99

In embodiment 101 provided herein is a cell or a progeny thereof comprising a composition of any one of embodiments 80 through 98 or a polynucleotide generated by a method of embodiment 99.

In embodiment 102 provided herein is the method of claim 100, wherein the editing efficiency as measured by the number of edits in a population of cells treated with the composition is at least 5, 10, 20, 25, 30, 40, 50, 60, 70, 80, 90, 100, 200, or 500% greater than that of a population of cells treated with a standard composition.

### VIII. EXAMPLES

### A. Example 1

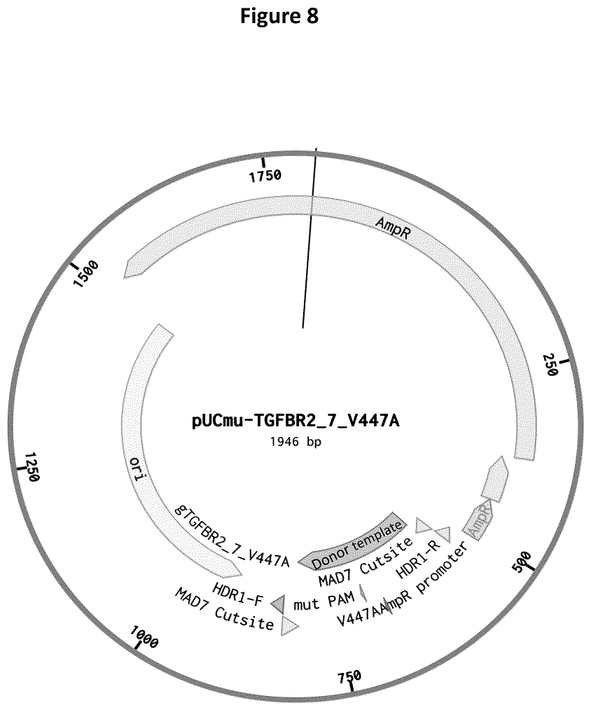

This example demonstrates the utility of miniplasmids comprising differently oriented PAM+gRNAs for the delivery and knock in of one or more heterologous genes in combination with a nucleic acid-guided nuclease complex. Briefly, miniplasmids comprising a donor template were generated wherein the donor template was flanked by upstream and downstream PAMs and target nucleotide sequences. Each upstream and downstream PAM and target nucleotide sequence were either on the sense or antisense strand with respect to the coding sequence of the donor template, resulting in four miniplasmid constructs. Schematics of the orientation of the target nucleotide sequences (T) and PAMs (P) with respect to the donor template (D) are shown in FIGS. 4-7. Specifically, FIG. 4 shows a donor template flanked by an upstream PAM and target nucleotide sequence on the sense strand (P₁⁺T₁⁺D⁺) and a downstream PAM and target nucleotide sequence on the antisense strand (D⁺T₂⁻P₂⁻). FIG. 5 shows a donor template flanked by an upstream PAM and target nucleotide sequence on the antisense strand (T₁⁻P₁⁻D⁺) and a downstream PAM and target nucleotide sequence on the antisense strand (D⁺T₂⁻P₂⁻). FIG. 6 shows a donor template flanked by an upstream PAM and target nucleotide sequence on the sense strand (P₁⁺T₁⁺D⁺) and a downstream PAM and target nucleotide sequence on the sense strand (D⁺P₂⁺T₂⁺). FIG. 7 shows a donor template flanked by an upstream PAM and target nucleotide sequence on the antisense strand (T₁⁻P₁⁻D⁺) and a downstream PAM and target nucleotide sequence on the sense strand (D⁺P₂⁺T₂⁺). FIG. 8 shows an exemplary plasmid map (pUCmu-TGFBR2) of one or the four miniplasmids. Not wishing to be bound by theory, the nucleic acid-guided nuclease complex can interact with the PAM-proximal cleavage product after cleavage and nucleic acid-guided nuclease complex comprising one or more functional moieties, for example a nuclear localization signal, can aid in the import of one or more bound polynucleotides into the nucleus. This is also illustrated in FIGS. 4-7, wherein (1) the donor template is distal to both the upstream and downstream PAMs; (2) the donor template is proximal to the upstream PAM and distal to the downstream PAM; (3) the donor template is distal to the upstream PAM and proximal to the downstream PAM; and (4) the donor template is proximal to both the upstream and downstream PAMs. Each of the 4 miniplasmids were tested for cell knock-in efficiency at 3 different loci in primary T-cells. 48 hours after isolation, T cells were harvested by centrifugation (300 g, RT, 5 minutes) and 1×10⁶ cells were re-suspended in 20 μL in supplemented P3 Primary Cell Nucleofector Kit buffer (Lonza). The cells were mixed with the 1 ug respective miniplasmid and an RNP solution (100 pmol RNPs (1:1 gRNA/target to Mad7 ratio) gRNA:gTGFBR2_007, gTGFBR2_008, gFAS_94) was added to the cells immediately before transfection (Nucleofection program EH-115). After transfection, 80 μL of pre-warmed cultivation medium without IL-2 was added to the electroporation cuvettes. After 10 minutes of incubation at 37° C., T-cells were transferred onto 96-well, flat-bottom, non-cell culture treated plates (Falcon) containing pre-warmed cultivation medium pretreated with 12.5 ng mL⁻¹ IL-2. The cells were seeded at a density of 0.25×10⁶ cells mL⁻¹ and kept at 37° C. in 5% CO₂ incubators. The viability assay was carried out 24 hours post-transfection after which the cells were reseeded in the fresh cultivation medium containing IL-2.

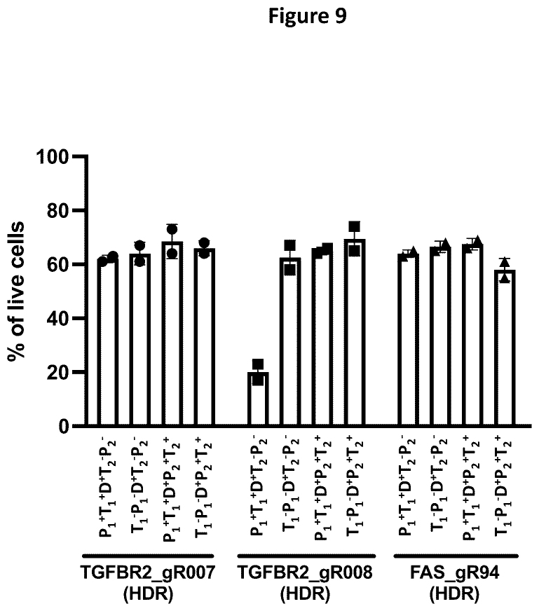

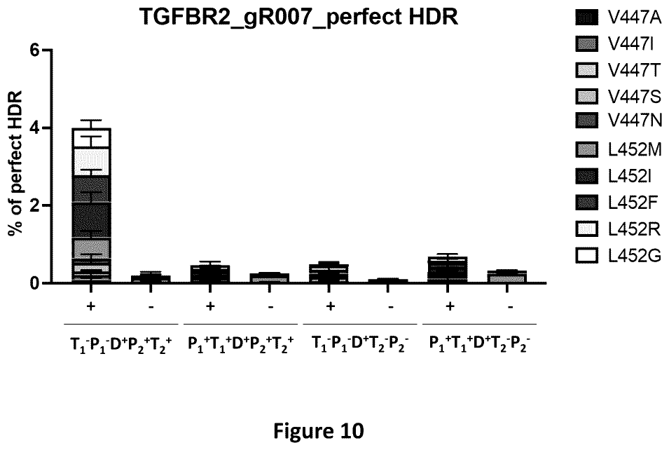

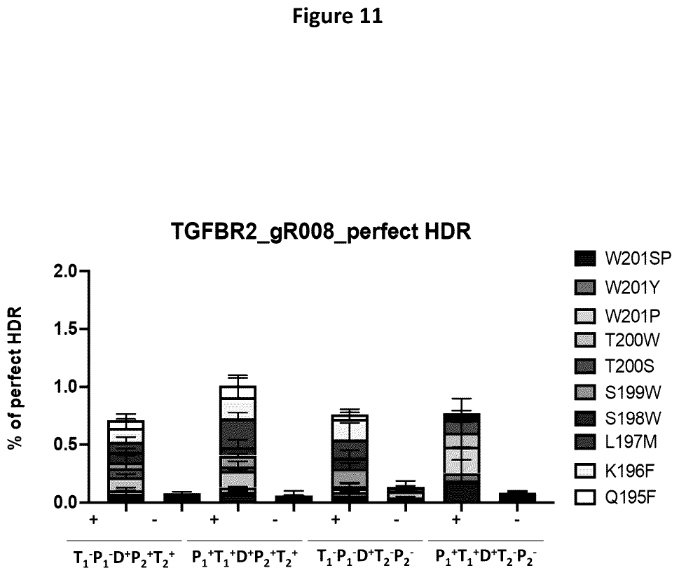

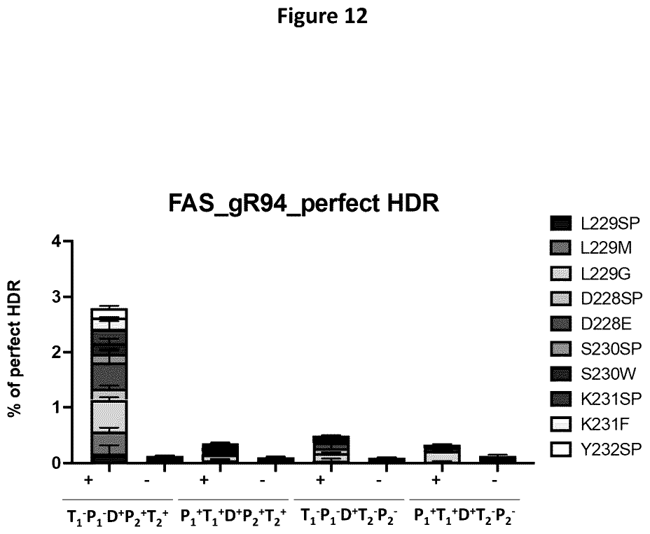

On day 2 post electroporation, the cell viability was measured using Cellaca, DNA was isolated and sequenced. Cell viability (% of live cells, y-axis) is shown in FIG. 9 for each of the treatment conditions (x-axis). Notably, cell viability was >60% after treatment at day 2 for each of the treatment conditions besides P₁⁺T₁⁺D⁺T₂⁻P₂⁻ at the TGFBR2 locus using MAD7 complexed with gR008. Knock in efficiency for each of the treatment conditions are shown in FIGS. 10-12. Specifically, FIG. 10 shows knock-in efficiency as measured by % of perfect HDR (y-axis) at the TGFBR2 locus using MAD7 complexed with gR007 for each miniplasmid (x-axis). Each miniplasmid was tested with (+) and without (−) transfection of the RNP. FIG. 11 shows knock-in efficiency as measured by % of perfect HDR (y-axis) at the TGFBR2 locus using MAD7 complexed with gR008 for each miniplasmid (x-axis). Each miniplasmid was tested with (+) and without (−) transfection of the RNP. FIG. 12 shows knock-in efficiency as measured by % of perfect HDR (y-axis) at the FAS locus using MAD7 complexed with gR94 for each miniplasmid (x-axis). Each miniplasmid was tested with (+) and without (−) transfection of the RNP. Notably, at the TGFBR2 locus using MAD7 complexed with gR007 (FIG. 10, left bar) and the FAS locus using MAD7 complexed with gR94 (FIG. 12, left bar), 3-4% of all edits were through perfect HDR using the T₁⁻P₁⁻D⁺P₂⁺T₂⁺ miniplasmid, ˜4-8-fold higher than for the other 3 miniplasmids. Interestingly, the TGFBR2 locus using MAD7 complexed with gR008 showed similar results for each miniplasmid type (FIG. 11).

### B. Example 2

This example demonstrates reduced cellular toxicity and improved gene editing with double stranded DNA repair templates. dsDNA is often a preferred repair template due to the case of manufacture and cost-effective renewable properties. dsDNA can result in cellular toxicity and lowered cell viability after introduction in cells, for example primary human T-cells.

Here, we demonstrate that minimized plasmids and linear double-stranded DNA with closed/circularized ends result in improved gene editing and lowered cell toxicity when used as a repair template in mammalian cells. As linear double stranded DNA with closed/circularized ends are protected from exonucleases, it is possible to use less repair template as compared to linear double stranded DNA.

Briefly, human Pan T-cells were isolated from Leukopaks (StemCell Technology) using EasySep Direct Human T cell Isolation Kit (StemCell Technology Catalog #19661) and cryopreserved using CryoStor CS10 (StemCell Technology Catalog #07930). The cells were thawed and activated with ImmunoCult Human CD3/CD28 T Cell Activator (StemCell Technology Catalog #10991) and cultivated in ImmunoCult-XF T Cell Expansion Medium (StemCell Technology, Catalog #10981) supplemented with IL2 (StemCell Technlogy Catalog #78036.3) at 37° C. in a 5% CO2 environment, and transfected after approximately 72 hours with RNPs, consisting of artSTAR1.0 protein and synthetic dual gRNA comprising a modulator sequence and a AAVS1 or TRAC targeter sequence, and Miniplasmids, ldsDNA or ldsDNA with closed ends with (HDRT variant 2) or without (HDRT variant 1) a flanking gRNA target site as HDR templates. Sequences are shown in Table 7 below. artSTAR1.0 protein, which contained NLS sequences at the N-terminus, was expressed in E. coli and purified by fast protein liquid chromatography (FPLC). RNP complexes were prepared by incubating artSTAR1.0 protein with chemically synthesized gRNA for 10 minutes at room temperature. The RNPs were mixed with HDR template. The RNPs+HDR templates were mixed with 1 000,000 Pan T-cells resuspended in nucleofection buffer P3 (Lonza) in a final volume of 25 μL. Electroporation was carried out on a 4D-Nucleofector (Lonza) using program EH-115. Following electroporation, the cells were cultured for 2-3 days.

Flowcytometry was used to determine the cell population expressing the GOI from the integrated HDR template. In case of non-flourescence GOI, antibody staining of the cells was used to stain the expressed protein. Briefly, 1,000,000 cells/ml were harvested and washed with Cell Staining Buffer (Biolegend, catalog #420201), incubated with a fluorophore tagged antibody against the protein of interest or an indirect marker for the protein of interest, washed with Cell Staining Buffer (Biolegend, catalog #420201), resuspended in 1×PBS and analyzed by Flow cytometry. The data were analyzed using Flowjo, gated for viable, single cells and the positive cell population expressing the fluorescence protein or containing the stained protein.

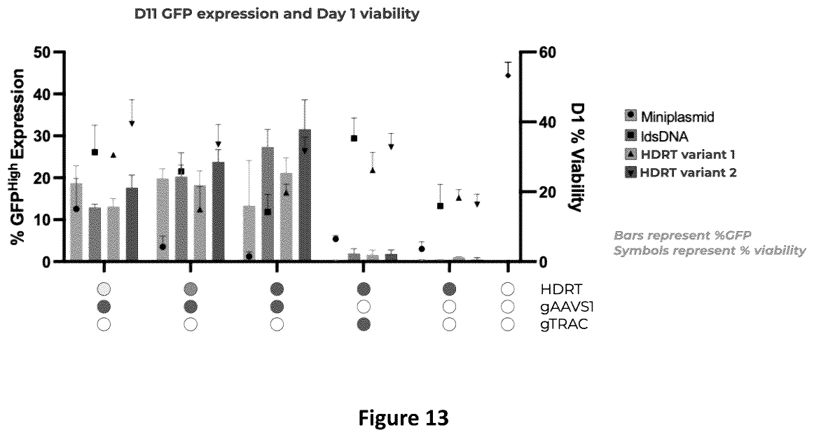

The data is shown in FIG. 13 with condition on the x-axis and % GFP expression shown on the left y-axis and viability on the right y-axis. Briefly, control cells transfected with only buffer demonstrated ˜55% viability at day 1 (6^{th} data set). Cells transfected with only HDRT and lacking any guide RNA demonstrated negligible GFP expression (5^{th} data set). Viability post transfection correlated with HDRT length as shown with miniplasmid HDRT demonstrated lower viability post transfection as compared to ldsDNA or either ldsDNA with closed ends. Inclusion of a guide RNA for an off-target locus (gTRAC; 4^{th} data set) showed no GFP expression. Cell viability with inclusion of the gTRAC guide RNA was ˜2-fold higher than without. Data sets 1-3 show GFP expression via GFP integration into the AAVS1 locus and subsequent viability post transfection when treated with HDRT and gAAVS1 guide RNAs. Increasing amounts of HDRT are used with the 1^{st} data set having the least HDRT and the 3^{rd} having the most. The increase in HDRT correlates with a decrease in viability post transfection with ldsDNA with closed ends having the highest viability post treatment. ldsDNA with closed ends flanked by nuclease binding sites showed the higher GFP integrated efficiency as noted by the highest GFP expression post transfection.

This example demonstrates improved editing and increased cell viability with use of ldsDNA with closed ends.

TABLE 7
sequences of oligonucleotides

| Name | Sequence |
|---|---|
| Modulator | mU*A*AUUCCUACUC |
| Targeter-gAAVS1_3 | UUGUAGGU UUAGGAUGGCCUUCUCCGAC*mG |
| Targeter-gTRAC043 | UUGUAGGU GAGUCUCUCAGCUGGUACAC*mG |
| ldsDNA | TCAGCTGCGCTGCCCTCCTCTCGCCCCCGAGTGCCCTTGCTGTG |
| | CCGCCGGAACTCTGCCCTCTAACGCTGCCGTCTCTCTCCTGAGT |
| | CCGGACCACTTTGAGCTCTACTGGCTTCTGCGCCGCCTCTGGCC |
| | CACTGTTTCCCCTTCCCAGGCAGGTCCTGCTTTCTCTGACCTGC |
| | ATTCTCTCCCCTGGGCCTGTGCCGCTTTCTGTCTGCAGCTTGTG |
| | GCCTGGGTCACCTCTACGGCTGGCCCAGATCCTTCCCTGCCGCC |
| | TCCTTCAGGTTCCGTCTTCCTCCACTCCCTCTTCCCCTTGCTCT |
| | CTGCTGTGTTGCTGCCCAAGGATGCTCTTTCCGGAGCACTTCCT |
| | TCTCGGCGCTGCACCACGTGATGTCCTCTGAGCGGATCCTCCCC |
| | GTGTCTGGGTCCTCTCCGGGCATCTCTCCTCCCTCACCCAACCC |
| | CATGCCGTCTTCACTCGCTGGGTTCCCTTTTCCTTCTCCTTCTG |
| | GGGCCTGTGCCATCTCTCGTTGATATCTCGACTAGTTATTAATA |
| | GTAATCAATTACGGGGTCATTAGTTCATAGCCCATATATGGAGT |
| | TCCGCGTTACATAACTTACGGTAAATGGCCCGCCTGGCTGACCG |
| | CCCAACGACCCCCGCCCATTGACGTCAATAATGACGTATGTTCC |
| | CATAGTAACGCCAATAGGGACTTTCCATTGACGTCAATGGGTGG |
| | AGTATTTACGGTAAACTGCCCACTTGGCAGTACATCAAGTGTAT |
| | CATATGCCAAGTACGCCCCCTATTGACGTCAATGACGGTAAATG |
| | GCCCGCCTGGCATTATGCCCAGTACATGACCTTATGGGACTTTC |
| | CTACTTGGCAGTACATCTACGTATTAGTCATCGCTATTACCATG |
| | GTCGAGGTGAGCCCCACGTTCTGCTTCACTCTCCCCATCTCCCC |
| | CCCCTCCCCACCCCCAATTTTGTATTTATTTATTTTTTAATTAT |
| | TTTGTGCAGCGATGGGGGCGGGGGGGGGGGGGGGGCGCGCGCCA |
| | GGCGGGGCGGGGCGGGGCGAGGGGCGGGGCGGGGCGAGGCGGAG |
| | AGGTGCGGCGGCAGCCAATCAGAGCGGCGCGCTCCGAAAGTTTC |
| | CTTTTATGGCGAGGCGGCGGCGGCGGCGGCCCTATAAAAAGCGA |
| | AGCGCGCGGCGGGCGGGGAGTCGCTGCGACGCTGCCTTCGCCCC |
| | GTGCCCCGCTCCGCCGCCGCCTCGCGCCGCCCGCCCCGGCTCTG |
| | ACTGACCGCGTTACTCCCACAGGTGAGCGGGCGGGACGGCCCTT |
| | CTCCTCCGGGCTGTAATTAGCGCTTGGTTTAATGACGGCTTGTT |
| | TCTTTTCTGTGGCTGCGTGAAAGCCTTGAGGGGCTCCGGGAGGG |
| | CCCTTTGTGCGGGGGGAGCGGCTCGGGGGGTGCGTGCGTGTGTG |
| | TGTGCGTGGGGAGCGCCGCGTGCGGCTCCGCGCTGCCCGGCGGC |
| | TGTGAGCGCTGCGGGCGCGGCGCGGGGCTTTGTGCGCTCCGCAG |
| | TGTGCGCGAGGGGAGCGCGGCCGGGGGCGGTGCCCCGCGGTGCG |
| | GGGGGGGCTGCGAGGGGAACAAAGGCTGCGTGCGGGGTGTGTGC |
| | GTGGGGGGGTGAGCAGGGGGTGTGGGCGCGTCGGTCGGGCTGCA |
| | ACCCCCCCTGCACCCCCCTCCCCGAGTTGCTGAGCACGGCCCGG |
| | CTTCGGGTGCGGGGCTCCGTACGGGGCGTGGCGCGGGGCTCGCC |
| | GTGCCGGGCGGGGGGTGGCGGCAGGTGGGGGTGCCGGGCGGGGC |
| | GGGGCCGCCTCGGGCCGGGGAGGGCTCGGGGGAGGGGCGCGGCG |
| | GCCCCCGGAGCGCCGGCGGCTGTCGAGGCGCGGCGAGCCGCAGC |
| | CATTGCCTTTTATGGTAATCGTGCGAGAGGGCGCAGGGACTTCC |
| | TTTGTCCCAAATCTGTGCGGAGCCGAAATCTGGGAGGCGCCGCC |
| | GCACCCCCTCTAGCGGGCGCGGGGCGAAGCGGTGCGGCGCCGGC |
| | AGGAAGGAAATGGGCGGGGAGGGCCTTCGTGCGTCGCCGCGCCG |
| | CCGTCCCCTTCTCCCTCTCCAGCCTCGGGGCTGTCCGCGGGGGG |
| | ACGGCTGCCTTCGGGGGGGACGGGGCAGGGCGGGGTTCGGCTTC |
| | TGGCGTGTGACCGGCGGCTCTAGAGCCTCTGCTAACCATGTTCA |
| | TGCCTTCTTCTTTTTCCTACAGCTCCTGGGCAACGTGCTGGTTA |
| | TTGTGCTGTCTCATCATTTTGGCAAAGAATTAATTCGGATCCAC |
| | CATGGTGAGCAAGGGCGAGGAGCTGTTCACCGGGGTGGTGCCCA |
| | TCCTGGTCGAGCTGGACGGCGACGTAAACGGCCACAAGTTCAGC |
| | GTGTCCGGCGAGGGCGAGGGCGATGCCACCTACGGCAAGCTGAC |
| | CCTGAAGTTCATCTGCACCACCGGCAAGCTGCCCGTGCCCTGGC |
| | CCACCCTCGTGACCACCCTGACCTACGGCGTGCAGTGCTTCAGC |
| | CGCTACCCCGACCACATGAAGCAGCACGACTTCTTCAAGTCCGC |
| | CATGCCCGAAGGCTACGTCCAGGAGCGCACCATCTTCTTCAAGG |
| | ACGACGGCAACTACAAGACCCGCGCCGAGGTGAAGTTCGAGGGC |
| | GACACCCTGGTGAACCGCATCGAGCTGAAGGGCATCGACTTCAA |
| | GGAGGACGGCAACATCCTGGGGCACAAGCTGGAGTACAACTACA |
| | ACAGCCACAACGTCTATATCATGGCCGACAAGCAGAAGAACGGC |
| | ATCAAGGTGAACTTCAAGATCCGCCACAACATCGAGGACGGCAG |
| | CGTGCAGCTCGCCGACCACTACCAGCAGAACACCCCCATCGGCG |
| | ACGGCCCCGTGCTGCTGCCCGACAACCACTACCTGAGCACCCAG |
| | TCCGCCCTGAGCAAAGACCCCAACGAGAAGCGCGATCACATGGT |
| | CCTGCTGGAGTTCGTGACCGCCGCCGGGATCACTCTCGGCATGG |
| | ACGAGCTGTACAAGTAACTGTGCCTTCTAGTTGCCAGCCATCTG |
| | TTGTTTGCCCCTCCCCCGTGCCTTCCTTGACCCTGGAAGGTGCC |
| | ACTCCCACTGTCCTTTCCTAATAAAATGAGGAAATTGCATCGCA |
| | TTGTCTGAGTAGGTGTCATTCTATTCTGGGGGGTGGGGTGGGGC |
| | AGGACAGCAAGGGGGAGGATTGGGAAGACAATAGCAGGCATGCT |
| | GGGGATGCGGTGGGCTCTATGGCGACGGATGTCTCCCTTGCGTC |
| | CCGCCTCCCCTTCTTGTAGGCCTGCATCATCACCGTTTTTCTGG |
| | ACAACCCCAAAGTACCCCGTCTCCCTGGCTTTAGCCACCTCTCC |
| | ATCCTCTTGCTTTCTTTGCCTGGACACCCCGTTCTCCTGTGGAT |
| | TCGGGTCACCTCTCACTCCTTTCATTTGGGCAGCTCCCCTACCC |
| | CCCTTACCTCTCTAGTCTGTGCTAGCTCTTCCAGCCCCCTGTCA |
| | TGGCATCTTCCAGGGGTCCGAGAGCTCAGCTAGTCTTCTTCCTC |
| | CAACCCGGGCCCCTATGTCCACTTCAGGACAGCATGTTTGCTGC |
| | CTCCAGGGATCCTGTGTCCCCGAGCTGGGACCACCTTATATTCC |
| | CAGGGCCGGTTAATGTGGCTCTGGTTCTGGGTACTTTTATCTGT |
| | CCCCTCCACCCCACAGTGGGGCCACTAGGGACAGGATTGGTGAC |
| | AGAAAAGCCCCATCCTTAGGCCTCCTCCTTCCTAGTCTC |
| Miniplasmid | CGCGCACCCACACCCAGGCCAGGGTGTTGTCCGGCACCACCTGG |
| | TCCTGGACCGCGCTGATGAACAGGGTCACGTCGTCCCGGACCAC |
| | ACCGGCGAAGTCGTCCTCCACGAAGTCCCGGGAGAACCCGAGCC |
| | GGTCGGTCCAGAACTCGACCGCTCCGGCGACGTCGCGCGCGGTG |
| | AGCACCGGAACGGCACTGGTCAACTTGGCCATACTCTTCCTTTT |
| | TCAATATTATTGAAGCATTTATCAGGGTTATTGTCTCATGAGCG |
| | GATACATAACGCGCGTCGGAGAAGGCCATCCTAAGAAACCTCAG |
| | CTGCGCTGCCCTCCTCTCGCCCCCGAGTGCCCTTGCTGTGCCGC |
| | CGGAACTCTGCCCTCTAACGCTGCCGTCTCTCTCCTGAGTCCGG |
| | ACCACTTTGAGCTCTACTGGCTTCTGCGCCGCCTCTGGCCCACT |
| | GTTTCCCCTTCCCAGGCAGGTCCTGCTTTCTCTGACCTGCATTC |
| | TCTCCCCTGGGCCTGTGCCGCTTTCTGTCTGCAGCTTGTGGCCT |
| | GGGTCACCTCTACGGCTGGCCCAGATCCTTCCCTGCCGCCTCCT |
| | TCAGGTTCCGTCTTCCTCCACTCCCTCTTCCCCTTGCTCTCTGC |
| | TGTGTTGCTGCCCAAGGATGCTCTTTCCGGAGCACTTCCTTCTC |
| | GGCGCTGCACCACGTGATGTCCTCTGAGCGGATCCTCCCCGTGT |
| | CTGGGTCCTCTCCGGGCATCTCTCCTCCCTCACCCAACCCCATG |
| | CCGTCTTCACTCGCTGGGTTCCCTTTTCCTTCTCCTTCTGGGGC |
| | CTGTGCCATCTCTCGTTGATATCTCGACTAGTTATTAATAGTAA |
| | TCAATTACGGGGTCATTAGTTCATAGCCCATATATGGAGTTCCG |
| | CGTTACATAACTTACGGTAAATGGCCCGCCTGGCTGACCGCCCA |
| | ACGACCCCCGCCCATTGACGTCAATAATGACGTATGTTCCCATA |
| | GTAACGCCAATAGGGACTTTCCATTGACGTCAATGGGTGGAGTA |
| | TTTACGGTAAACTGCCCACTTGGCAGTACATCAAGTGTATCATA |
| | TGCCAAGTACGCCCCCTATTGACGTCAATGACGGTAAATGGCCC |
| | GCCTGGCATTATGCCCAGTACATGACCTTATGGGACTTTCCTAC |
| | TTGGCAGTACATCTACGTATTAGTCATCGCTATTACCATGGTCG |
| | AGGTGAGCCCCACGTTCTGCTTCACTCTCCCCATCTCCCCCCCC |
| | TCCCCACCCCCAATTTTGTATTTATTTATTTTTTAATTATTTTG |
| | TGCAGCGATGGGGGCGGGGGGGGGGGGGGGGCGCGCGCCAGGCG |
| | GGGCGGGGCGGGGCGAGGGGCGGGGCGGGGCGAGGCGGAGAGGT |
| | GCGGCGGCAGCCAATCAGAGCGGCGCGCTCCGAAAGTTTCCTTT |
| | TATGGCGAGGCGGCGGCGGCGGCGGCCCTATAAAAAGCGAAGCG |
| | CGCGGCGGGCGGGGAGTCGCTGCGACGCTGCCTTCGCCCCGTGC |
| | CCCGCTCCGCCGCCGCCTCGCGCCGCCCGCCCCGGCTCTGACTG |
| | ACCGCGTTACTCCCACAGGTGAGCGGGCGGGACGGCCCTTCTCC |
| | TCCGGGCTGTAATTAGCGCTTGGTTTAATGACGGCTTGTTTCTT |
| | TTCTGTGGCTGCGTGAAAGCCTTGAGGGGCTCCGGGAGGGCCCT |
| | TTGTGCGGGGGGAGCGGCTCGGGGGGTGCGTGCGTGTGTGTGTG |
| | CGTGGGGAGCGCCGCGTGCGGCTCCGCGCTGCCCGGCGGCTGTG |
| | AGCGCTGCGGGCGCGGCGCGGGGCTTTGTGCGCTCCGCAGTGTG |
| | CGCGAGGGGAGCGCGGCCGGGGGCGGTGCCCCGCGGTGCGGGGG |
| | GGGCTGCGAGGGGAACAAAGGCTGCGTGCGGGGTGTGTGCGTGG |
| | GGGGGTGAGCAGGGGGTGTGGGCGCGTCGGTCGGGCTGCAACCC |
| | CCCCTGCACCCCCCTCCCCGAGTTGCTGAGCACGGCCCGGCTTC |
| | GGGTGCGGGGCTCCGTACGGGGCGTGGCGCGGGGCTCGCCGTGC |
| | CGGGCGGGGGGTGGCGGCAGGTGGGGGTGCCGGGCGGGGCGGGG |
| | CCGCCTCGGGCCGGGGAGGGCTCGGGGGAGGGGCGCGGCGGCCC |
| | CCGGAGCGCCGGCGGCTGTCGAGGCGCGGCGAGCCGCAGCCATT |
| | GCCTTTTATGGTAATCGTGCGAGAGGGCGCAGGGACTTCCTTTG |
| | TCCCAAATCTGTGCGGAGCCGAAATCTGGGAGGCGCCGCCGCAC |
| | CCCCTCTAGCGGGCGCGGGGCGAAGCGGTGCGGCGCCGGCAGGA |
| | AGGAAATGGGCGGGGAGGGCCTTCGTGCGTCGCCGCGCCGCCGT |
| | CCCCTTCTCCCTCTCCAGCCTCGGGGCTGTCCGCGGGGGGACGG |
| | CTGCCTTCGGGGGGGACGGGGCAGGGCGGGGTTCGGCTTCTGGC |
| | GTGTGACCGGCGGCTCTAGAGCCTCTGCTAACCATGTTCATGCC |
| | TTCTTCTTTTTCCTACAGCTCCTGGGCAACGTGCTGGTTATTGT |
| | GCTGTCTCATCATTTTGGCAAAGAATTAATTCGGATCCACCATG |
| | GTGAGCAAGGGCGAGGAGCTGTTCACCGGGGTGGTGCCCATCCT |
| | GGTCGAGCTGGACGGCGACGTAAACGGCCACAAGTTCAGCGTGT |
| | CCGGCGAGGGCGAGGGCGATGCCACCTACGGCAAGCTGACCCTG |
| | AAGTTCATCTGCACCACCGGCAAGCTGCCCGTGCCCTGGCCCAC |
| | CCTCGTGACCACCCTGACCTACGGCGTGCAGTGCTTCAGCCGCT |
| | ACCCCGACCACATGAAGCAGCACGACTTCTTCAAGTCCGCCATG |
| | CCCGAAGGCTACGTCCAGGAGCGCACCATCTTCTTCAAGGACGA |
| | CGGCAACTACAAGACCCGCGCCGAGGTGAAGTTCGAGGGCGACA |
| | CCCTGGTGAACCGCATCGAGCTGAAGGGCATCGACTTCAAGGAG |
| | GACGGCAACATCCTGGGGCACAAGCTGGAGTACAACTACAACAG |
| | CCACAACGTCTATATCATGGCCGACAAGCAGAAGAACGGCATCA |
| | AGGTGAACTTCAAGATCCGCCACAACATCGAGGACGGCAGCGTG |
| | CAGCTCGCCGACCACTACCAGCAGAACACCCCCATCGGCGACGG |
| | CCCCGTGCTGCTGCCCGACAACCACTACCTGAGCACCCAGTCCG |
| | CCCTGAGCAAAGACCCCAACGAGAAGCGCGATCACATGGTCCTG |
| | CTGGAGTTCGTGACCGCCGCCGGGATCACTCTCGGCATGGACGA |
| | GCTGTACAAGTAACTGTGCCTTCTAGTTGCCAGCCATCTGTTGT |
| | TTGCCCCTCCCCCGTGCCTTCCTTGACCCTGGAAGGTGCCACTC |
| | CCACTGTCCTTTCCTAATAAAATGAGGAAATTGCATCGCATTGT |
| | CTGAGTAGGTGTCATTCTATTCTGGGGGGTGGGGTGGGGCAGGA |
| | CAGCAAGGGGGAGGATTGGGAAGACAATAGCAGGCATGCTGGGG |
| | ATGCGGTGGGCTCTATGGCGACGGATGTCTCCCTTGCGTCCCGC |
| | CTCCCCTTCTTGTAGGCCTGCATCATCACCGTTTTTCTGGACAA |
| | CCCCAAAGTACCCCGTCTCCCTGGCTTTAGCCACCTCTCCATCC |
| | TCTTGCTTTCTTTGCCTGGACACCCCGTTCTCCTGTGGATTCGG |
| | GTCACCTCTCACTCCTTTCATTTGGGCAGCTCCCCTACCCCCCT |
| | TACCTCTCTAGTCTGTGCTAGCTCTTCCAGCCCCCTGTCATGGC |
| | ATCTTCCAGGGGTCCGAGAGCTCAGCTAGTCTTCTTCCTCCAAC |
| | CCGGGCCCCTATGTCCACTTCAGGACAGCATGTTTGCTGCCTCC |
| | AGGGATCCTGTGTCCCCGAGCTGGGACCACCTTATATTCCCAGG |
| | GCCGGTTAATGTGGCTCTGGTTCTGGGTACTTTTATCTGTCCCC |
| | TCCACCCCACAGTGGGGCCACTAGGGACAGGATTGGTGACAGAA |
| | AAGCCCCATCCTTAGGCCTCCTCCTTCCTAGTCTCTCGAGTTTC |
| | TTAGGATGGCCTTCTCCGACGTCGACTCTAGAGGATCCCGGGTA |
| | CCGAGCTCGAATTCGGATATCCTCGAGACTAGTGGGCCCGTTTA |
| | AACACATGTGTTTTTCCATAGGCTCCGCCCCCCTGACGAGCATC |
| | ACAAAAATCGACGCTCAAGTCAGAGGTGGCGAAACCCGACAGGA |
| | CTATAAAGATACCAGGCGTTTCCCCCTGGAAGCTCCCTCGTGCG |
| | CTCTCCTGTTCCGACCCTGCCGCTTACCGGATACCTGTCCGCCT |
| | TTCTCCCTTCGGGAAGCGTGGCGCTTTCTCATAGCTCACGCTGT |
| | AGGTATCTCAGTTCGGTGTAGGTCGTTCGCTCCAAGCTGGGCTG |
| | TGTGCACGAACCCCCCGTTCAGCCCGACCGCTGCGCCTTATCCG |
| | GTAACTATCGTCTTGAGTCCAACCCGGTAAGACACGACTTATCG |
| | CCACTGGCAGCAGCCACTGGTAACAGGATTAGCAGAGCGAGGTA |
| | TGTAGGCGGTGCTACAGAGTTCTTGAAGTGGTGGCCTAACTACG |
| | GCTACACTAGAAGGACAGTATTTGGTATCTGCGCTCTGCTGAAG |
| | CCAGTTACCTTCGGAAAAAGAGTTGGTAGCTCTTGATCCGGCAA |
| | ACAAACCACCGCTGGTAGCGGTGGTTTTTTTGTTTGCAAGCAGC |
| | AGATTACGCGCAGAAAAAAAGGATCTCAAGAAGATCCTTTGATC |
| | TTTTCTACGTCAGTCCTGCTCCTCGGCCACGAAGTGCACGCAGT |
| | TGCCGGCCGGGTCGCGCAGGGCGAACTCCCGCCCCCACGGCTGC |
| | TCGCCGATCTCGGTCATGGCCGGCCCGGAGGCGTCCCGGAAGTT |
| | CGTGGACACGACCTCCGACCACTCGGCGTACAGCTCGTCCAGGC |
| dbDNA (HDRT | gcgtataatggactattgtgtgctgatatgtacaCCTgAGGACA |
| variant 1) | CAGGTACAGGACTCAGCCAGCTGCGCTGCCCTCCTCTCGCCCCC |
| | GAGTGCCCTTGCTGTGCCGCCGGAACTCTGCCCTCTAACGCTGC |
| | CGTCTCTCTCCTGAGTCCGGACCACTTTGAGCTCTACTGGCTTC |
| | TGCGCCGCCTCTGGCCCACTGTTTCCCCTTCCCAGGCAGGTCCT |
| | GCTTTCTCTGACCTGCATTCTCTCCCCTGGGCCTGTGCCGCTTT |
| | CTGTCTGCAGCTTGTGGCCTGGGTCACCTCTACGGCTGGCCCAG |
| | ATCCTTCCCTGCCGCCTCCTTCAGGTTCCGTCTTCCTCCACTCC |
| | CTCTTCCCCTTGCTCTCTGCTGTGTTGCTGCCCAAGGATGCTCT |
| | TTCCGGAGCACTTCCTTCTCGGCGCTGCACCACGTGATGTCCTC |
| | TGAGCGGATCCTCCCCGTGTCTGGGTCCTCTCCGGGCATCTCTC |
| | CTCCCTCACCCAACCCCATGCCGTCTTCACTCGCTGGGTTCCCT |
| | TTTCCTTCTCCTTCTGGGGCCTGTGCCATCTCTCGTTGATATCT |
| | CGACTAGTTATTAATAGTAATCAATTACGGGGTCATTAGTTCAT |
| | AGCCCATATATGGAGTTCCGCGTTACATAACTTACGGTAAATGG |
| | CCCGCCTGGCTGACCGCCCAACGACCCCCGCCCATTGACGTCAA |
| | TAATGACGTATGTTCCCATAGTAACGCCAATAGGGACTTTCCAT |
| | TGACGTCAATGGGTGGAGTATTTACGGTAAACTGCCCACTTGGC |
| | AGTACATCAAGTGTATCATATGCCAAGTACGCCCCCTATTGACG |
| | TCAATGACGGTAAATGGCCCGCCTGGCATTATGCCCAGTACATG |
| | ACCTTATGGGACTTTCCTACTTGGCAGTACATCTACGTATTAGT |
| | CATCGCTATTACCATGGTCGAGGTGAGCCCCACGTTCTGCTTCA |
| | CTCTCCCCATCTCCCCCCCCTCCCCACCCCCAATTTTGTATTTA |
| | TTTATTTTTTAATTATTTTGTGCAGCGATGGGGGCGGGGGGGGG |
| | GGGGGGGCGCGCGCCAGGCGGGGCGGGGCGGGGCGAGGGGCGGG |
| | GCGGGGCGAGGCGGAGAGGTGCGGCGGCAGCCAATCAGAGCGGC |
| | GCGCTCCGAAAGTTTCCTTTTATGGCGAGGCGGCGGCGGCGGCG |
| | GCCCTATAAAAAGCGAAGCGCGCGGCGGGCGGGGAGTCGCTGCG |
| | ACGCTGCCTTCGCCCCGTGCCCCGCTCCGCCGCCGCCTCGCGCC |
| | GCCCGCCCCGGCTCTGACTGACCGCGTTACTCCCACAGGTGAGC |
| | GGGCGGGACGGCCCTTCTCCTCCGGGCTGTAATTAGCGCTTGGT |
| | TTAATGACGGCTTGTTTCTTTTCTGTGGCTGCGTGAAAGCCTTG |
| | AGGGGCTCCGGGAGGGCCCTTTGTGCGGGGGGAGCGGCTCGGGG |
| | GGTGCGTGCGTGTGTGTGTGCGTGGGGAGCGCCGCGTGCGGCTC |
| | CGCGCTGCCCGGCGGCTGTGAGCGCTGCGGGCGCGGCGCGGGGC |
| | TTTGTGCGCTCCGCAGTGTGCGCGAGGGGAGCGCGGCCGGGGGC |
| | GGTGCCCCGCGGTGCGGGGGGGGCTGCGAGGGGAACAAAGGCTG |
| | CGTGCGGGGTGTGTGCGTGGGGGGGTGAGCAGGGGGTGTGGGCG |
| | CGTCGGTCGGGCTGCAACCCCCCCTGCACCCCCCTCCCCGAGTT |
| | GCTGAGCACGGCCCGGCTTCGGGTGCGGGGCTCCGTACGGGGCG |
| | TGGCGCGGGGCTCGCCGTGCCGGGCGGGGGGTGGCGGCAGGTGG |
| | GGGTGCCGGGCGGGGCGGGGCCGCCTCGGGCCGGGGAGGGCTCG |
| | GGGGAGGGGCGCGGCGGCCCCCGGAGCGCCGGCGGCTGTCGAGG |
| | CGCGGCGAGCCGCAGCCATTGCCTTTTATGGTAATCGTGCGAGA |
| | GGGCGCAGGGACTTCCTTTGTCCCAAATCTGTGCGGAGCCGAAA |
| | TCTGGGAGGCGCCGCCGCACCCCCTCTAGCGGGCGCGGGGCGAA |
| | GCGGTGCGGCGCCGGCAGGAAGGAAATGGGGGGGGAGGGCCTTC |
| | GTGCGTCGCCGCGCCGCCGTCCCCTTCTCCCTCTCCAGCCTCGG |
| | GGCTGTCCGCGGGGGGACGGCTGCCTTCGGGGGGGACGGGGCAG |
| | GGCGGGGTTCGGCTTCTGGCGTGTGACCGGCGGCTCTAGAGCCT |
| | CTGCTAACCATGTTCATGCCTTCTTCTTTTTCCTACAGCTCCTG |
| | GGCAACGTGCTGGTTATTGTGCTGTCTCATCATTTTGGCAAAGA |
| | ATTAATTCGGATCCACCATGGTGAGCAAGGGCGAGGAGCTGTTC |
| | ACCGGGGTGGTGCCCATCCTGGTCGAGCTGGACGGCGACGTAAA |
| | CGGCCACAAGTTCAGCGTGTCCGGCGAGGGCGAGGGCGATGCCA |
| | CCTACGGCAAGCTGACCCTGAAGTTCATCTGCACCACCGGCAAG |
| | CTGCCCGTGCCCTGGCCCACCCTCGTGACCACCCTGACCTACGG |
| | CGTGCAGTGCTTCAGCCGCTACCCCGACCACATGAAGCAGCACG |
| | ACTTCTTCAAGTCCGCCATGCCCGAAGGCTACGTCCAGGAGCGC |
| | ACCATCTTCTTCAAGGACGACGGCAACTACAAGACCCGCGCCGA |
| | GGTGAAGTTCGAGGGCGACACCCTGGTGAACCGCATCGAGCTGA |
| | AGGGCATCGACTTCAAGGAGGACGGCAACATCCTGGGGCACAAG |
| | CTGGAGTACAACTACAACAGCCACAACGTCTATATCATGGCCGA |
| | CAAGCAGAAGAACGGCATCAAGGTGAACTTCAAGATCCGCCACA |
| | ACATCGAGGACGGCAGCGTGCAGCTCGCCGACCACTACCAGCAG |
| | AACACCCCCATCGGCGACGGCCCCGTGCTGCTGCCCGACAACCA |
| | CTACCTGAGCACCCAGTCCGCCCTGAGCAAAGACCCCAACGAGA |
| | AGCGCGATCACATGGTCCTGCTGGAGTTCGTGACCGCCGCCGGG |
| | ATCACTCTCGGCATGGACGAGCTGTACAAGTAACTGTGCCTTCT |
| | AGTTGCCAGCCATCTGTTGTTTGCCCCTCCCCCGTGCCTTCCTT |
| | GACCCTGGAAGGTGCCACTCCCACTGTCCTTTCCTAATAAAATG |
| | AGGAAATTGCATCGCATTGTCTGAGTAGGTGTCATTCTATTCTG |
| | GGGGGTGGGGTGGGGCAGGACAGCAAGGGGGAGGATTGGGAAGA |
| | CAATAGCAGGCATGCTGGGGATGCGGTGGGCTCTATGGCGACGG |
| | ATGTCTCCCTTGCGTCCCGCCTCCCCTTCTTGTAGGCCTGCATC |
| | ATCACCGTTTTTCTGGACAACCCCAAAGTACCCCGTCTCCCTGG |
| | CTTTAGCCACCTCTCCATCCTCTTGCTTTCTTTGCCTGGACACC |
| | CCGTTCTCCTGTGGATTCGGGTCACCTCTCACTCCTTTCATTTG |
| | GGCAGCTCCCCTACCCCCCTTACCTCTCTAGTCTGTGCTAGCTC |
| | TTCCAGCCCCCTGTCATGGCATCTTCCAGGGGTCCGAGAGCTCA |
| | GCTAGTCTTCTTCCTCCAACCCGGGCCCCTATGTCCACTTCAGG |
| | ACAGCATGTTTGCTGCCTCCAGGGATCCTGTGTCCCCGAGCTGG |
| | GACCACCTTATATTCCCAGGGCCGGTTAATGTGGCTCTGGTTCT |
| | GGGTACTTTTATCTGTCCCCTCCACCCCACAGTGGGGCCACTAG |
| | GGACAGGATTGGTGACAGAAAAGCCCCATCCTTAGGCCTCCTCC |
| | TTCCTAGTCTCTCGAGGAGGTGAGATTGTGTTCGGCATGCCtca |
| | caGGCagatctatcagcacacaatagtccattatacgc |
| dbDNA with | gcgtataatggactattgtgtgctgatatgtacaCCTgAGGACA |
| flanking gRNA | CAGGTACAGGACTCAGCCAGCTGCGCTGCCCTCCTCTCGCCCCC |
| (HDRT variant 2) | GAGTGCCCTTGCTGTGCCGCCGGAACTCTGCCCTCTAACGCTGC |
| | CGTCTCTCTCCTGAGTCCGGACCACTTTGAGCTCTACTGGCTTC |
| | TGCGCCGCCTCTGGCCCACTGTTTCCCCTTCCCAGGCAGGTCCT |
| | GCTTTCTCTGACCTGCATTCTCTCCCCTGGGCCTGTGCCGCTTT |
| | CTGTCTGCAGCTTGTGGCCTGGGTCACCTCTACGGCTGGCCCAG |
| | ATCCTTCCCTGCCGCCTCCTTCAGGTTCCGTCTTCCTCCACTCC |
| | CTCTTCCCCTTGCTCTCTGCTGTGTTGCTGCCCAAGGATGCTCT |
| | TTCCGGAGCACTTCCTTCTCGGCGCTGCACCACGTGATGTCCTC |
| | TGAGCGGATCCTCCCCGTGTCTGGGTCCTCTCCGGGCATCTCTC |
| | CTCCCTCACCCAACCCCATGCCGTCTTCACTCGCTGGGTTCCCT |
| | TTTCCTTCTCCTTCTGGGGCCTGTGCCATCTCTCGTTGATATCT |
| | CGACTAGTTATTAATAGTAATCAATTACGGGGTCATTAGTTCAT |
| | AGCCCATATATGGAGTTCCGCGTTACATAACTTACGGTAAATGG |
| | CCCGCCTGGCTGACCGCCCAACGACCCCCGCCCATTGACGTCAA |
| | TAATGACGTATGTTCCCATAGTAACGCCAATAGGGACTTTCCAT |
| | TGACGTCAATGGGTGGAGTATTTACGGTAAACTGCCCACTTGGC |
| | AGTACATCAAGTGTATCATATGCCAAGTACGCCCCCTATTGACG |
| | TCAATGACGGTAAATGGCCCGCCTGGCATTATGCCCAGTACATG |
| | ACCTTATGGGACTTTCCTACTTGGCAGTACATCTACGTATTAGT |
| | CATCGCTATTACCATGGTCGAGGTGAGCCCCACGTTCTGCTTCA |
| | CTCTCCCCATCTCCCCCCCCTCCCCACCCCCAATTTTGTATTTA |
| | TTTATTTTTTAATTATTTTGTGCAGCGATGGGGGCGGGGGGGGG |
| | GGGGGGGCGCGCGCCAGGCGGGGGGGGGCGGGGCGAGGGGGGGG |
| | GCGGGGCGAGGCGGAGAGGTGCGGCGGCAGCCAATCAGAGCGGC |
| | GCGCTCCGAAAGTTTCCTTTTATGGCGAGGCGGCGGCGGCGGCG |
| | GCCCTATAAAAAGCGAAGCGCGCGGCGGGCGGGGAGTCGCTGCG |
| | ACGCTGCCTTCGCCCCGTGCCCCGCTCCGCCGCCGCCTCGCGCC |
| | GCCCGCCCCGGCTCTGACTGACCGCGTTACTCCCACAGGTGAGC |
| | GGGCGGGACGGCCCTTCTCCTCCGGGCTGTAATTAGCGCTTGGT |
| | TTAATGACGGCTTGTTTCTTTTCTGTGGCTGCGTGAAAGCCTTG |
| | AGGGGCTCCGGGAGGGCCCTTTGTGCGGGGGGAGCGGCTCGGGG |
| | GGTGCGTGCGTGTGTGTGTGCGTGGGGAGCGCCGCGTGCGGCTC |
| | CGCGCTGCCCGGCGGCTGTGAGCGCTGCGGGCGCGGCGCGGGGC |
| | TTTGTGCGCTCCGCAGTGTGCGCGAGGGGAGCGCGGCCGGGGGC |
| | GGTGCCCCGCGGTGCGGGGGGGGCTGCGAGGGGAACAAAGGCTG |
| | CGTGCGGGGTGTGTGCGTGGGGGGGTGAGCAGGGGGTGTGGGCG |
| | CGTCGGTCGGGCTGCAACCCCCCCTGCACCCCCCTCCCCGAGTT |
| | GCTGAGCACGGCCCGGCTTCGGGTGCGGGGCTCCGTACGGGGCG |
| | TGGCGCGGGGCTCGCCGTGCCGGGCGGGGGGTGGCGGCAGGTGG |
| | GGGTGCCGGGCGGGGCGGGGCCGCCTCGGGCCGGGGAGGGCTCG |
| | GGGGAGGGGCGCGGCGGCCCCCGGAGCGCCGGCGGCTGTCGAGG |
| | CGCGGCGAGCCGCAGCCATTGCCTTTTATGGTAATCGTGCGAGA |
| | GGGCGCAGGGACTTCCTTTGTCCCAAATCTGTGCGGAGCCGAAA |
| | TCTGGGAGGCGCCGCCGCACCCCCTCTAGCGGGCGCGGGGCGAA |
| | GCGGTGCGGCGCCGGCAGGAAGGAAATGGGCGGGGAGGGCCTTC |
| | GTGCGTCGCCGCGCCGCCGTCCCCTTCTCCCTCTCCAGCCTCGG |
| | GGCTGTCCGCGGGGGGACGGCTGCCTTCGGGGGGGACGGGGCAG |
| | GGCGGGGTTCGGCTTCTGGCGTGTGACCGGCGGCTCTAGAGCCT |
| | CTGCTAACCATGTTCATGCCTTCTTCTTTTTCCTACAGCTCCTG |
| | GGCAACGTGCTGGTTATTGTGCTGTCTCATCATTTTGGCAAAGA |
| | ATTAATTCGGATCCACCATGGTGAGCAAGGGCGAGGAGCTGTTC |
| | ACCGGGGTGGTGCCCATCCTGGTCGAGCTGGACGGCGACGTAAA |
| | CGGCCACAAGTTCAGCGTGTCCGGCGAGGGCGAGGGCGATGCCA |
| | CCTACGGCAAGCTGACCCTGAAGTTCATCTGCACCACCGGCAAG |
| | CTGCCCGTGCCCTGGCCCACCCTCGTGACCACCCTGACCTACGG |
| | CGTGCAGTGCTTCAGCCGCTACCCCGACCACATGAAGCAGCACG |
| | ACTTCTTCAAGTCCGCCATGCCCGAAGGCTACGTCCAGGAGCGC |
| | ACCATCTTCTTCAAGGACGACGGCAACTACAAGACCCGCGCCGA |
| | GGTGAAGTTCGAGGGCGACACCCTGGTGAACCGCATCGAGCTGA |
| | AGGGCATCGACTTCAAGGAGGACGGCAACATCCTGGGGCACAAG |
| | CTGGAGTACAACTACAACAGCCACAACGTCTATATCATGGCCGA |
| | CAAGCAGAAGAACGGCATCAAGGTGAACTTCAAGATCCGCCACA |
| | ACATCGAGGACGGCAGCGTGCAGCTCGCCGACCACTACCAGCAG |
| | AACACCCCCATCGGCGACGGCCCCGTGCTGCTGCCCGACAACCA |
| | CTACCTGAGCACCCAGTCCGCCCTGAGCAAAGACCCCAACGAGA |
| | AGCGCGATCACATGGTCCTGCTGGAGTTCGTGACCGCCGCCGGG |
| | ATCACTCTCGGCATGGACGAGCTGTACAAGTAACTGTGCCTTCT |
| | AGTTGCCAGCCATCTGTTGTTTGCCCCTCCCCCGTGCCTTCCTT |
| | GACCCTGGAAGGTGCCACTCCCACTGTCCTTTCCTAATAAAATG |
| | AGGAAATTGCATCGCATTGTCTGAGTAGGTGTCATTCTATTCTG |
| | GGGGGTGGGGTGGGGCAGGACAGCAAGGGGGAGGATTGGGAAGA |
| | CAATAGCAGGCATGCTGGGGATGCGGTGGGCTCTATGGCGACGG |
| | ATGTCTCCCTTGCGTCCCGCCTCCCCTTCTTGTAGGCCTGCATC |
| | ATCACCGTTTTTCTGGACAACCCCAAAGTACCCCGTCTCCCTGG |
| | CTTTAGCCACCTCTCCATCCTCTTGCTTTCTTTGCCTGGACACC |
| | CCGTTCTCCTGTGGATTCGGGTCACCTCTCACTCCTTTCATTTG |
| | GGCAGCTCCCCTACCCCCCTTACCTCTCTAGTCTGTGCTAGCTC |
| | TTCCAGCCCCCTGTCATGGCATCTTCCAGGGGTCCGAGAGCTCA |
| | GCTAGTCTTCTTCCTCCAACCCGGGCCCCTATGTCCACTTCAGG |
| | ACAGCATGTTTGCTGCCTCCAGGGATCCTGTGTCCCCGAGCTGG |
| | GACCACCTTATATTCCCAGGGCCGGTTAATGTGGCTCTGGTTCT |
| | GGGTACTTTTATCTGTCCCCTCCACCCCACAGTGGGGCCACTAG |
| | GGACAGGATTGGTGACAGAAAAGCCCCATCCTTAGGCCTCCTCC |
| | TTCCTAGTCTCTCGAGTTTCTTAGGATGGCCTTCTCCGACGTCG |
| | ACGAGGTGAGATTGTGTTCGGCATGCCtcacaGGCagatctatc |
| | agcacacaatagtccattatacgc |

Modification abbreviations:
m: 2′-O-methoxy modification
*: phosphorothioate modification

### C. Example 3

This example demonstrates improved cell viability and editing efficiency with cells treated with polynucleotides as described herein.

Briefly, human Pan T-cells were isolated from Leukopaks (StemCell Technology) using EasySep Direct Human T cell Isolation Kit (StemCell Technology Catalog #19661) and cryopreserved using CryoStor CS10 (StemCell Technology Catalog #07930). The cells were thawed and activated with ImmunoCult Human CD3/CD28 T Cell Activator (StemCell Technology Catalog #10991) and cultivated in ImmunoCult-XF T Cell Expansion Medium (StemCell Technology, Catalog #10981) supplemented with IL2 (StemCell Technlogy Catalog #78036.3) at 37° C. in a 5% CO2 environment, and transfected after approximately 72 hours with RNPs, consisting of artSTAR1.0 protein and synthetic dual gRNA comprising a modulator sequence and TRAC targeter sequence, and Miniplasmids or ldsDNA with closed ends. Sequences are shown in Table 8 below. artSTAR1.0 protein, which contained NLS sequences at the N-terminus, was expressed in E. coli and purified by fast protein liquid chromatography (FPLC). Different amounts of the substrates were used for the transections: 5, 10 and 25pmol nuclease; 4:1 and 2:1 gRNA:nuclease ratio; 50, 100 and 200pmol ssODN; and 0.1, 0.3 and 0.5pmol HDR template. RNP complexes were prepared by incubating artSTAR1.0 protein with chemically synthesized gRNA for 10 minutes at room temperature. The RNPs were mixed with ssODNS and with HDR templates. The obtained transfection substrates were mixed with 1 000,000 Pan T-cells resuspended in nucleofection buffer P3 (Lonza) in a final volume of 20 μL. Electroporation was carried out on a 4D-Nucleofector (Lonza) using program EH-115. Following electroporation, the cells were cultured for up to 14 days and cells were harvested for different assays at different timepoints.

Flowcytometry was used to determine the cell population expressing the CD19-CAR from the integrated HDR template. Antibody staining of the cells was used to stain the expressed protein. Briefly, 1,000,000 cells/ml were harvested and washed with Cell Staining Buffer (Biolegend, catalog #420201), incubated with a fluorophore tagged antibody against the protein of interest, washed with Cell Staining Buffer (Biolegend, catalog #420201), resuspended in 1x PBS and analyzed by Flow cytometry. The data were analyzed using Flowjo, gated for viable, single cells and the positive cell population expressing the fluorescence protein or containing the stained protein.

For the viability analysis 25 ul of staining dye were mixed with 25 ul of cells and measured with the Celleca instrument.

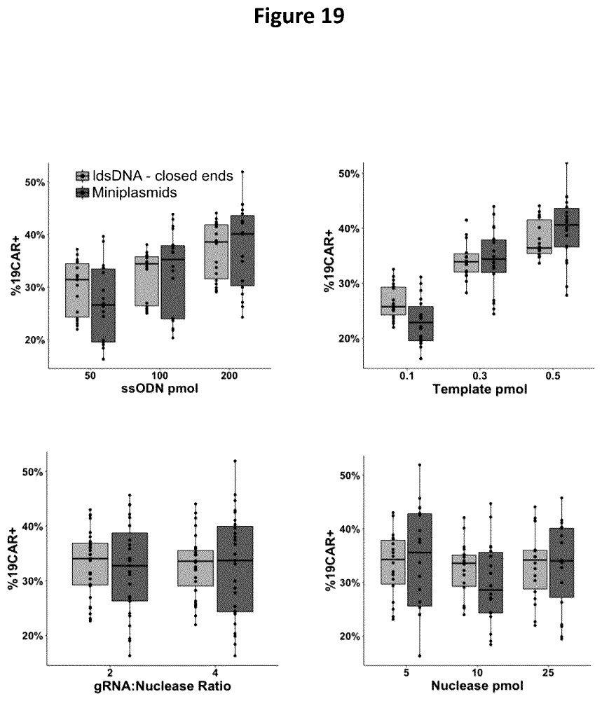

The data shown in FIG. 19 are a summary of all tested conditions (ssODN amounts, HDR template amounts, gRNA:nuclease ratios and Nuclease amounts) in boxplot diagrams. Overall, ldsDNA with closed ends as HDR template achieved similar results for CAR expression in Pan T-cells in comparison to the use of Miniplasmid as HDR templates. In addition, the variability was reduced for the ldsDNA with closed ends. In addition, ssODN and HDR template amounts might be the determining factors to achieve an increased cell population with CAR expression.

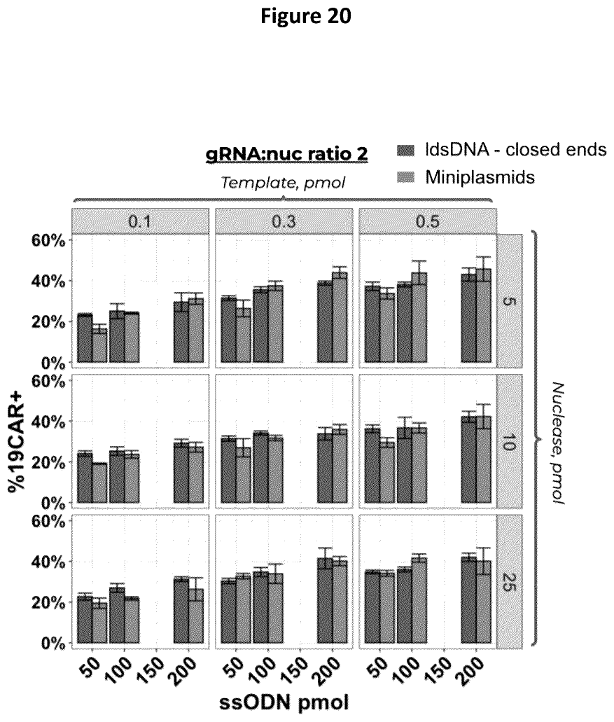

The data shown in FIG. 20 represents the conditions for the gRNA:nuclease ratio 2:1 in detail for the CAR expression readout. An increased CAR positive cell population was observed by increased amounts of ssODN under both tested conditions, HDR template and nuclease amount. In addition, the increase of HDR templates increased the CAR expressing cell population further.

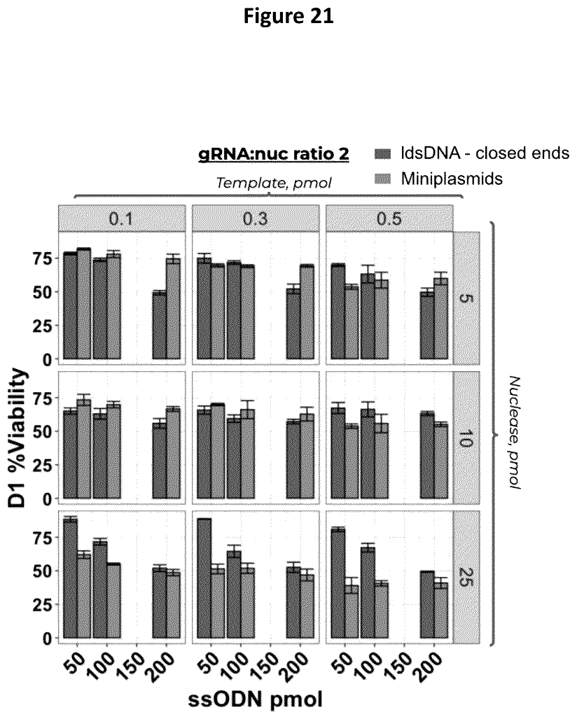

The data shown in FIG. 21 represents the conditions for the gRNA:nuclease ratio 2:1 in detail for the viability readout. There is a tendency of lower viability with increased amount of ssODNs and increased HDR template amount. The viability of the cells with ldsDNA with closed ends with 0.5pmol HDR template or 25pmol Nuclease was higher than for cells transfected with the Miniplasmids.

TABLE 8
exemplary sequences

| Name | Sequence |
|---|---|
| Modulator | mU*A*AUUCCUACUC |
| Targeter-gTRAC043 | UUGUAGGU GAGUCUCUCAGCUGGUACAC*mG |
| ldsDNA with | gcgtataatggactattgtgtgctgatatgtacaCCTgAGGACA |
| closed ends | CAGGTACAGGACTCAGCCCTCAGCAATGCCAACATACCATAAAC |
| | CTCCCATTCTGCTAATGCCCAGCCTAAGTTGGGGAGACCACTCC |
| | AGATTCCAAGATGTACAGTTTGCTTTGCTGGGCCTTTTTCCCAT |
| | GCCTGCCTTTACTCTGCCAGAGTTATATTGCTGGGGTTTTGAAG |
| | AAGATCCTATTAAATAAAAGAATAAGCAGTATTATTAAGTAGCC |
| | CTGCATTTCAGGTTTCCTTGAGTGGCAGGCCAGGCCTGGCCGTG |
| | AACGTTCACTGAAATCATGGCCTCTTGGCCAAGATTGATAGCTT |
| | GTGCCTGTCCCTGAGTCCCAGTCCATCACGAGCAGCTGGTTTCT |
| | AAGATGCTATTTCCCGTATAAAGCATGAGACCGTGACTTGCCAG |
| | CCCCACAGAGCCCCGCCCTTGTCCATCACTGGCATCTGGACTCC |
| | AGCCTGGGTTGGGGCAAAGAGGGAAATGAGATCATGTCCTAACC |
| | CTGATCCTCTTGTCCCACAGATATCCAGAACCCTGACCCTGCCG |
| | TGGGCAGCGGCGCTACTAACTTCAGCCTGCTGAAGCAGGCTGGC |
| | GACGTGGAGGAGAACCCTGGACCTATGGCTCTCCCAGTGACTGC |
| | CCTACTGCTTCCCCTAGCGCTTCTCCTGCATGCAGAGGTGAAGC |
| | TGCAGCAGTCTGGGGCTGAGCTGGTGAGGCCTGGGTCCTCAGTG |
| | AAGATTTCCTGCAAGGCTTCTGGCTATGCATTCAGTAGCTACTG |
| | GATGAACTGGGTGAAGCAGAGGCCTGGACAGGGTCTTGAGTGGA |
| | TTGGACAGATTTATCCTGGAGATGGTGATACTAACTACAATGGA |
| | AAGTTCAAGGGTCAAGCCACACTGACTGCAGACAAATCCTCCAG |
| | CACAGCCTACATGCAGCTCAGCGGCCTAACATCTGAGGACTCTG |
| | CGGTCTATTTCTGTGCAAGAAAGACCATTAGTTCGGTAGTAGAT |
| | TTCTACTTTGACTACTGGGGCCAAGGGACCACGGTCACCGTCTC |
| | CTCAGGTGGAGGTGGATCAGGTGGAGGTGGATCTGGTGGAGGTG |
| | GATCTGACATTGAGCTCACCCAGTCTCCAAAATTCATGTCCACA |
| | TCAGTAGGAGACAGGGTCAGCGTCACCTGCAAGGCCAGTCAGAA |
| | TGTGGGTACTAATGTAGCCTGGTATCAACAGAAACCAGGACAAT |
| | CTCCTAAACCACTGATTTACTCGGCAACCTACCGGAACAGTGGA |
| | GTCCCTGATCGCTTCACAGGCAGTGGATCTGGGACAGATTTCAC |
| | TCTCACCATCACTAACGTGCAGTCTAAAGACTTGGCAGACTATT |
| | TCTGTCAACAATATAACAGGTATCCGTACACGTCCGGAGGGGGG |
| | ACCAAGCTGGAGATCAAACGGGCGGCCGCAATTGAAGTTATGTA |
| | TCCTCCTACTTACCTAGACAATGAGAAGAGCAATGGAACCATTA |
| | TCCATGTGAAAGGGAAACACCTTTGTCCAAGTCCCCTATTTCCC |
| | GGACCTTCTAAGCCCTTTTGGGTGCTGGTGGTGGTTGGTGGAGT |
| | CCTGGCTTGCTATAGCTTGCTAGTAACAGTGGCCTTTATTATTT |
| | TCTGGGTGAGGAGTAAGAGGAGCAGGCTCCTGCACAGTGACTAC |
| | ATGAACATGACTCCCCGCCGCCCCGGGCCCACCCGCAAGCATTA |
| | CCAGCCCTATGCCCCACCACGCGACTTCGCAGCCTATCGCTCCA |
| | GAGTGAAGTTCAGCAGGAGCGCAGAGCCCCCCGCGTACCAGCAG |
| | GGCCAGAACCAGCTCTATAACGAGCTCAATCTAGGACGAAGAGA |
| | GGAGTACGATGTTTTGGACAAGAGACGTGGCCGGGACCCTGAGA |
| | TGGGGGGAAAGCCGAGAAGGAAGAACCCTCAGGAAGGCCTGTAC |
| | AATGAACTGCAGAAAGATAAGATGGCGGAGGCCTACAGTGAGAT |
| | TGGGATGAAAGGCGAGCGCCGGAGGGGCAAGGGGCACGATGGCC |
| | TTTACCAGGGTCTCAGTACAGCCACCAAGGACACCTACGACGCC |
| | CTTCACATGCAGGCCCTGCCCCCTCGCTAACGACTGTGCCTTCT |
| | AGTTGCCAGCCATCTGTTGTTTGCCCCTCCCCCGTGCCTTCCTT |
| | GACCCTGGAAGGTGCCACTCCCACTGTCCTTTCCTAATAAAATG |
| | AGGAAATTGCATCGCATTGTCTGAGTAGGTGTCATTCTATTCTG |
| | GGGGGTGGGGTGGGGCAGGACAGCAAGGGGGAGGATTGGGAAGA |
| | CAATAGCAGGCATGCTGGGGATACCAGCTGAGAGACTCTAATTC |
| | CAGTGACAAGTCTGTCTGCCTATTCACCGATTTTGATTCTCAAA |
| | CAAATGTGTCACAAAGTAAGGATTCTGATGTGTATATCACAGAC |
| | AAAACTGTGCTAGACATGAGGTCTATGGACTTCAAGAGCAACAG |
| | TGCTGTGGCCTGGAGCAACAAATCTGACTTTGCATGTGCAAACG |
| | CCTTCAACAACAGCATTATTCCAGAAGACACCTTCTTCCCCAGC |
| | CCAGGTAAGGGCAGCTTTGGTGCCTTCGCAGGCTGTTTCCTTGC |
| | TTCAGGAATGGCCAGGTTCTGCCCAGAGCTCTGGTCAATGATGT |
| | CTAAAACTCCTCTGATTGGTGGTCTCGGCCTTATCCATTGCCAC |
| | CAAAACCCTCTTTTTACTAAGAAACAGTGAGCCTTGTTCTGGCA |
| | GTCCAGAGAATGACACGGGAAAAAAGCAGATGAAGAGAAGGTGG |
| | CAGGAGAGGGCACCGTGTACCAGCTGAGAGACTCTAAACAGGTC |
| | GACGAGGTGAGATTGTGTTCGGCATGCCtcacaGGCagatctat |
| | cagcacacaatagtccattatacgc |
| Miniplasmid- | TGATCCCCCATGTTGTGCAAAAAAGCGGTTAGCTCCTTCGGTCC |
| PLA118 | TCCGATCGTTGTCAGAAGTAAGTTGGCCGCAGTGTTATCACTCA |
| | TGGTTATGGCAGCACTGCATAATTCTCTTACTGTCATGCCATCC |
| | GTAAGATGCTTTTCTGTGACTGGTGAGTACTCAACCAAGTCATT |
| | CTGAGAATAGTGTATGCGGCGACCGAGTTGCTCTTGCCCGGCGT |
| | CAATACGGGATAATACCGCGCCACATAGCAGAACTTTAAAAGTG |
| | CTCATCATTGGAAAACGTTCTTCGGGGCGAAAACTCTCAAGGAT |
| | CTTACCGCTGTTGAGATCCAGTTCGATGTAACCCACTCGTGCAC |
| | CCAACTGATCTTCAGCATCTTTTACTTTCACCAGCGTTTCTGGG |
| | TGAGCAAAAACAGGAAGGCAAAATGCCGCAAAAAAGGGAATAAG |
| | GGCGACACGGAAATGTTGAATACTCATACTCTTCCTTTTTCAAT |
| | ATTATTGAAGCATTTATCAGGGTTATTGTCTCATGAGCGGATAC |
| | ATAACGCGTCGCGAGGCCATATGGGTTAACTTTAGAGTCTCTCA |
| | GCTGGTACACGCCTCAGCAATGCCAACATACCATAAACCTCCCA |
| | TTCTGCTAATGCCCAGCCTAAGTTGGGGAGACCACTCCAGATTC |
| | CAAGATGTACAGTTTGCTTTGCTGGGCCTTTTTCCCATGCCTGC |
| | CTTTACTCTGCCAGAGTTATATTGCTGGGGTTTTGAAGAAGATC |
| | CTATTAAATAAAAGAATAAGCAGTATTATTAAGTAGCCCTGCAT |
| | TTCAGGTTTCCTTGAGTGGCAGGCCAGGCCTGGCCGTGAACGTT |
| | CACTGAAATCATGGCCTCTTGGCCAAGATTGATAGCTTGTGCCT |
| | GTCCCTGAGTCCCAGTCCATCACGAGCAGCTGGTTTCTAAGATG |
| | CTATTTCCCGTATAAAGCATGAGACCGTGACTTGCCAGCCCCAC |
| | AGAGCCCCGCCCTTGTCCATCACTGGCATCTGGACTCCAGCCTG |
| | GGTTGGGGCAAAGAGGGAAATGAGATCATGTCCTAACCCTGATC |
| | CTCTTGTCCCACAGATATCCAGAACCCTGACCCTGCCGTGGGCA |
| | GCGGCGCTACTAACTTCAGCCTGCTGAAGCAGGCTGGCGACGTG |
| | GAGGAGAACCCTGGACCTATGGCTCTCCCAGTGACTGCCCTACT |
| | GCTTCCCCTAGCGCTTCTCCTGCATGCAGAGGTGAAGCTGCAGC |
| | AGTCTGGGGCTGAGCTGGTGAGGCCTGGGTCCTCAGTGAAGATT |
| | TCCTGCAAGGCTTCTGGCTATGCATTCAGTAGCTACTGGATGAA |
| | CTGGGTGAAGCAGAGGCCTGGACAGGGTCTTGAGTGGATTGGAC |
| | AGATTTATCCTGGAGATGGTGATACTAACTACAATGGAAAGTTC |
| | AAGGGTCAAGCCACACTGACTGCAGACAAATCCTCCAGCACAGC |
| | CTACATGCAGCTCAGCGGCCTAACATCTGAGGACTCTGCGGTCT |
| | ATTTCTGTGCAAGAAAGACCATTAGTTCGGTAGTAGATTTCTAC |
| | TTTGACTACTGGGGCCAAGGGACCACGGTCACCGTCTCCTCAGG |
| | TGGAGGTGGATCAGGTGGAGGTGGATCTGGTGGAGGTGGATCTG |
| | ACATTGAGCTCACCCAGTCTCCAAAATTCATGTCCACATCAGTA |
| | GGAGACAGGGTCAGCGTCACCTGCAAGGCCAGTCAGAATGTGGG |
| | TACTAATGTAGCCTGGTATCAACAGAAACCAGGACAATCTCCTA |
| | AACCACTGATTTACTCGGCAACCTACCGGAACAGTGGAGTCCCT |
| | GATCGCTTCACAGGCAGTGGATCTGGGACAGATTTCACTCTCAC |
| | CATCACTAACGTGCAGTCTAAAGACTTGGCAGACTATTTCTGTC |
| | AACAATATAACAGGTATCCGTACACGTCCGGAGGGGGGACCAAG |
| | CTGGAGATCAAACGGGCGGCCGCAATTGAAGTTATGTATCCTCC |
| | TACTTACCTAGACAATGAGAAGAGCAATGGAACCATTATCCATG |
| | TGAAAGGGAAACACCTTTGTCCAAGTCCCCTATTTCCCGGACCT |
| | TCTAAGCCCTTTTGGGTGCTGGTGGTGGTTGGTGGAGTCCTGGC |
| | TTGCTATAGCTTGCTAGTAACAGTGGCCTTTATTATTTTCTGGG |
| | TGAGGAGTAAGAGGAGCAGGCTCCTGCACAGTGACTACATGAAC |
| | ATGACTCCCCGCCGCCCCGGGCCCACCCGCAAGCATTACCAGCC |
| | CTATGCCCCACCACGCGACTTCGCAGCCTATCGCTCCAGAGTGA |
| | AGTTCAGCAGGAGCGCAGAGCCCCCCGCGTACCAGCAGGGCCAG |
| | AACCAGCTCTATAACGAGCTCAATCTAGGACGAAGAGAGGAGTA |
| | CGATGTTTTGGACAAGAGACGTGGCCGGGACCCTGAGATGGGGG |
| | GAAAGCCGAGAAGGAAGAACCCTCAGGAAGGCCTGTACAATGAA |
| | CTGCAGAAAGATAAGATGGCGGAGGCCTACAGTGAGATTGGGAT |
| | GAAAGGCGAGCGCCGGAGGGGCAAGGGGCACGATGGCCTTTACC |
| | AGGGTCTCAGTACAGCCACCAAGGACACCTACGACGCCCTTCAC |
| | ATGCAGGCCCTGCCCCCTCGCTAACGACTGTGCCTTCTAGTTGC |
| | CAGCCATCTGTTGTTTGCCCCTCCCCCGTGCCTTCCTTGACCCT |
| | GGAAGGTGCCACTCCCACTGTCCTTTCCTAATAAAATGAGGAAA |
| | TTGCATCGCATTGTCTGAGTAGGTGTCATTCTATTCTGGGGGGT |
| | GGGGTGGGGCAGGACAGCAAGGGGGAGGATTGGGAAGACAATAG |
| | CAGGCATGCTGGGGATACCAGCTGAGAGACTCTAATTCCAGTGA |
| | CAAGTCTGTCTGCCTATTCACCGATTTTGATTCTCAAACAAATG |
| | TGTCACAAAGTAAGGATTCTGATGTGTATATCACAGACAAAACT |
| | GTGCTAGACATGAGGTCTATGGACTTCAAGAGCAACAGTGCTGT |
| | GGCCTGGAGCAACAAATCTGACTTTGCATGTGCAAACGCCTTCA |
| | ACAACAGCATTATTCCAGAAGACACCTTCTTCCCCAGCCCAGGT |
| | AAGGGCAGCTTTGGTGCCTTCGCAGGCTGTTTCCTTGCTTCAGG |
| | AATGGCCAGGTTCTGCCCAGAGCTCTGGTCAATGATGTCTAAAA |
| | CTCCTCTGATTGGTGGTCTCGGCCTTATCCATTGCCACCAAAAC |
| | CCTCTTTTTACTAAGAAACAGTGAGCCTTGTTCTGGCAGTCCAG |
| | AGAATGACACGGGAAAAAAGCAGATGAAGAGAAGGTGGCAGGAG |
| | AGGGCACCGTGTACCAGCTGAGAGACTCTAAACAGGTCGACTCT |
| | AGAGGATCCCCAGGAACCGTAAAAAGGCCGCGTTGCTGGCGTTT |
| | TTCCATAGGCTCCGCCCCCCTGACGAGCATCACAAAAATCGACG |
| | CTCAAGTCAGAGGTGGCGAAACCCGACAGGACTATAAAGATACC |
| | AGGCGTTTCCCCCTGGAAGCTCCCTCGTGCGCTCTCCTGTTCCG |
| | ACCCTGCCGCTTACCGGATACCTGTCCGCCTTTCTCCCTTCGGG |
| | AAGCGTGGCGCTTTCTCATAGCTCACGCTGTAGGTATCTCAGTT |
| | CGGTGTAGGTCGTTCGCTCCAAGCTGGGCTGTGTGCACGAACCC |
| | CCCGTTCAGCCCGACCGCTGCGCCTTATCCGGTAACTATCGTCT |
| | TGAGTCCAACCCGGTAAGACACGACTTATCGCCACTGGCAGCAG |
| | CCACTGGTAACAGGATTAGCAGAGCGAGGTATGTAGGCGGTGCT |
| | ACAGAGTTCTTGAAGTGGTGGCCTAACTACGGCTACACTAGAAG |
| | GACAGTATTTGGTATCTGCGCTCTGCTGAAGCCAGTTACCTTCG |
| | GAAAAAGAGTTGGTAGCTCTTGATCCGGCAAACAAACCACCGCT |
| | GGTAGCGGTGGTTTTTTTGTTTGCAAGCAGCAGATTACGCGCAG |
| | AAAAAAAGGATCTCAAGAAGATCCTTTGATCTTTTCTACGGGGT |
| | CTGACGCTCAGTGGAACGAAAACTCACGTTAAGGGATTTTGGTC |
| | ATGAGATTATCAAAAAGGATCTTCACCTAGATCCTTTTAAATTA |
| | AAAATGAAGTTTTAAATCAATCTAAAGTATATATGAGTAAACTT |
| | GGTCTGACAGTTACCAATGCTTAATCAGTGAGGCACCTATCTCA |
| | GCGATCTGTCTATTTCGTTCATCCATAGTTGCCTGACTCCCCGT |
| | CGTGTAGATAACTACGATACGGGAGGGCTTACCATCTGGCCCCA |
| | GTGCTGCAATGATACCGCGAGACCCACGCTCACCGGCTCCAGAT |
| | TTATCAGCAATAAACCAGCCAGCCGGAAGGGCCGAGCGCAGAAG |
| | TGGTCCTGCAACTTTATCCGCCTCCATCCAGTCTATTAATTGTT |
| | GCCGGGAAGCTAGAGTAAGTAGTTCGCCAGTTAATAGTTTGCGC |
| | AACGTTGTTGCCATTGCTACAGGCATCGTGGTGTCACGCTCGTC |
| | GTTTGGTATGGCTTCATTCAGCTCCGGTTCCCAACGATCAAGGC |
| | GAGTTACA |

### IX. EQUIVALENTS

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, or the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A composition comprising:
(A) a double-stranded DNA polynucleotide; and
(B) a polypeptide comprising a nuclear localization signal (NLS) bound to the polynucleotide.

2. The composition of claim 1, wherein the double-stranded DNA polynucleotide comprises a plasmid.

3. The composition of claim 1, wherein the double-stranded DNA polynucleotide comprises a linear double-stranded DNA polynucleotide with covalently closed ends.

4. The composition of any one of claims 1 through 3, wherein the double-stranded DNA polynucleotide comprises a donor template (D).

5. The composition of any one of claims 1 through 4, wherein the polypeptide comprises a nucleic acid-guided nuclease complex comprising:
(1) a nucleic acid-guided nuclease; and
(2) a guide nucleic acid (gNA).

6. The composition of claim 5, wherein the double-stranded DNA polynucleotide comprises a first PAM (P₁) recognized by a nucleic acid-guided nuclease and a first target nucleotide sequence (T₁) adjacent to but not within the donor template (D).

7. The composition of claim 6, wherein the double-stranded DNA polynucleotide further comprises a second PAM (P₂) recognized by a nucleic acid-guided nuclease and a second target nucleotide sequence (T₂) adjacent to but not within the donor template (D).

8. The composition of claim 6 or 7, wherein the first PAM and the first target nucleotide sequence are oriented 5′ P₁⁺T₁⁺D⁺ 3′.

9. The composition of claim 6 or 7, wherein the first PAM and the first target nucleotide sequence are oriented 5′ T₁⁻P₁⁻D⁺ 3′.

10. The composition of any one of claims 7 through 9, wherein the second PAM and the second target nucleotide sequence are oriented 5′ D⁺P₂⁺T₂⁺ 3′.

11. The composition of any one of claims 7 through 9, wherein the second suitable PAM and the second target nucleotide sequence are oriented 5′ D⁺T₂⁻P₂⁻ 3′.

12. The composition of claim 7, wherein the first and second PAM target nucleotide sequences are oriented 5′ T₁⁻P₁⁻D⁺P₂⁺T₂⁺ 3′.

13. The composition of claim 7, wherein the first and second PAM target nucleotide sequences are oriented 5′ P₁⁺T₁⁺D⁺T₂⁻P₂⁻ 3′.

14. The composition of claim 5, wherein the nucleic acid-guided nuclease comprises an engineered, non-naturally occurring nuclease.

15. The composition of any one of claims 5 through 14, wherein the nucleic acid-guided nuclease complex comprises a Class 1 or a Class 2 nucleic acid-guided nuclease complex.

16. The composition of claim 15, wherein the nucleic acid-guided nuclease complex comprises a Type II or a Type V nucleic acid-guided nuclease.

17. The composition of claim 16, wherein the nucleic acid-guided nuclease comprises a Type V-A, V-B, V-C, V-D, or V-E nucleic acid-guided nuclease.

18. The composition of claim 17, wherein the nucleic acid-guided nuclease comprises a Type V-A nucleic acid-guided nuclease.

19. The composition of claim 18, nucleic acid-guided nuclease comprises a MAD nuclease, an ART nuclease, or an ABW nucleic acid-guided nuclease.

20. The composition of claim 19, wherein the nucleic acid-guided nuclease comprises an amino acid sequence at least 80, 85, 90, 95, 99, or 100% identical to an amino acid sequence of a MAD, ART, or ABW nucleic acid-guided nuclease.

21. The composition of claim 19, wherein the nucleic acid-guided nuclease comprises MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, or MAD20.

22. The composition of claim 19, wherein the nucleic acid-guided nuclease comprises ART1, ART2, ART3, ART4, ART5, ART6, ART7, ART8, ART9, ART10, ART11, ART11*, ART12, ART13, ART14, ART15, ART16, ART17, ART18, ART19, ART20, ART21, ART22, ART23, ART24, ART25, ART26, ART27, ART28, ART29, ART30, ART31, ART32, ART33, ART34, or ART35.

23. The composition of claim 19, wherein the nucleic acid-guided nuclease comprises an amino acid sequence at least 80, 85, 90, 95, 99, or 100% identical to the amino acid sequence of MAD2, MAD7, ART2, ART11, or ART11*.

24. The composition of claim 19, wherein the nucleic acid-guided nuclease comprises an amino acid sequence that is at least 80, 85, 90, 95, 99, or 100% identical to the amino acid sequence of SEQ ID NO: 37.

25. The composition of any one of claims 6 through 24, wherein the first PAM (P₁) is a PAM recognized by a Type V nucleic acid-guided nuclease.

26. The composition of claim 25, wherein the PAM comprises a sequence of CTTN.

27. The composition of any one of claims 7 through 26, wherein the second PAM (P₂) is a PAM recognized by a Type V nucleic acid-guided nuclease.

28. The composition of any one of claims 5 through 27, wherein the gNA is an engineering, non-naturally occurring gNA.

29. The composition of any one of claims 5 through 28, wherein the gNA comprises a single polynucleotide.

30. The composition of any one of claims 5 through 28, wherein the gNA comprises a dual gNA comprising a targeter nucleic acid and a modulator nucleic acid, wherein the targeter nucleic acid and the modulator nucleic acid are separate polynucleotides.

31. The composition of claim 30, wherein the dual gNA is capable of binding to and activating a nucleic acid-guided nuclease, that, in a naturally occurring system, is activated by a single crRNA in the absence of a tracrRNA.

32. The composition of any one of claims 28 through 31, wherein the gNA comprises a heterologous spacer sequence that shares complementarity with a first target sequence in the double-stranded DNA polynucleotide and a second target sequence in a human genome.

33. The composition of any one of claims 28 through 31, wherein the gNA comprises a heterologous spacer sequence that does not share complementarity to a target sequence in a human genome.

34. The composition of any one of claims 1 through 31, wherein the nucleic acid-guided nuclease comprises at least 4 NLS.

35. The composition of claim 34, wherein the nucleic acid-guided nuclease comprises one N-terminal and three C-terminal NLS.

36. The composition of claim 34, wherein the nucleic acid-guided nuclease comprises five or more N-terminal NLS.

37. The composition of any one of claims 1 through 35, wherein the NLS comprise any one of SEQ ID NOs: 40-56.

38. The composition of claim 37, wherein the NLS comprise SEQ ID NOs: 40, 51, and 56.

39. The composition of any one of claims 1 through 38, further comprising at least one of
(1) a buffer;
(2) a RNP stabilizer.

40. The composition of claim 39, wherein the buffer is magnesium deficient.

41. The composition of claim 39 or 40, wherein the RNP stabilizer comprises a peptide, poly-L-glutamic acid (PGA), or a single-stranded oligodeoxynucleotide (ssODN).

42. A composition comprising a polynucleotide comprising:
(A) a donor template (D); and
(B) a first PAM (P₁) recognized by a Type V nucleic acid-guided nuclease and a first target nucleotide sequence (T₁) adjacent to but not within the donor template (D).

43. The composition of claim 42, wherein the polynucleotide comprises double-stranded DNA.

44. The composition of claim 42 or 43, wherein the polynucleotide comprises circular DNA.

45. The composition of any one of claims 42 through 44, wherein the polynucleotide is a plasmid.

46. The composition of any one of claims 42 through 45, wherein the polynucleotide further comprises a second PAM (P₂) recognized by a Type V nucleic acid-guided nuclease and a second target nucleotide sequence (T₂) adjacent to but not within the donor template (D).

47. The composition of any one of claims 42 through 46, wherein the first PAM and the first target nucleotide sequence are oriented 5′ P₁⁺T₁⁺D⁺ 3′.

48. The composition of any one of claims 42 through 46, wherein the first PAM and the first target nucleotide sequence are oriented 5′ T₁⁻P₁⁻D⁺ 3′.

49. The composition of any one of claims 46 through 48, wherein the second PAM and the first target nucleotide sequence are oriented 5′ D⁺P₂⁺T₂⁺ 3′.

50. The composition of any one of claims 46 through 48, wherein the second suitable PAM and the second target nucleotide sequence are oriented 5′ D⁺T₂⁻P₂⁻ 3′.

51. The composition of claim 46, wherein the first and second PAM target nucleotide sequences are oriented 5′ T₁⁻P₁⁻D⁺P₂⁺T₂⁺ 3′.

52. The composition of any one of claims 42 through 51, wherein the polynucleotide further comprises a selectable marker and/or a replication origin.

53. The composition of any one of claims 1 through 52, wherein the donor template comprises a first sequence encoding a first polypeptide comprising a first CAR or portion thereof.

54. The composition of claim 53, wherein the donor template comprises a second sequence encoding a second polypeptide comprising a second CAR or portion thereof.

55. The composition of claim 54, wherein the second sequence encoding a second polypeptide comprising a second CAR or portion thereof is different from the first sequence encoding a first polypeptide comprising a first CAR or portion thereof.

56. The composition of claim 54 or 55, wherein the first and second polypeptides are the same polypeptide.

57. The composition of claim 56, wherein the first and second polypeptides are linked by one or more amino acids.

58. The composition of claim 54 or 55, wherein the first and second polypeptides are separate polypeptides.

59. The composition of any one of claims 53 through 58, wherein the first and/or second CARs or portions thereof binds to a binding partner comprising B7H3, BCMA, GPRC5D, CD8, CD8a, CD19, CD20, CD22, CD28, 4-1BB, or CD3zeta or portion thereof.

60. The composition of any one of claims 53 through 59, wherein the first or second polypeptide comprise a polypeptide that is at least 60, 70, 75, 80, 85, 90, 95, 96, 97, 98, 99, 99.5, or 100% identical to any one of the amino acid sequences of SEQ ID NOs: 86-124.

61. A composition comprising a plurality of polynucleotides of any one of claims 42 through 60, wherein, for each integer x, the polynucleotide comprises:
(A) a donor template (D)ₓ;
(B) a first suitable PAM (P₁)ₓ and a first target nucleotide sequence (T₁)ₓ adjacent to but not within the donor template (D)ₓ; and
(C) a second suitable PAM (P₂)ₓ and a first target nucleotide sequence (T₂)ₓ adjacent to but not within the donor template (D)ₓ.

62. The composition of claim 61, wherein (D)ₓ comprises a polynucleotide encoding a polypeptide comprising a CAR or portion thereof that binds a binding partner comprising B7H3, BCMA, GPRC5D, CD8, CD8a, CD19, CD20, CD22, CD28, 4-1BB, or CD3zeta or a portion thereof.

63. The composition of claim 62, wherein (D)ₓ comprises a polynucleotide encoding a CAR or portion thereof that comprises a polypeptide at least 60, 70, 75, 80, 85, 90, 95, 96, 97, 98, 99, 99.5, or 100% identical to any one of the amino acid sequences of SEQ ID NOs: 86-124.

64. The composition of any one of claims 61 through 63, wherein the number of different integers x is at least 2, 3, 4, 5, 6, 7, 8, or 9 and/or no more than 10, 9, 8, 6, 5, 4, or 3.

65. The composition of claim 64, wherein the number of different integers x is 2-10.

66. The composition of claim 65, wherein the number of different integers x is 2-5.

67. A cell comprising a composition comprising of any one of the proceeding, a progeny of a cell comprising a composition of any one of the proceeding claims, or a progeny of a cell comprising one or more genetic modifications, wherein the one or more genetic modifications were generated after contacting the cell with a composition of any one of the proceeding claims.

68. The cell of claim 67, wherein the cell is a human cell.

69. The cell of claim 68, wherein the human cell is an immune cell or a stem cell.

70. The cell of claim 68, wherein the human cell is an immune cell comprising a neutrophil, eosinophil, basophil, mast cell, monocyte, macrophage, dendritic cell, natural killer cell, or a lymphocyte.

71. The cell of claim 68, wherein the human cell is a T cell.

72. The cell of claim 68, wherein the human cell is a stem cell that is a human pluripotent, multipotent stem cell, embryonic stem cell, induced pluripotent stem cell, hematopoietic stem cell, CD34+ cell.

73. The cell of claim 68, wherein the human cell is an induced pluripotent stem cell.

74. The cell of any one of claims 68 through 73, wherein the cell is a cell demonstrating reduced immunogenicity when placed in an allogeneic host.

75. The cell of claim 74, wherein the cell is non-immunogenic when placed in an allogeneic host.

76. A method for preparing a linearized polynucleotide comprising contacting a polynucleotide of any one of claims 42 through 66 with a nucleic acid-guided nuclease complex, wherein the nucleic acid-guided nuclease complexes binds to a target site on the polynucleotide and generates at least one strand break.

77. A method for engineering a genome of a cell comprising delivering to the cell a composition comprising:
(A) a polynucleotide of any one of claims 42 through 66; and
(B) a nucleic acid-guided nuclease system comprising
(1) a nucleic acid-guided nuclease, and
(2) a gNA.

78. A method of introducing a plurality of exogenous nucleic acids into the genome of a target cell comprising contacting the target cell with a composition comprising:
(A) a plurality of polynucleotides, wherein, for each integer x, the DNA polynucleotide comprises:
(1) a donor template (D)ₓ;
(2) a first suitable PAM (P₁)ₓ and a first target nucleotide sequence (T₁)ₓ adjacent to the 5′ end of (D)ₓ but not within (D)ₓ; and
(3) a second suitable PAM (P₂)ₓ and a first target nucleotide sequence (T₂)ₓ adjacent to the 3′ end of (D)ₓ but not within (D)ₓ; and
(4) a first homology arm (HA₁)ₓ between (P₁)ₓ(T₁)ₓ and (D)ₓ and a second homology arm (HA₂)ₓ between (P₂)ₓ(T₂)ₓ and (D)ₓ, where (HA₁)ₓ and (HA₂)ₓ are capable of initiating host cell mediated recombination of at least a portion of (D)ₓ at a target site (TS)ₓ selected from a plurality of target sites of the genome of the target cell; and
(B) for each target site (TS)ₓ, a plurality of nucleic acid-guided nuclease complexes (N)ₓ capable of cleaving at (TS)ₓ and at least one of (T₁)ₓ and (T₂)ₓ, wherein cleaving of (TS)ₓ and at least one of (T₁)ₓ and (T₂)ₓ results in homologous recombination of at least a portion of (D)ₓ at (TS)ₓ.

79. A method of introducing a plurality of exogenous nucleic acids into the genome of a target cell comprising contacting the target cell with a composition comprising:
(A) a plurality of polynucleotides, wherein, for each integer x, the DNA polynucleotide comprises:
(1) a donor template (D)ₓ, wherein at least one donor template comprises a polynucleotide encoding for a first CAR;
(2) a first suitable PAM (P₁)ₓ and a first target nucleotide sequence (T₁)ₓ adjacent to the 5′ end of (D)ₓ but not within (D)ₓ; and
(3) a second suitable PAM (P₂)ₓ and a first target nucleotide sequence (T₂)ₓ adjacent to the 3′ end of (D)ₓ but not within (D)ₓ; and
(4) a first homology arm (HA₁)ₓ between (P₁)ₓ(T₁)ₓ and (D)ₓ and a second homology arm (HA₂)ₓ between (P₂)ₓ(T₂)ₓ and (D)ₓ, where (HA₁)ₓ and (HA₂)ₓ are capable of initiating host cell mediated recombination of at least a portion of (D)ₓ at a target site (TS)ₓ selected from a plurality of target sites of the genome of the target cell; and
(B) for each target site (TS)ₓ, a plurality of nucleic acid-guided nuclease complexes (N)ₓ capable of cleaving at (TS)ₓ and at least one of (T₁)ₓ and (T₁)ₓ, wherein cleaving of (TS)ₓ and at least one of (T₁)ₓ and (T₁)ₓ results in homologous recombination of at least a portion of (D)ₓ at (TS)ₓ.

80. A composition comprising:
(A) a linear double-stranded DNA polynucleotide with covalently closed ends; and
(B) a polypeptide comprising a nuclear localization signal (NLS) bound to the polynucleotide.

81. The composition of claim 80, wherein the double-stranded DNA polynucleotide comprises a donor template (D).

82. The composition of claim 80 or 81, wherein the polypeptide comprises a nucleic acid-guided nuclease complex comprising:
(1) a nucleic acid-guided nuclease; and
(2) a guide nucleic acid (gNA).

83. The composition of claim 82, wherein the double-stranded DNA polynucleotide comprises a first PAM (P₁) recognized by a nucleic acid-guided nuclease and a first target nucleotide sequence (T₁) adjacent to but not within the donor template (D).

84. The composition of claim 83, wherein the double-stranded DNA polynucleotide further comprises a second PAM (P₂) recognized by a nucleic acid-guided nuclease and a second target nucleotide sequence (T₂) adjacent to but not within the donor template (D).

85. The composition of claim 83 or 84, wherein the first PAM and the first target nucleotide sequence are oriented 5′ P₁⁺T₁⁺D⁺ 3′.

86. The composition of claim 83 or 84, wherein the first PAM and the first target nucleotide sequence are oriented 5′ T₁⁻P₁⁻D⁺3′.

87. The composition of any one of claims 84 through 86, wherein the second PAM and the nucleotide sequence are oriented 5′ D⁺P₂⁺T₂⁺ 3′.

88. The composition of any one of claims 84 through 86, wherein the second suitable PAM and the second target nucleotide sequence are oriented 5′ D⁺T₂⁻P₂⁻⁰ 3′.

89. The composition of claim 84, wherein the first and second PAM target nucleotide sequences are oriented 5′ T₁⁻P₁⁻D⁺P₂⁺T₂⁺ 3′.

90. The composition of claim 84, wherein the first and second PAM target nucleotide sequences are oriented 5′ P₁⁺T₁⁺D⁺T₂⁻P₂⁻ 3′.

91. A composition comprising a polynucleotide comprising:
(A) a linear double stranded DNA donor template (D) with covalently closed ends;
(B) a first PAM (P₁) recognized by a Type V nucleic acid-guided nuclease and a first target nucleotide sequence (T₁) adjacent to but not within the donor template (D); and, optionally,
(C) a second PAM (P₂) recognized by a Type V nucleic-acid guided nuclease and a second target nucleotide sequence (T₂) adjacent to but not within the donor template (D).

92. The composition of claim 91, wherein the first PAM and the first target nucleotide sequence are oriented 5′ P₁⁺T₁⁺D⁺ 3′.

93. The composition of claim 91, wherein the first PAM and the first target nucleotide sequence are oriented 5′ T₁⁻P₁⁻D⁺ 3′.

94. The composition of claim 92 or 93, wherein the second PAM and the second target nucleotide sequence are oriented 5′ D⁺P₂⁺T₂⁺ 3′.

95. The composition of claim 92 or 93, wherein the second suitable PAM and the second target nucleotide sequence are oriented 5′ D⁺T₂⁻P₂⁻ 3′.

96. The composition of claim 91, wherein the first and second PAM target nucleotide sequences are oriented 5′ T₁⁻P₁⁻D⁺P₂⁺T₂⁺ 3′.

97. The composition of claim 91, wherein the first and second PAM target nucleotide sequences are oriented 5′ P₁⁺T₁⁺D⁺T₂⁻P₂⁻ 3′.

98. The composition of any one of claims 91 through 97, further comprising a polypeptide comprising a nuclear localization signal (NLS) bound to the polynucleotide.

99. A method comprising generating a linear double stranded DNA polynucleotide with covalently closed ends wherein the polynucleotide comprises:
(1) a linear double stranded DNA donor template (D) with covalently closed ends;
(2) a first PAM (P₁) recognized by a Type V nucleic acid-guided nuclease and a first target nucleotide sequence (T₁) adjacent to but not within the donor template (D); and, optionally,
(3) a second PAM (P₂) recognized by a Type V nucleic-acid guided nuclease and a second target nucleotide sequence (T₂) adjacent to but not within the donor template (D),
wherein the polynucleotide is generated from a circular double stranded DNA polynucleotide comprising protelomerase recognition sites flanking the donor template, the PAMs and the target nucleotide sequences and, optionally, comprises a polypeptide comprising a nuclear localization signal (NLS) bound to the polynucleotide.

100. A method comprising contacting a cell with a composition of any one of claims 80 through 98 or a polynucleotide generated by a method of claim 99.

101. A cell or a progeny thereof comprising a composition of any one of claims 80 through 98 or a polynucleotide generated by a method of claim 99.

102. The method of claim 100, wherein the editing efficiency as measured by the number of edits in a population of cells treated with the composition is at least 5, 10, 20, 25, 30, 40, 50, 60, 70, 80, 90, 100, 200, or 500% greater than that of a population of cells treated with a standard composition.
